(19) United States
(12) Patent Application Publication
(10) Pub. No.: US 2026/0256921 A1
(43) Pub. Date: Sep. 3, 2026

(54) BIFUNCTIONAL MOLECULES TO TARGET DEGRADATION OF TUMOR NECROSIS FACTOR

(71) Applicant: Biohaven Therapeutics Ltd., New Haven, CT (US)

(72) Inventors: Alexander BAYDEN, Hamden, CT (US); Briana BELTON-ROBINSON, Hamden, CT (US); Vladimir CORIC, Madison, CT (US); Gene M. DUBOWCHIK, Killingworth, CT (US); Lawrence G. IBEN, Bethany, CT (US); Wieslaw KAZMIERSKI, Raleigh, NC (US); Richard PRACITTO, Wallingford, CT (US); Matthew TODD, Spring House, PA (US); Christian M. VIDAL, Plantsville, CT (US)

(21) Appl. No.: 19/151,594

(22) PCT Filed: Feb. 6, 2024
(86) PCT No.: PCT/US2024/014588

§ 371 Date: Jul. 28, 2025

Related U.S. Application Data
(60) Provisional application No. 63/483,329, filed on Feb. 6, 2023.

(51) Int. Cl. A61K 47/54; A61K 47/55; C07H 15/26

(52) U.S. Cl. CPC A61K 47/545; A61K 47/55; C07H 15/26

(57) ABSTRACT

The present disclosure described a bifunctional Tumor Necrosis Factor-alpha (TNF-α) binding compound that includes a 1 or more Tumor Necrosis Factor-Alpha (TNF-α) Binding Moieties (TNFABM) that are capable of binding to TNF-α which (TNFABM) is covalently linked to 1 to 15 Asialoglycoprotein Receptors Binding Moieties (ASPGPRM) that binds to an asialoglycoprotein receptor (ASGPR) on an outer surface of a cell (e.g., a hepatocyte or other degrading cell, such as a cell in a subject) through 1 to 15 optional chemical moieties (LINKER) and 1 to 15 connectivity groups (CON) that are a bond or a chemical connecting moiety. Alternatively, the TNFABM can be bound via one or more LINKER or CON groups to a MATE reagent that specifically and covalently links the TNFABM to a TNF-α antibody. Further described are methods of making and using the bifunctional compounds of the present disclosure and methods for treating a patients having a disorder in which TNF-α is implicated, such as an autoimmune disorder or inflammatory disorder, e.g. rheumatoid arthritis, psoriatic arthritis, inflammatory bowel disease (IBD), Crohn's disease, ulcerative colitis, psoriasis, and noninfectious or autoimmune uveitis.

### CROSS REFERENCE TO RELATED APPLICATION

This application claims priority to U.S. Provisional Appl. No. 63/483,329, filed Feb. 6, 2023, which is hereby incorporated by reference in its entirety.

### BACKGROUND

Tumor Necrosis Factor-alpha (TNF-α) is a member of the TNF superfamily that is an adipokine and a cytokine. The TNF superfamily consists of transmembrane proteins having homologous TNF domain. As a cytokine, TNF is involved in immune system signaling. For example, macrophages and certain white blood cells release TNF-α when an infection is detected to trigger an inflammatory response by altering other cells of the immune system. As an adipokine, TNF-α is thus associated with obesity-induced type 2 diabetes by promoting insulin resistance.

Dysregulation of TNF-α production is implicated in a variety of human diseases including Alzheimer's disease, cancer, atherosclerosis, major depression (such as major depressive disorder (MDD)), sepsis, diabetes, and obesity, as well as autoimmune and inflammatory diseases or disorders, such as psoriasis and inflammatory bowel disease (IBD). Therapeutics that counteract TNF-α are used to treat various autoimmune and inflammatory diseases (such as rheumatoid arthritis, psoriatic arthritis, inflammatory bowel disease (IBD) (e.g. Crohn's disease and ulcerative colitis), psoriasis, noninfectious or autoimmune uveitis, etc.), and cancers that cause overproduction of TNF-α (such as, breast cancer, pancreatic cancer, ovarian cancer, endometrial cancer, oral cancer, gastric cancer, liver cancer, prostate cancer, bladder cancer, colorectal cancer, multiple myeloma, lymphoma, and leukemia).

Applicants disclose herein a different approach that includes bifunctional compounds that include a small molecule that is capable of binding directly to TNF-α covalently coupled, either by direct covalent bond, or through a linker, to moiety that targets the bifunctional molecule together with the TNF-α to which it is bound for degradation.

### SUMMARY

The disclosure provides a bifunctional Tumor Necrosis Factor-alpha (TNF-α) binding and/or degrading compound of formula I:

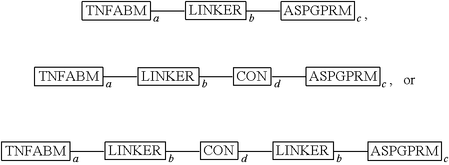

wherein,
[TNFABM] is a Tumor Necrosis Factor-Alpha (TNF-α) Binding Moiety that is capable of binding to TNF-α;
[ASPGPRM] is an Asialoglycoprotein Receptors Binding Moiety that binds to an asialoglycoprotein receptor (ASGPR) on an outer surface of a cell (e.g., a hepatocyte or other degrading cell, such as a cell in a subject);
[LINKER] is an optional chemical moiety that is covalently attached to one or more [TNFABM] groups and either one or more [CON] groups or one or more [ASGPRM] groups;
[CON] is an optional connecting group that is covalently attached to one or more [ASPGPRM] groups and one or more [LINKER] groups;
a is an integer from 1 to 15 (e.g. 1, 2, 3, 4, 5, 6, 7, 8, 9, 10, 11, 12, 13, 14, or 15);
b is an integer, independently selected at each occurrence from 0 to 15 (e.g. 0, 1, 2, 3, 4, 5, 6, 7, 8, 9, 10, 11, 12, 13, 14, or 15);
c is an integer of from 1 to 15 (e.g. 1, 2, 3, 4, 5, 6, 7, 8, 9, 10, 11, 12, 13, 14, or 15); and
d is an integer from 0 to 15 (e.g. 0, 1, 2, 3, 4, 5, 6, 7, 8, 9, 10, 11, 12, 13, 14, or 15, preferably 0, 1, 2, or 3); or a pharmaceutically acceptable salt thereof.

A bifunctional Tumor Necrosis Factor Alpha (TNF-α) binding compound of the formula II.

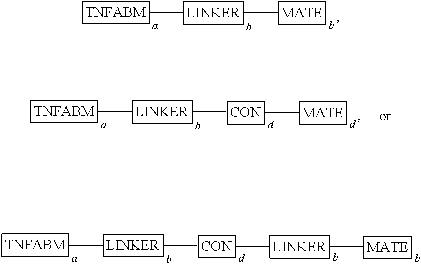

[TNFABM] is a Tumor Necrosis Factor Alpha (TNF-α) Binding Moiety that is capable of binding to TNF-α
[MATE] is Fc binding reagent comprising an Fc binding moiety [ABM] and a reactive group [RG] capable of covalently and specifically binding to a Lys in the Fc domain of an TNF-α antibody and releasing the [ABM];
[LINKER] is an optional chemical moiety that is covalently attached to one or more [TNFABM] groups and either one or more [CON] groups or a [MATE] groups;
[CON] is an optional chemical connecting moiety that is covalently attached to one or more [MATE] groups and one or more [LINKER] groups;
a is an integer from 1 to 15 (e.g. 1, 2, 3, 4, 5, 6, 7, 8, 9, 10, 11, 12, 13, 14, or 15);
b is an integer, independently selected at each occurrence from 0 to 15 (e.g. 0, 1, 2, 3, 4, 5, 6, 7, 8, 9, 10, 11, 12, 13, 14, or 15), preferably 0 or 1; and
d is an integer from 0 to 15 (e.g. 0, 1, 2, 3, 4, 5, 6, 7, 8, 9, 10, 11, 12, 13, 14, or 15, preferably 0, 1, 2, or 3); or a pharmaceutically acceptable salt thereof. In certain embodiments at least one of a is 1, 2 or 3; b is 1, 2 or 3; or d is 1, 2 or 3.

In certain embodiments the [RG] covalently and specifically binds to a Lys 246 or Lys 248 of an IgG1 heavy chain of the Fc domain, K251 or K253 of an IgG2 heavy chain, or K239 or K241 of an IgG4 heavy chain, of an TNF-α antibody. For example, the TNF-α etanercept (ENBREL), infliximab (REMICADE or its biosimilars, e.g., INFLECTRA, RENFLEXIS, AVOLSA), adalimumab (CYLTEZO or its biosimilars, e.g. HULIO), certolizumab pegol (CIMZIA), and golimumab (SIMPONI). These compounds can also be used in combination with a compound of Formula I.

### DETAILED DESCRIPTION

### Terminology

Prior to describing the disclosure in detail, the following terms may be helpful. Unless otherwise specified all terms have their ordinary meaning, accepted in the art of pharmaceutical formulations or methods of treating bacterial infections in patients.

Recitation of ranges of values are merely intended to serve as a shorthand method of referring individually to each separate value falling within the range, unless otherwise indicated, and each separate value is incorporated into the specification as if it were individually recited. The endpoints of all ranges are included within the range and independently combinable. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc.

The use of any and all examples, or language indicating an example (e.g., “such as”), is intended merely for illustration and does not pose a limitation on the claimed scope unless explicitly claimed. No language in the specification should be construed as indicating any non-claimed element as essential to the practice of the embodiments of the claims.

Groupings of alternative elements or embodiments of the present disclosure are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The symbols

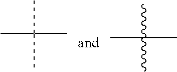

and are used to indicate a point of attachment to an indicated group. A dash “-” between two letters or atom symbols indicates a covalent bond. A dash that is not between two letters or symbols is used to indicate a point of attachment for a substituent. For example, —C(O)NH₂ is attached through the carbon of the keto C(O) group.

A [LINKER] or [CON] may be present in either orientation. For example, in [LINKER]

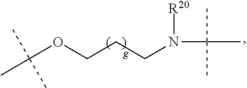

wherein [CON] is a bond, the nitrogen may be covalently bound to either the [TNFABM] or the [ASGPRBM].

The terms “a”, “an”, “the”, and similar referents used in the context of describing inventions of the present disclosure (especially in the context of the following claims) are to be construed to cover both the singular and the plural (for example, “one or more of” or “at least one”) of the grammatical object of the articles, unless otherwise indicated herein or clearly contradicted by context. By way of example, “an element” means one element or more than one element, unless otherwise indicated.

The term “about,” means approximately. When the term “about” is used in conjunction with a numerical range, it modifies that range by extending the boundaries above and below the numerical values set forth. Illustratively, the use of the term “about” indicates that values slightly outside the cited values, i.e., plus or minus 0.1% to 10%, which are also effective and safe are included in the value. For example, the phrase “about 200” includes plus or minus 10% of 200, or from 180 to 220, unless clearly contradicted by context. Numerical ranges recited herein by endpoints include all numbers and fractions subsumed within that range (e.g. 1 to 5 includes 1, 1.5, 2, 2.75, 3, 3.90, 4, and 5).

“Administration of” and “administering” mean giving, providing, applying, or dispensing the compound, for example, of the present disclosure, by any suitable route. In this disclosure suitable routes of administration include oral, intravenous, intramuscular, subcutaneous, inhaled, buccal, transmucosal, and intranasal administration. In the context of administering compounds in the form of a combination, such as a combination of a bifunctional compound of the disclosure and another active agent compound, the amount of each compound, when administered in combination may be different from that compound administered alone.

“Alkoxy” is an alkyl group as defined herein with the indicated number of carbon atoms covalently bound to the group it substitutes by an oxygen bridge (—O—). Examples of alkoxy groups include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Alkyl” is a branched or straight chain saturated aliphatic hydrocarbon group, having the specified number of carbon atoms, generally from 1 to 8 carbon atoms. The term C₁-C₈-alkyl as used herein indicates an alkyl group having from 1, 2, 3, 4, 5, 6, 7, or 8 carbon atoms. Other embodiments include alkyl groups having from 1 to 6 carbon atoms, 1 to 4 carbon atoms or 1 or 2 carbon atoms, e.g. C₁-C₆-alkyl, C₁-C₄-alkyl, and C₁-C₂-alkyl. Examples of alkyl groups include, but are not limited to, methyl, ethyl, n-propyl, isopropyl, n-butyl, 3-methylbutyl, t-butyl, n-pentyl, and sec-pentyl.

“Alkylene” is a bivalent saturated aliphatic radical having the indicated number of carbons, generally from 1 to 8 carbon atoms. The alkylene may be straight or branched.

“Alleviating a disease or disorder symptom,” in a subject means reducing the severity, the frequency, or both of one or more symptoms experienced by a patient.

The term “and/or” means “either or both” of the elements so conjoined—i.e. elements that are conjunctively present in some cases and disjunctively present in other cases. Multiple elements listed with “and/or” should be construed in the same fashion—i.e. “one or more” of the elements so conjoined. Other elements may optionally be present other than the elements specifically identified by the “and/or” clause, whether related or unrelated to the specifically identified elements. Therefore, as a non-limiting example, a reference to “A and/or B”, when used in conjunction with open-ended language such as “comprising” can refer, in one embodiment, to A only (optionally including elements other than B); in another embodiment, to B only (optionally including elements other than A); and in yet another embodiment, to both A and B (optionally including other elements).

“Aromatic” is a substituted (as otherwise described herein) or unsubstituted monovalent aromatic radical (e.g., a 5-16 membered ring) having a single ring (e.g., benzene, phenyl, benzyl, or 5, 6, 7, or 8 membered ring) or condensed rings (e.g., naphthyl, anthracenyl, phenanthrenyl, 10-16 membered ring, etc.) and can be bound to the compound according to the present disclosure at any available stable position on the ring(s) or as otherwise indicated in the chemical structure presented. In any aspect or embodiment described herein, aromatic includes 5-, 6-, 7-, 8-, 9-, 10-, 11-, and/or 12-membered monocyclic or bicyclic aromatic groups. In any aspect or embodiment described herein, aromatic may be an aryl or a heteroaryl.

“Aryl” indicates aromatic groups containing only carbon in the aromatic ring or rings. Typical aryl groups contain 1 to 3 separate, fused, or pendant rings, and from 6 to 18 ring atoms, without heteroatoms as ring members. When indicated, such aryl groups may be further substituted with carbon or non-carbon atoms or groups. Aryl groups include, for example, phenyl, naphthyl, including 1-naphthyl, 2-naphthyl, and bi-phenyl.

The phrase “at least one” means at least one element selected from anyone or more of the elements in the list of elements, but not necessarily including at least one of each and every element specifically listed within the list of elements and not excluding any combinations of elements in the list of elements. This definition optionally permits elements other than the elements specifically identified within the list of elements to which the phrase “at least one” refers, whether related or unrelated to those elements specifically identified. As a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); and in yet another embodiment, to at least one, optionally including more than one, A, and at least one, optionally including more than one, B (and optionally including other elements).

A “carbocyclyl” is a monocyclic or bicyclic saturated, partially unsaturated, or aromatic ring system in which all ring atoms are carbon. Usually, each ring of the carbocyclyl group contains from 3-6 ring atoms and a bicyclic carbocyclyl group contains from 7 to 10 ring atoms, but some other number of ring atoms may be specified. Unless otherwise indicated, the carbocycle may be attached to the group it substitutes at any carbon atom that results in a stable structure. When indicated the carbocyclic rings described herein may be substituted at any carbon atom if the resulting compound is stable. Examples of carbocyclyl groups include phenyl, naphthyl, tetrahydronaphthyl, cyclopropyl, cyclohexyl, and cyclohexenyl.

The terms “comprises,” “comprising,” and the alternate transitional phrases “includes,” “including,” “have,” “contain,” “containing,” and variations thereof, are non-limiting—i.e., are to be interpreted inclusively or open-ended (that is, to mean including but not limited to). That is, these words are intended to convey the possible inclusion of other elements or integers not specifically recited, where the context allows. “Comprises” and the other open-ended terms encompass the intermediate term “consisting essentially of” and the closed ended terms “consisting of” and “consists of” Claims reciting one of the open-ended transitional phrases can be written with any other transitional phrase, which may be more limiting, unless clearly precluded by the context or art. No language in the specification should be construed as indicating any non-claimed element essential to the practice of the inventions of the present disclosure. Only the transitional phrases “consisting of” and “consisting essentially of” shall be closed or semi-closed transitional phrases, respectively, as set forth in the United States Patent Office Manual of Patent Examining Procedures, Section 2111.03.

“Cycloalkyl” is a saturated hydrocarbon ring group, having the specified number of carbon atoms. Monocyclic cycloalkyl groups typically have from 3 to 8 carbon ring atoms, or from 3 to 6 (3, 4, 5, or 6) carbon ring atoms. Cycloalkyl substituents may be pendant from a substituted nitrogen, oxygen, or carbon atom, or a substituted carbon atom that may have two substituents, which may have a cycloalkyl group that is attached as a spiro group. Examples of cycloalkyl groups include cyclopropyl, cyclobutyl, cyclopentyl, and cyclohexyl.

An “effective amount” or “therapeutically effective amount” is an amount sufficient to produce a selected effect, such as alleviating or ameliorating one or more symptoms of a disease or disorder.

As used in the specification and the appended claims, the terms “for example,” “for instance,” “such as,” “including” and the like are meant to introduce examples that further clarify more general subject matter. Unless otherwise specified, these examples are provided only as an aid for understanding the disclosure and are not meant to be limiting in any fashion.

“Halo” or “halogen” indicates any of fluoro, chloro, bromo, and iodo.

“Haloalkyl” indicates both branched and straight-chain alkyl groups, as described herein, having the specified number of carbon atoms, substituted with one or more halogen atoms, up to the maximum allowable number of halogen atoms. Examples of haloalkyl include, but are not limited to, trifluoromethyl, difluoromethyl, 2-fluoroethyl, 2,2,2-trifluoroethyl, and penta-fluoroethyl.

“Haloalkoxy” indicates a haloalkyl group as described herein that is attached through an oxygen bridge (—O—).

“Heteroaryl” is a stable monocyclic aromatic ring having the indicated number of ring atoms which contains from 1 to 4, or in some embodiments from 1 to 2, heteroatoms independently selected from N, O, and S, with remaining ring atoms being carbon, or a stable bicyclic or tricyclic system containing at least one 5- to 7-membered aromatic ring which contains from 1 to 4, or in some embodiments from 1 to 2, heteroatoms independently selected from N, O, and S, with remaining ring atoms being carbon. Monocyclic heteroaryl groups typically have from 5 to 7 ring atoms. In any aspect or embodiment described herein, the heteroaryl group is a 5- or 6-membered heteroaryl group having 1, 2, 3, or 4 heteroatoms independently selected from N, O, and S, with no more than two O atoms and one S atom.

The terms “heterocycle,” “heterocyclyl,” and “heterocyclic” indicate a monocyclic saturated, partially unsaturated, or aromatic ring containing from 1 to 4 heteroatoms independently selected from N, O, and S, with remaining ring atoms being carbon, or a bicyclic saturated, partially unsaturated, or aromatic heterocycle containing at least 1 heteroatom selected from N, O, and S in one of the two rings of the two ring system and containing up to about 4 heteroatoms independently selected from N, O, and S in each ring of the two ring system. Usually, each ring of the heterocycle contains from 4-6 ring atoms but some other number of ring atoms may be specified. Unless otherwise indicated, the heterocycle may be attached to its pendant group at any heteroatom or carbon atom that results in a stable structure. When indicated the heterocycles described herein may be substituted on carbon, sulfur, or nitrogen atom if the resulting compound is stable. It is preferred that the total number of heteroatoms in a heterocycle is not more than 4 and that the total number of S and O atoms in a heterocycle is not more than two, more preferably not more than one. Examples of heterocyclyl groups include, pyridyl, indolyl, pyrimidinyl, pyridazinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, dihydroisoindolyl, 5,6,7,8-tetrahydroisoquinoline, pyrazolyl, pyrrolidinyl, morpholinyl, piperazinyl, piperidinyl, and pyrrolidinyl. In certain embodiments a heterocycle is chosen from pyridinyl, pyrimidinyl, furanyl, thienyl, and pyrrolyl.

Additional examples of heterocyclyl groups include, but are not limited to, phthalazinyl, indolizinyl, indazolyl, benzothiazolyl, benzimidazolyl, benzofuranyl, benzoisoxolyl, dihydro-benzodioxinyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanonyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromanyl, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N oxide, indazolyl N-oxide, benzothiazolyl N-oxide, benzimidazolyl N-oxide, pyrrolyl N-oxide, oxadiazolyl N-oxide, thiadiazolyl N-oxide, tetrazolyl N-oxide, benzothiopyranyl S-oxide, and benzothiopyranyl S,S-dioxide. In certain embodiments the heterocyclyl group is a pyrimidinyl, oxazolyl, morpholinyl, piperidinyl, piperazinyl, thiomorpholinyl, isothiazolyl, pyrrolidinyl, oxadiazolyl, oxadiazolyl, oxadiazolyl substituted with benzyl, pyrazolyl, pyrazinyl, oxazolidinyl, isothiazolidinyl, imidiazolyl, pyridazinyl, pyridinyl, pyrrolyl, thiazolyl, thienyl, or furanyl group.

“Heterocycloalkyl” is a saturated heterocyclic group having the indicated number of ring atoms, for example 3-7 ring atoms, 4-7 ring atoms, 4-6 ring atoms, or 5-6 ring atoms, and 1, 2, or 3 heteroatoms independently selected from N, O, and S. It is preferred that not more than two of the heteroatoms are O and S.

The term “inhibit,” refers to the ability of a compound of the disclosure to reduce or impede a described function, such as cell growth or reproduction. Preferably, inhibition is by at least 10%, more preferably by at least 25%, even more preferably by at least 50%, and most preferably, the function is inhibited by at least 75%.

The term “mono- and/or di-alkylamino” indicates secondary or tertiary alkyl amino groups, wherein the alkyl groups are independently chosen alkyl groups, as defined herein, having the indicated number of carbon atoms. The point of attachment of the alkylamino group is on the nitrogen. Examples of mono- and di-alkylamino groups include ethylamino, dimethylamino, and methyl-propyl-amino.

As used herein, “optional” or “optionally” means that the subsequently described event or circumstance may or may not occur, and that the description includes instances where said event or circumstance occurs and instances where it does not.

The term “or” as used herein has the same meaning as “and/or,” as defined above. For example, when separating items in a list, “or” and “and/or” are interpreted as being inclusive—i.e. the inclusion of at least one, but also including more than one of any number of the listed elements, and, optionally, additional unlisted items. Only terms clearly indicated to the contrary, such as “only one of” or “exactly one of,” or, when used in the claims, “consisting of,” refer to the inclusion of exactly one element of a number or list of elements. Generally, the term “or” as used herein shall only be interpreted as indicating exclusive alternatives (i.e. “one or the other but not both”) when preceded by terms of exclusivity—e.g. “one of,” “only one of,” or “either.”

A “patient” is a human or non-human animal in need of medical treatment. In any aspect or embodiment described herein, the patient is a human patient.

The term “pharmaceutical composition” shall mean a composition comprising at least one active ingredient, such as tebipenem pivoxil hydrobromide, and at least one other substance, such as a pharmaceutically acceptable carrier or excipient. Pharmaceutical compositions optionally contain one or more additional active agents. Pharmaceutical compositions meet the U.S. FDA's GMP (good manufacturing practice) standards for human or non-human drugs.

A “pharmaceutically acceptable excipient” means an excipient that is useful in preparing a pharmaceutical composition/combination that is generally safe, non-toxic and neither biologically nor otherwise undesirable, and includes an excipient that is acceptable for veterinary use as well as human pharmaceutical use. The term also encompasses any of the inactive agents approved for use pharmaceutical compositions in by a regulatory agency of the US Federal government or listed in the US Pharmacopeia or other generally recognized pharmacopeia for use in animals, including humans. An excipient can be a carrier, filler, diluent, bulking agent or other inactive or inert ingredients.

A “pharmaceutically acceptable carrier” is a diluent, adjuvant, excipient, other ingredient, or combination of ingredients that alone or together provide a carrier or vehicle with which a compound, such as a bifunctional compound of the disclosure is formulated and/or administered, and in which every ingredient of the carrier as a whole is pharmaceutically acceptable. The pharmaceutically acceptable carrier includes a carrier that is acceptable for veterinary use as well as human pharmaceutical use. Also included are all suitable solvents, dispersion media, coatings, antibacterial and antifungal agents, and isotonic and absorption delaying agents. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. A “pharmaceutically acceptable carrier” includes both one and more than one such carrier.

“Pharmaceutically acceptable” means compositions that are physiologically tolerable for, and do not typically produce an allergic or similar untoward reaction when administered, for either human or veterinary application. Preferably, as used herein, the term “pharmaceutically acceptable” means approved by a regulatory agency of a federal or state government or listed in the U.S. Pharmacopeia or other generally recognized pharmacopeia for use in animals, and more particularly in humans. As used herein, “pharmaceutical compositions” include formulations for human and veterinary use.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Salts of the present compounds further include solvates of the compounds and of the compound salts.

Examples of pharmaceutically acceptable salts include, but are not limited to, mineral or organic acid salts of basic residues such as amines or nitrogen-containing heteroaryl rings (e.g. pyridine, quinoline, isoquinoline); alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HO₂C—(CH₂)~-CO₂H where n is 0-4, and the like. Lists of additional suitable salts may be found, e.g., in G. Steffen Paulekuhn, et al., Journal of Medicinal Chemistry 2007, 50, 6665 and Handbook of Pharmaceutical Salts: Properties, Selection and Use, P. Heinrich Stahl and Camille G. Wermuth Editors, Wiley-VCH, 2002.

The term “prevent,” as used herein, means to stop something from happening or to significantly reduce the likelihood of something happening, such as by taking advance measures against something possible or probable outcome.

A “subject” of analysis, diagnosis, or method of treatment is an animal. Such animals include mammals, preferably a human. As subject can be a human patient or a veterinary patient. A “subject” can be a human, a livestock animal (e.g. a bovine, sheep, equine, or porcine subject), or a companion animal (e.g. cat or dog).

The term “substituted”, as used herein, means that any one or more hydrogens on the designated atom or group is replaced with a selection from the indicated group, provided that the designated atom's normal valence is not exceeded. When the substituent is oxo (i.e., ═O) then two hydrogens on the atom are replaced. When an oxo group substitutes a heteroaromatic moiety, the resulting molecule can sometimes adopt tautomeric forms. For example, a pyridyl group substituted by oxo at the 2- or 4-position can sometimes be written as a pyridine or hydroxypyridine. Combinations of substituents and/or variables are permissible only if such combinations result in stable compounds or useful synthetic intermediates. A stable compound or stable structure is meant to imply a compound that is sufficiently robust to survive isolation from a reaction mixture and subsequent formulation into an effective therapeutic agent. Unless otherwise specified, substituents are named into the core structure. For example, it is to be understood that aminoalkyl means the point of attachment of this substituent to the core structure is in the alkyl portion and alkylamino means the point of attachment is a bond to the nitrogen of the amino group.

The term “symptom,” means any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

The terms “treat”, “treating”, and “treatment”, etc., as used herein, refer to any action providing a benefit to a patient at risk for a disease state or condition for which TNF-α modulation may beneficial, such as an autoimmune or inflammatory disease including rheumatoid arthritis (RA) or inflammatory bowel disease (IBD), among others, atherosclerosis, heart disease, and cancer, including recurrence and/or metastasis of cancer, improvement in the condition through lessening or suppression of at least one symptom of the disease state or condition, inhibition of one or more manifestations of the disease state (e.g., heart disease, cancer growth, reduction in cancer cells or tissue), and/or prevention (i.e. reduction in the likelihood or delay in progression of a disease state or condition or manifestation of the disease state or condition such as deterioration of tissue and inflammation in rheumatoid arthritis, further damage to cardiovascular tissue in heart disease, further damage to central nervous tissue in stroke, recurrence or metastasis of the cancer, prevention or delay in the onset of disease states or conditions which occur secondary to the disease state or condition including cancer recurrence or metastasis, among others). Treatment, as used herein, encompasses both prophylactic and therapeutic treatment, depending on the context of the treatment. The term “prophylactic” when used, means to reduce the likelihood of an occurrence or the severity of an occurrence within the context of treatment of disease state or condition, as otherwise described hereinabove.

### Chemical Description

An aspect of the present disclosure relates to a bifunctional Tumor Necrosis Factor-alpha (TNF-α) binding compound having the structure:

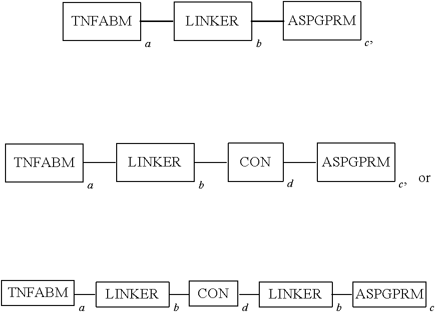

wherein,
[TNFABM] is a Tumor Necrosis Factor-Alpha (TNF-α) Binding Moiety that is capable of binding to TNF-α;
[ASPGPRM] is an Asialoglycoprotein Receptors Binding Moiety that binds to an asialoglycoprotein receptor (ASGPR) on an outer surface of a cell (e.g., a hepatocyte or other degrading cell, such as a cell in a subject);
[LINKER] is an optional chemical group that is covalently attached to one or more [TNFABM] groups and either one or more [CON] groups or one or more [ASPGPRM] group;
[CON] is an optional chemical connecting moiety that is covalently attached to one or more [ASPGPRM] groups and one or more [LINKER] groups;
a is an integer from 1 to 15 (e.g. 1, 2, 3, 4, 5, 6, 7, 8, 9, 10, 11, 12, 13, 14, or 15);
b is an integer from 0 to 15 (e.g. 0, 1, 2, 3, 4, 5, 6, 7, 8, 9, 10, 11, 12, 13, 14, or 15), preferably 0 or 1;
c is an integer from 1 to 15 (e.g. 1, 2, 3, 4, 5, 6, 7, 8, 9, 10, 11, 12, 13, 14, or 15); and
d is an integer from 0 to 15 (e.g. 0, 1, 2, 3, 4, 5, 6, 7, 8, 9, 10, 11, 12, 13, 14, or 15, preferably 0, 1, 2, or 3); or a pharmaceutically acceptable salt thereof.

In any aspect or embodiment described herein, a is 1 to 15, 1 to 10, 1 to 5, 1 to 3, or 1, 2 or 3.

In any aspect or embodiment described herein, b is 1 to 15, 1 to 10, 1 to 5, 1 to 3, or 1, 2 or 3, preferably 1.

In any aspect or embodiment described herein, c is 1 to 15, 1 to 10, 1 to 5, 1 to 3, or 1, 2 or 3, preferably 1, 2, or 3.

In any aspect or embodiment described herein, d is 1 to 15, 1 to 10, 1 to 5, 1 to 3, or 1, 2 or 3, preferably 1, 2, or 3.

In any aspect or embodiment described herein, a is 1, 2 or 3; b is 1, 2 or 3; c is 1, 2 or 3; or d is 1, 2 or 3.

The term “asialoglycoprotein receptor binding moiety” or [ASPGPRM] refers to a binding moiety which binds to asialoglycoprotein receptor on the surface of a cell, such as a hepatocyte or other degrading cell. The ASGPRBM group selectively binds to asialoglycoprotein receptor on the surface of, e.g., hepatocytes. It is through this moiety that bifunctional compounds complexed with circulating TNF-α bind to hepatocytes or other degrading cells. Once bound to the hepatocyte or other degrading cells, the circulating protein is taken into the cell via a phagocytosis mechanism wherein the circulating protein is degraded through lysosomal degradation.

In any aspect or embodiment described herein, each [ASGPRBM] is independently represented/selected from:

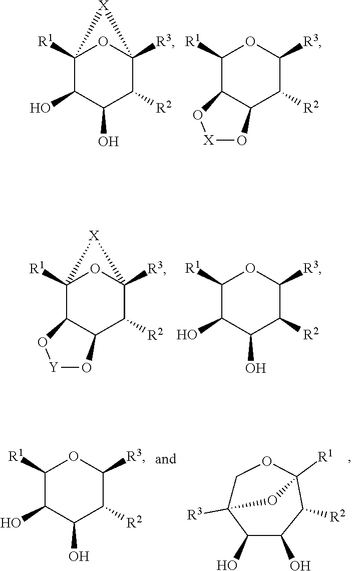

wherein:
X is 1, 2, 3, or 4 atoms in length, comprising one or more groups independently selected from O, S, N(R^{N1}), and C(R^{C1})(R^{C1}), wherein
when X is 1 atom in length, X is O, S, N(R^{N1}), or C(R^{C1})(R^{C1});
when X is 2 atoms in length, no more than 1 atom of X is O, S, or N(R^{N1});
when X is 3 or 4 atoms in length, no more than 2 (e.g., 0, 1, or 2) atoms of X are O, S, or N(R^{N1});
Y is 1 or 2 atoms in length, comprising one or two independently chosen 0 and C(R^{C1})(R^{C1}) groups;
each R^{N1} is independently H or C₁-C₃ alkyl optionally substituted with 1, 2, or 3 independently selected halogen atoms; and
each R^{C1} is independently hydrogen, halogen, or C₁-C₃ alkyl optionally substituted with 1, 2, or 3 independently selected halogen atoms;
the point of attachment of the [ASGPRBM] to the [LINKER] or [CON] is through R¹ or R³.

R¹ and R³ are each independently selected from (i), (ii), (iii), (iv), and (v):
(i) hydrogen which is replaced by a covalent bond when the R¹ or R³ is the point of attachment to the [LINKER] or [CON], or hydroxyl in which the hydrogen is replaced by a covalent bond when the R¹ or R³ is the point of attachment to the [LINKER] or [CON];
(ii) straight or branched C₁-C₈alkyl, optionally containing 1 or 2 double or triple bonds, optionally including 1 or 2 groups in the alkyl chain independently selected from O, S, NH, and N(C₁-C₄alkyl), and optionally substituted with one or more substituents independently selected from hydroxyl (—OH), halogen, cyano (—CN), oxo (═O), amino (—NH₂), imino (═NH), and —COOH, and is covalently bound to [LINKER] or [CON] via a terminal N, O, or C atom when the R¹ or R³ is the point of attachment to the [LINKER] or [CON];
(iii) a (5- or 6-membered heterocycle)(CH₂)_{K}— that is optionally substituted with one or more (e.g., 1, 2, 3, 4, 5, 6, or more) R^{A}, wherein
R^{A} is one or more C₁-C₄alkyl groups optionally substituted with halogen or hydroxyl;
R^{A}, when bound to a nitrogen, is no more than one substituent chosen from (phenyl)(CH₂)_{K}—, (C₃-C₇cycloalkyl)(CH₂)_{K}—,

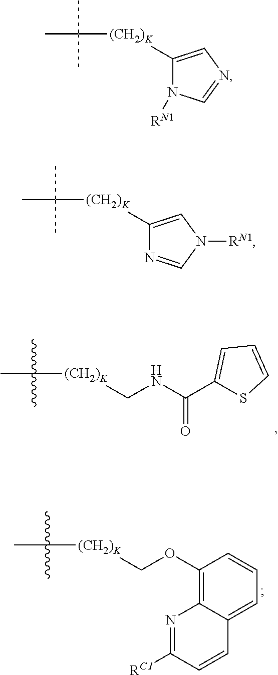

and
R^{A}, when bound to carbon atom, is selected from any of previously listed groups and is also selected from halogen, hydroxyl, C₁-C₄alkoxy optionally substituted with 1-3 (e.g. 1, 2, or 3) independently selected halogen atoms or 1-2 (e.g. 1 or 2) hydroxyl groups, and mono- and di(C₁-C₄alkyl)amino,
wherein any (5- or 6-membered heterocycle)(CH₂)_{K}— group within the (iv) definition is covalently bound to [LINKER] or [CON] via a terminal N, O, or C atom when the R¹ or R³ is the point of attachment to the [LINKER] or [CON];
(iv) a (phenyl)(CH₂)_{K}— that is: optionally substituted with 1 or more substituents independently chosen from halogen, hydroxyl, amino (—NH₂), —CN, and a straight or branched C₁-C₈alkyl that optionally contains 1 or 2 double or triple bonds, optionally includes 1 or 2 groups in the alkyl chain independently selected from O, S, NH, and N(C₁-C₄alkyl), wherein the N(C₁-C₄ alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently chosen from hydroxyl, halogen, cyano, oxo, imino, and —COOH, wherein any (phenyl)(CH₂)_{K}— group within the (iv) definition is covalently bound to [LINKER] or [CON] via a terminal N, 0, or C atom when the R¹ or R³ is the point of attachment to the [LINKER] or [CON];
(v) a C₃-C₈cyclolalkyl-(CH₂)k- optionally substituted with R^{A} and is covalently bound to [LINKER] or [CON] via a ring C atom, or a terminal N, 0, or C atom when the R¹ or R³ is the point of attachment to the [LINKER] or [CON],
wherein each K is independently 0, 1, 2, 3, or 4, preferably 0 or 1; or
R₂ is selected from (i), (ii), (iii), (iv), (v), (vi), and (vii).
(i) hydroxyl, halogen, amino, N₃, or —COOH;
(ii) straight or branched C1-C8alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently chosen from hydrogen, hydroxyl, halogen, oxo, and —COOH;
(iii)

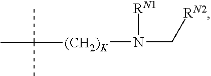

where R^{N1} and K are the same as above;
R^{N2} is H, —C₀-C₄alkylNR^{N3}R^{N1}, or straight or branched C₁-C₈alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently selected from hydroxyl, halogen, cyano, oxo, amino, imino (═NH), and —COOH; and
R^{N3} is H,

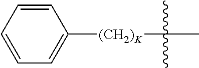

group, or C₁-C₃ alkyl group optionally substituted with 1-3 independently selected halogen atoms or 1-2 hydroxyl groups, or
(iv)

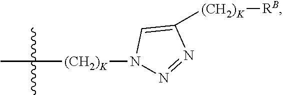

wherein
R^{B} is H, CN, straight or branched C₁-C₈alkyl that optionally contains 1 or 2 double or triple bonds, optionally includes 1 or 2 groups in the alkyl chain selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently selected from hydroxyl, halogen, cyano, oxo, amino, imino (═NH), and —COOH; or
R^{B} is a C₃-C₁₀ aryl or a three- to ten-membered heteroaryl group containing up to 5 heteroaryl atoms, each of said aryl or heteroaryl groups is optionally substituted with one or more (preferably 1) halogen, hydroxyl, CN, NR^{N1}R^{N2}, straight or branched C₁-C₈alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently selected from hydroxyl, halogen, cyano, oxo, and —COOH; or
R^{B} is

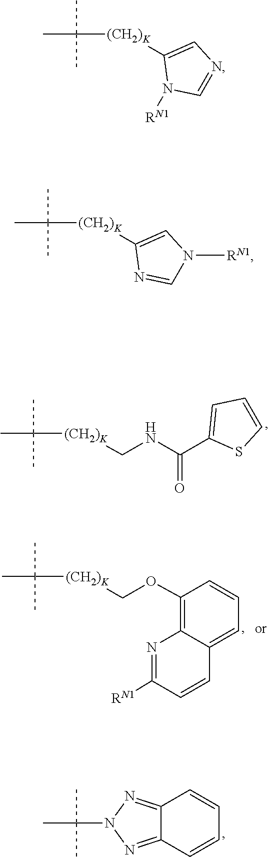

each of which is optionally substituted with up to three (e.g., 1, 2, or 3) substituents independently selected from halogen, hydroxyl, cyano, C₁-C₃alkyl, C₁-C₃alkoxy, C₁-C₃haloalkyl, and C₁-C₃haloalkoxy; or
(v)

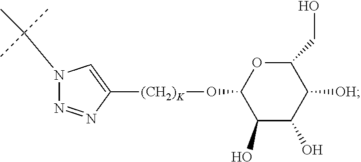

(vi) a carbocyclic or a heterocyclic group that is directly bound to the [ASGPRBM] group or bound to the [ASGPRBM] group via a O, S, NH, NHC(═O), C(═O)NH, or C₁-C₄alkylene that optionally has 1 double or triple bond and optionally has one —CH₂— group replaced by a 0, S, or NH, wherein each of which carbocyclic or heterocyclic group is unsubstituted or substituted with one or more substituents independently selected from halogen, hydroxyl, amino, azido, cyano, —COOH, oxo, imino, and a straight, branched, or cyclic C₁-C₈alkyl that optionally contains 1 or 2 double or triple bonds, optionally includes 1 or 2 groups in the alkyl chain selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is unsubstituted or substituted with one or more substituents independently selected from hydroxyl, halogen, SF₅, cyano, oxo, amino, azido, imino (═NH), and —COOH, and is substituted with 0 or 1 substituents selected from an aryl and a heterocyclic substituents which 1 aryl or heterocyclic substituent is optionally substituted with one or more R^{C1} substituents;
(vii) —NR^{N4}—S(O)—R^{D}, —NR^{N4}—C(S)—R^{D}, —NR^{N4}—S(O)(NR^{N4})R^{N4}, —N═S(O)(R^{D})₂, —NR^{N4}C(O)NR^{N4}S(O)₂R^{D}, —NR^{N4}—S(O)₂—R^{D}, —S(═O)(═NR^{N4})R^{D}, or —NR^{N4}C(NR^{N4})R^{N4}, wherein each R^{D} is independently selected from
(a) hydrogen,
(b) heterocycle-(CH₂)_{K} or carbocycle-(CH₂)_{K}, each of which is optionally substituted with one or more substituents independently selected from halogen, hydroxyl, cyano, amino, C₁-C₄alkyl, C₁-C₄alkoxy, C₁-C₂haloalkyl, and C₁-C₂haloalkoxy,
(c) -ORE, —NR^{N4}R^{N4}, and
(d) straight or branched C₁-C₆alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the straight or branched C₁-C₆alkyl is optionally substituted with one or more substituents independently chosen from hydroxyl, halogen, cyano, oxo, amino, imino (═NH), and —COOH;

Each R^{N4} and R^{E} is independently selected from:
(a) hydrogen,
(b) heterocycloalkyl-(CH₂)_{K}, heteroaryl-(CH₂)_{K}, aryl-(CH₂)_{K}, C₃-C₆cycloalkyl-(CH₂)_{K}, each of which is optionally substituted with one or more substituents independently selected from halogen, hydroxyl, cyano, amino, C₁-C₄alkyl, C₁-C₄alkoxy, C₁-C₂haloalkyl, and C₁-C₂haloalkoxy, and
(c) straight or branched C₁-C₆alkyl that optionally contains 1 or 2 double or triple bonds, optionally includes 1 or 2 groups in the alkyl chain independently selected from O, S, NH, and N(C₁-C₄alkyl) which N(C₁-C₄alkyl) is optionally substituted with 1-3 (e.g. 1, 2, or 3) independently selected halogen atoms or 1-2 (e.g. 1 or 2) hydroxyl groups, and the C₁-C₆alkyl is optionally substituted with one or more substituents independently chosen from hydroxyl, halogen, cyano, oxo, amino, imino (═NH), and —COOH.

Or, R² and R³ are joined to form a 5- to 6-membered saturated or partially unsaturated cyclic group optionally containing 1, 2, or 3 heteroatoms independently selected from N, O, and S, and optionally substituted with 1 or more substituents independently selected from oxo, halogen, hydroxyl, cyano, amino, and straight or branched C₁-C₆alkyl that optionally contains 1 or 2 double or triple bonds, optionally includes 1 or 2 groups in the alkyl chain selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently selected from hydroxyl, halogen, cyano, oxo, and —COOH.

In any aspects or embodiment of this disclosure, each [ASGPRM] is independently selected from

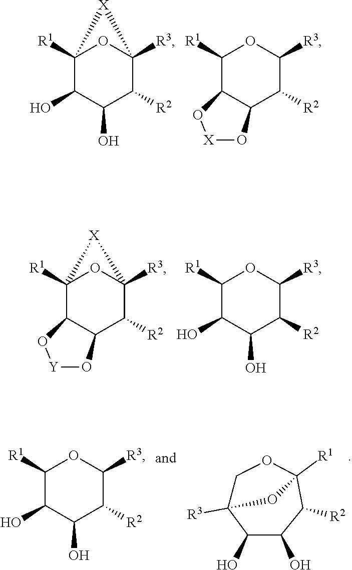

Where, X and Y are independently selected from —CH₂—, —CH₂CH₂—, —CH₂O—, and —OCH₂—.

R¹ and R³ are independently:
(i) hydrogen, which is replaced by a covalent bond when the R¹ or R³ is the point of attachment to the [LINKER] or [CON], or hydroxyl in which the hydrogen is replaced by a covalent bond when the R¹ or R³ is the point of attachment to the [LINKER] or [CON];
(ii) straight or branched C₁-C₈alkyl, optionally containing 1 or 2 double or triple bonds, optionally including 1 or 2 groups in the alkyl chain independently selected from O, S, NH, and N(C₁-C₄alkyl), and optionally substituted with one or more substituents independently selected from hydroxyl (—OH), halogen, cyano (—CN), oxo (═O), amino (—NH₂), imino (═NH), and —COOH, and is covalently bound to [LINKER] or [CON] via a terminal N, O, or C atom when the R¹ or R³ is the point of attachment to the [LINKER] or [CON].

R² is
(i) hydroxyl, halogen, amino, N₃, or —COOH; or
(ii) straight or branched C₁-C₈alkyl that optionally contains 1 or 2 double or triple bonds, optionally includes 1 or 2 groups in the alkyl chain independently selected from O, S, NH, and N(C₁-C₄alkyl) which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently chosen from hydrogen, hydroxyl, halogen, oxo, and —COOH.

In any aspect or embodiment described herein, least one of, or each of, the [ASPGPRM] is:

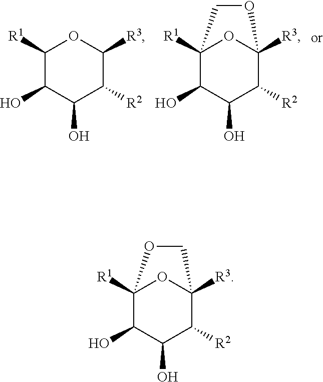

In any aspect or embodiment described herein, R¹ and R³, in at least one of or each of the [ASPGPRM], are independently chosen from definitions (i), (ii), and (iii) above.

In any aspect or embodiment described herein, R¹, in at least one of or each of the [ASPGPRM], is [L]-OCH₂—, [L]-CH₂CH₂—, or [L]-NHCH₂—, where [L] represent the point of attachment to the [LINKER] or [CON], and the dash (“-”) is the point of attachment of the R¹ substituent to the group it substitutes.

In any aspect or embodiment described herein, at least one of, or each of, the [ASGPRBM] is

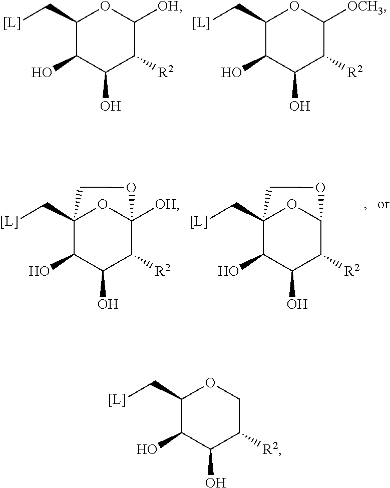

wherein [L] denotes the point of attachment to the [LINKER] or [CON].

In any aspect or embodiment described herein, R², in at least one of or each of the [ASPGPRM], is a carbocyclic or heterocyclic group that is directly bound to the [ASGPRBM] group or bound to the [ASGPRBM] group via a O, S, NH, NHC(═O), C(═O)NH, or C₁-C₄alkylene group, the C₁-C₄alkylene group optionally having 1 double or triple bond and optionally having one —CH₂— group replaced by a O, S, or NH, where the carbocyclic or heterocyclic group is phenyl, pyridine, pyridazine, pyrimidine, pyrazine, triazine, furan, pyrrole, thiadiazole, thiatriazole, pyrazole, triazole, isoxazole, oxazole, oxadiazole, imidazole, thiazole, cyclopropane, cyclobutane, cyclopentane, cyclohexane, oxetane, piperazine, piperidine, morpholine, azasilinane, diazaspiro[3.3]heptane, benzodiazole, benzimidazole, benzothiazole, pyrrolotriazine, pyrrolopyridine, pyrrolopyrimidine, each of which is unsubstituted or substituted with one or more substituents independently selected from halogen, hydroxyl, amino, azido, cyano, —COOH, oxo, imino, and straight, branched, or cyclic C₁-C₈alkyl, wherein the C₁-C₈alkyl optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g., 1, 2, or 3) independently selected halogen atoms or 1-2 (e.g., 1 or 2) hydroxyl groups, and the C₁-C₈alkyl is unsubstituted or substituted with one or more substituents independently chosen from hydrogen, hydroxyl, halogen, SF₅, cyano, oxo, amino, azido, imino (═NH), and —COOH substituted with 0 or 1 aryl or heterocyclic substituent which 0 or 1 aryl or heterocyclic substituent is optionally substituted with one or more R^{C1} substituents.

In any aspect or embodiment described herein, R², in at least one of or each of the [ASPGPRM], is a carbocyclic or heterocyclic group independently selected from

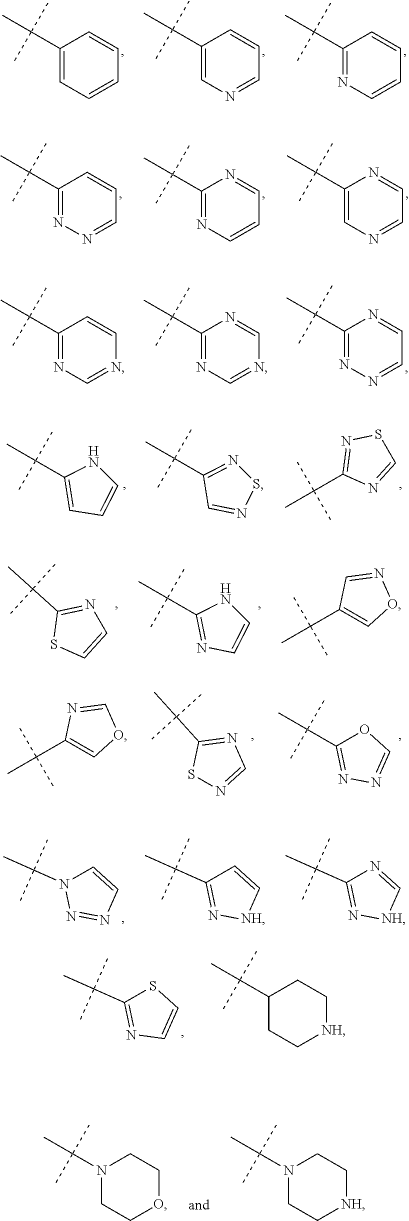

Each of which is either directly bound to the [ASGPRBM] group or bound to the [ASGRPBM] group via a O, S, NH, or C₁-C₄alkylene group, the C₁-C₄alkylene group optionally having 1 double bond and optionally having one —CH₂— group replaced by a 0, S, or NH, and the carbocyclic or heterocyclic group is unsubstituted or substituted with one or more substituents independently chosen from halogen, hydroxyl, amino, azido, cyano, —COOH, oxo, imino, and straight, branched, or cyclic C₁-C₈alkyl, that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g., 1, 2, or 3) independently selected halogen atoms or 1-2 hydroxyl groups, and the C₁-C₈alkyl is optionally substituted with one or more substituents independently chosen from hydroxyl, halogen, SF₅, cyano, oxo, amino, azido, imino (═NH), and —COOH and is substituted with 0 or 1 aryl or heterocyclic substituent which 0 or 1 aryl or heteroaryl substituent is a phenyl, piperazine, piperidine, or morpholine substituent, optionally substituted with one or more R^{C1} substituents.

In any aspect or embodiment described herein, R², in at least one of or each of the [ASPGPRM] is

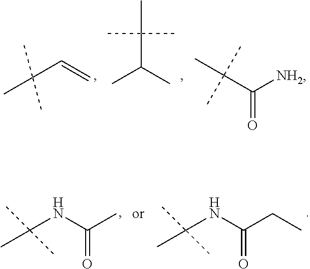

In any aspect or embodiment described herein, R², in at least one of or each of the [ASPGPRM], is

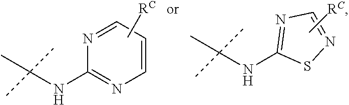

wherein R^{C} is 0, 1, or more substituents independently selected from halogen, hydroxyl cyano, amino, C₁-C₄alkyl, C₁-C₄alkoxy, C₁-C₂haloalkyl, and C₁-C₂haloalkoxy.

In any aspect or embodiment described herein, R² and R³, in at least one of or each of the [ASPGPRM], are joined to form a ring such that the [ASGPRBM] has a structure of formula

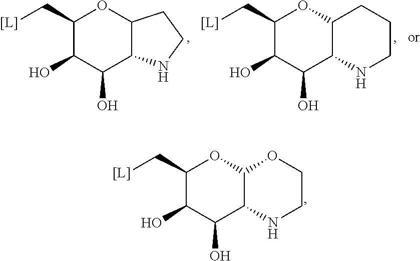

wherein [L] denotes the point of attachment to the [LINKER] or [CON], and the [ASGPRBM] is optionally substituted with 1 or more substituents (e.g., 1, 2, 3, 4, 5, or more) independently selected from oxo, halogen, hydroxyl, cyano, amino, and straight or branched C₁-C₆alkyl, wherein the alkyl group
(i) optionally contains 1 or 2 double or triple bonds,
(ii) optionally includes 1 or 2 groups in the alkyl chain independently selected from O, S, NH, and N(C₁-C₄alkyl), and
(iii) optionally substituted with one or more substituents (e.g., 1, 2, 3, 4, 5, or more) independently chosen from hydroxyl, halogen, cyano, oxo, and —COOH.

In any aspect or embodiment described herein, at least one of, or each of, the [ASGPRBM] is

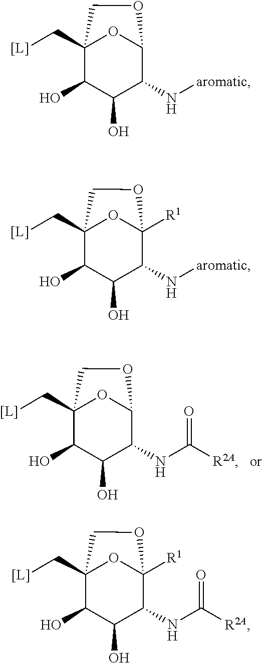

Wherein, [L] denotes the point of attachment to the [LINKER] or [CON]; each aromatic is independently a 5-membered to 10-membered aromatic ring (e.g., a 6-membered to 10-membered aryl or a 5-membered to 10-membered heteroaryl) that optionally has one or more (e.g., 1, 2, 3, 4, or 5) independently selected heteroatoms (e.g., O, N, or S) and is optionally substituted with one or more substituents independently selected from halogen, hydroxyl, amino, CN, straight, branched, or cyclic C₁-C₈alkyl (e.g. a C₁-C₈alkyl) that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) halogen atoms or 1-2 (e.g. 1 or 2) hydroxyl groups, and the C₁-C₈alkyl is optionally substituted with one or more substituents independently chosen from hydroxyl, halogen, SF₅, cyano, oxo, amino, imino (═NH), and —COOH and is substituted with 0 or 1 aryl or heterocyclic substituent which 0 or 1 aryl or heterocyclic substituent is optionally substituted with one or more R^{C1} substituents; and each R^{2A} is independently a C₁-C₄alkyl that is optionally substituted with 1 to 5 (e.g., 1, 2, 3, 4, or 5) independently selected halogen atoms.

In any aspect or embodiment described herein, at least one of, or each of, the [ASGPRBM] is

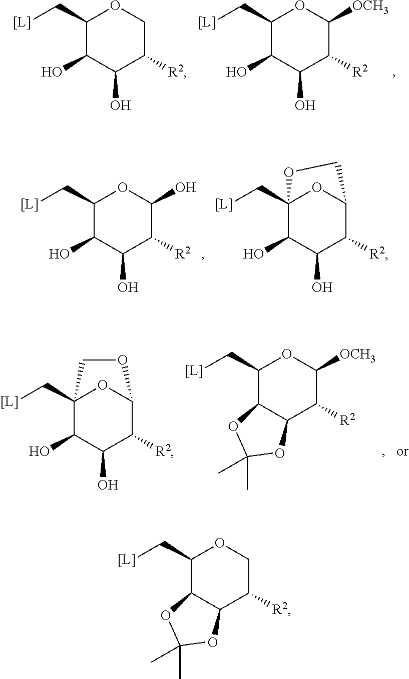

[L] denotes the point of attachment to the [LINKER] or [CON].

Each R² is independently selected from —CF₃, —CH₃, —N₃, —OCH₃, —COOH, —C(═O)CH₃, —C(═O)OCH₃, —CH₂N₃, —CH₂OH, —(CH₂)₂OH, —(CH₂)₃OH, —C(═O)CF₃, —C(═O)NHCH₃, —(CH₂)₃OH, —(CH₂)C(═O)NH₂, —CH₂NHC(═O)NH₂, —(CH₂)₂C(═O)CH₃, —NHC(═O)CH₃, —NHC(═O)CH₂—CH₃, —NHC(═O)CH₂F, —NHC(═O)CHF₂, —NHC(═O)CF₃, —NHC(═O)cyclopropyl, —NHC(═O)cyclobutyl, —NHC(═O)cyclopentyl, —NHC(═O)cyclohexyl, —NHSO₂CH₃, —NHSO₂CH₃, —NHSO₂CF₃, —NHC(═O)NHSO₂CH₃,

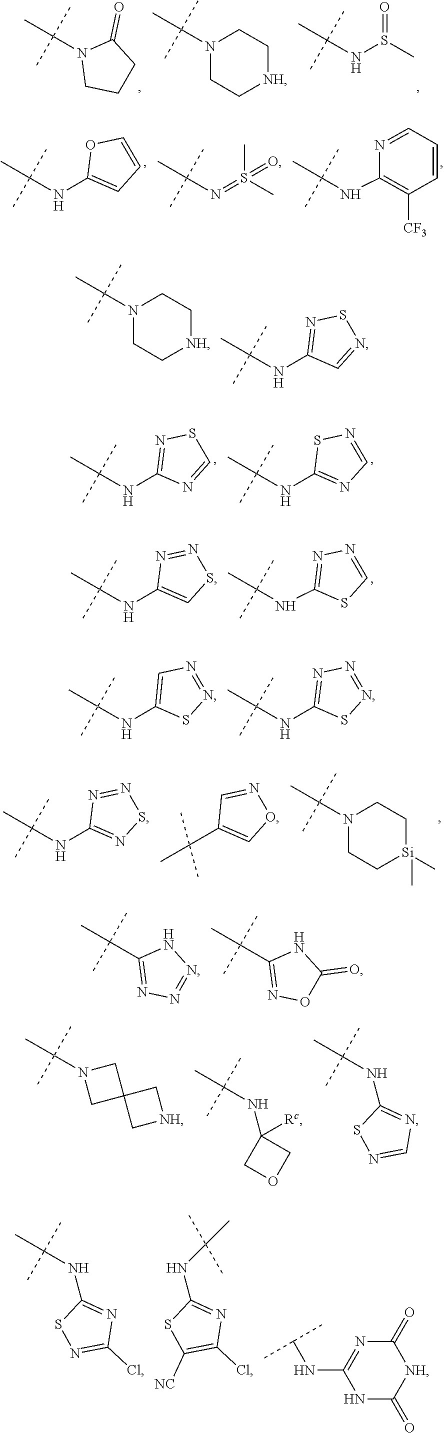

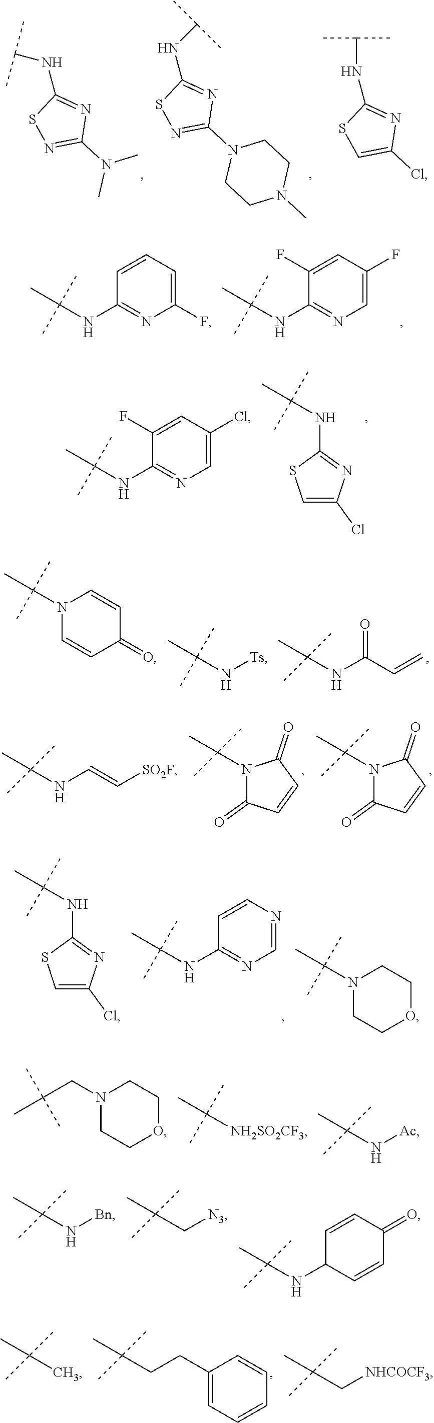

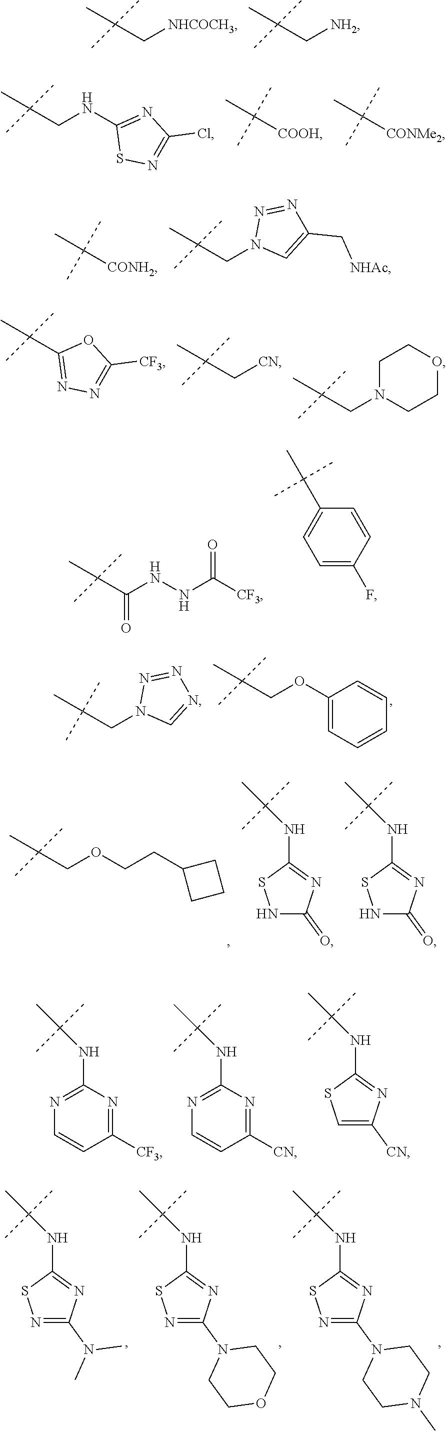

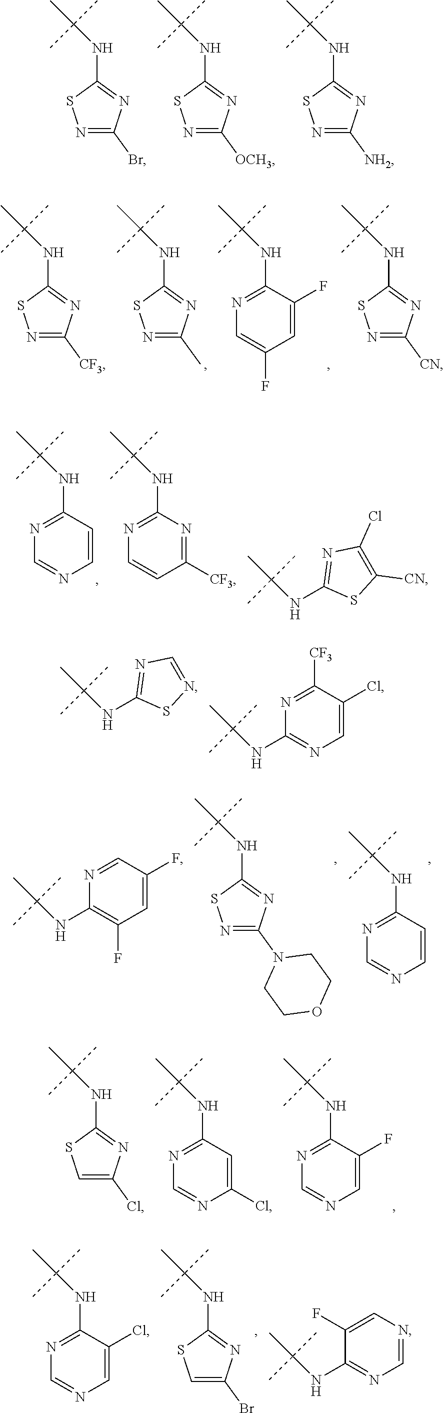

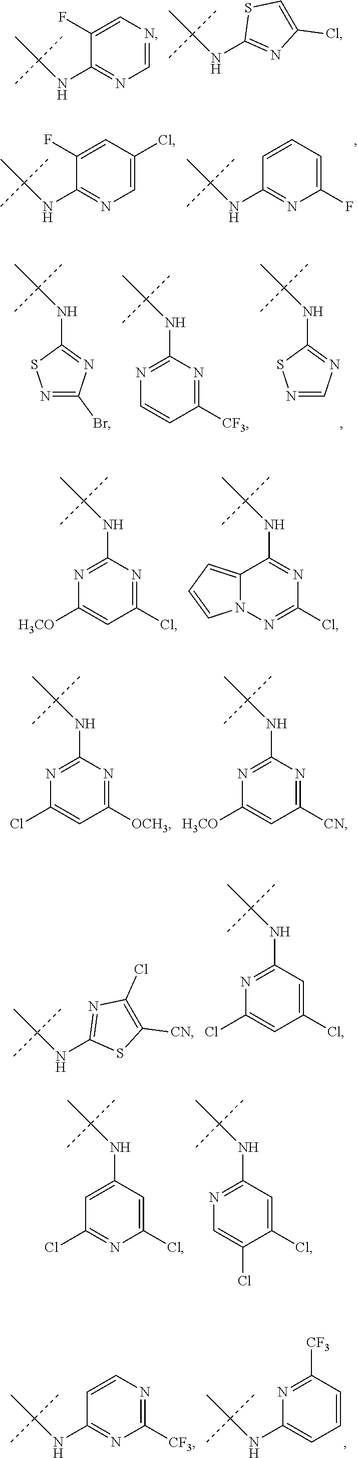

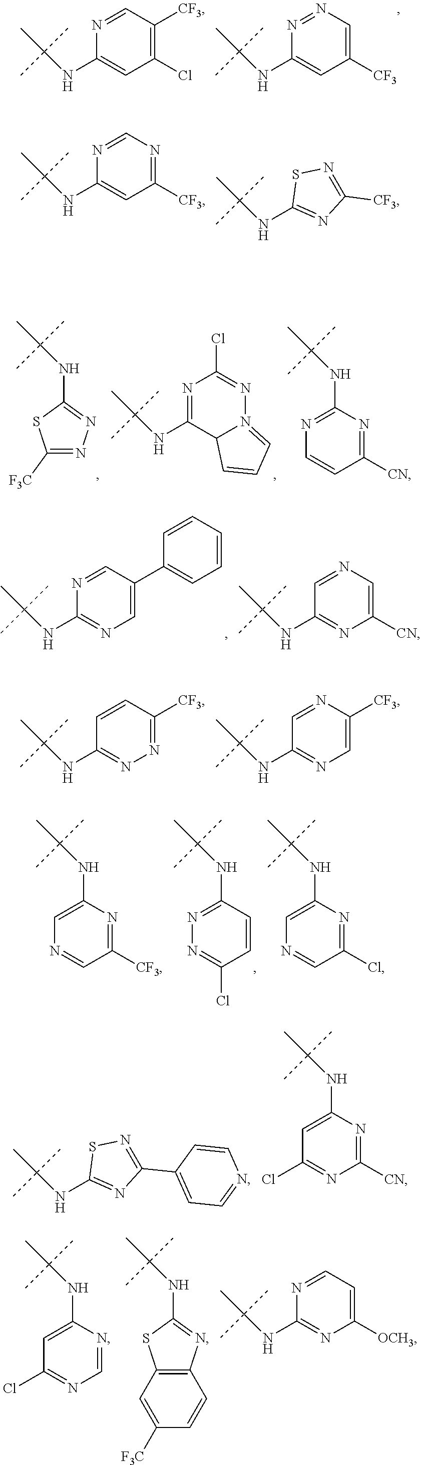

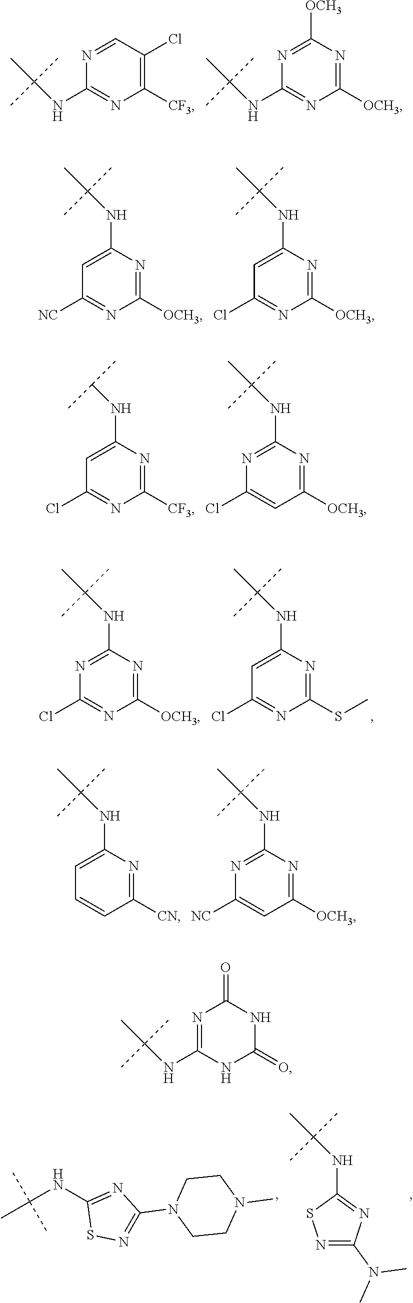

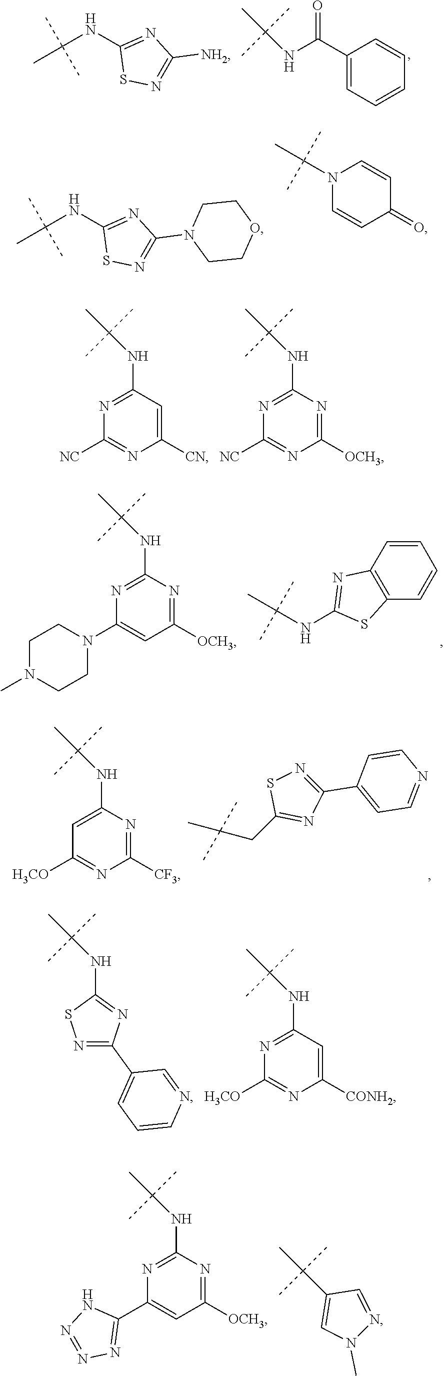

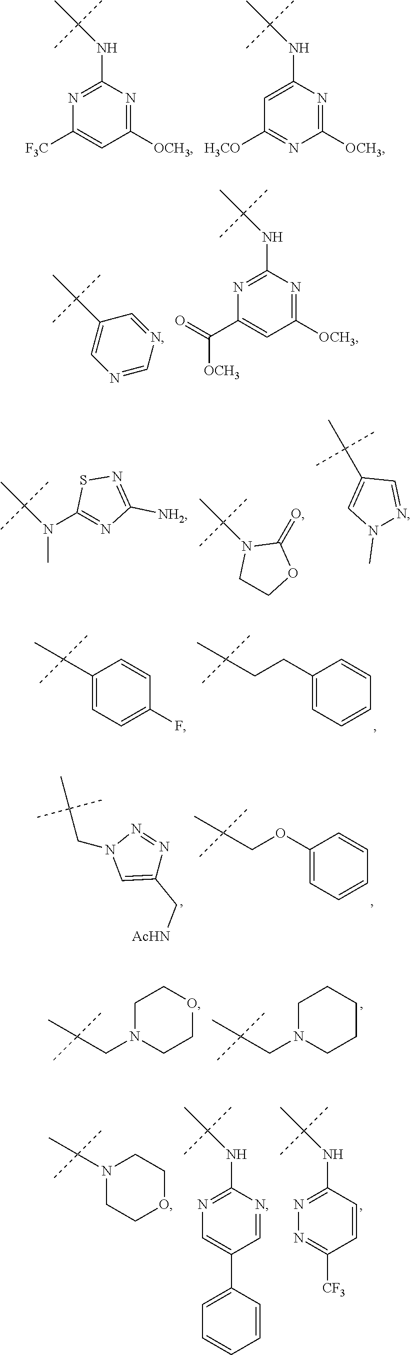

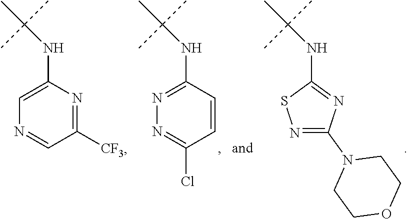

In any aspect or embodiment described herein, at least one of, or each of, the [ASGPRBM] is

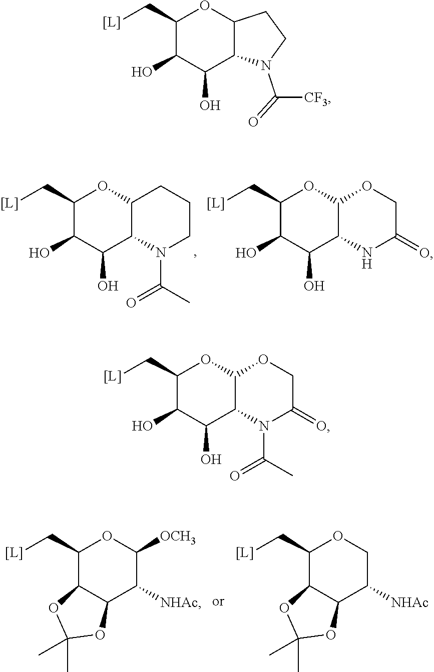

wherein [L] denotes the point of attachment to the [LINKER] or [CON].

In any aspect or embodiment described herein, at least one of, or each of, the [ASGPRBM] is

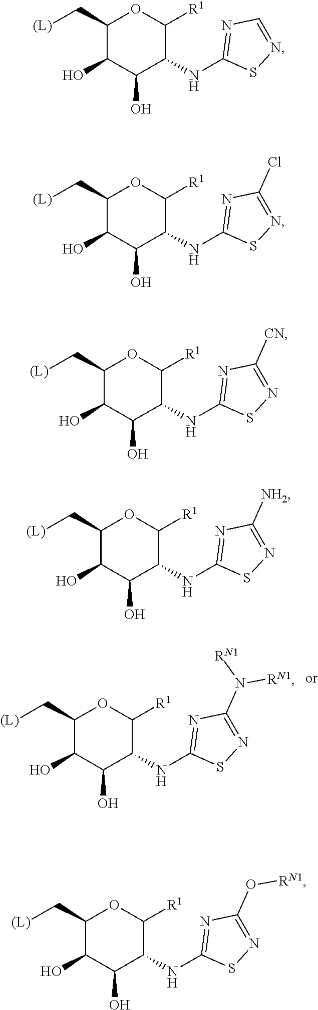

wherein [L] denotes the point of attachment to the [LINKER] or [CON].

In any aspect or embodiment described herein, R², in at least one of or each of the [ASPGPRM], is

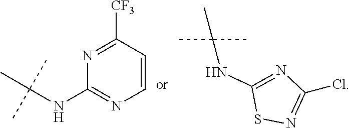

In any aspect or embodiment described herein, R¹ or R³, in at least one of or each of the [ASPGPRM], is
(iii) a (5- or 6-membered heterocycle)(CH₂)_{K}— that is optionally substituted with one or more (e.g., 1, 2, 3, 4, 5, 6, or more) R^{A}, wherein
R^{A} is one or more C₁-C₄alkyl groups optionally substituted with halogen or hydroxyl;
R^{A}, when bound to a nitrogen, is no more than one substituent chosen from (phenyl)(CH₂)_{K}—, (C₃-C₇cycloalkyl)(CH₂)_{K}—,

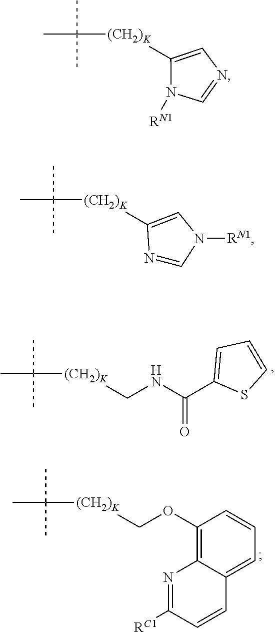

R^{A}, when bound to carbon atom, is selected from any of previously listed groups and is also selected from halogen, hydroxyl, C₁-C₄alkoxy optionally substituted with 1-3 (e.g. 1, 2, or 3) independently selected halogen atoms or 1-2 (e.g. 1 or 2) hydroxyl groups, and mono- and di(C₁-C₄alkyl)amino, wherein any (5- or 6-membered heterocycle)(CH₂)_{K}— group within the (iii) definition is covalently bound to [LINKER] or [CON] via a terminal N, 0, or C atom when the R¹ or R³ is the point of attachment to the [LINKER] or [CON]; or
(iv) a (phenyl)(CH₂)_{K}— that is: optionally substituted with 1 or more substituents independently chosen from halogen, hydroxyl, amino (—NH₂), —CN, and a straight or branched C₁-C₈alkyl that optionally contains 1 or 2 double or triple bonds, optionally includes 1 or 2 groups in the alkyl chain independently selected from O, S, NH, and N(C₁-C₄alkyl), wherein the C₁-C₄ alkyl is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently chosen from hydroxyl, halogen, cyano, oxo, imino, and —COOH, wherein any (phenyl)(CH₂)_{K}— group within the (v) definition is covalently bound to [LINKER] or [CON] via a terminal N, O, or C atom when the R¹ or R³ is the point of attachment to the [LINKER] or [CON].

In any aspect or embodiment described herein, R¹ or R³, in at least one of or each of the [ASPGPRM], is

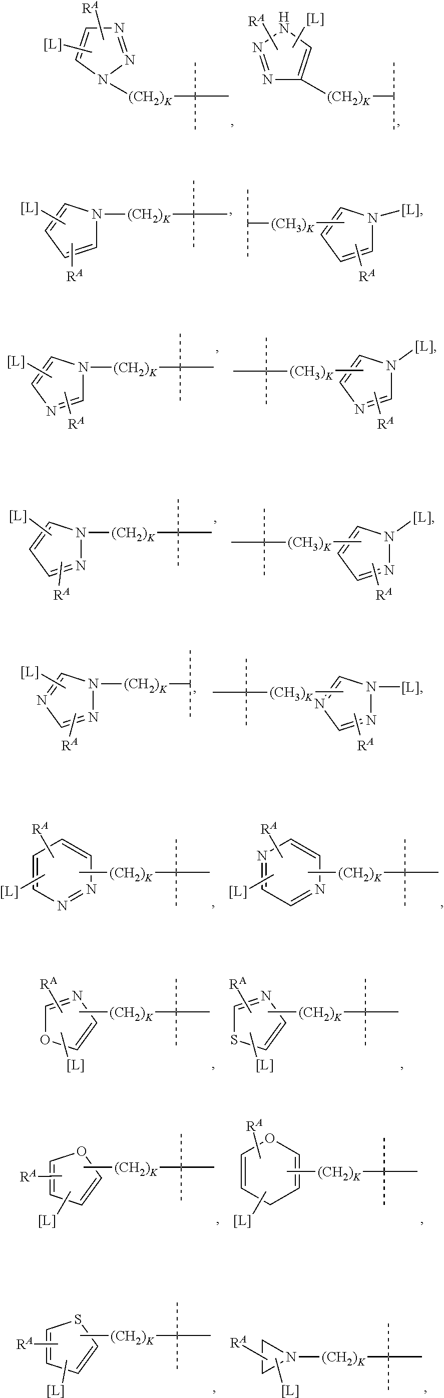

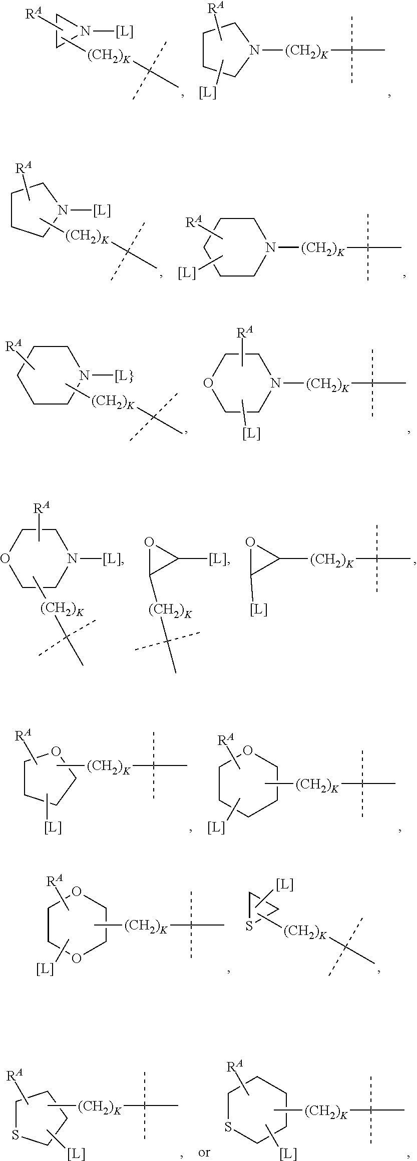

wherein [L] denotes the point of attachment to the [LINKER] or [CON].

In any aspect or embodiment described herein, at least one of, or each of, the [ASGPRBM] is independently selected from

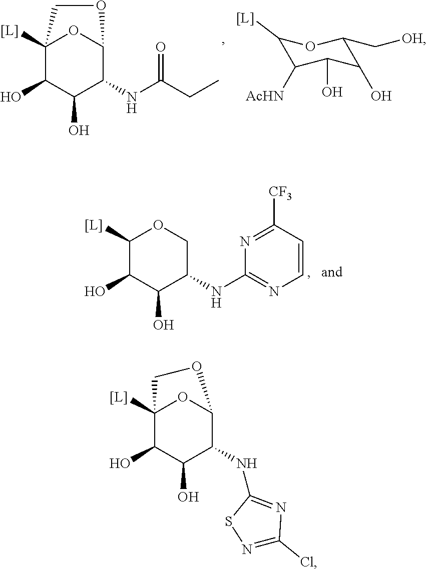

wherein [L] denotes the point of attachment to the [LINKER] or [CON].

The term “Tumor Necrosis Factor-alpha binding moiety” or [TNFABM] refers to a binding moiety which binds to Tumor Necrosis Factor-α. The [TNFABM] selectively binds to TNF-α. Bifunctional compounds complexed with circulating TNF-α bind to hepatocytes or other degrading cells through the [ASPGPRM]. Once bound to the hepatocyte or other degrading cells, the TFN-α is taken into the cell via a phagocytosis mechanism wherein the circulating protein is degraded through lysosomal degradation.

In any aspect or embodiment described herein, the [TNFABM] is

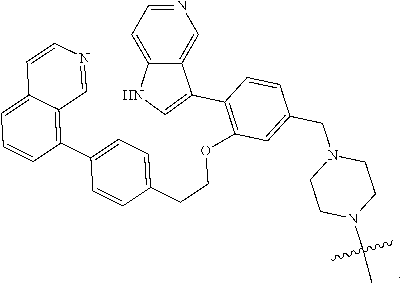 [SMILES: *N1CCN(Cc2ccc(-c3c[nH]c4ccncc34)c(OCCc3ccc(-c4cccc5ccncc45)cc3)c2)CC1]

In any aspect or embodiment described herein, the [TNFABM] is

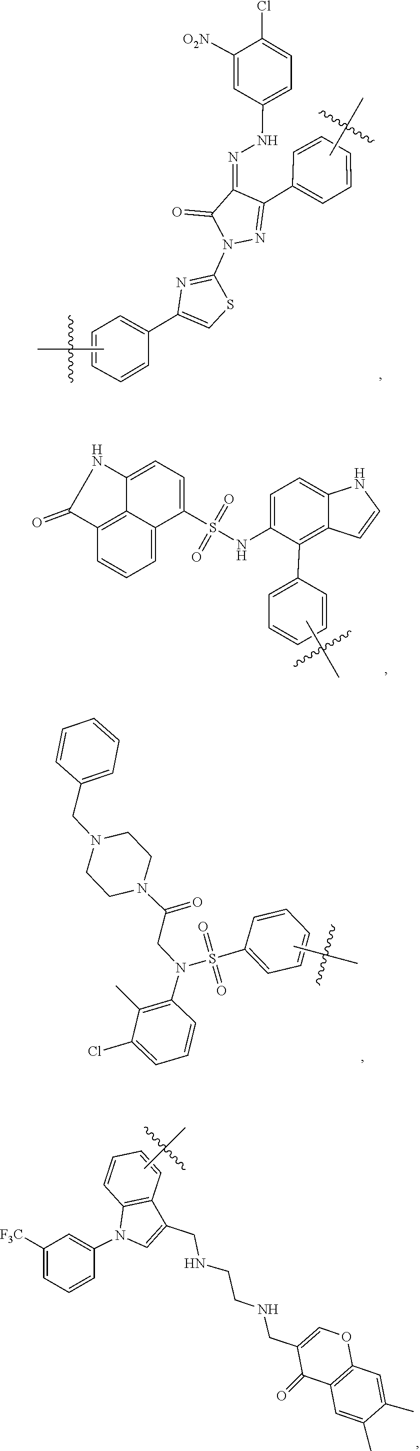

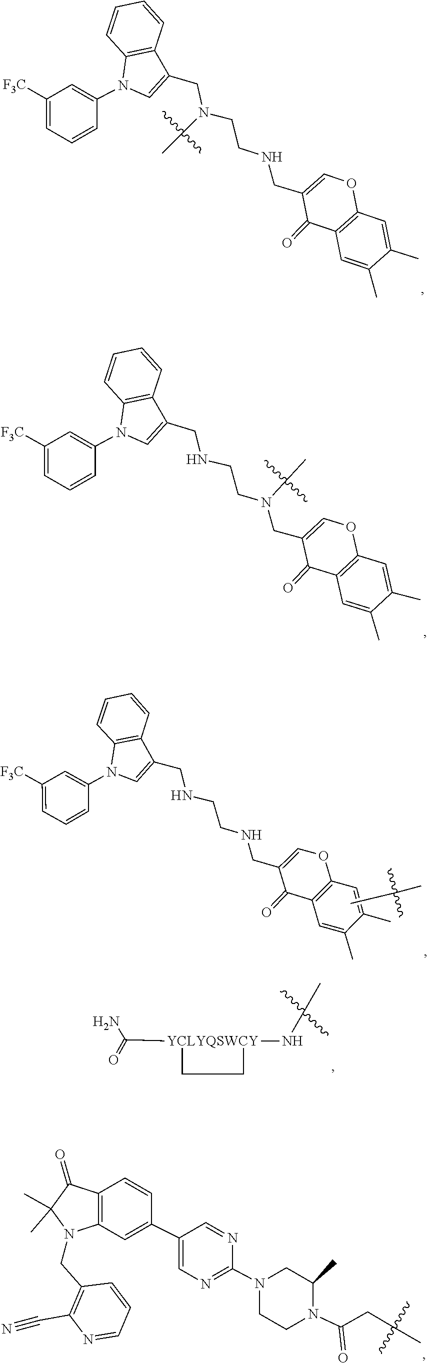

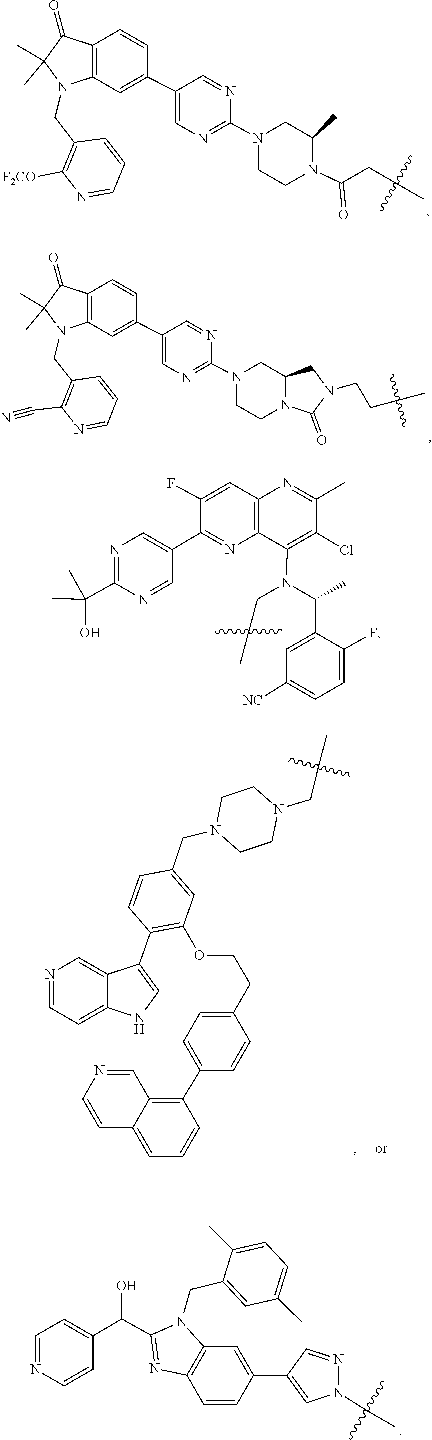

The term “linker” or [LINKER] refers to an optional chemical moiety that covalently attached to one or more [TNFABM] groups and [CON], thereby linking the one or more [TNFABM] groups and one or more [ASGPRBM] groups.

In any aspect or embodiment described herein, the [LINKER] is an optionally substituted C₁-C₅₀ alkylene group (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkylene), wherein:
the [LINKER] optionally comprises one to fourteen —C₁-C₂₅ alkylene-[TNFABM] covalently linked to the C₁-C₅₀ alkylene group; and
the [LINKER] optionally comprises one to fourteen —C₁-C₂₅ alkylene-[ASPGPRM] covalently linked to the C₁-C₅₀ alkylene group;
each CH₂ group is optionally replaced with a group independently selected from O, S, SO, SO₂, NH, CO, CH═CH, C≡C, 5-7 membered heterocycle, 3-8 membered carbocycle, and [CON]; and
the [LINKER] is optionally substituted with one or more (e.g., 1, 2, 3, 4, 5, 6, 7, 8, 9, 10 or more) groups independently selected from H, halogen, hydroxyl, cyano, oxo (═O), amino, imino (═NH), —COOH, C₁-C₄alkyl, C₁-C₄haloalkyl, and C₁-C₄alkoxy.

In any aspect or embodiment described herein, the [LINKER] is selected from:

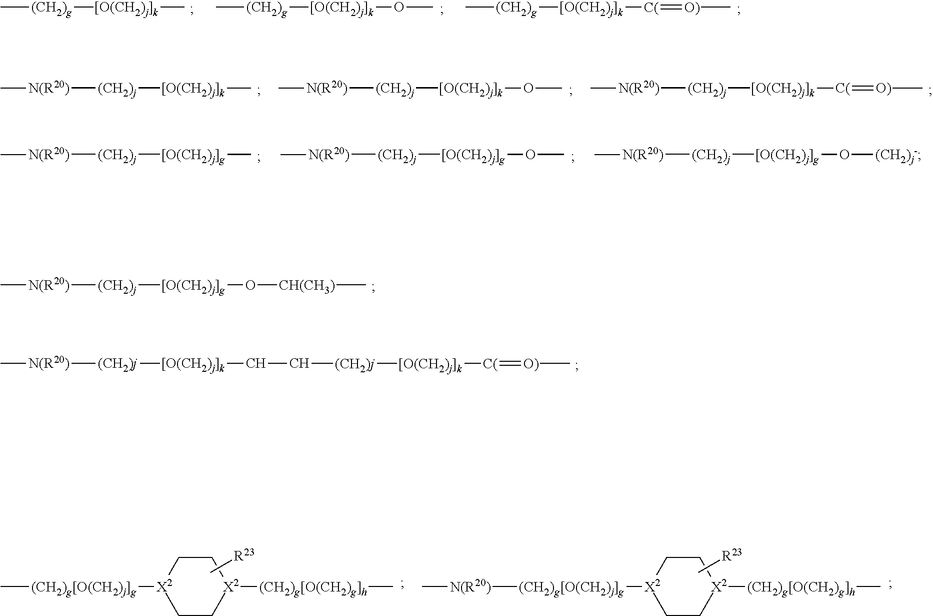

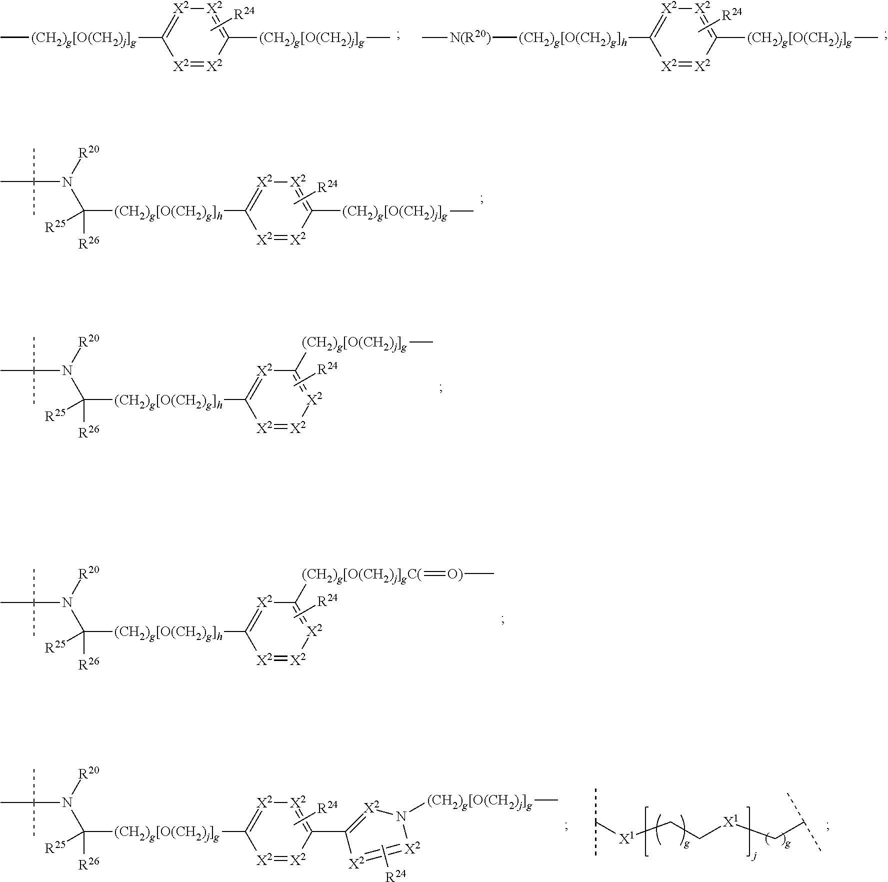

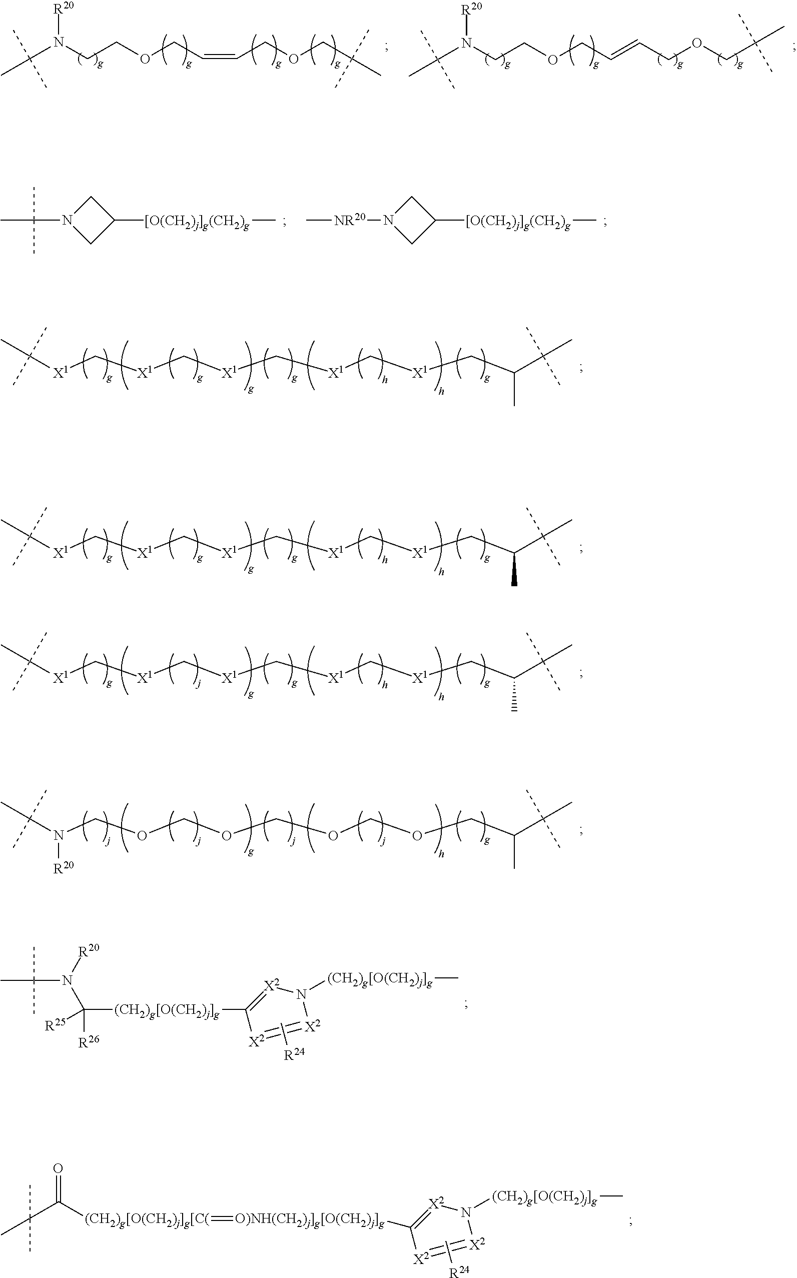

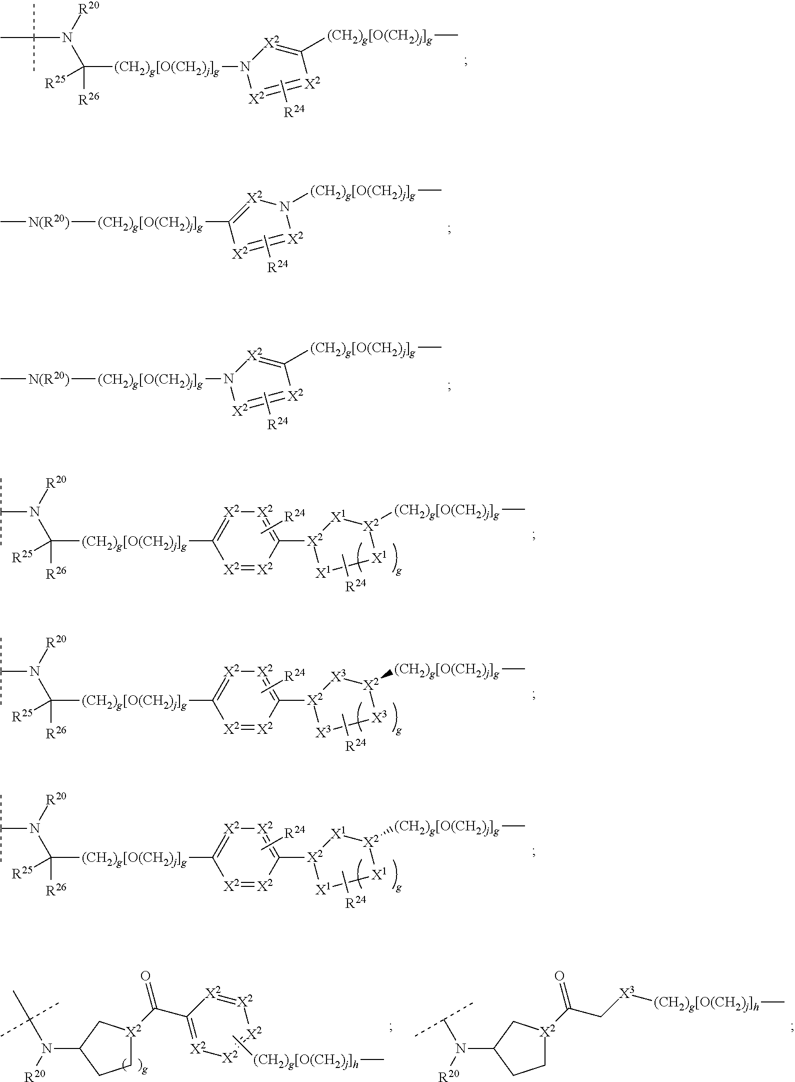

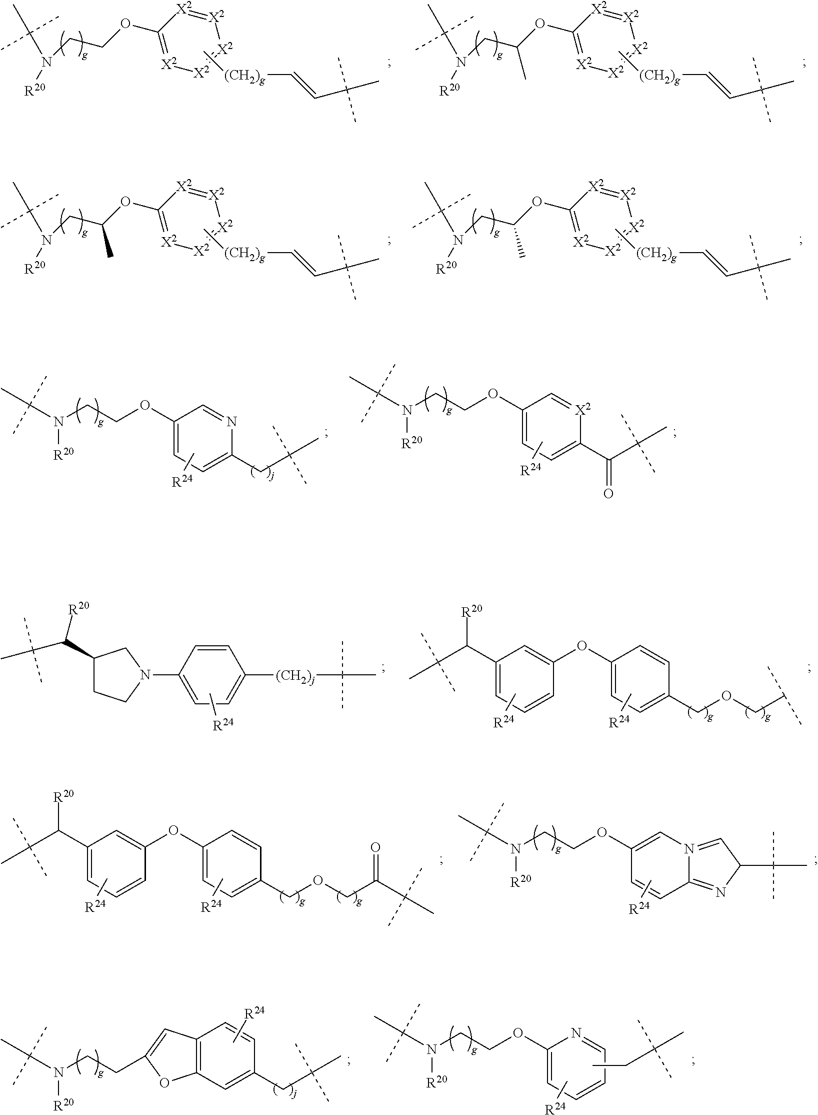

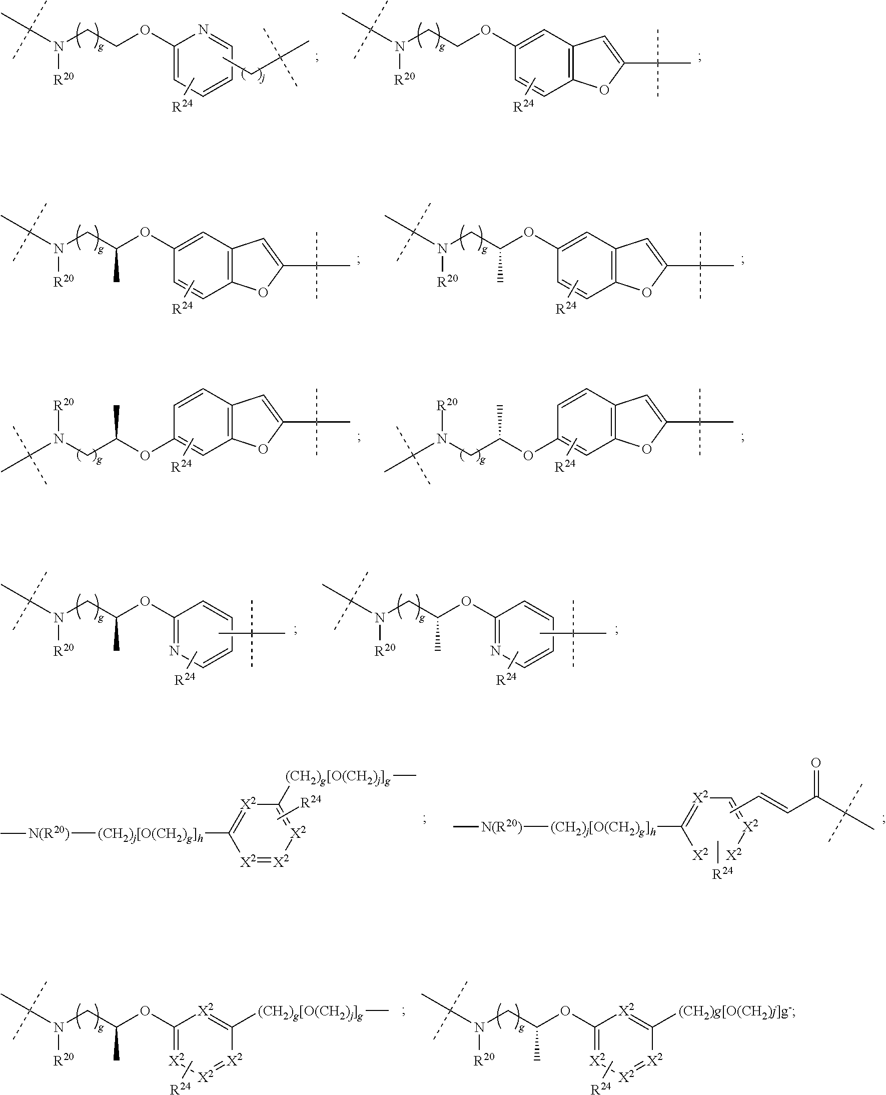

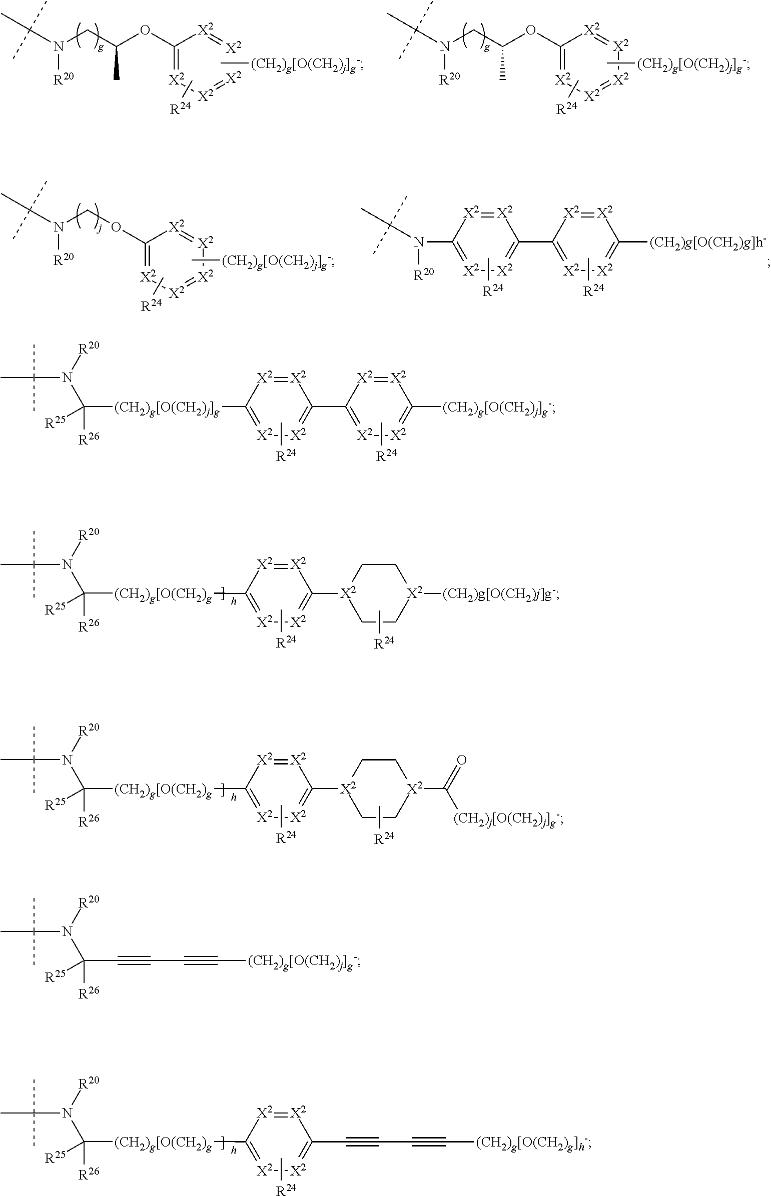

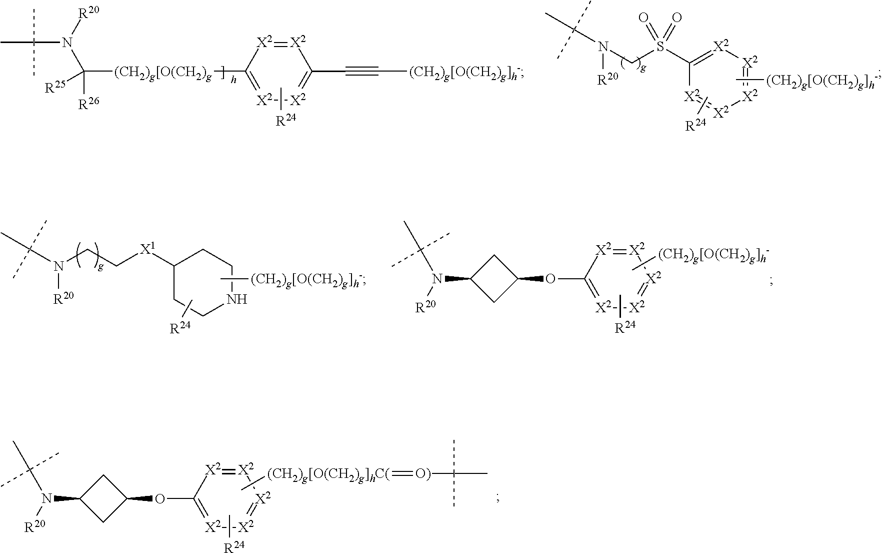

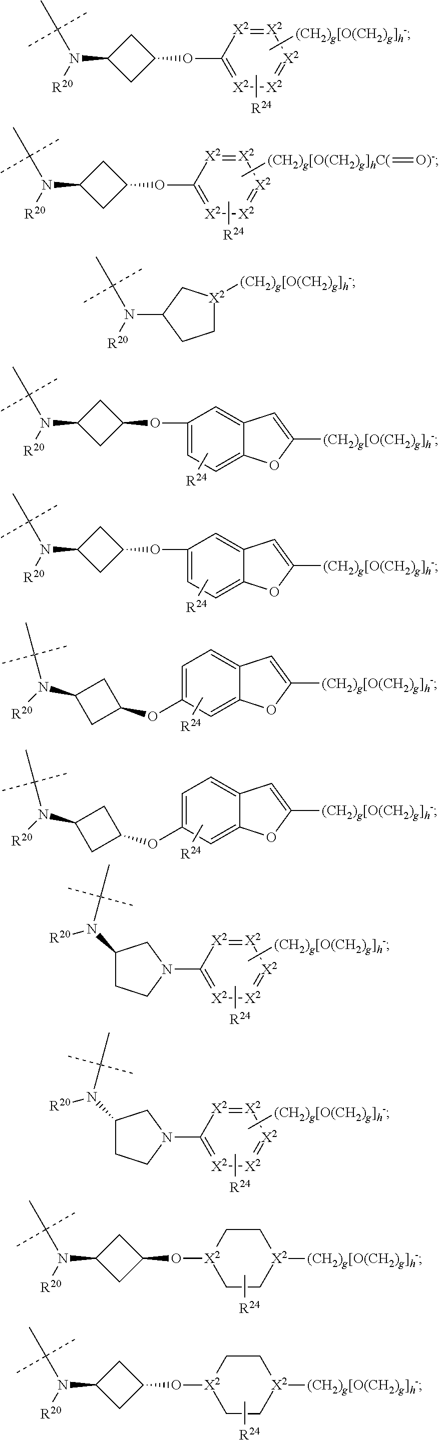

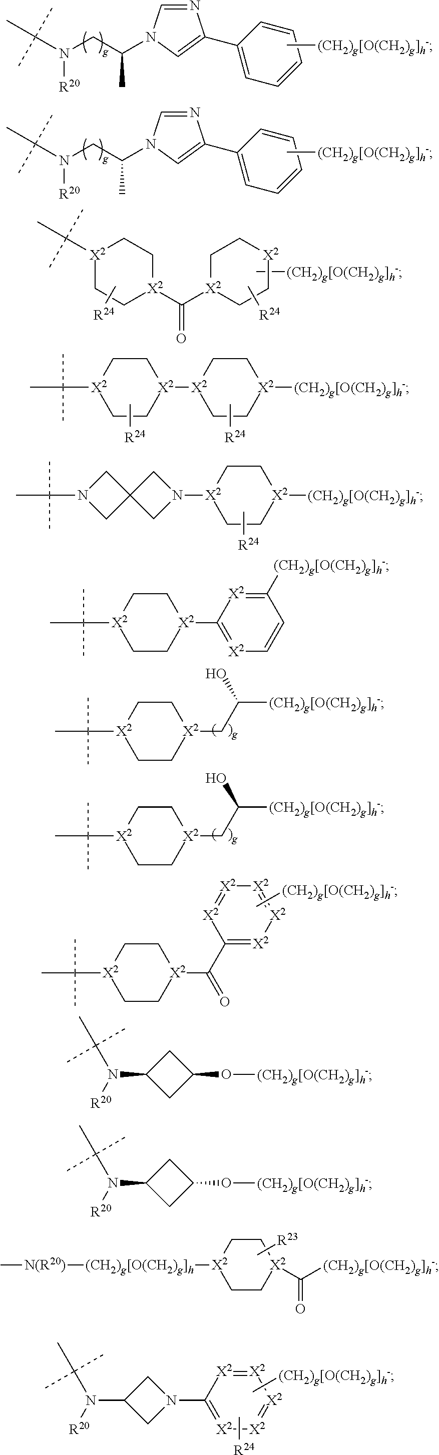

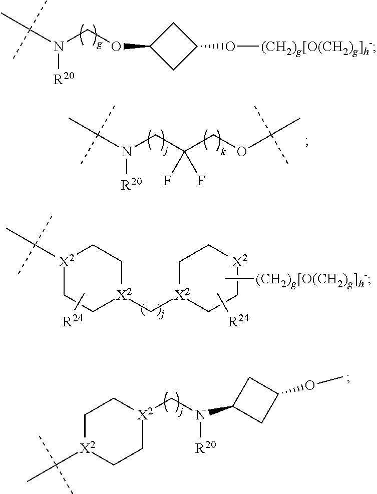

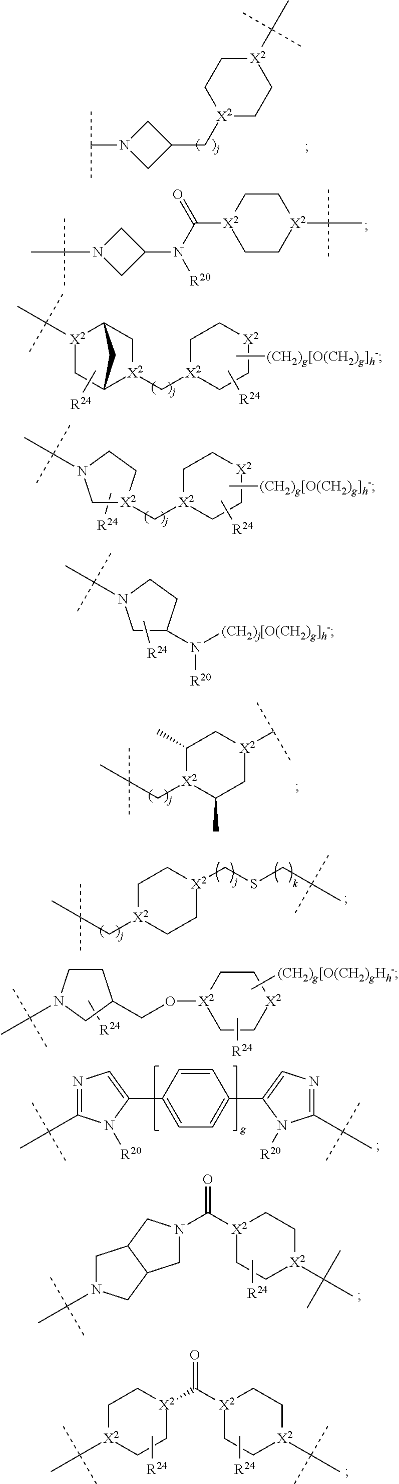

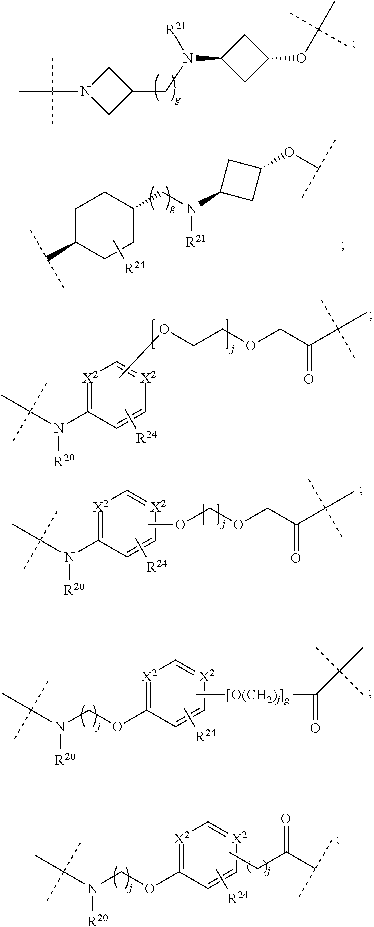

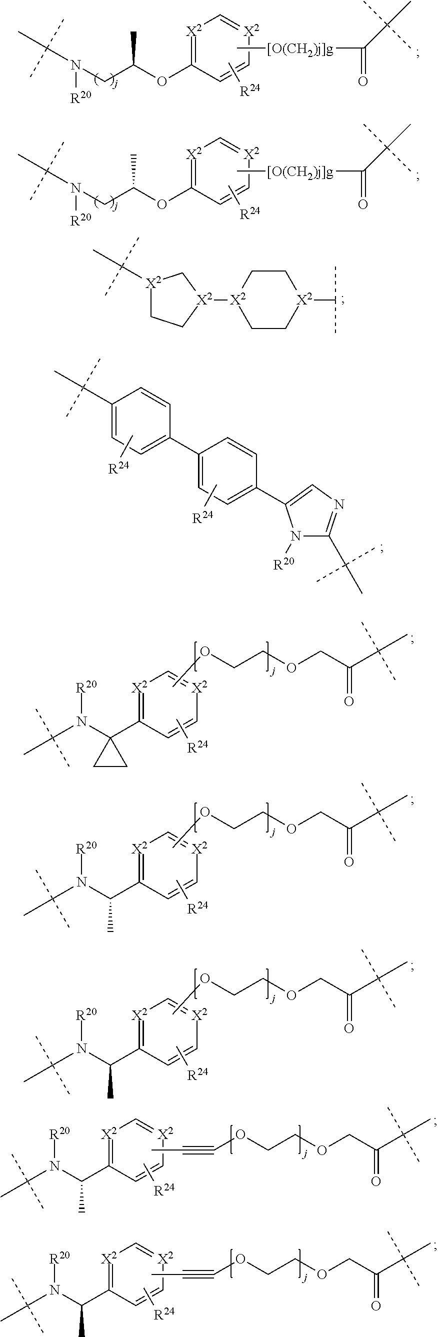

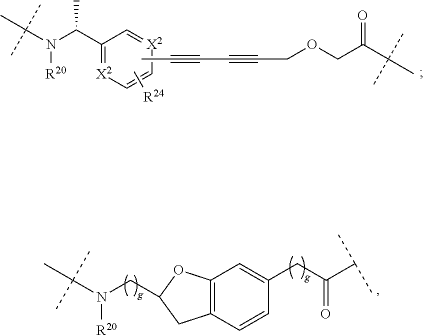

wherein
each R²⁰ is independently selected from hydrogen, C₁-C₄alkyl, and (C₃-C₆alkyl)C₀-C₂alkyl;
each R²¹ is independently selected from hydroxyl, halogen, C₁-C₄alkyl, and C₁-C₄alkoxy;
R²³ is 0 to 4 substituents independently selected from halogen, hydroxyl, amino, C₁-C₂alkyl, and C₁-C₂alkoxy;
R²⁴ is 0 to 4 substituents independently selected from halogen, hydroxyl, amino, cyano, C₁-C₄alkyl, C₁-C₄alkoxy, C₁-C₂haloalkyl, and C₁-C₂haloalkoxy;
each R²⁵ and R²⁶ is independently selected from hydrogen and methyl, or R²⁵ and R²⁶ are joined to form a spiro attached C₃-C₅cycloalkyl group, or R²⁵ and R²⁶ can be taken together along with the carbon atom to which they are bound form a 3- to 8-membered monocyclic or bicyclic cycloalkyl ring;
each X¹ is independently selected from CH₂, NR²⁰, O, SO, SO₂, and S;
each X² is independently selected from CH and N;
each X³ is independently selected from CH₂, NR²⁰, O, SO, SO₂, and S;
each g is independently selected from 0, 1, 2, 3, 4, 5, 6, 7, or 8;
each h is independently selected from 0, 1, 2, 3, 4, 5, 6, 7, or 8;
each j is independently selected from 1, 2, 3, 4, 5, 6, 7, or 8; and
each k is independently selected from 1, 2, 3, 4, 5, 6, 7, or 8.

In any aspect or embodiment described herein, the [LINKER] is

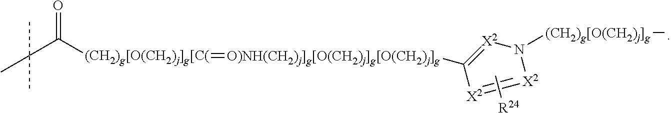

In any aspect or embodiment described herein, the [LINKER] is

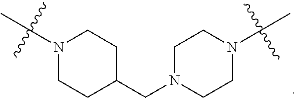 [SMILES: *N1CCC(CN2CCN(*)CC2)CC1]

In any aspect or embodiment described herein, the [LINKER] is a polyalkyleneglycol (e.g. polyethyleneglycol) containing linker having from 1 to 12 alkylene (e.g. ethylene) glycol residues.

In any aspect or embodiment described herein, the [LINKER] is a polypropylene glycol or polypropylene-co-polyethylene glycol linker containing any combination of the groups

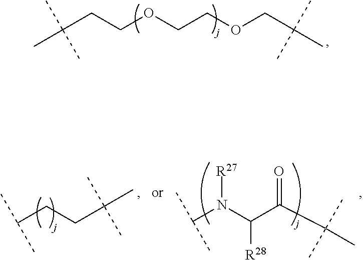

and optionally containing 1 group

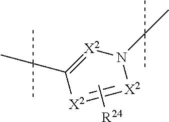

wherein
X² is independently chosen from N and CH;
R²⁴ is 0 to 4 substituents independently selected from halogen, hydroxyl, amino, cyano, C₁-C₄alkyl, C₁-C₄alkoxy, C₁-C₂haloalkyl, and C₁-C₂haloalkoxy;
R²⁷ is H, C₁-C₃ alkyl, C₁-C₃ alkanol, or forms a cyclic ring with R³ to form a pyrrolidine or hydroxypyrroline group;
R²⁸ is a side chain derived from a D- or L amino acid selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline, hydroxyproline, serine (methanol), threonine (ethanol, 1-hydroxyethane), tryptophan (methyleneindole), tyrosine (methylene phenol), or valine (isopropyl); and
j is 0, 1, 2, 3, 4, 5, 6, 7, 8, 9, 10, 11, 12, 13, 14, or 15.

In any aspect or embodiment described herein, the [LINKER] comprises:

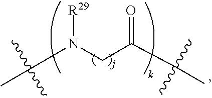

wherein each R²⁹ is H or a C₁-C₃alkyl that is optionally substituted with one or two hydroxyl groups; each j is independently 1, 2, 3, 4, 5, 6, 7, 8, 9, 10, 11, 12, 13, 14, or 15; and k is an integer from 1 to 100.

In any aspect or embodiment described herein, the [LINKER] comprises:

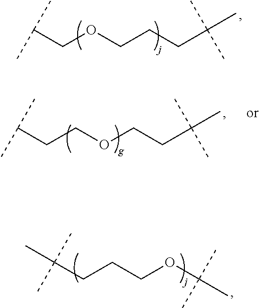

wherein j is an integer from 1 to 25, 1 to 15, 1 to 12, 2 to 11, 2 to 10, 2 to 8, 2 to 6, 2 to 5, 2 to 4 or 2 to 3 (e.g. 1, 2, 3, 4, 5, 6, 7, or 8); and g is 0, 1, 2, 3, 4, 5, 6, 7, or 8.

In any aspect or embodiment described herein, the [LINKER] is:

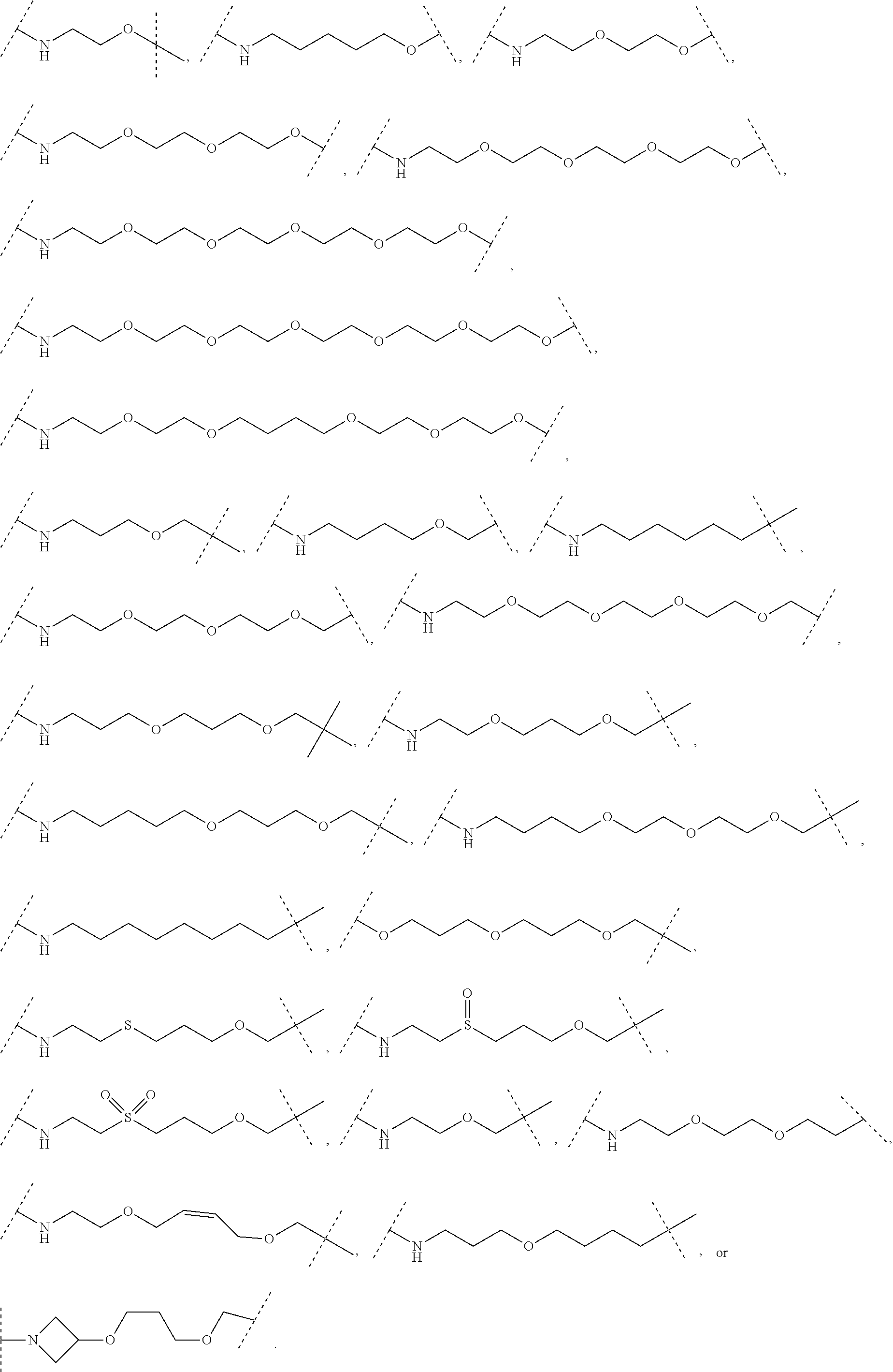

In any aspect or embodiment described herein, the [LINKER] is:

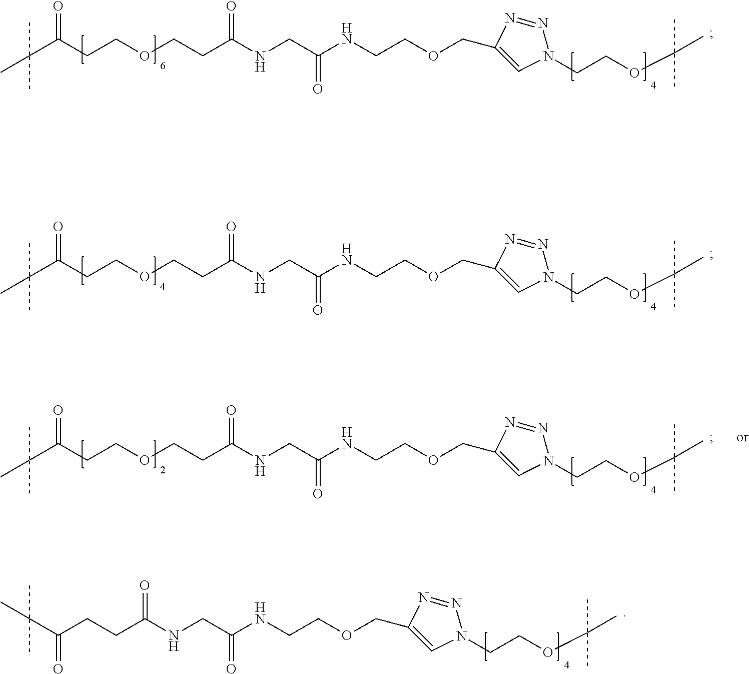

In any aspect or embodiment described herein, the [LINKER] is a C₁-C₅₀ alkylene group (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkylene), wherein:

The compound comprises one to fourteen —C₁-C₂₅ alkylene-[TNFABM] covalently bound to the [LINKER];
the compound comprises one to fourteen —C₁-C₂₅ alkylene-[ASPGPRM] covalently bound to the [LINKER];
each C₁-C₂₅alkylene group of the [LINKER] optionally contains 0 or 1 or more double or triple bonds, each CH₂ group of the [LINKER] is optionally replaced with a group independently selected from O, S, SO, SO₂, NH, CH═CH, C≡C, 5-7 membered heterocycle, and 3-8 membered carbocycle, and is optionally bound to a [CON]; and
the [LINKER] is optionally substituted with one or more (e.g., 1, 2, 3, 4, 5, 6, 7, 8, 9, 10 or more) groups independently selected from halogen, hydroxyl, cyano, oxo (═O), amino, imino (═NH), —COOH, C₁-C₄alkyl, C₁-C₄haloalkyl, and C₁-C₄alkoxy.

The term “connector”, symbolized in the generic formulas by [CON], is used to describe a bond or an optional chemical moiety included in bifunctional compounds of this disclosure which forms from the reaction product of an activated linker with a TNF-α binding moiety ([TNFABM]), which also is preferably activated for covalently bonding the linker with the moiety, or a [ASGPRBM] with an activated linker. The connector group is often the resulting moiety which forms from the facile condensation of two or more separate chemical fragments which contain reactive groups which can provide connector groups as otherwise described to produce bifunctional or multifunctional compounds of this disclosure. It is noted that a connector may be distinguishable from a [LINKER] in that the [CON] is the result of a specific chemistry which is used to provide bifunctional compounds of this disclosure wherein the reaction product of these groups results in an identifiable connector group or part of a connector group which is distinguishable from the [LINKER] group, although in certain instances, the connector group is incorporated into and integral with the [LINKER] group as otherwise described herein. It is noted also that a connector group may be linked to a number of linkers to provide multifunctionality (i.e., more than one [TNFABM] and/or more than one [ASGPRBM]) within the same molecule. It is noted that there may be some overlap between the description of the connector group and the [LINKER] group such that the connector group is actually incorporated or forms part of the [LINKER], especially with respect to more common connector groups such as amide groups, oxygen (ether), sulfur (thioether) or amine linkages, urea or carbonate —OC(O)O— groups or as otherwise described herein. It is further noted that a [CON](or [LINKER]) may be connected to [TNFABM], [ASGPRBM], or a [LINKER] at positions which are represented as being linked to another group using the symbol

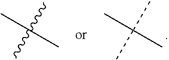

Where two or more such groups are present in a linker or connector, any of an [ASGPRBM], a [LINKER], or a [TNFABM] group may be bonded to such a group. Where that symbol is not used, the [LINKER] may be at one or more positions of a moiety.

In any aspect or embodiment described herein, one or more [CON] is a bond.

It is noted that each connector may be extended with one or more methylene groups to facilitate connection to a linker group, another CON group, or a [ASGPRBM]. It is noted that in certain instances, within context the diamide group may also function independently as a linker group.

In any aspect or embodiment described herein, the bifunctional compounds comprise a [LINKER]-[CON] group in either orientation, which [LINKER]-[CON] group includes at least one [CON] comprising:

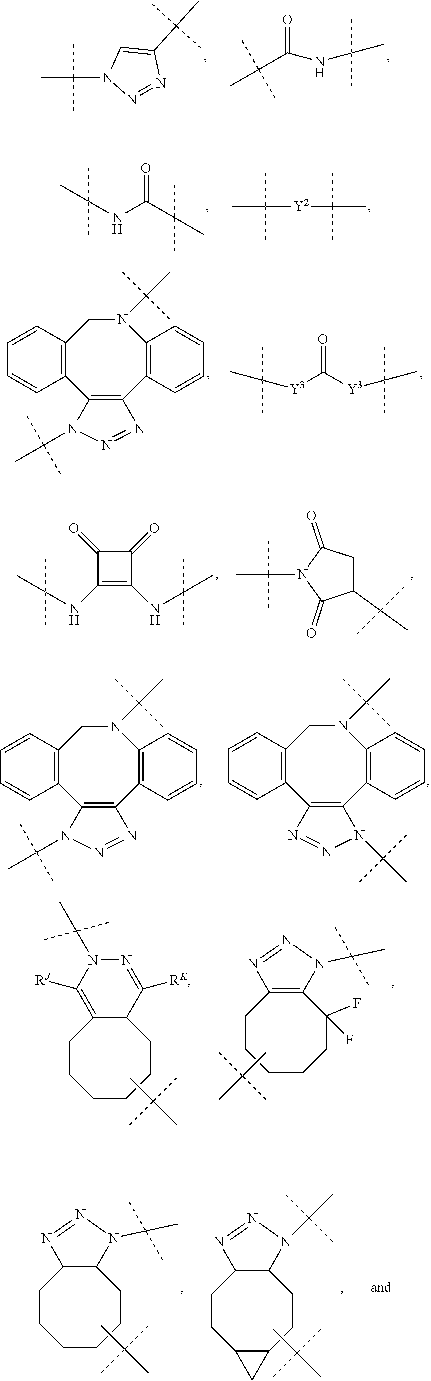

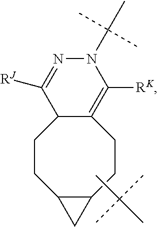

wherein:
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group;
each R⁸ is independently H or C₁-C₃ alkyl group (preferably H); and
R^{J} and R^{K} are each independently H, methyl, a bond (e.g., for attachment to [TNFABM], [LINKER], or [ASGPRBM]); or a dioxo or diamide group according to the structure:

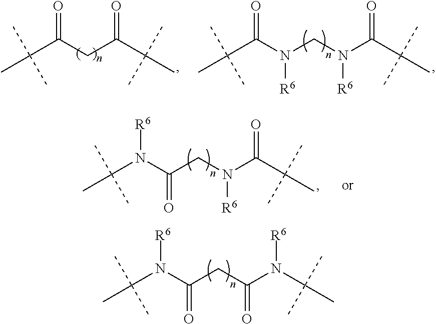

(e.g., one end is for attachment to [TNFABM], [LINKER], or [ASGPRBM]), wherein each R is independently H or a C₁-C₃ alkyl group (preferably H); and n is an integer from 1 to 10 (n is preferably 1, 2, 3, 4, 5, or 6.

In any aspect or embodiment described herein, one or more [CON] has the formula [C1]-PAG, wherein the compound comprises a [CON]-[LINKER] group in either orientation of the formula [C1]-[PAG], wherein each [PAG] is a polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein the [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) 0 atoms of the [PAG] is optionally replaced with NH or NC₁-C₃alkyl; and each [C1] is independently selected from:

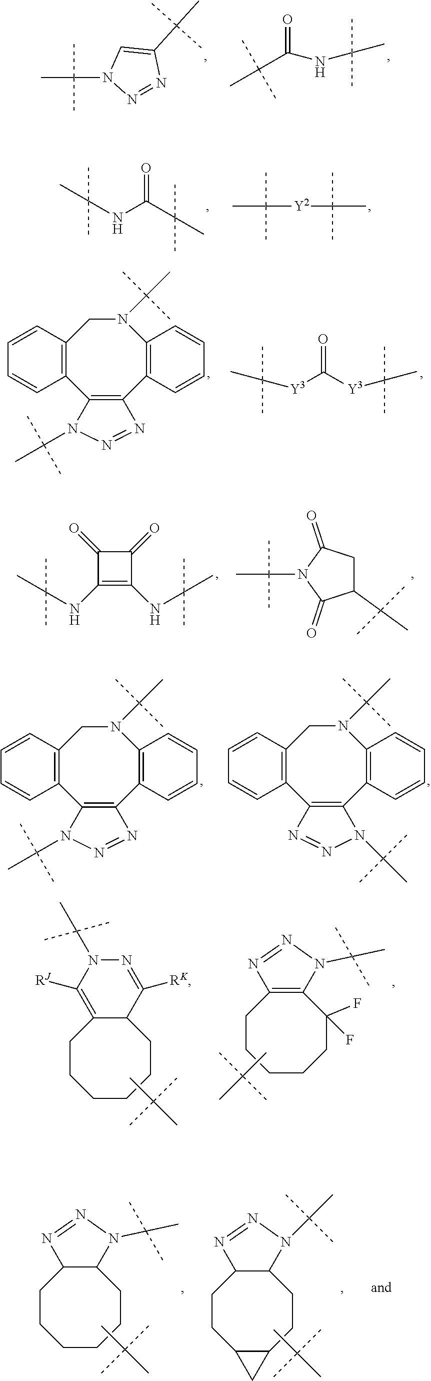

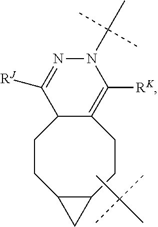

wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure:

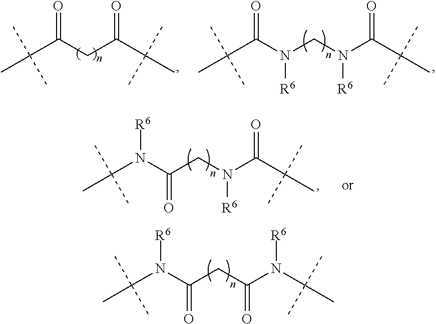

(e.g., one end is for attachment to [TNFABM], [PAG], [LINKER], or [ASGPRBM]), wherein each R⁶ is independently H or a C₁-C₃ alkyl group (preferably H); and n is an integer from 1 to 10, or 1 to 8, or 1 to 6 (e.g. 1, 2, 3, 4, 5, or 6);

In any aspect or embodiment described herein, one or more [CON] has the formula [C1]-PAG, wherein the [CON]-[LINKER] group has the formula —CH₂(CH₂O—CH₂-[C1]-[PAG])₁, —CH₁(CH₂O—CH₂-[C1]-[PAG])₂, or —C(CH₂O—CH₂-[C1]-PAG)₃, wherein each [PAG] is an independently chosen polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein the [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) 0 atoms of the [PAG] is optionally replaced with NH or NC₁-C₃alkyl; and each [C1] is independently selected from:

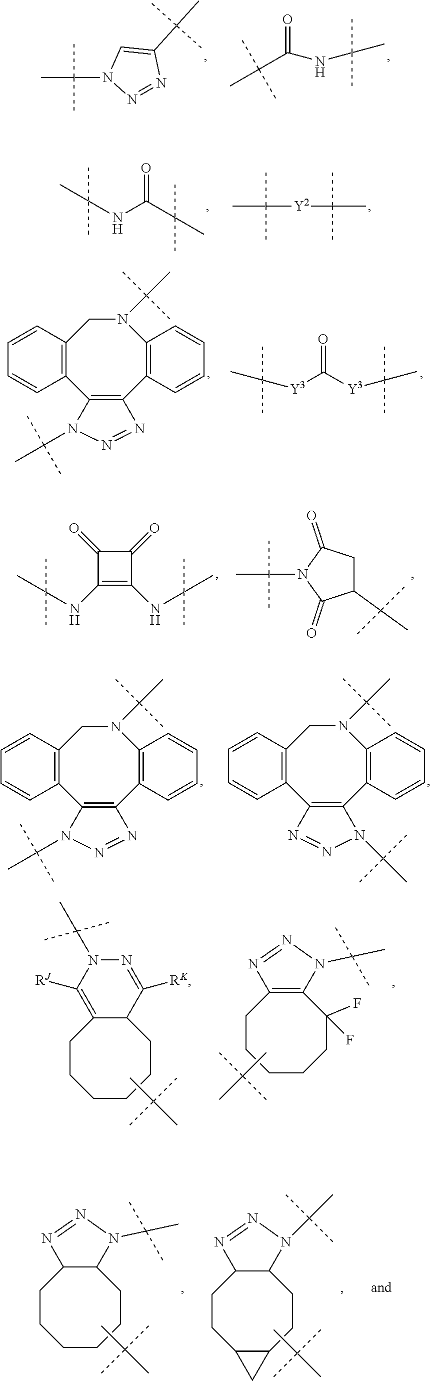

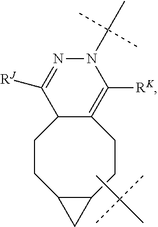

In any aspect or embodiment described herein, one or more [CON] has the formula [C1]-PAG, wherein the [CON] is:

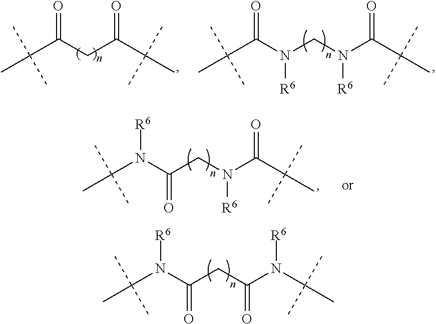

In any aspect or embodiment described herein, one or more [CON] has the formula [C1]-PAG, wherein the [CON] has formula:

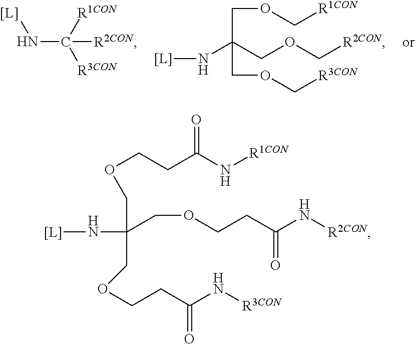

wherein
[L] represent the point of attachment to a first [LINKER]; and
R^{1CON}, R^{2CON}, and R^{3CON} are each independently H, a bond, or a chemical moiety, wherein the bond and the chemical moiety are covalently attached to an [ASPGPRM] or a second, third, or fourth independently chosen [LINKER], and at least one of R^{1CON}, R^{2CON}, and R^{3CON} is not H).

In any aspect or embodiment described herein, one or more (e.g., 1, 2, or 3) of R^{1CON}, R^{2CON}, and R^{3CON} includes a group selected from:

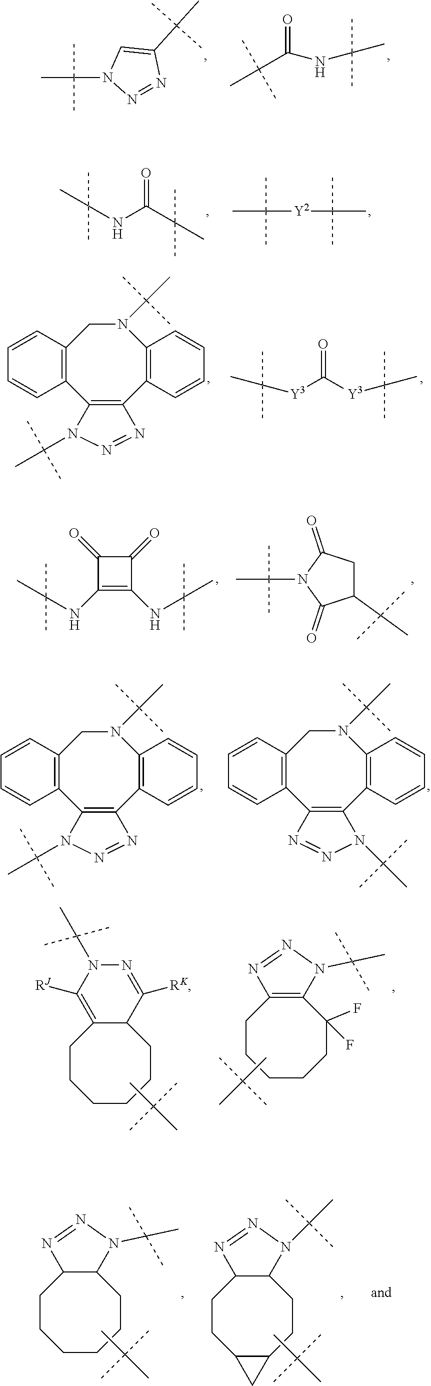

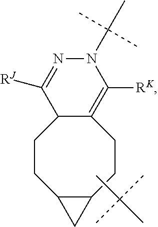

wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure:

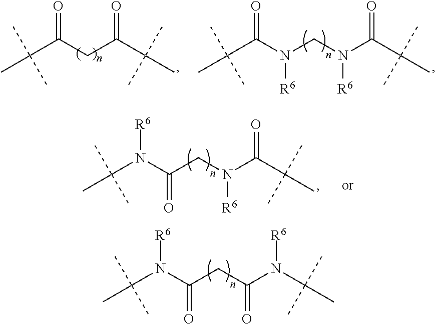

(e.g., one end is for attachment to [TNFABM], [LINKER], or [ASGPRBM]), wherein each R⁶ is independently H or a C₁-C₃ alkyl group (preferably H); and n is independently an integer from 0 to 10, 1 to 8, 0 to 8 or 1 to 7 (e.g. 1, 2, 3, 4, 5, or 6).

In any aspect or embodiment described herein, one or more [CON] is

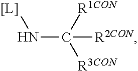

wherein at least one of R^{1CON}, R^{2CON}, and R^{3CON} is not H, and each of R^{1CON}, R^{2CON}, and R^{3CON} is independently selected from: H, bond,

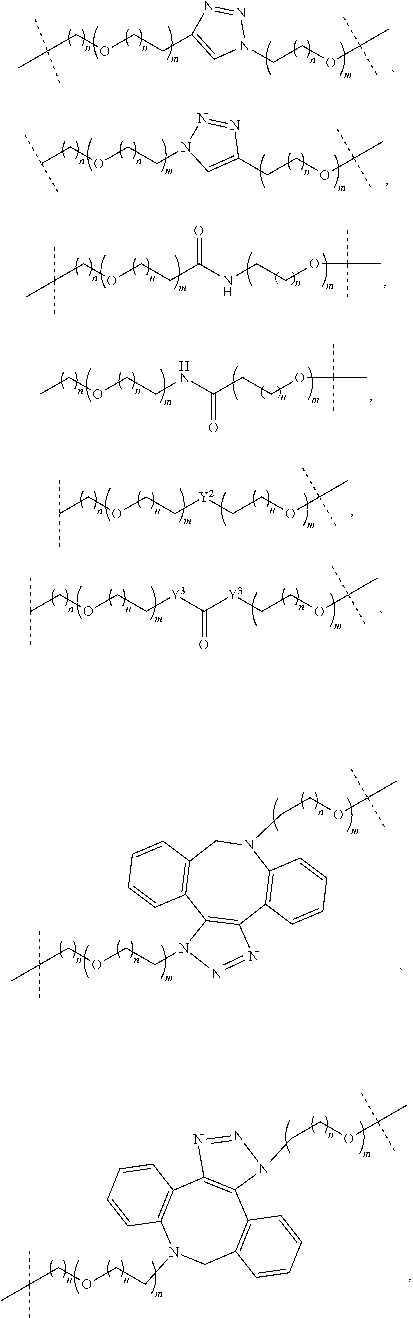

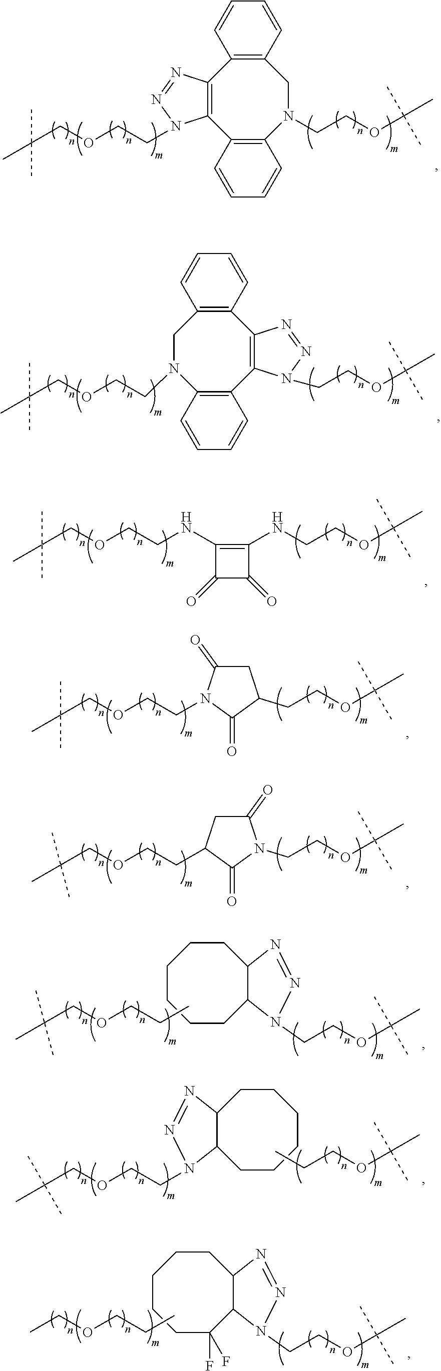

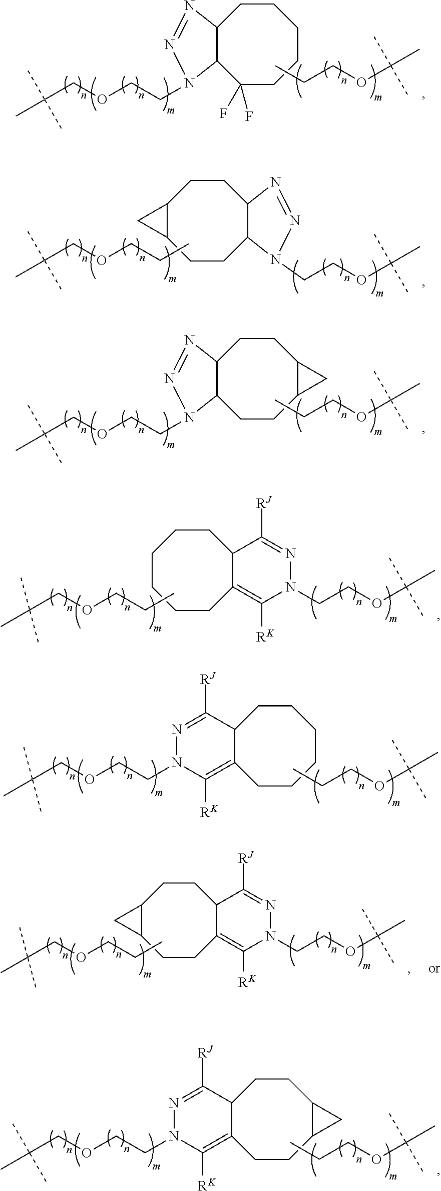

wherein
each n is independently 1, 2, 3, 4, 5, 6, 7, 8, 9, or 10;
each m is independently 0, 1, 2, 3, 4, 5, 6, 7, 8, 9, or 10;
each Y² is independently CH₂, O, S, NR⁷, C(O), S(O), S(O)₂, —S(O)₂O, —OS(O)₂, or OS(O)₂O;
each Y³ is independently O, S, or NR⁷; and
each R⁷ is independently H, C₁-C₃ alkyl, C₁-C₃ alkanoyl, or —C(O)(C₁-C₃alkyl) group.
R^{J} and R^{K} are each independently H, methyl, a bond (e.g., for attachment to [TNFABM], [LINKER], or [ASGPRBM]), or a dioxo or diamide group according to the structure:

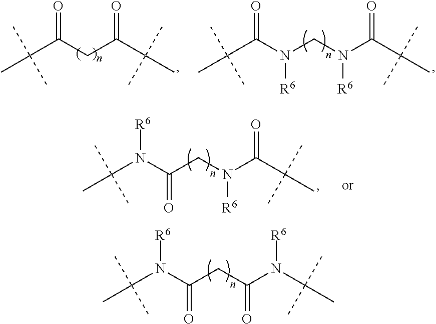

(e.g., one end is for attachment to [TNFABM], [LINKER], or [ASGPRBM]), wherein each R⁶ is independently H or a C₁-C₃ alkyl group (preferably H) n is an integer from 1 to 10 (is preferably and integer from 0 to 8, or 0 to 7, or 0, 1, 2, 3, 4, 5, or 6).

In any aspect or embodiment described herein, one or more [CON] has the formula [C1]-PAG, wherein R^{1CON}, R^{2CON}, and R^{3CON} are each independently H, —(CH₂)ᵣ—, —(CH₂)ₛC(O)(NR)—(CH₂)ₛ—, —(CH₂)ₛ(NR⁴)_{XA}C(O)_{XA}—(CH₂)ₛ—, and —(CH₂)ₛO—(CH₂)ₜ—C(O)NR⁸—, with the proviso that R^{1CON}, R^{2CON}, and R^{3CON} are not simultaneously H, wherein
each r is independently 1, 2, 3, or 4 (preferably 1 or 2);
each s is independently 0, 1, 2, 3, or 4 (preferably 0, 1 or 2);
each XA is independently 0 or 1; and
   each R⁸ is independently H, C₁-C₃ alkyl, C₁-C₃ alkanol, or —C(O)(C₁-C₃), with the proviso that s and XA in a moiety are not all simultaneously 0.

In any aspect or embodiment described herein, one or more the [LINKER]-[CON] has the formula: [PEG]-[C1]-[PEG], wherein each [PEG] is independently a polyethylene glycol group containing from 1, 2, 3, 4, 5, 6, 7, 8, 9, 10, 11, or 12 ethylene glycol residues, and [C1] is atriazole group

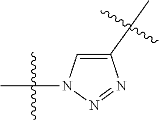 [SMILES: *c1cn(*)nn1]

In any aspect or embodiment described herein, the [LINKER]-[CON] has the formula: [PAG]-[C1]-[PAG], wherein each [PAG] is independently a polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein each [PAG] is optionally substituted with one or more (e.g. 1, 2, 3, 4, or 5) groups independently selected from hydroxyl, halogen, cyano, oxo, amino, C1-C₃alkyl, and —COOH, and one or more (e.g., 1, 2, 3, 4, or 5) 0 atoms of each [PAG] is optionally replaced with NH or NC₁-C₃alkyl; and [C1] is selected from:

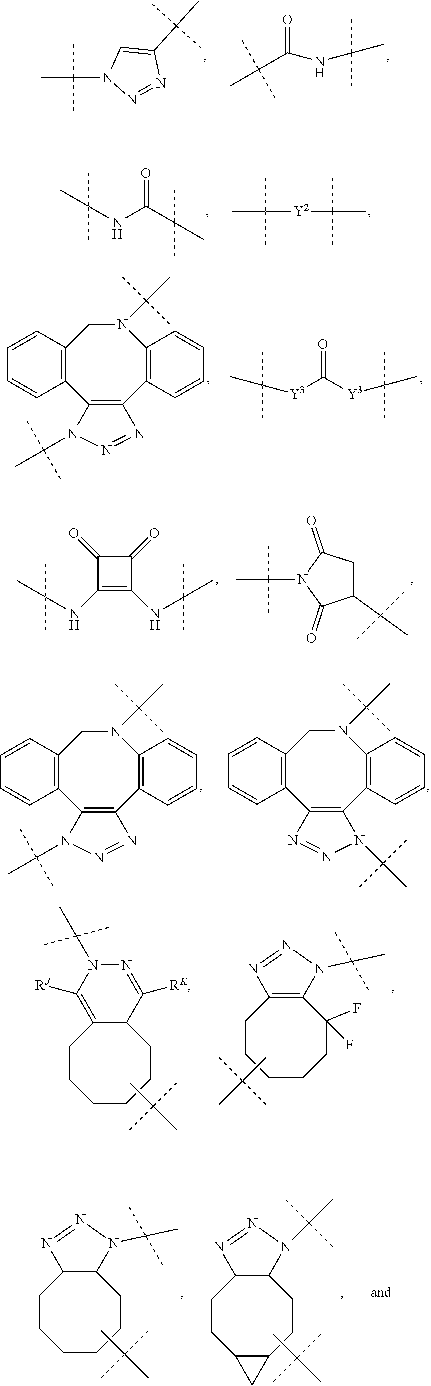

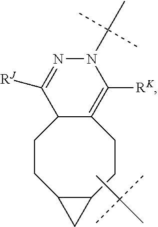

wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure:

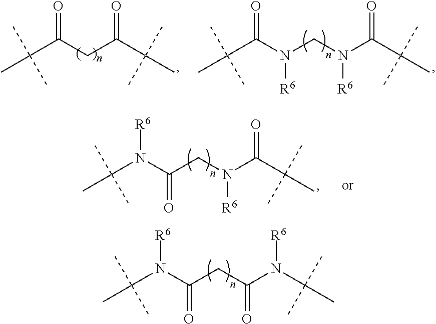

(e.g., one end is for attachment to [TNFABM], [LINKER], or [ASGPRBM]), wherein each R⁶ is independently H or a C₁-C₃ alkyl group (preferably H); and z is independently an integer from 0 to 8 or 1 to 7 (e.g. 1, 2, 3, 4, 5, or 6).

In any aspect or embodiment described herein, the [LINKER]-[CON] has the formula [PAG]-CH₂(CH₂O—CH₂-[C1]-[PAG])₁, [PAG]-CH₁(CH₂O—CH₂-[C1]-[PAG])₂, or [PAG]-C(CH₂O—CH₂-[C1]-[PAG])₃, wherein
each [PAG] is a polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein the [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) O atoms of the [PAG] is optionally replaced with NH or NC₁-C₃alkyl; and
each [C1] is independently selected from:

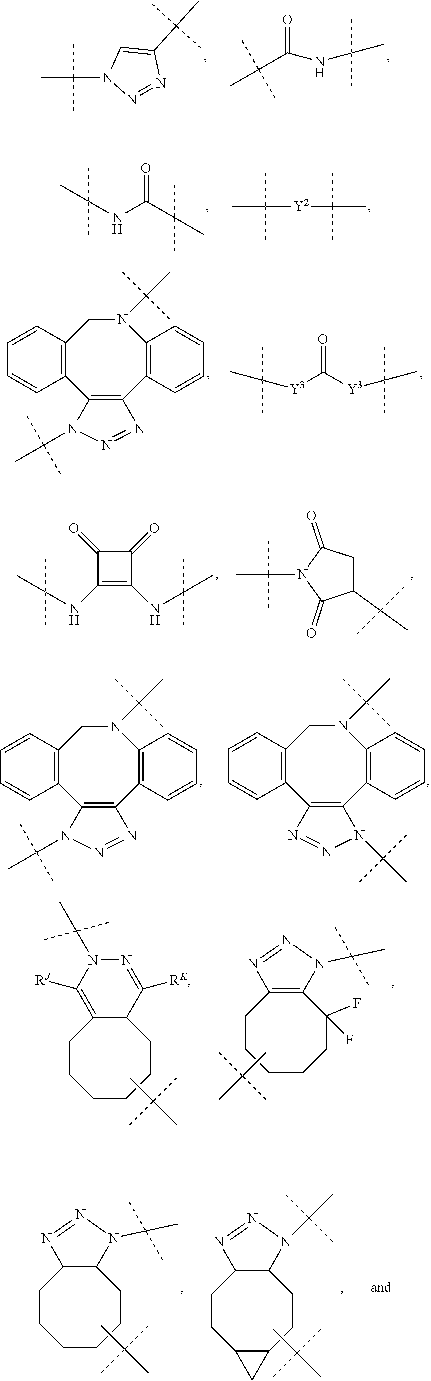

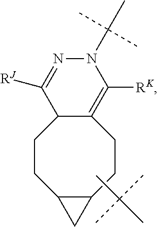

wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure:

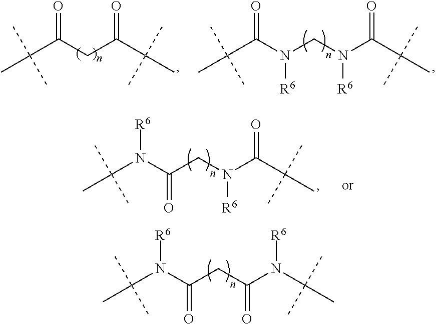

(e.g., one end is for attachment to [TNFABM], [LINKER], or [ASGPRBM])

In any aspect or embodiment described herein, the bifunctional compound or salt thereof contains at least one [CON]-[LINKER]₃-[ASPGPRM]₃ group selected from (a) and (b) or at least one [CON]-[LINKER]-[ASGPRM] group:

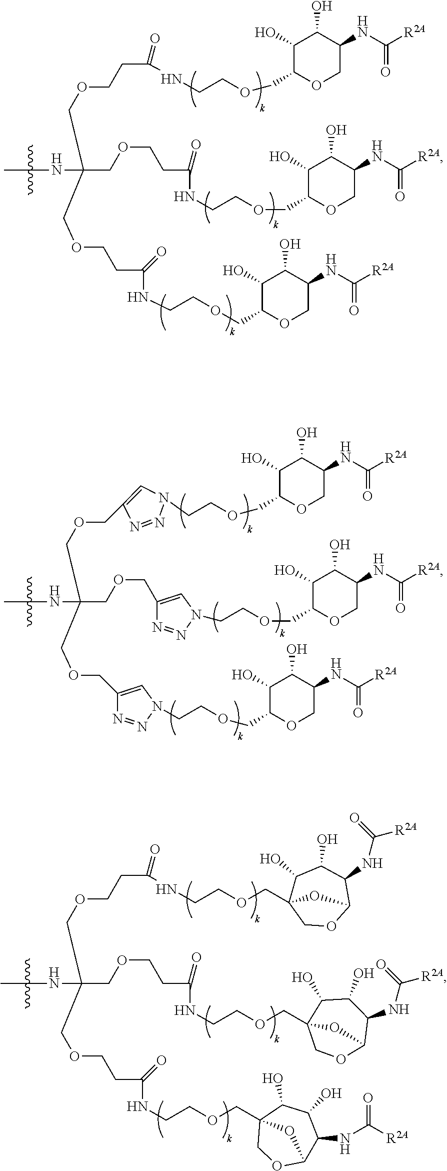

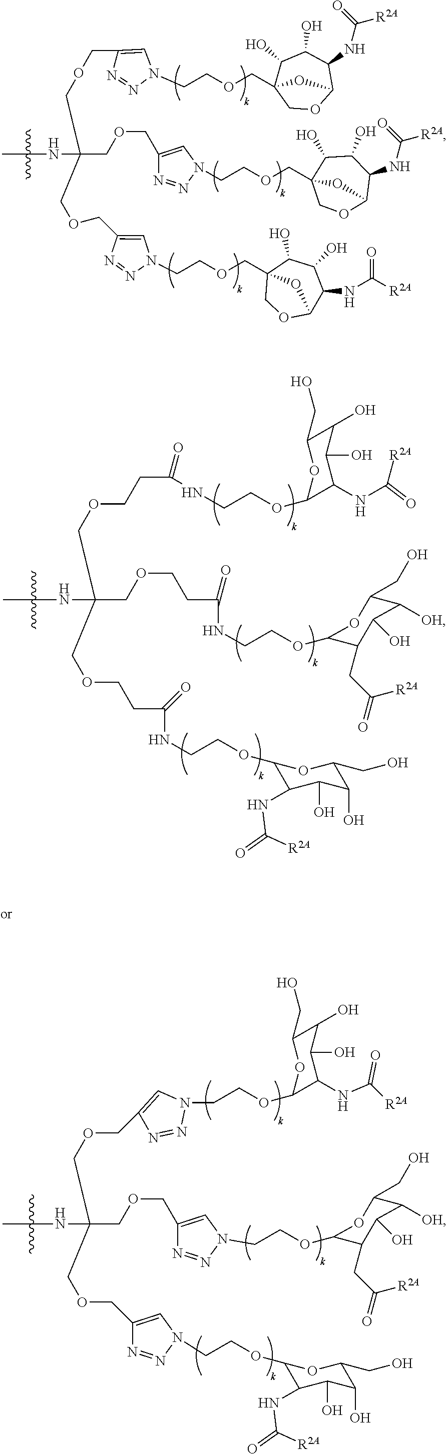

wherein each k is independently 0, 1, 2, 3, 4, 5, 6, 7, 8, 9, or 10 (preferably, 0, 1, 2, 3, 4, or 5), and each R^{2A} is independently a C₁-C₄alkyl (e.g., C1-C₂alkyl) that is optionally substituted with 1 to 5 (e.g., 1, 2, 3, 4, or 5) independently selected halogen atoms.

In any aspect or embodiment described herein, one or more [CON]-[ASPGPRM] is

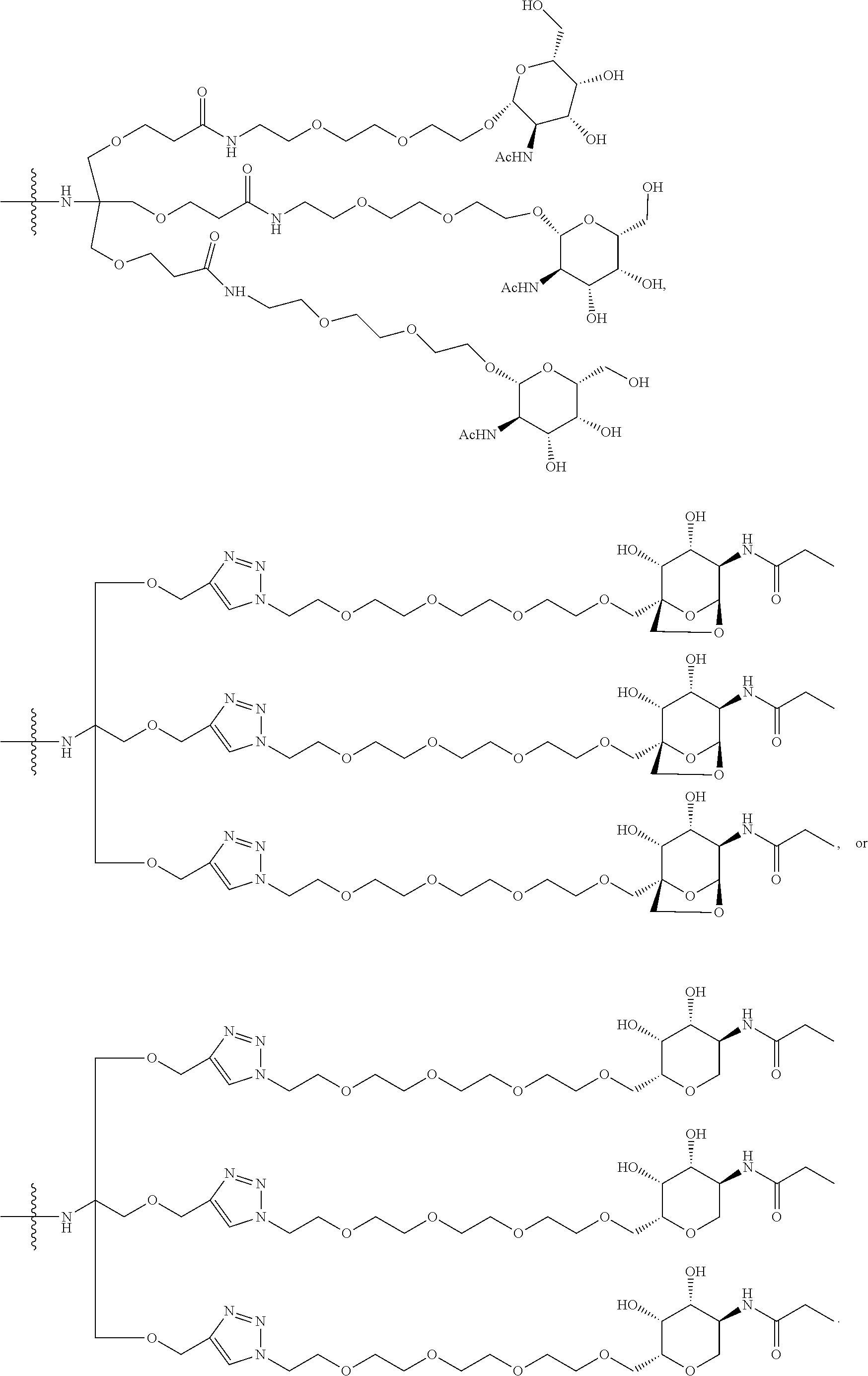

In any aspect or embodiment described herein, one or more [CON]-[ASPGPRM] is:

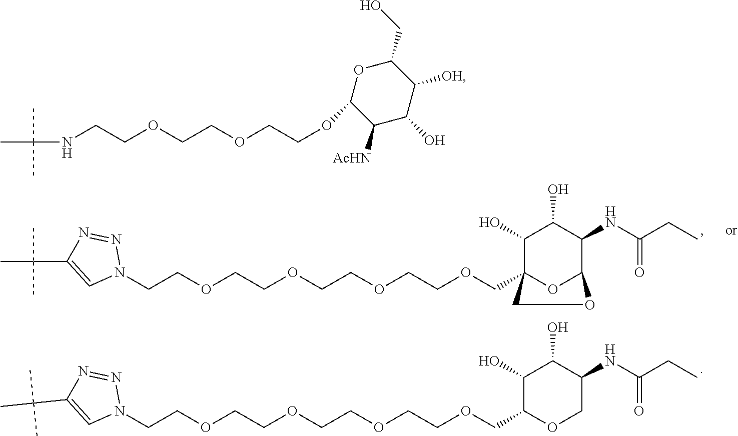

In any aspect or embodiment described herein, the bifunctional compound is selected from:

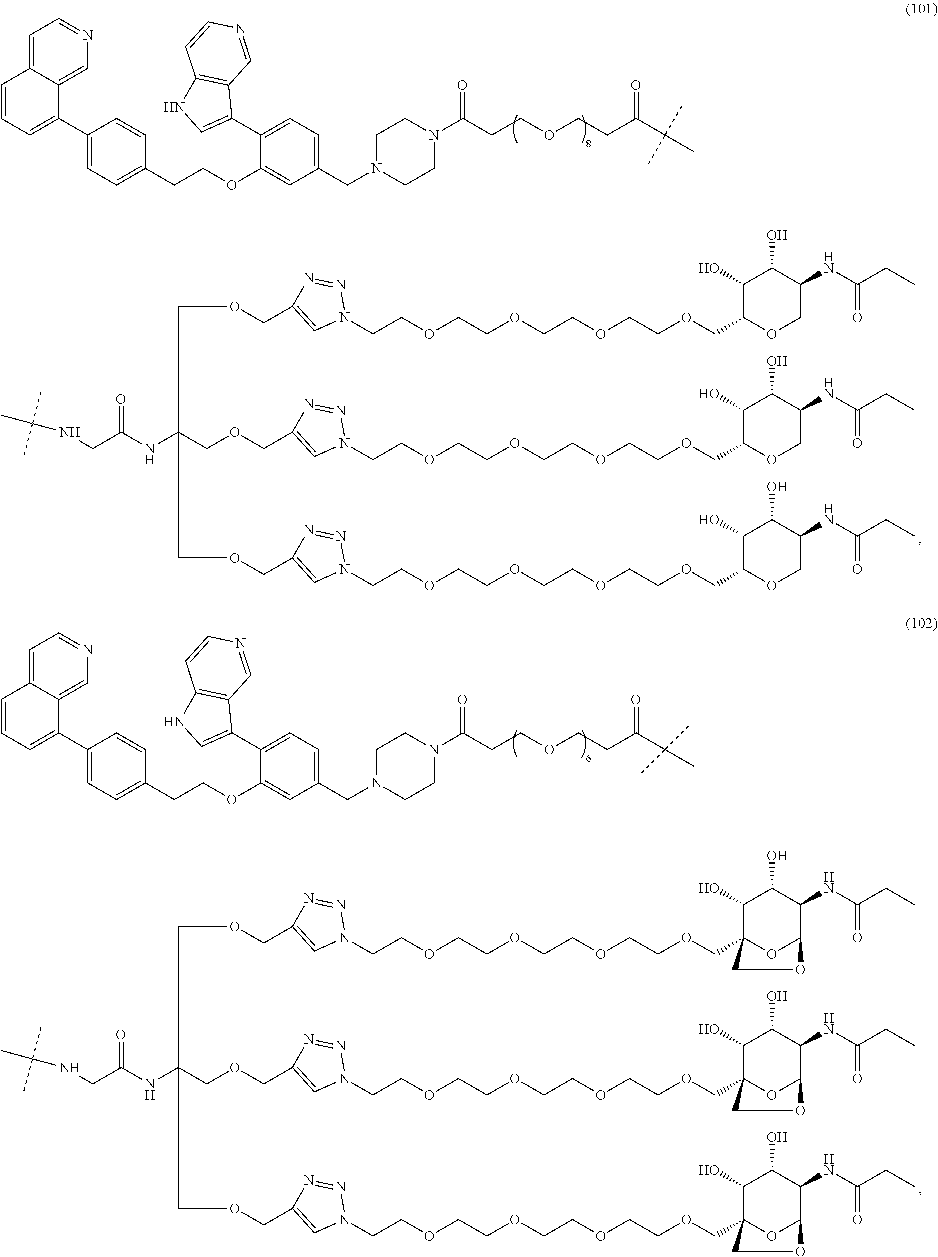

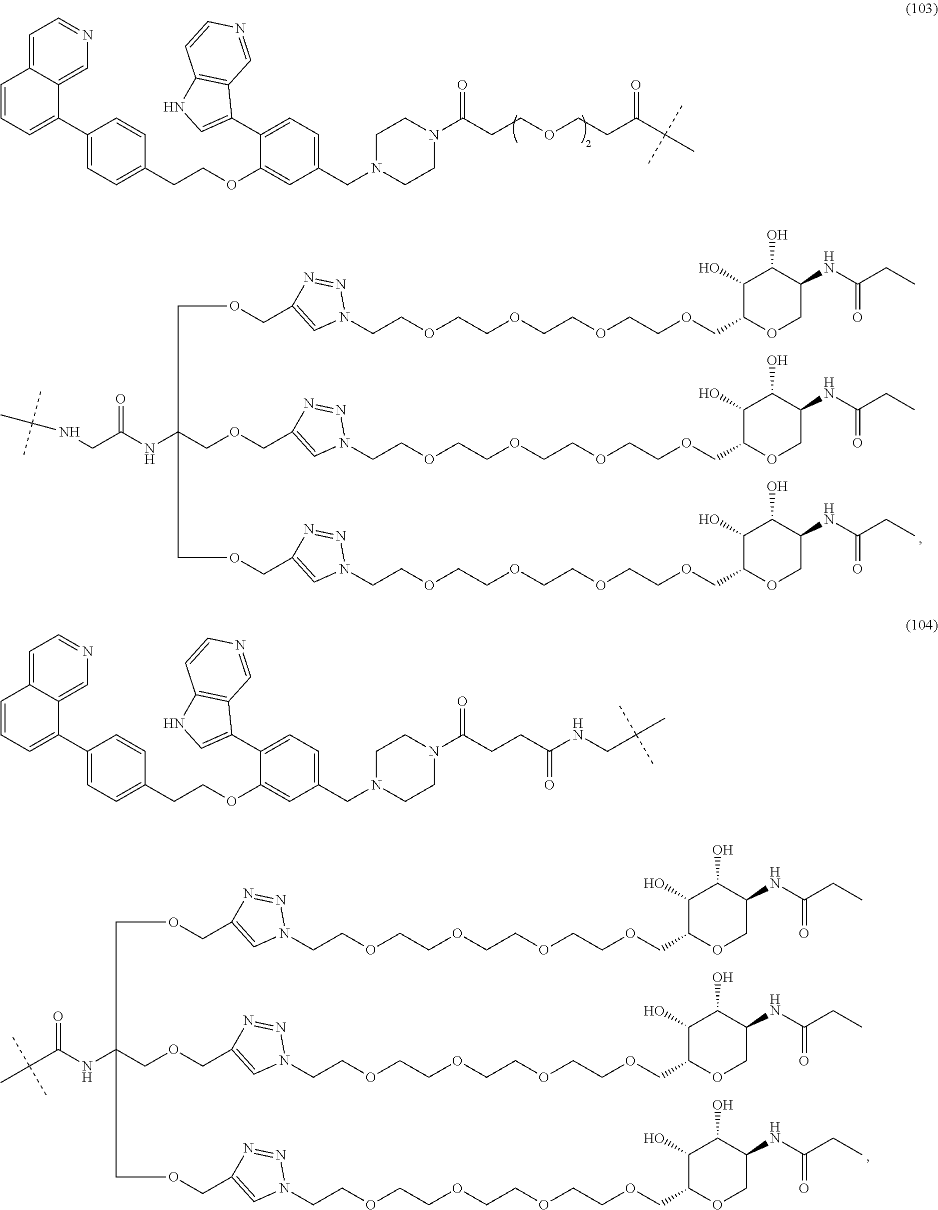

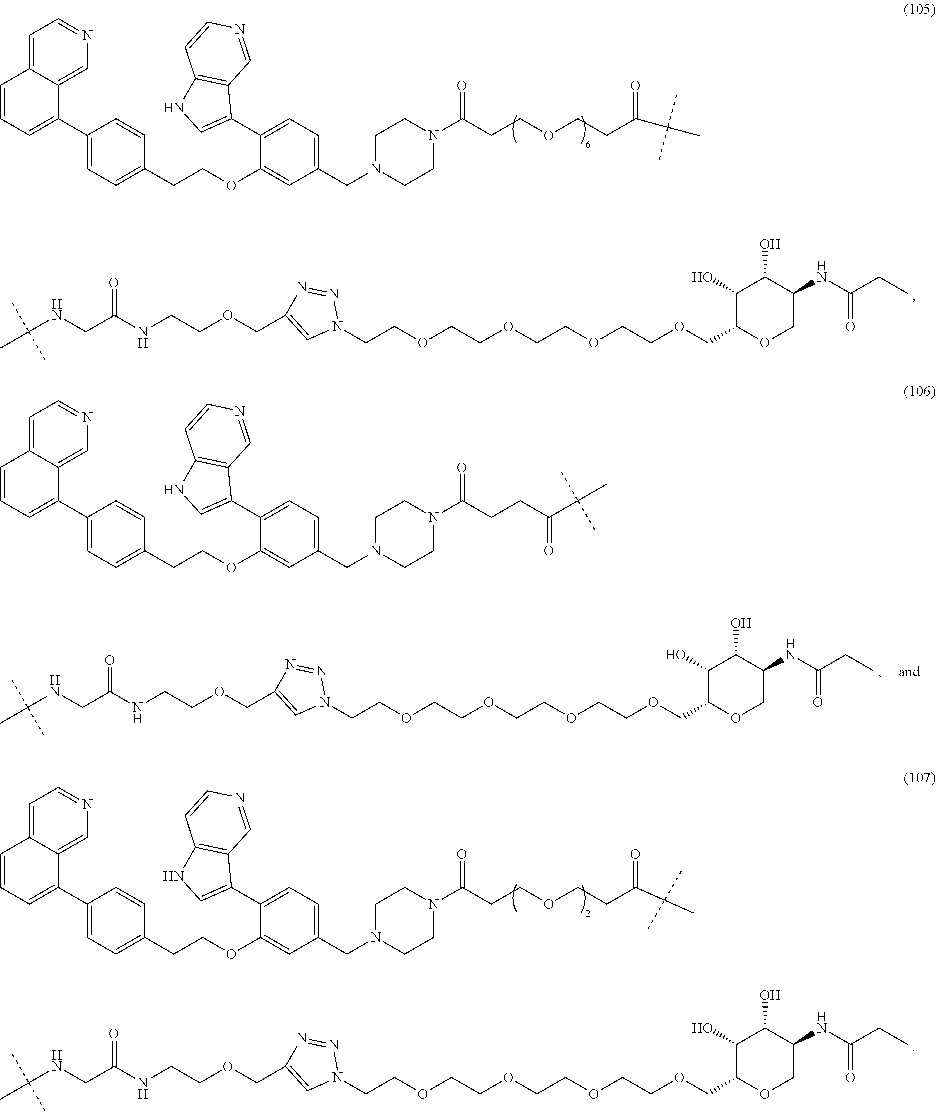

or a pharmaceutically acceptable salt thereof.

For compounds which contain a [MATE] group, the [LINKER] and [CON] groups can be any of the above groups. The [MATE] can be

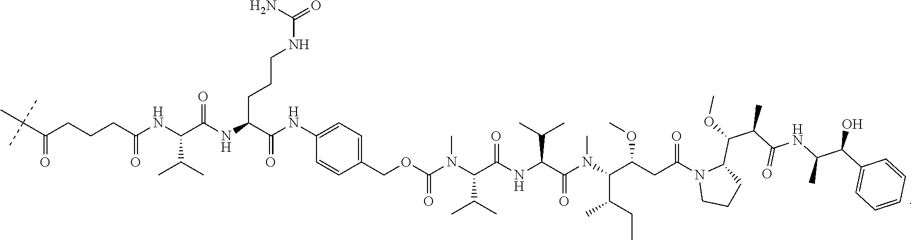

The [RG] within the [MATE] moiety can be

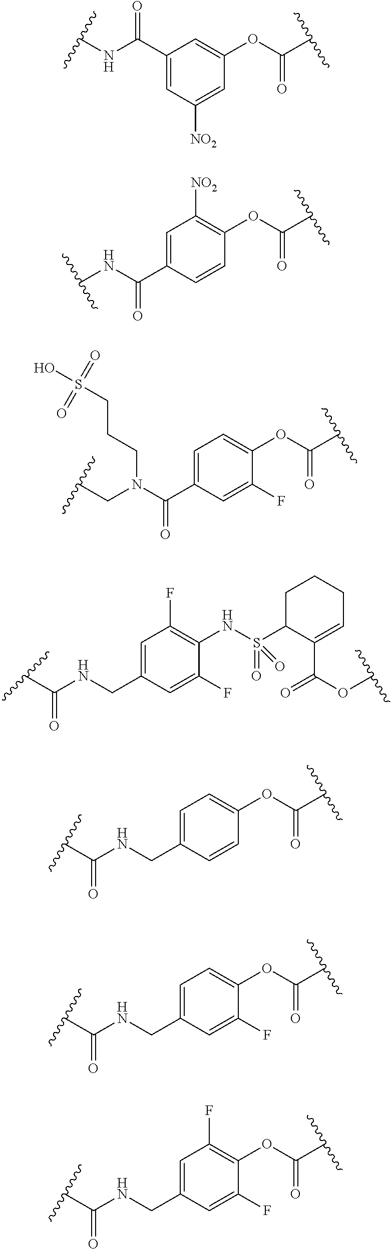

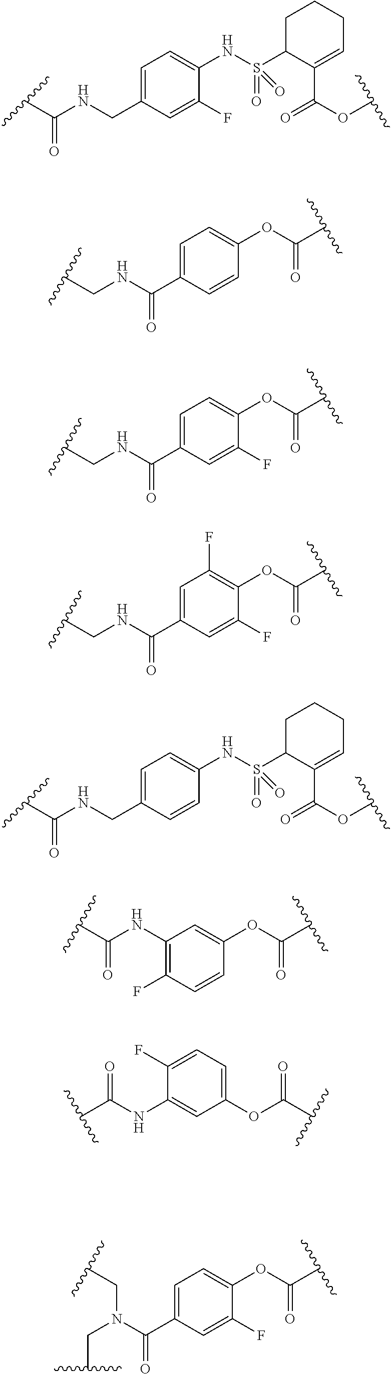

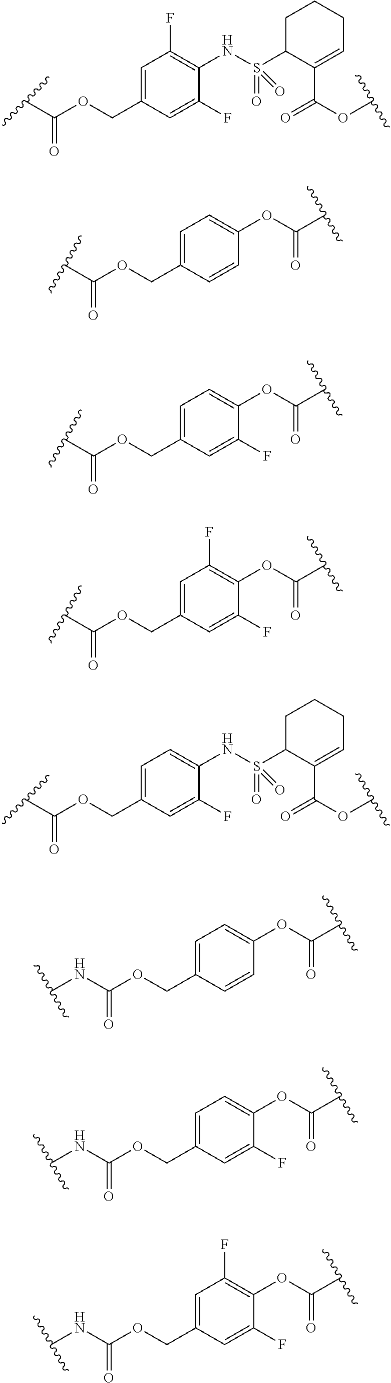

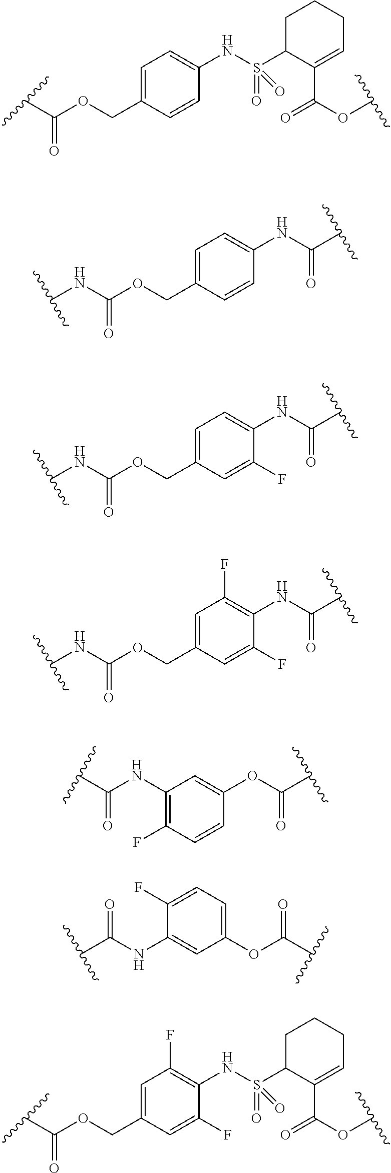

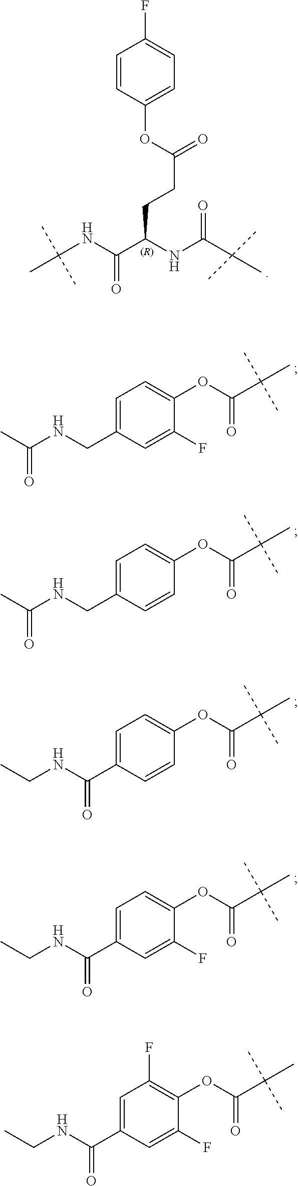

The [ABM](antibody binding moiety) within the [MATE] group can be

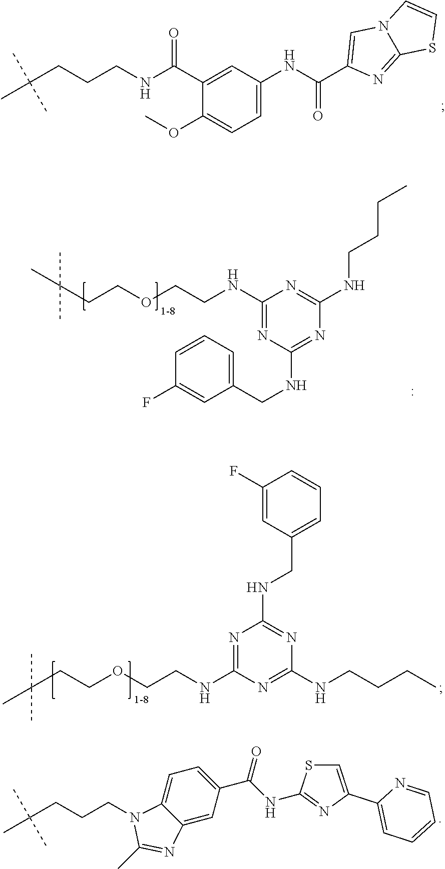

In certain embodiments the [MATE] is

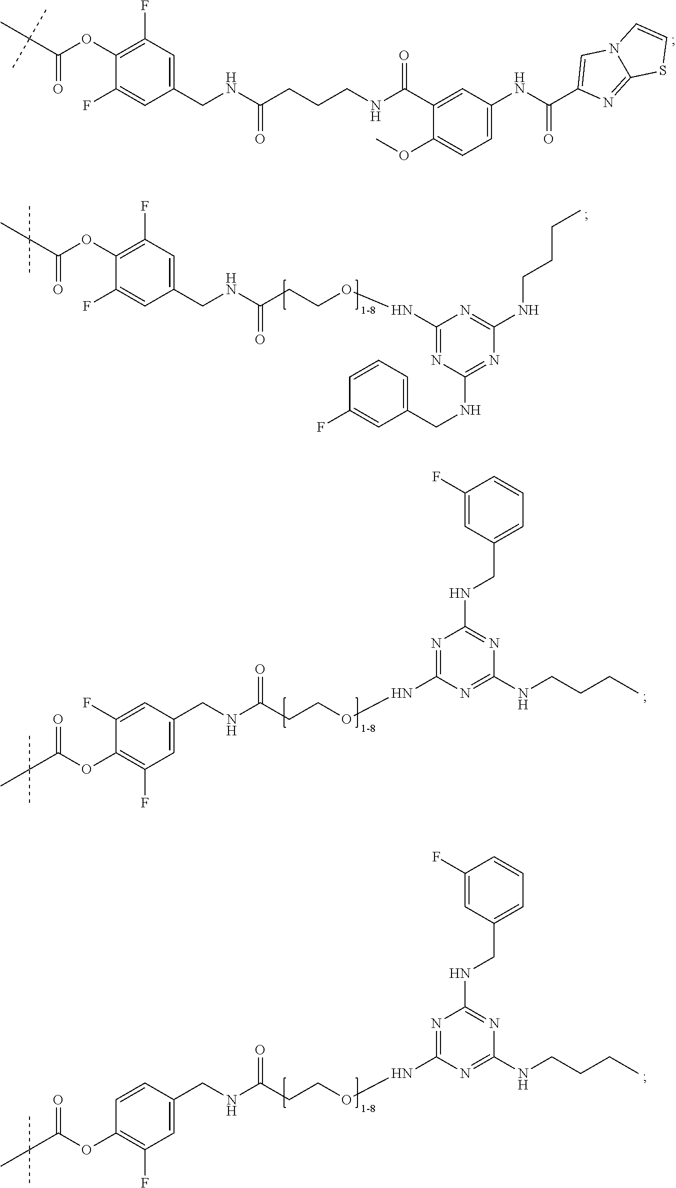

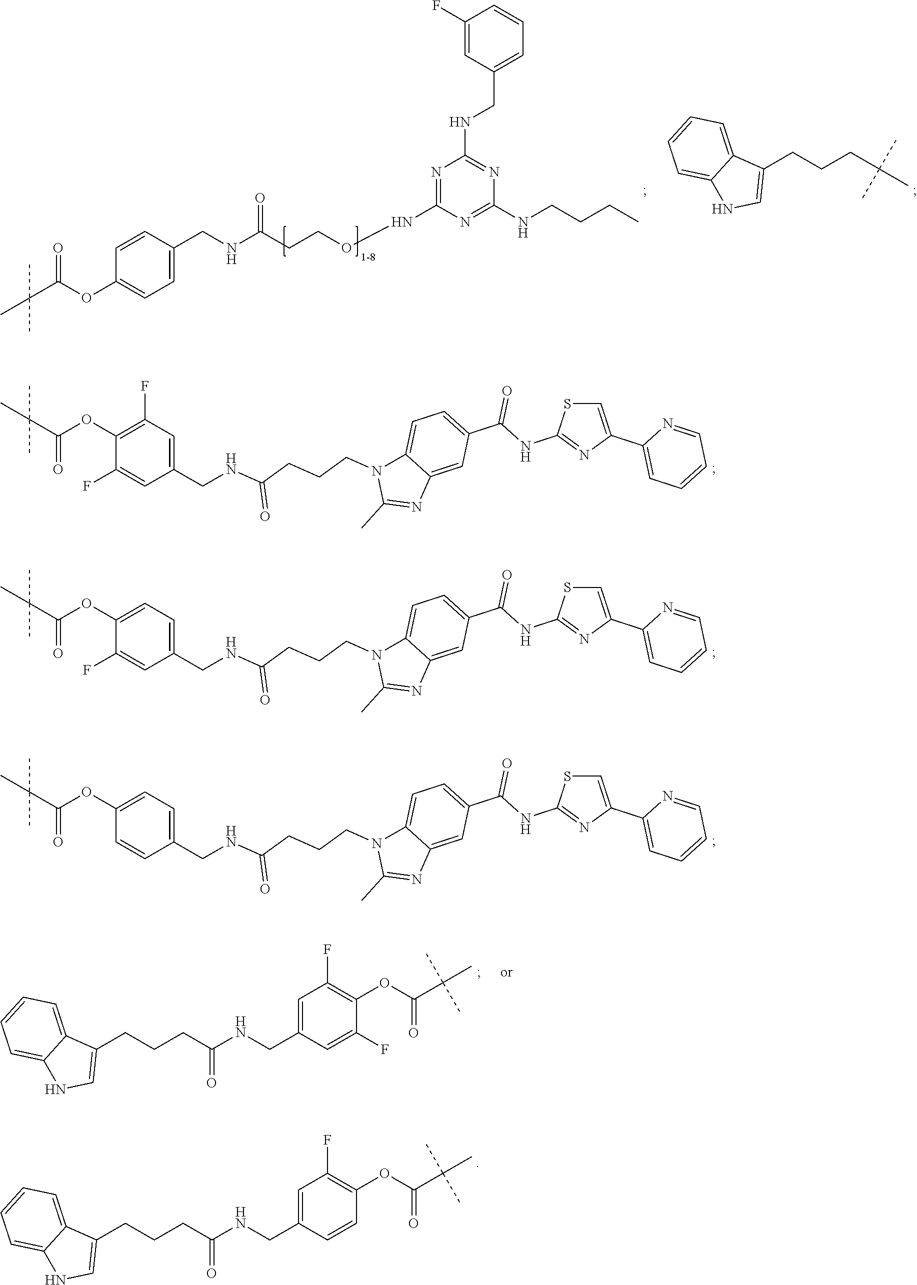

A further aspect of the present disclosure relates to a pharmaceutical composition comprising one or more (e.g., 1, 2, or 3) bifunctional compound of the present disclosure, or a pharmaceutically acceptable salt thereof, and a pharmaceutically acceptable carrier or excipient.

### Methods of Treatment

The disclosure includes methods of treating a disease or disorder responsive to TNF-α inhibition in a patient having a disease or disorder responsive to TNF-α inhibition comprising administering a therapeutically effective amount of a bifunctional compound of the disclosure to the patient. Thus, an aspect of the present disclosure relates to a method of treating a disease or disorder responsive to TNF-α inhibition in a patient or subject having a disease or disorder responsive to TNF-α inhibition, the method comprising administering a therapeutically effective amount of a bifunctional compound of the present disclosure, a pharmaceutically acceptable salt thereof, or a pharmaceutical composition of the present disclosure, to the patient or subject. In any aspect or embodiment describe herein, the disease or disorder can be disorder is Alzheimer's disease, cancer, atherosclerosis, depression (such as, major depressive disorder), sepsis, diabetes, obesity, or autoimmune or inflammatory disease or disorder. In any aspect or embodiment described herein, the bifunctional compound is effective at ameliorating one or more symptoms of, or treating, the disease or disorder. In any aspect or embodiment described herein, the method effectuates a decrease in the level of circulating TNF-α. In any aspect or embodiment described herein, the decrease in TNF-α is effected by internalizing the circulating TNF-α into a cell (e.g. hepatocyte) through an ASGPR. In any aspect or embodiment described herein, the internalized TNF-α is degraded.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the “cancer” includes all cancerous disease states and embraces or encompasses the pathological process associated with malignant hematogenous, ascitic and solid tumors. Cancers include, without limitation, morphological irregularities in cells in tissue of a subject or host, as well as pathologic proliferation of cells in tissue of a subject, as compared with normal proliferation in the same type of tissue. Additionally, cancers include benign tumors and malignant tumors (e.g., breast cancer, pancreatic cancer, ovarian cancer, endometrial cancer, oral cancer, gastric cancer, liver cancer, prostate cancer, bladder cancer, colorectal cancer, multiple myeloma, lymphoma, and/or leukemia) that are either invasive or noninvasive. Examples of cancers that may be treated with a bifunctional compound of the disclosure include, without limitation, carcinomas (e.g., squamous-cell carcinomas, adenocarcinomas, hepatocellular carcinomas, choriocarcinoma, and renal cell carcinomas), particularly leukemias (e.g. acute lymphocytic leukemia, acute myelogenous leukemia, hairy cell leukemia); benign and malignant lymphomas (e.g. Burkitt's lymphoma and Non-Hodgkin's lymphoma); benign and malignant melanomas; non-melanoma skin cancer; myeloproliferative diseases; sarcomas (e.g. Ewing's sarcoma, hemangiosarcoma, Kaposi's sarcoma, liposarcoma, myosarcomas, peripheral neuroepithelioma, rhabdomyosarcoma, and synovial sarcoma); tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer (e.g., metastatic prostate cancer), cervical cancer, uterine cancer, lung cancer (e.g., small cell lung cancer), ovarian cancer, testicular cancer, thyroid cancer, astrocytoma, esophageal cancer, pancreatic cancer, stomach cancer, liver cancer, colon cancer, rectal cancer, head and neck cancer, kidney cancer, laryngeal cancer, mouth cancer, oropharyngeal cancer, neck cancer, throat cancer and melanoma); mixed types of neoplasias, particularly carcinosarcoma and Hodgkin's disease; endometrial cancer; bladder cancer, renal cancer, brain/CNS cancers, multiple myeloma and tumors of mixed origin, such as Wilms' tumor and teratocarcinomas. Benign neoplasm, e.g. meilanocytorna. Particularly, the disclosure includes a method of treating lymphomas, ovarian carcinoma, multiple myeloma, and non-small cell lung cancer in a patient comprising administering a therapeutically effective amount of a bifunctional compound of the disclosure to the patient.

Because of TNF-α inhibitory activity of the bifunctional compounds of the disclosure, these compound may be used to treat cancer in virtually any cell type or tissue so long as TNF-α plays a role has general applicability treating virtually any cancer in any tissue, thus the compounds, compositions and methods of the present disclosure are generally applicable to the treatment of cancer and in reducing the likelihood of development of cancer and/or the metastasis of an existing cancer. In any aspect or embodiment described herein, the cancer which is treated is metastatic cancer, a recurrent cancer or a drug resistant cancer, especially including a multiple drug resistant cancer. In any aspect or embodiment described herein, the disease or disorder is a cancer selected from breast cancer, pancreatic cancer, ovarian cancer, endometrial cancer, oral cancer, gastric cancer, liver cancer, prostate cancer, bladder cancer, colorectal cancer, multiple myeloma, lymphoma, and leukemia.

The term “tumor” is used to describe a malignant or benign growth.

More than one active agent is often used when treating cancer. The disclosure includes methods of treating cancer in which a bifunctional compound of the disclosure is administered as the only active agent and in which a bifunctional compound of the disclosure is administered together with one or more additional active agents.

In any aspect or embodiment described herein, the additional anti-cancer agent selected from trabectedin, abraxane, canfosfamide, ficlatuzamab calcitriol, pazopanib, berubicin, rigosertib, selumetinib, nilotinib, dovitinib, barasertib, vandetanib, tivantinib, tozasertib, danusertib, pemetrexed, erlotinib, dasatanib, decatanib, panitumumab, amrubicin, oregovomab, Lep-etu, nolatrexed, cediranib, batabulin, ofatumumab (Arzerra), zanolimumab, edotecarin, tetrandrine, rubitecan, tesmilifene, oblimersen, ticilimumab, gossypol, ¹³¹I-Chlorotoxin, cilengitide, gimatecan, cintredekin besudotox, lucanthone, enzastaurin, neuradiab, vitespan, L-alanosine, talampanel, atrasentan, romidepsin, 5-fluorouracil, vorinostat, etoposide, gemcitabine, doxorubicin, irinotecan, liposomal doxorubicin, 5′-deoxy-5-fluorouridine, vincristine, temozolomide, seliciclib; mirdametinib, capecitabine, L-Glutamic acid, disodium salt of N-[4-[2-(2-amino-4,7-dihydro-4-oxo-1H-pyrrolo[2,3-d]pyrimidin-5-yl)ethyl]benzoyl]-heptahydrate, camptothecin, PEG-labeled irinotecan, tamoxifen, toremifene citrate, anastrazole, exemestane, letrozole, DES(diethylstilbestrol), estradiol, estrogen, conjugated estrogen, dovitinib; 3-[5-(methylsulfonylpiperadinemethyl)-indolyl]-quinolone, vatalanib, axitinib, aflibercept, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate; mubritinib, neratinib, lapatanib, canertinib, ABX-EGF antibody, erbitux, pelitinib, lapatinib, Ionafarnib, tipifarnib; amifostine, dacinostat, suberoyl analide hydroxamic acid, valproic acid, trichostatin A, romidepsin, sunitinib, sorafenib, tivozanib, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, metastat, neovastat, rebimastat, squalamine, endostatin, semaxanib, orantinib, cilengitide, interleukin-12, angiostatin, vitaxin, droloxifene, idoxyfene, spironolactone, finasteride, cimitidine, trastuzumab, denileukin diftitox, gefitinib, bortezimib, paclitaxel, topotecan, doxorubicin, docetaxel, vinorelbine, bevacizumab (monoclonal antibody) and erbitux, cremophor-free paclitaxel, epithilone B, ixabepilone, droloxifene, 4-hydroxytamoxifen, pipendoxifene, pipendoxifene, arzoxifene, fulvestrant, acolbifene, lasofoxifene, idoxifene, bazedoxifene, vatalanib, neflamapimod, PD 184352, MEK162, refametinib, pimasertib, alisertib (MLN8237), sotorasib (AMG 510), rapamycin, 40-O-(2-hydroxyethyl)-rapamycin, temsirolimus, ridaforolimus, everolimus, zotarolimus, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, abemaciclib, abiraterone acetate, afatinib, apalutamide, brentuximab vedotin, cemiplimab-rwlc, dabrafenib, trametinib, dacomitinib, duvelisib, encorafenib, binimetinib, enzalutamide, iobenguane 1131, ivosidenib, lenvatinib, linatumomab, lutetium Lu 177 dotatate, olaparib, osimertinib, ribociclib, rucaparib, tisagenlecleuce, venetoclax, acalabrutinib, alectinib, avelumab, axicabtagene ciloleucel, blinatumomab, bosutinib, brigatinib, cabozantinib, ceritinib, copanlisib, enasidenib, gemtuzumab ozogamicin, l-encap-dau+ara-c, midostaurin, niraparib, obinutuzumab, palbociclib, pertuzumab, abozantinib, daratumumab, eenetoclax, eribulin, eribulin mesylate, rizotinib, carfilzomib, cobimetinib, irinotecan liposome, ixazomib, lotuzumab, necitumumab, panobinostat, ramucirumab, sonidegib, trifluridine/tipiracil, amucirumab, belinostat, ibrutinib, idelalisib, lanreotide, laparib, ramucirumabado-tras emtansine, crizotinib, pomalidomide, Ra 223 dichloride, regorafenib, vincristine sulfate, vismodegib, ziv-aflibercept, asparaginase Erwinia chrysanthemi (Erwinaze), denosumab, cabazitaxel, tykerb, erdafitinib, alpelisib, polatuzumab vedotin, selinexor, darolutamide, pexidartnib, entrectinib, gallium dotatoc, avapritinib, pralsetinib, tafasitamab, pemigatinib, ripretinib, selpercatinib, isatuximab, capmatinib, tazemetostat, sacituzumab govitecan, tucatinib, lurbinectedin, sipuleucel-T, denusomab, radium 223 dichloride, calaspargase pegol-mknl, inotuzumab ozogamicin, zanubrutinib, belantamab mafodotin-blmf, naxitamab-gqgk, glasdegib, copper Cu 64 dotatate, elotuzumab, fam-trastuzumab deruxtecan-nxki, emapalumab-lzsg, cedazuridine, fedratinib, olaratumab, lorlatinib, moxetumomab pasudotox-tdfk, lutetium 177 dotate, margetuximab-cmkb, relugolix, enfortumab vedotin-ejfv, talazoparib, dinutuximab, epoetin alfa and darbepoetin alfa, vemurafenib, Indoximod (NLG-8187), Navoximod (GDC-0919, and NLG802, including, nivolumab, durvalumab and atezolizumab, pembrolizumab (Merck), ipilimumab, and tremelimumab.

The term “autoimmune disease” refers to a disease or illness that occurs when the body tissues are attacked by its own immune system. The immune system is a complex organization within the body that is designed normally to “seek and destroy” invaders of the body, including infectious agents. In diseases which are described as autoimmune diseases, TNF-α is often implicated. The term “inflammatory disease” is used to describe a disease or illness with acute or hyperacute, but more often chronic inflammation as a principal manifestation of the disease or illness. In an embodiment the inflammation is a result of strain, sprain, cartilage damage, trauma, orthopedic surgery, infection, or other disease processes. The inflammation can also be due to acute infection (e.g. septic shock), meningitis, pneumonia, or severe burns. The present disclosure seeks to inhibit or lower TNF-α in patients with autoimmune disease (as well as inflammatory diseases and conditions and cancer) and by decreasing TNF-α levels, ameliorate many of the symptoms and secondary effects of these disease states and conditions. Examples of autoimmune or inflammatory diseases or disorders in which TNF-α is implicated include rheumatoid arthritis, psoriatic arthritis, inflammatory bowel disease (IBD), Crohn's disease, ulcerative colitis, psoriasis, and noninfectious or autoimmune uveitis. For example, in any aspect or embodiment described herein, the disease or disorder in an autoimmune or inflammatory disease or disorder is selected from rheumatoid arthritis, psoriatic arthritis, inflammatory bowel disease (IBD), Crohn's disease, ulcerative colitis, psoriasis, and noninfectious or autoimmune uveitis.

A more complete list of autoimmune diseases which may be treated by compounds and pharmaceutical compositions of the disclosure includes Addison's Disease, Autoimmune polyendodrine syndrome (APS) types 1, 2 and 3, autoimmune pancreatitis (AIP), diabetes mellitus type 1, autoimmune thyroiditis, Cushing's syndrome, Ord's thyroiditis, Grave's disease, autoimmune oophoritis, endometriosis, autoimmune orchitis, Sjogren's syndrome, autoimmune enteropathy, coeliac disease, Crohns' disease, microscopic colitis, ulcerative colitis, autophospholipid syndrome (APlS), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans sndrome, pernicious anemia, pure red cell aplasia, thrombocytopenia, adiposis, adiposis dolorosa, adult-onset Still's disease, anklyosing spondylitis, CREST syndrome, drug-induced lupus, enthesitis-related arthritis, esosiniphilic fasciitis, Felty syndrome, AgG4-related disease, hyperlipidemia, juvenile arthritis, Lyme disease (chronic), mixed connective tissue dease (MCTD), palindromic rheurmatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, collagenosis (e.g., lupus erythematosus, including systemic lupus erythematosus, pachyderma, polyarteritis), undifferentiated connective tissue disease (UCTD), dematomyositis, fibromyalgia, myositis, inclusion body myositis, myasthenia gravis, neuromyotonia, paraneoplastic cerebellar degeneration, polymysositis, acute disseminated encephalomyelitis (ADEM), acute motor axonic neuropathy, anti-NMDA receptor encephalitis, Balo concentric sclerosis, Bickerstaffs encephalitis, chronic inflammatory demyelinating polyneuropathy, Guillain-Barré syndrome, Hashimoto's encephalopathy, idiopathic inflammatory demyelinating diseases, Lambert-Eaton myasthenic syndrome, multiple sclerosis, pattern II, Oshtoran Syndrome, Pendiatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Méniére's disease, Behçet's disease, Eosiniphilic granulomatosis with polyangiitis (EGPA), giant cell arteritis, granulomatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosiniphilic esopagitis, gastritis, interstitial lung disease, POEMS syndrome, Raynaud's syndrome, primary immunodeficiency and pyoderma gangrenosum, among others.

Inflammatory diseases and conditions include diseases of neurodegeneration (including, for example, Alzheimer's disease, Creutzfeldt-Jakob disease, Lewy body dementia, Parkinson's disease, Huntington's disease; other ataxias, anmyotrophic lateral sclerosis, AIDS encephalopathy), diseases of compromised immune response causing inflammation (e.g., dysregulation of T cell maturation, B cell and T cell homeostasis, counters damaging inflammation), diabetic complications such as retinopathy, nephropathy, and neuropathy, postoperative/posttraumatic inflammation, swelling, pharyngitis, cystitis, inflammation due to infection (e.g. pneumonia, meningitis, tuberculosis), inflammation due to viral infection (e.g., infections with cytomegalovirus, influenzae virus, herpes virus and the like), toxemia (e.g., sepsis, septic shock, endotoxic shock, grain negative sepsis, toxin shock syndrome), atopic dermatitis, inflamnmatory ophthalmic disease, conjunctivitis, climacteric failure, chronic inflammatory diseases including, for example, airway inflammation, including asthma, allergy, anaphylaxis, allergic rhinitis, pollinosis, digestive tract allergy, inflammatory bowel disease, including Crohn's disease, rheumatoid arthritis, lupus, multiple sclerosis, chronic obstructive pulmonary disease/COPD, pulmonary fibrosis, inflanimatory pulmonary disease (e.g. silicotuberculosis, pulmonary sarcoidosis), cystic fibrosis, Sjogren's disease; hepatic disease such as hepatitis including chronic disease and cirrhosis, pancreatic disease such as pancreatitis, hyperglycemic disorders, diabetes (I and II), affecting lipid metabolism islet function and/or structure, pancreatic f-cell death and related hyperglycemic disorders, including severe insulin resistance, abnormal glucose tolerance, hyperinsulinemia, insulin-resistant diabetes (e.g. Mendenhall's Syndrome, Werner Syndrome, leprechaunism, and lipoatrophic diabetes) and dyshpidemia (e.g. hyperlipidemia as expressed by obese subjects, elevated low-density lipoprotein (LDL), depressed high-density lipoprotein (HDL), elevated triglycerides and metabolic syndrome, liver disease, renal disease (apoptosis in plaques, glomerular disease), cardiovascular disease (especially including infarction, ischemia, stroke, pressure overload and complications during reperfusion, myocarditis, cardiomyopathy, hypercholestcrenia), circulatory disease (e.g., chronic heart failure including arrhythmia, angina pectris, mnyocardial infarction, cardiac insufficiency and congestive heart failure, arteriosclerosis including atherosclerosis, hypertension (e.g. portal hypertension, pulmonary hypertension), deep vein thrombosis, occlusive peripheral circulation failure, ischemic cerebral circulation failure, disseminated intravascular coagulation syndrome, Raynaud's disease, Buerger disease), post-percutaneous coronary arerioplasty vascular hypertrophy or occlusion, post-PTCA/stenting/bypass surgery vascular reocclusion/restenosis, post-intervention vascular hypertrophy or occlusion, suppression of implantatin-induced vascular failure and rejection, muscle degeneration and atrophy, low grade inflammation, gout, silicosis, atherosclerosis and associated conditions such as cardiac and neurological (both central and peripheral) manifestations including stroke, cerebral hemorrhage, cerebral infarction and its sequela, cranial trauma, spinal damage, cerebral edema, age-associated dementia and sporadic form of Alzheimer's disease, and psychiatric conditions including depression), stroke and spinal cord injury, arteriosclerosis, among others, bone disease (e.g. fracture, re-fracture, osteoporosis, osteomalacia, Behçet's disease of the bone). In these diseases, elevated MIF is very often observed, making these disease states and/or conditions response to therapy using compounds and/or pharmaceutical compositions according to the present invention. It is noted that there is some overlap between certain autoimmune diseases and inflammatory diseases as described herein.

TNF-α has been particularly implicated in the following autoimmune/inflammatory disorders: arthritis (including rheumatoid arthritis, rheumatoid myelitis, psoriatic arthritis, osteoarthritis, periosteosis, spondylitis, osteogonarthritis as wvell as articular tissue destruction in disease related thereto) inflammatory bowel disease (IBD), Crohn's disease, ulcerative colitis, psoriasis, and noninfectious or autoimmune uveitis.

Methods of treatment include administering a therapeutically effective dose of between about about 0.01, about 0.1, about 1, about 5, about 10, about 15, about 20, about 25, about 30, about 35, about 40, about 45, about 50, about 55, about 60, about 65, about 70, about 75, about 80, about 85, about 90 or about 100 mg/kg per patient/day or in some embodiments, greater than about 100, about 110, about 120, about 130, about 140, about 150, about 160, about 170, about 180, about 190 or about 200 mg/kg of a compound of the disclosure can be administered to a patient. A preferred dose of the compound of the disclosure for all of the herein-mentioned conditions is in the range from about 10 ng/kg to about 300 mg/kg, preferably about 0.1 to about 100 mg/kg per day, more generally about 0.5 to about 25 mg per kilogram body weight of the recipient/patient per day. A typical topical dosage will range from about 0.01 to about 3% wt/wt in a suitable carrier.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

In any aspect or embodiment described herein, a patient or subject (e.g. a human, or a livestock animal, such as a swine, bovine, goat, horse, or sheep or a companion animal, such as a dog, rabbit, or cat) suffering from Alzheimer's disease, cancer, atherosclerosis, depression (such as, major depressive disorder), sepsis, diabetes, obesity, or autoimmune or inflammatory disease or disorder can be treated by administering to the patient (subject) an effective amount of a chimeric/bi-functional compound according to the present invention including pharmaceutically acceptable salts, solvates or polymorphs, thereof optionally in a pharmaceutically acceptable carrier or diluent, either alone, or in combination with other known pharmaceutical agents, preferably agents which can assist in treating autoimmune and/or inflammatory diseases or cancer. This treatment can also be administered in conjunction with other conventional therapies, such as radiation treatment or surgery for cancer.

The compounds of the present disclosure, alone or in combination with other agents as described herein, can be administered by any appropriate route, for example, orally, parenterally, intravenously, intradermally, subcutaneously, or topically, in liquid, cream, gel, or solid form, or by aerosol form.

In any aspect or embodiment described herein, the compounds of the present disclosure are conveniently administered in any suitable unit dosage form, including but not limited to one containing less than 1 mg, about 1 mg to about 3000 mg, preferably about 5 to about 500 mg of active ingredient per unit dosage form. In any aspect or embodiment described herein, an oral dosage of about 25 to about 500 mg is often convenient.

In any aspect or embodiment described herein, the active ingredient is preferably administered to achieve peak plasma concentrations of the active compound of about 0.00001 to about 30 mM, preferably about 0.1 to about 30 M. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are for exemplification only and are not intended to limit the scope or practice of the claimed composition. The active ingredient may be administered at once, or may be divided into a number of smaller doses to be administered at varying intervals of time.

### Glossary of Markush Variables

### 1. Variables in the First Markush Structure

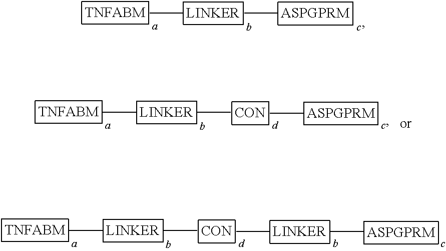

[TNFABM] is a Tumor Necrosis Factor-Alpha (TNF-α) Binding Moiety that is capable of binding to TNF-α.

[ASPGPRM] is an Asialoglycoprotein Receptors Binding Moiety that binds to an asialoglycoprotein receptor (ASGPR) on an outer surface of a cell (e.g., a hepatocyte or other degrading cell, such as a cell in a subject).

[LINKER] is an optional chemical moiety that is covalently attached to one or more [TNFABM] groups and the [CON].

[CON] is a bond or a chemical connecting moiety that is covalently attached to one or more [ASPGPRM] groups and the [LINKER].

a is an integer from 1 to 15 (e.g. 1, 2, 3, 4, 5, 6, 7, 8, 9, 10, 11, 12, 13, 14, or 15).

b is an integer from 0 to 15 (e.g. 0, 1, 2, 3, 4, 5, 6, 7, 8, 9, 10, 11, 12, 13, 14, or 15), preferably 0 or 1.

c is an integer from 1 to 15 (e.g. 1, 2, 3, 4, 5, 6, 7, 8, 9, 10, 11, 12, 13, 14, or 15).

d is an integer from 0 to 15 (e.g. 0, 1, 2, 3, 4, 5, 6, 7, 8, 9, 10, 11, 12, 13, 14, or 15, preferably 0, 1, 2, or 3).

### 2. Variables Used to Define [ASGPRBM]

The [ASGPRBM], or asialoglycoprotein receptors binding, is a portion of the bifunctional compound that can binds to an ASGPR on an outer surface of a hepatocyte or other degrading cell in the subject and cause the bifunctional molecule along with TNFBM bound to the [TNFABM] to be degraded.

A representative [ASGPRBM] core structure is

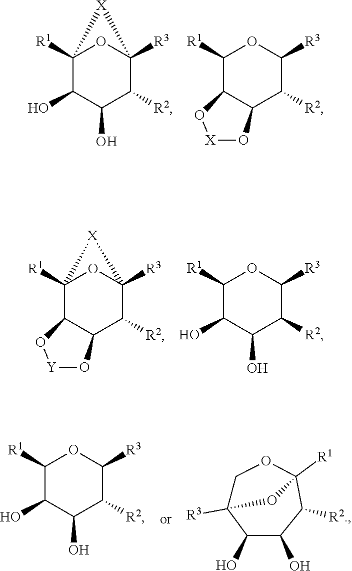

R¹, R², R³, X, and Y are substituents directly bound to an [ASGPRBM] core structure.

[L] is used to show the point of attachment of an [ASGPRBM] to a [LINKER].

R^{N1} is a variable within the definition of X and R¹-R³ used for groups bound to a nitrogen.

R^{N2} is a variable within the definition of R² used for groups bound to a nitrogen.

R^{N3} is a variable within the definition of R^{N2} used for groups bound to a nitrogen.

R^{N4} is a variable within the definition or R² used for groups bound to nitrogen, where the nitrogen is covalently bound to [ASGPRBM] at the point of attachment for R² or part of a nitrogen-containing group bound to [ASGPRBM] at the point of attachment for R².

R^{C1} is a variable within the definition of X and R¹-R³ used for groups bound to a carbon.

K is a variable with the definitions of R¹-R³ to state the length of alkylene chain. The alkylene chain may contain unsaturated bonds or heteroatoms if stated or the context indicates.

R^{A} is a variable within the definitions of R¹-R³ to describe substituents on cyclic groups within the definition of R¹-R³.

R^{B} is a variable within the definition of R^{N3}.

R^{C} is a substituent of R², when R² is a cyclic group.

R^{D} is a variable within the definition of R² used when the R² group includes a nitrogen or sulfur bound to [ASGPRBM] at the point of attachment for R². R^{D} groups are covalently bound to a nitrogen or sulfur.

R^{E} is a variable within the definition of R^{D} and is covalently bound to an oxygen.

R^{2A} is a variable within the definition of R².

### 3. Variables Used to Define [CON]

Y² and Y³ are variables within the definition of [CON].

X⁴ and X⁵ are variables used in the definition of [CON] to define a chain or ring atom that can be carbon or a heteroatom.

R^{J} and R^{K} are variables within the definition of [CON] used to denote positions of attachment to of the [CON] to the [LINKER], [TNFABM], or [ASGPRBM].

R⁷ is a variable within a chemical structure of [CON]·R⁶ is a variable in the definition of [CON].

R⁷ is a variable within the definition of Y², X², and X³.

R⁸ is an integer from 0 to 8 within the definition of [CON].

variables in the definition of [CON].

R^{1CON}, R^{2CON}, and R^{3CON} are each variables within a chemical structure of [CON].nn is an integer variable in the definition of [CON].

p is an integer from 0 to 8 within the definition of certain [CON] groups.

XA is 1 or 2 within the definition of [CON].

r is an integer from 1 to 4 within the definition of [CON];

s is an integer from 0 to 4 within the definition of [CON].

k is an integer from 0 to 10 within the definition of [CON].

n and m are integers from 1 to 10 within the definition of [CON].

### 4. Variables Used to Define [LINKER]

g, h, j, k are integers in the definition of [LINKER] used to denote repeating groups in the [LINKER].

R²⁰ is a variable in the definition of [LINKER] that substitutes nitrogen.

R²¹ is a variable in the definition of [LINKER].

R²³ and R²⁴ are variables used to substitute cyclic groups in the [LINKER].

R²⁵ and R²⁶ are variables used to substitute carbon atoms in the [LINKER].

R²⁷, R²⁸, and R²⁹ are variables used in the definitions of certain [LINKER] groups.

X¹, X² and X³ are variables in the definition of [LINKER] used to denote ring atoms or groups.

### EXAMPLES

The following abbreviations are used in the procedures set forth in this disclosure.
AIBN azobisisobutyronitrile
DCM dichloromethane
DIAD diisopropyl azodicarboxylate
DIEA,N-diisopropylethylamine
DMF dimethylformamide
DMSO dimethyl sulfoxide
EA ethyl alcohol
EDCI 1-Ethyl-3-(3-dimethylaminopropyl)carbodiimide
EtOAc Ethyl acetate
HATU hexafluorophosphate azabenzotriazole tetramethyl uronium
HOBt hydroxybenzotriazole
MTBE methyl tertiary-butyl ether
NMI N-methylimidazole
Pd(dppf)Cl₂ 1,1′-Bis(diphenylphosphino)ferrocene]dichloropalladium(II)
PE Petroleum ether
PPh₃ triphenylphosphine
TBAI tetra-n-butylammonium iodide
TCFH tetramethylchloroformamidinium hexafluorophosphate
THF tetrahydrofuran
TFA trifluoroacetic acid
Tol Toluene

### Example 1. Procedure for Preparation of Compound 101

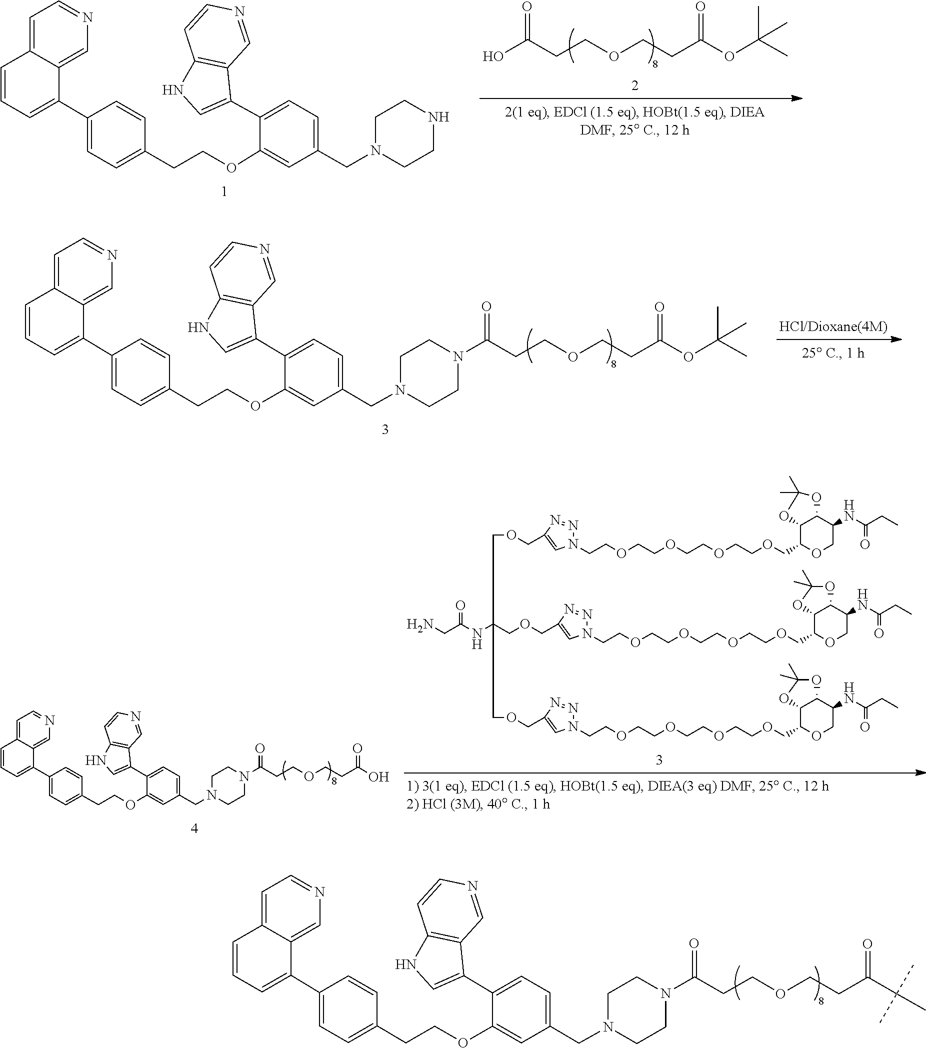

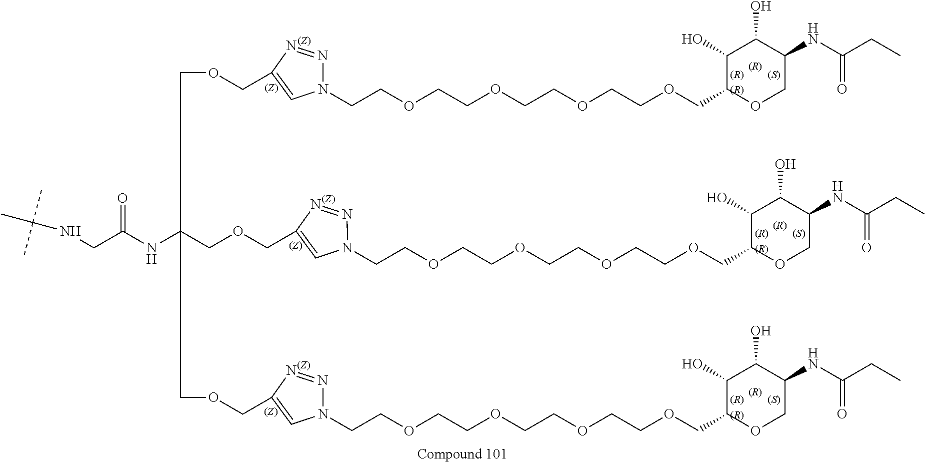

### Preparation of Intermediate 3

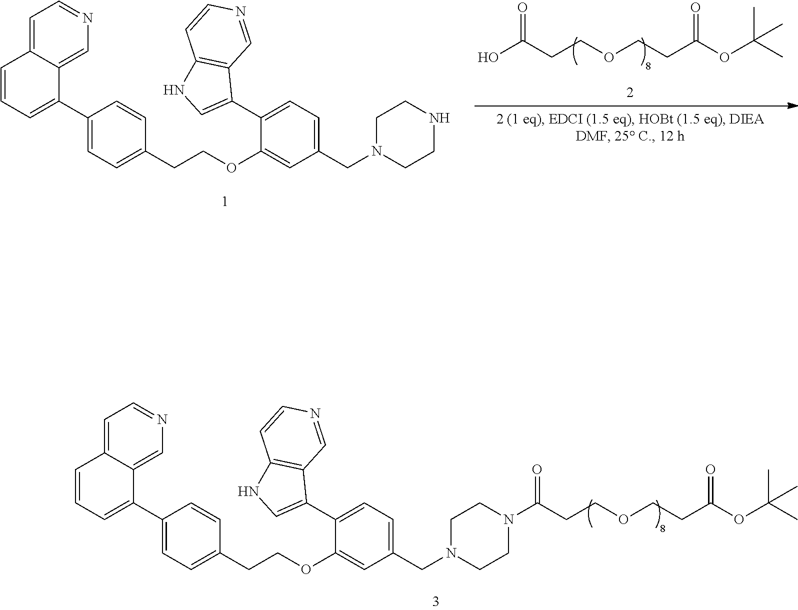

To a solution of Intermediate 1 (400 mg, 741.20 μmol, 1.00 equivalent) and Intermediate 2 (390.32 mg, 741.20 μmol, 1.00 equivalent) in dimethylformamide (DMF) (5 mL), 1-Ethyl-3-(3-dimethylaminopropyl)carbodiimide (EDCI) (213.13 mg, 1.11 mmol, 1.50 equivalent) and hydroxybenzotriazole (HOBt) (150.23 mg, 1.11 mmol, 1.50 equivalent) was added at 25° C. The reaction was stirred at 25° C. for 12 hours. LCMS: showed Intermediate 1 was consumed and the desired mass of the product was detected. The reaction mixture was concentrated under reduced pressure to give a residue. The residue was purified by column chromatography (SiO₂, dichloromethane (DCM): methanol=100/1 to 2/1) to afford Intermediate 3 (530 mg, 505.60 μmol, 68.21% yield) as yellow solid. LCMS: RT=3.168 min, MS cal.: 1047.6, found: [M/2+H]+=524.9. ¹H NMR (400 MHz, CD₃OD) δ=9.07 (s, 1H), 8.92 (s, 1H), 8.45 (d, J=5.8 Hz, 1H), 8.16 (d, J=5.9 Hz, 1H), 7.95 (d, J=8.3 Hz, 1H), 7.89 (d, J=5.6 Hz, 1H), 7.85-7.80 (m, 1H), 7.52 (d, J=7.8 Hz, 2H), 7.45 (d, J=5.9 Hz, 1H), 7.31-7.27 (m, 2H), 7.25-7.21 (m, 2H), 7.06-7.02 (m, 1H), 4.58 (br s, 4H), 4.39 (t, J=6.3 Hz, 2H), 3.74 (t, J=6.3 Hz, 2H), 3.68-3.62 (m, 10H), 3.61-3.57 (m, 25H), 3.13 (t, J=6.3 Hz, 2H), 2.66 (t, J=6.3 Hz, 2H), 2.57-2.50 (m, 4H), 2.45 (t, J=6.2 Hz, 2H), 1.43 (s, 9H). Theoretical number of H: 77, found: 77.

### Preparation of Intermediate 4

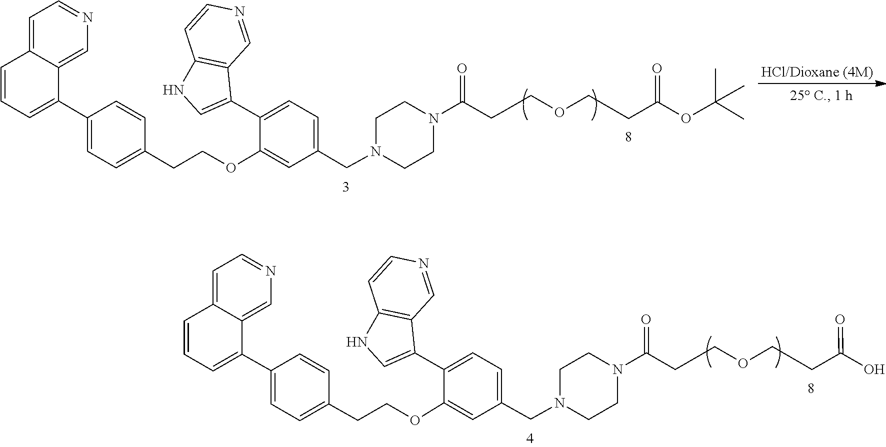

To a solution of Intermediate 3 (700 mg, 667.77 μmol, 1.00 equivalent), HCl/dioxane (4 M, 3.5 mL, 20.97 equivalent) was added at 25° C., and the reaction was stirred at 25° C. for 1 hour. LCMS: showed the reaction was completed. The reaction mixture was concentrated under reduced pressure to give a residue. The residue was purified by prep-HPLC (column: Waters Xbridge Prep OBD C18 150*40 mm*10 μm; mobile phase: [water (NH₄HCO₃)-ACN]; B %: 15%-45%, 8 min.) to afford Intermediate 4 (130 mg, 131.03 μmol, 34.34% yield) as yellow solid. LCMS: RT=2.605 min, MS cal.: 991.49, found: [M/2+H]+=496.9. ¹H NMR (400 MHz, DMSO-d₆) δ=11.64 (br s, 1H), 9.14 (s, 1H), 8.97 (d, J=0.8 Hz, 1H), 8.53 (d, J=5.6 Hz, 1H), 8.19 (d, J=5.8 Hz, 1H), 7.99 (d, J=8.3 Hz, 1H), 7.90 (d, J=5.3 Hz, 1H), 7.86-7.80 (m, 1H), 7.58-7.52 (m, 3H), 7.45-7.37 (m, 5H), 7.12 (s, 1H), 7.02-6.98 (m, 1H), 4.36 (t, J=6.6 Hz, 2H), 3.64-3.55 (m, 5H), 3.53 (s, 2H), 3.51-3.45 (m, 34H), 3.13 (brt, J=6.6 Hz, 2H), 2.55 (t, J=6.6 Hz, 2H), 2.41 (t, J=6.4 Hz, 4H). Theoretical number of H: 69, found: 69.

### Preparation of Compound 101

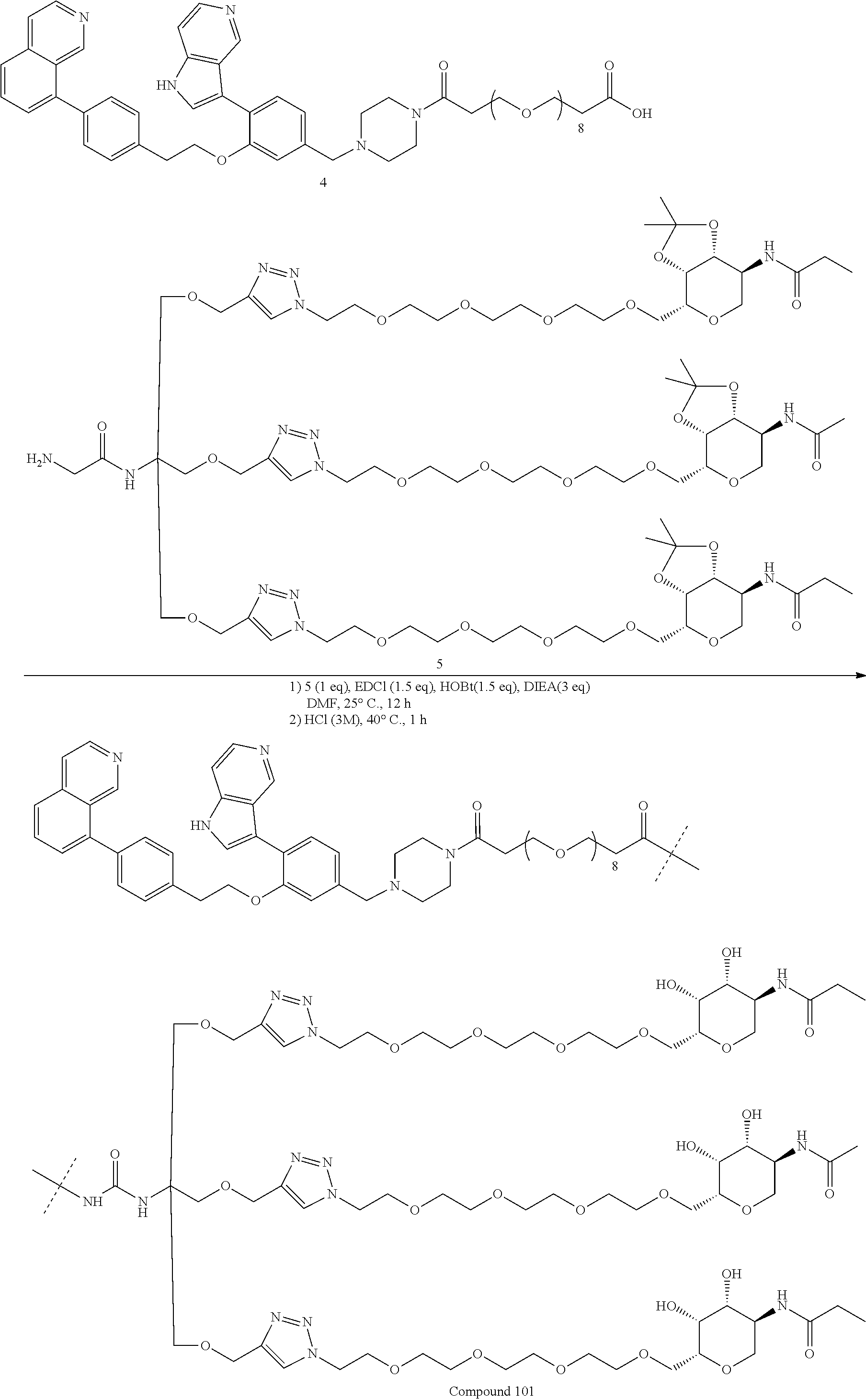

To a solution of Intermediate 4 (350 mg, 352.77 μmol, 1.00 equivalent) and Intermediate 5 (590.49 mg, 352.77 μmol, 1 equivalent) in DMF (4 mL), HOBt (71.50 mg, 529.15 μmol, 1.50 equivalent) and EDCI (101.44 mg, 529.15 μmol, 1.50 equivalent) was added at 25° C. Then N,N-diisopropylethylamine (DIEA) (136.78 mg, 1.06 mmol, 184.34 uL, 3.00 equivalent) in DMF (0.50 mL) was added dropwise to the above reaction mixture at 0° C. Then the reaction mixture was stirred at 25° C. for 12 hours. LCMS showed that Intermediate 4 was consumed completely and one main peak with desired mass of the product was detected. The reaction mixture was concentrated under reduced pressure to give a residue. The residue was used to next step directly without purification. Then HCl (3 M, 3.00 mL, 79.44 equivalent) was added to the above residue at 40° C. and the reaction solution was stirred at 40° C. for 1 hour. LCMS showed that the reactant was consumed completely and the desired mass of the product was detected. The reaction mixture was concentrated under reduced pressure to give a residue. The residue was purified by prep-HPLC (column: Waters Xbridge BEH C18 100*30 mm*10 μm; mobile phase: [water (NH₄HCO₃)-ACN]; B %: 40%-65%, 8 min.) to afford Compound 101 (42.78 mg, 16.16 μmol, 4.58% yield) as white solid. LCMS: RT=1.910 min, MS cal.: 2526.3, found: [M/3+H]+=843.5. [M/4+H]+=632.8. ¹H NMR (400 MHz, DMSO-d₆) δ=11.64 (br s, 1H), 9.14 (s, 1H), 8.97 (d, J=0.8 Hz, 1H), 8.53 (d, J=5.6 Hz, 1H), 8.19 (d, J=5.8 Hz, 1H), 7.99 (d, J=8.3 Hz, 1H), 7.90 (d, J=5.3 Hz, 1H), 7.86-7.80 (m, 1H), 7.58-7.52 (m, 3H), 7.45-7.37 (m, 5H), 7.12 (s, 1H), 7.02-6.98 (m, 1H), 4.36 (t, J=6.6 Hz, 2H), 3.64-3.55 (m, 5H), 3.53 (s, 2H), 3.51-3.45 (m, 34H), 3.13 (brt, J=6.6 Hz, 2H), 2.55 (t, J=6.6 Hz, 2H), 2.41 (t, J=6.4 Hz, 4H). Theoretical number of H: 183, found: 183.

### Example 2. Procedure for Preparation of Compound 102

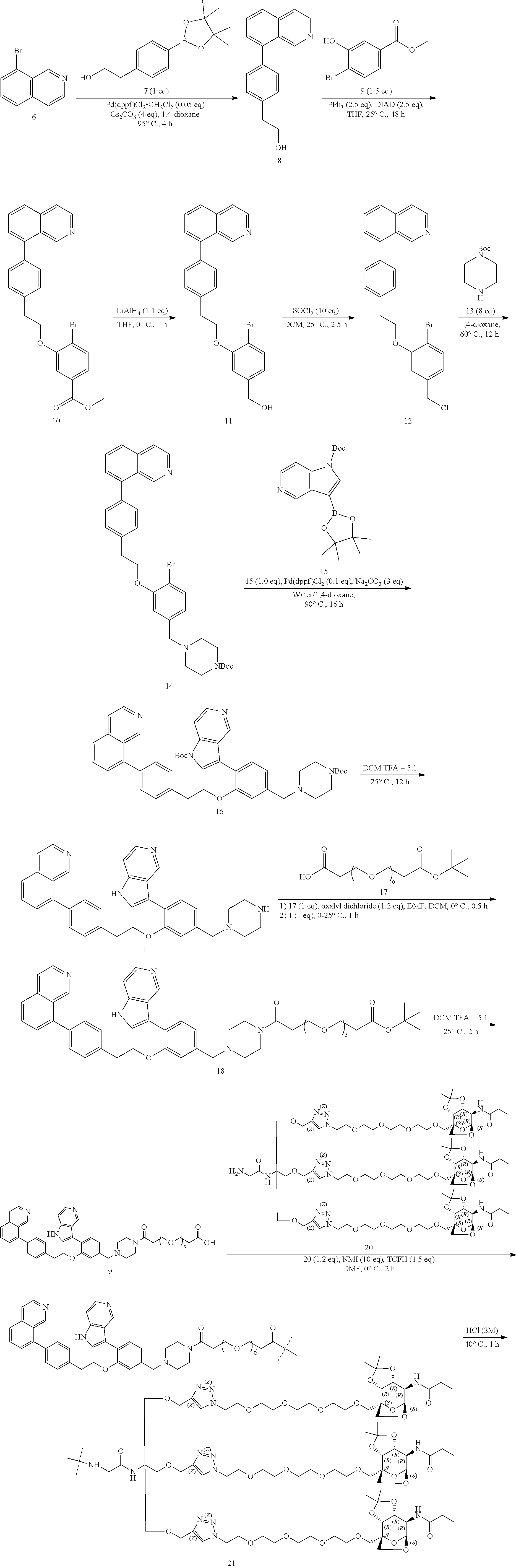

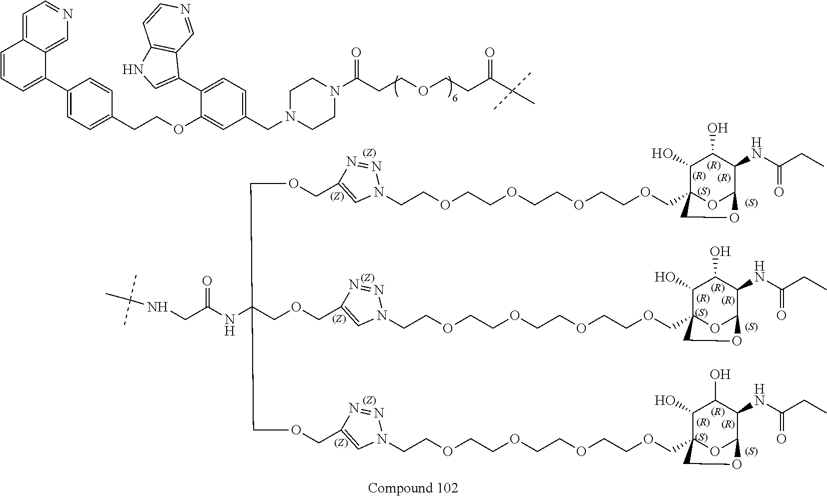

### Preparation of Intermediate 8

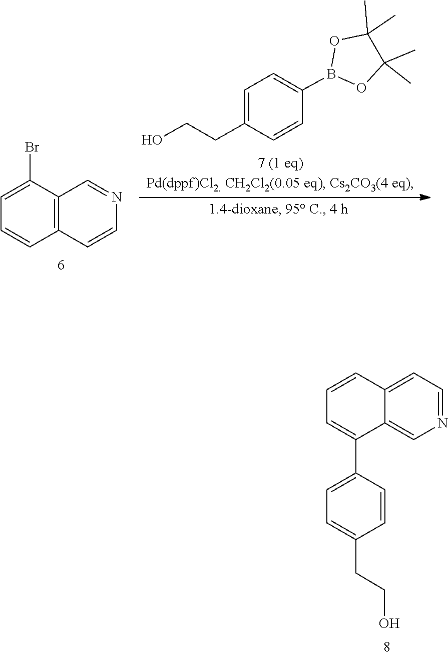 [SMILES: CC1(C)OB(c2ccc(CCO)cc2)OC1(C)C ; Brc1cccc2ccncc12 ; OCCc1ccc(-c2cccc3ccncc23)cc1]

A mixture of Intermediate 6 (6.5 g, 31.24 mmol, 1.00 equivalent), Intermediate 7 (7.75 g, 31.24 mmol, 1.00 equivalent), Pd(dppf)Cl₂·CH₂Cl₂ (1.28 g, 1.56 mmol, 0.05 equivalent), and Cs₂CO₃ (2 M, 62.48 mL, 4.00 equivalent) in dioxane (100 mL) was degassed and purged with N₂ three times, and then the mixture was stirred at 95° C. for 4 hours under N₂ atmosphere. LCMS showed that the reactant was completely consumed and desired mass of the product was detected. The residue was diluted with H₂O (40 mL) and extracted with ethyl acetate (EtOAc) (40 mL×3). The combined organic layers were dried over Na₂SO₄, filtered, and concentrated under reduced pressure to give a residue. The residue was purified by column chromatography (SiO₂, petroleum ether/ethyl acetate=10/1 to 0/1). Intermediate 8 (12 g, 48.13 mmol, 77.03% yield) was obtained as a yellow solid. LCMS: RT=0.789 min, MS cal.: 249.1, found: [M+H]⁺=250.0. ¹H NMR (400 MHz, CDCl₃) δ=9.32 (s, 1H), 8.54 (d, J=5.6 Hz, 1H), 7.86-7.80 (m, 1H), 7.77-7.68 (m, 2H), 7.51 (d, J=7.0 Hz, 1H), 7.48-7.43 (m, 1H), 7.49-7.43 (m, 1H), 7.42-7.37 (m, 2H), 3.98 (br t, J=6.5 Hz, 2H), 3.00 (t, J=6.5 Hz, 2H), 2.29 (br s, 1H). Theoretical number of H: 15, found: 15.

### Preparation of Intermediate 10

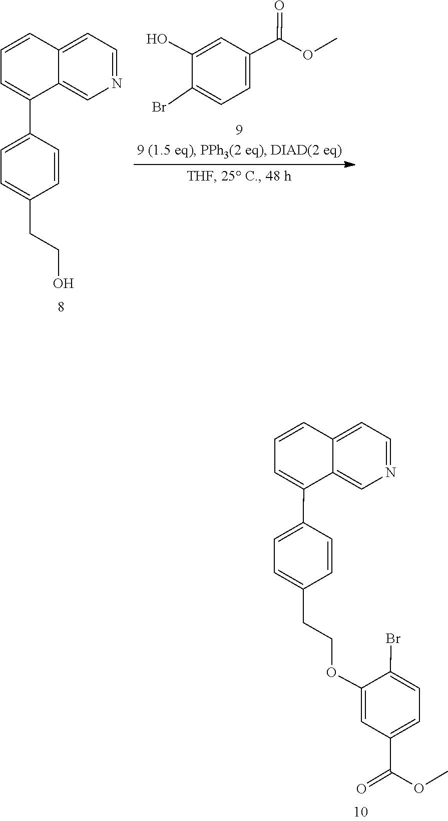 [SMILES: OCCc1ccc(-c2cccc3ccncc23)cc1 ; COC(=O)c1ccc(Br)c(O)c1 ; COC(=O)c1ccc(Br)c(OCCc2ccc(-c3cccc4ccncc34)cc2)c1]

A mixture of Intermediate 8 (5.00 g, 20.06 mmol, 1.00 equivalent), Intermediate 9 (6.95 g, 30.08 mmol, 1.5 equivalent), triphenylphosphine (PPh₃) (10.52 g, 40.11 mmol, 2.00 equivalent), diisopropyl azodicarboxylate (DIAD) (8.11 g, 40.11 mmol, 7.80 mL, 2.00 equivalent) in tetrahydrofuran (THF) (200 mL) was degassed and purged with N₂ for three times, and then the mixture was stirred at 25° C. for 48 hours under N₂ atmosphere. Thin-layer Chromatograph (TLC) (petroleum ether/ethyl acetate=0/1, product R_{f}=0.66) indicated reactant was consumed completely and one new spot formed. The reaction mixture filtered and diluted with H₂O (50 mL) and was extracted with EtOAc (50 mL×3). The combined organic layers were over Na₂SO₄, filtered, and concentrated under reduced pressure to give a residue. The residue was purified by column chromatography (SiO₂, petroleum ether/ethyl acetate=30/1 to 0/1). Intermediate 10 (18.00 g, 38.93 mmol, 97.06% yield) was obtained as a yellow oil. LCMS: RT=2.869 min, MS cal.: 461.0, found: [M+H]+=462.0. ¹H NMR (400 MHz, CDCl₃) δ=9.34 (s, 1H), 8.56 (d, J=5.8 Hz, 1H), 7.83 (d, J=8.1 Hz, 1H), 7.76-7.68 (m, 2H), 7.62 (d, J=8.1 Hz, 1H), 7.58-7.49 (m, 7H), 4.38 (t, J=6.6 Hz, 2H), 3.92 (s, 3H), 3.30 (t, J=6.6 Hz, 2H). Theoretical number of H: 20, found: 20.

### Preparation of Intermediate 11

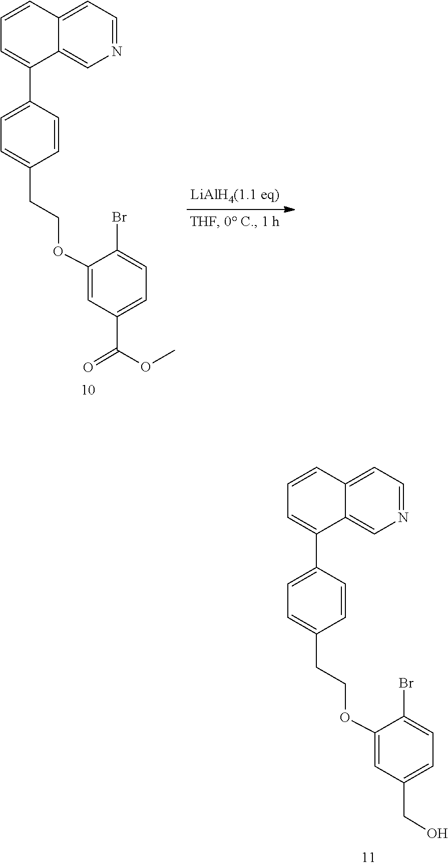 [SMILES: COC(=O)c1ccc(Br)c(OCCc2ccc(-c3cccc4ccncc34)cc2)c1 ; OCc1ccc(Br)c(OCCc2ccc(-c3cccc4ccncc34)cc2)c1]

To a solution of LiAl₄ (2.26 g, 59.48 mmol, 1.10 equivalent) in THF (100 mL), Intermediate 10 (25.00 g, 54.07 mmol, 1.00 equivalent) in THF (300 mL) was added. The mixture was stirred at 0° C. for 1 hour. TLC (Petroleum ether/Ethyl acetate=0/1, product R_{f}=0.20) indicated reactant was consumed completely and one new spot formed. The reaction mixture was quenched by addition of H₂O (2 mL) at 10° C. and NaOH (15% aq., 2 mL), filtered, and concentrated under reduced pressure to give a residue. The residue was purified by column chromatography (SiO₂, petroleum ether/ethyl acetate=10/1 to 0/1). Intermediate 11 (16.00 g, 36.84 mmol, 68.13% yield) was obtained as a colorless oil. LCMS: RT=0.929 min, MS cal.: 433.0, found: [M+H]⁺=434.0. ¹H NMR (400 MHz, CDCl₃) δ=9.22 (s, 1H), 8.52 (d, J=5.8 Hz, 1H), 7.85-7.81 (m, 1H), 7.77-7.70 (m, 2H), 7.56-7.42 (m, 7H), 6.91 (d, J=1.5 Hz, 1H), 6.86-6.81 (m, 1H), 4.65 (s, 2H), 4.36 (t, J=6.6 Hz, 2H), 3.27 (t, J=6.6 Hz, 2H). Theoretical number of H: 20, found: 20. Preparation of Intermediate 12:

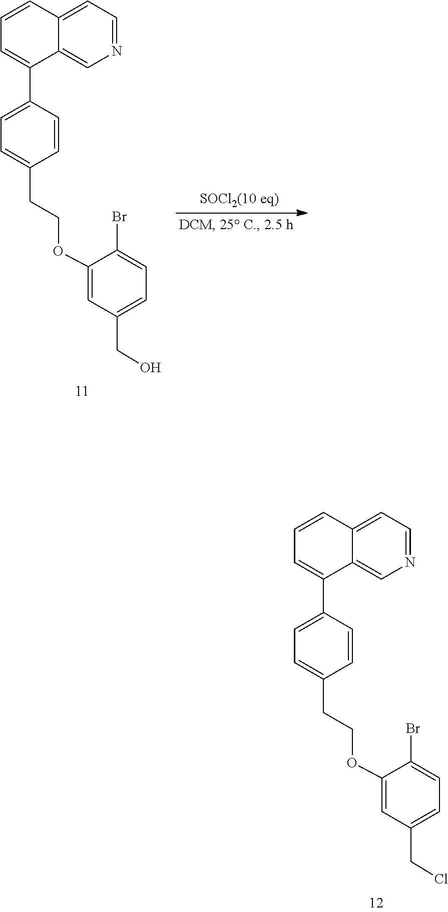 [SMILES: OCc1ccc(Br)c(OCCc2ccc(-c3cccc4ccncc34)cc2)c1 ; ClCc1ccc(Br)c(OCCc2ccc(-c3cccc4ccncc34)cc2)c1]

To a solution of Intermediate 11 (5.00 g, 11.51 mmol, 1.00 equivalent) in DCM (170 mL), SOCl₂ (13.70 g, 115.12 mmol, 8.35 mL, 10.00 equivalent) was added, and the mixture was stirred at 25° C. for 2.5 hours. LCMS showed the reaction was complete. The reaction product was concentrated under reduced pressure to give Intermediate 12 (5.21 g, 11.51 mmol, 100.00% yield) as a yellow solid, which was directly used in the next step without purification. LCMS: RT=2.932 min, MS cal.: 451.0, found: [M+H]⁺=452.0.

### Preparation of Intermediate 14

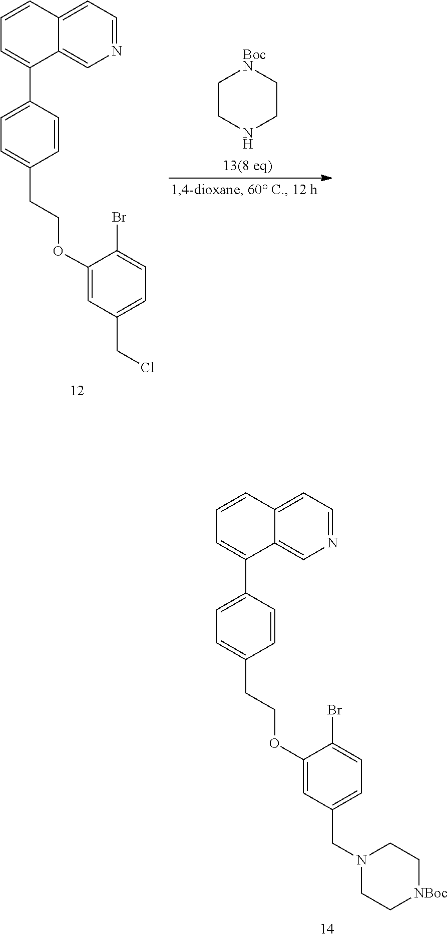 [SMILES: ClCc1ccc(Br)c(OCCc2ccc(-c3cccc4ccncc34)cc2)c1 ; CC(C)(C)OC(=O)N1CCNCC1 ; CC(C)(C)OC(=O)N1CCN(Cc2ccc(Br)c(OCCc3ccc(-c4cccc5ccncc45)cc3)c2)CC1]

To a solution of Intermediate 12 (5 g, 11.04 mmol, 1.00 equivalent) in 1, 4-dioxane (130 mL), Intermediate 13 (19.68 g, 88.35 mmol, 8.00 equivalent) was added, and the mixture was stirred at 60° C. for 12 hours. LCMS showed the reaction was complete. The reaction mixture was concentrated under reduced pressure to give product. The residue was purified by column chromatography (SiO₂, dichloromethane:methanol=100/1 to 10/1) and the product was eluted with dichloromethane:methanol=50/1. Intermediate 14 (6.10 g, 10.12 mmol, 91.67% yield) was obtained as a yellow oil. LCMS: RT=3.149 min, MS cal.: 601.2, found: [M+H]⁺=602.2. ¹H NMR (400 MHz, CDCl₃) δ=9.34 (s, 1H), 8.56 (d, J=5.6 Hz, 1H), 7.85-7.81 (m, 1H), 7.76-7.69 (m, 2H), 7.55-7.46 (m, 7H), 6.94 (d, J=1.4 Hz, 1H), 6.82-6.77 (m, 1H), 4.32 (t, J=6.8 Hz, 2H), 3.46-3.43 (m, 4H), 3.28 (t, J=6.7 Hz, 2H), 2.87-2.82 (m, 1H), 2.38 (br s, 4H), 1.46 (s, 9H). Theoretical number of H: 36, found: 36.

### Preparation of Intermediate 16

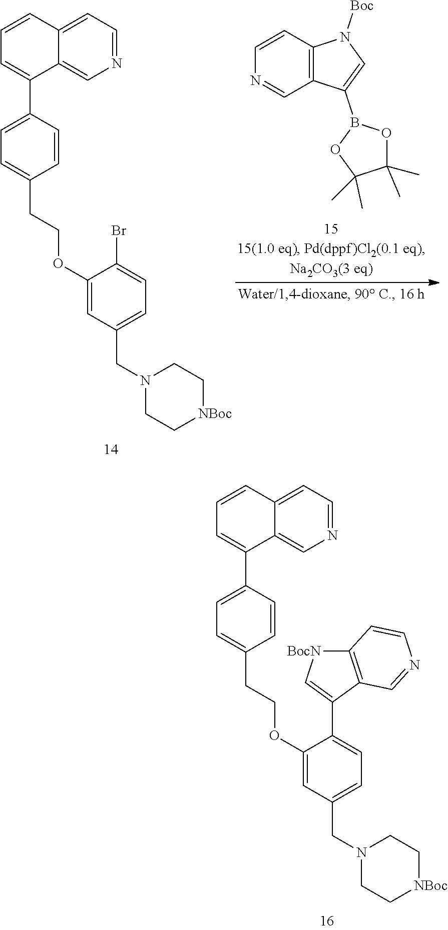 [SMILES: CC(C)(C)OC(=O)N1CCN(Cc2ccc(Br)c(OCCc3ccc(-c4cccc5ccncc45)cc3)c2)CC1 ; CC(C)(C)OC(=O)n1cc(B2OC(C)(C)C(C)(C)O2)c2cnccc21 ; CC(C)(C)OC(=O)N1CCN(Cc2ccc(-c3cn(C(=O)OC(C)(C)C)c4ccncc34)c(OCCc3ccc(-c4cccc5ccncc45)cc3)c2)CC1]

To a solution of Intermediate 14 (1.75 g, 2.90 mmol, 1.00 equivalent) in water (4.50 mL) and 1,4-dioxane (18 mL) was added Na₂CO₃ (923.46 mg, 8.71 mmol, 3.00 equivalent), Intermediate 15 (999.69 mg, 2.90 mmol, 1.00 equivalent), and Pd(dppf)Cl₂ (212.51 mg, 290.43 μμmol, 0.10 equivalent). The mixture was stirred at 90° C. for 16 hours. LCMS showed the reaction was complete. The reaction mixture was diluted with H₂O (200 mL) and extracted with Dichloromethane (100 mL*3). The combined organic layers were dried over Na₂SO₄, filtered, and concentrated under reduced pressure to give a residue. The residue was purified by column chromatography (SiO₂, dichloromethane:methanol=50/1 to 5/1). Intermediate 16 (2.10 g, 2.83 mmol, 97.46% yield) was obtained as yellow oil. LCMS: RT=3.185 min, MS cal.: 739.4, found: [M+H]⁺=740.4. ¹H NMR (400 MHz, CDCl₃) δ=9.29-9.13 (m, 1H), 8.94 (br d, J=1.0 Hz, 1H), 8.58-8.36 (m, 2H), 8.06-7.91 (m, 1H), 7.76-7.71 (m, 2H), 7.66-7.60 (m, 2H), 7.43-7.37 (m, 2H), 7.29 (s, 1H), 7.20-7.17 (m, 3H), 7.16 (s, 1H), 7.00 (s, 1H), 6.98 (s, 1H), 6.95 (s, 1H), 6.94 (s, 1H), 4.25 (t, J=7.1 Hz, 2H), 3.48 (s, 2H), 3.40 (br d, J=4.4 Hz, 3H), 3.05 (t, J=7.0 Hz, 2H), 2.37 (br s, 4H), 2.25-2.22 (m, 1H), 2.19 (s, 1H), 1.69-1.60 (m, 4H), 1.38 (s, 8H), 1.16 (s, 2H). Theoretical number of H: 49, found: 49.

### Preparation of Intermediate 1

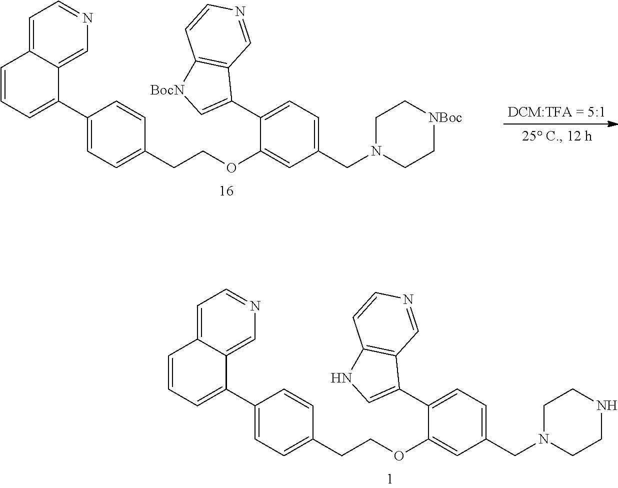 [SMILES: CC(C)(C)OC(=O)N1CCN(Cc2ccc(-c3cn(C(=O)OC(C)(C)C)c4ccncc34)c(OCCc3ccc(-c4cccc5ccncc45)cc3)c2)CC1 ; c1cc(-c2ccc(CCOc3cc(CN4CCNCC4)ccc3-c3c[nH]c4ccncc34)cc2)c2cnccc2c1]

To a solution of Intermediate 16 (1.91 g, 2.57 mmol, 1.00 equivalent) in DCM (20 mL) was added trifluoroacetic acid (TFA) (4 mL) and the reaction mixture was stirred at 25° C. for 12 hours. LCMS showed the reaction was completed. The reaction was concentrated under reduced pressure. The reaction mixture was poured into H₂O (180 mL) and extracted with methyl tertiary-butyl ether (MTBE) (90 mL×5). The combined aqueous phase was added NaOH (1 M) until pH=12-13. Then the aqueous phase was extracted with DCM (90 mL×2). The combined organic layers were dried over Na₂SO₄, filtered, and concentrated under reduced pressure to give a residue. Intermediate 1 (1.30 g, 2.41 mmol, 93.81% yield) was obtained as a yellow solid. LCMS: RT=1.922 min, MS cal.: 539.3, found: [M+H]⁺=540.3. ¹H NMR (400 MHz, CDCl₃) δ=9.73-9.63 (m, 1H), 9.12 (s, 1H), 9.00 (s, 1H), 8.58 (br d, J=5.6 Hz, 1H), 8.33 (br d, J=5.8 Hz, 1H), 7.86-7.78 (m, 1H), 7.73 (br d, J=5.5 Hz, 1H), 7.51-7.47 (m, 2H), 7.31 (br d, J=5.6 Hz, 1H), 7.20 (br d, J=7.6 Hz, 2H), 7.11-7.05 (m, 3H), 7.01 (br d, J=7.5 Hz, 1H), 5.31 (s, 1H), 4.41-4.32 (m, 2H), 3.56 (s, 2H), 3.14 (brt, J=5.6 Hz, 2H), 2.95 (br d, J=3.8 Hz, 4H), 2.50 (br s, 4H). Theoretical number of H: 33, found: 31, exchangeable H: 2.

### Preparation of Intermediate 18

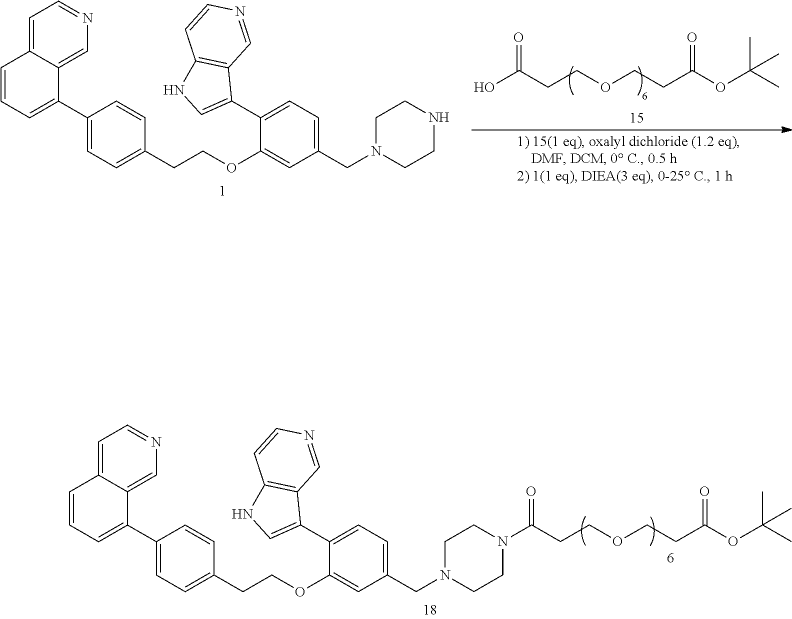

To a solution of Intermediate 17 (330 mg, 752.55 μmol, 1.00 equivalent) in DCM (5 mL), oxalyl dichloride (114.62 mg, 903.06 μmol, 79.05 uL, 1.20 equivalent) and DMF (0.20 mL) at 0° C. was added, and the reaction mixture was stirred for 30 minutes at 0° C. Then to a solution of Intermediate 1 (400 mg, 741.20 μmol, 1.00 equivalent) in DCM (4 mL), DIEA (287.38 mg, 2.22 mmol, 387.31 uL, 3.00 equivalent) was added dropwise to the above reaction mixture at 0° C., and the reaction mixture was stirred at 25° C. for 1 hour. LCMS showed the reactant was consumed and the desired mass of the product was obtained. The reaction was washed with HCl (1 M) (10 mL×2) and extracted with EtOAc (10 mL×3). The combined organic layers were washed with brine (10 mL), dried over Na₂SO₄, filtered, and concentrated under reduced pressure to give a residue. Intermediate 18 (170 mg, 177.05 μmol, 23.89% yield) was obtained as yellow oil. LCMS: RT=2.342 min, MS cal.: 959.5, found: [M+H]⁺=960.5. ¹H NMR (400 MHz, DMSO-d₆) δ=11.67-11.58 (m, 1H), 9.14 (s, 1H), 8.97 (s, 1H), 8.53 (d, J=5.6 Hz, 1H), 8.19 (d, J=5.6 Hz, 1H), 7.99 (d, J=8.3 Hz, 1H), 7.91 (d, J=5.4 Hz, 1H), 7.83 (t, J=7.7 Hz, 1H), 7.58-7.55 (m, 1H), 7.54-7.51 (m, 1H), 7.47-7.35 (m, 5H), 7.12 (s, 1H), 7.00 (d, J=7.6 Hz, 1H), 4.35 (brt, J=6.4 Hz, 2H), 3.63-3.53 (m, 7H), 3.50-3.46 (m, 24H), 3.13 (brt, J=6.4 Hz, 2H), 2.58-2.55 (m, 2H), 2.39 (brt, J=6.3 Hz, 6H), 1.41-1.35 (m, 9H). Theoretical number of H: 69, found: 69.

### Preparation of Intermediate 19

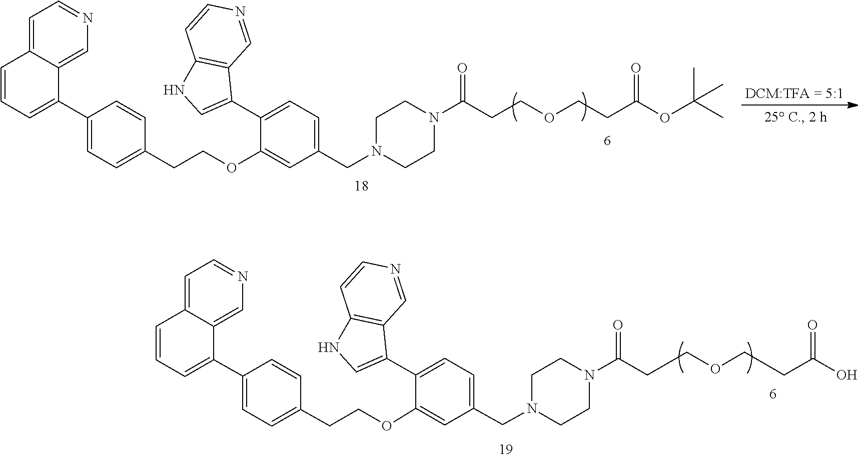

To a solution of Intermediate 18 (107 mg, 111.44 μmol, 1.00 equivalent), TFA (0.80 mL) and DCM (4 mL) was added at 25° C. for 2 hours. LCMS showed the reactant was consumed and the desired mass of the product was observed. The reaction was concentrated under reduced pressure. The residue was used to the next step without purification. Intermediate 19 (100.7 mg, 111.39 μmol, 99.95% yield) was obtained as a yellow oil. LCMS: RT=1.773 min, MS cal.: 903.4, found: [M+H]+=904.4. ¹H NMR (400 MHz, DMSO-d₆) δ=11.63 (br s, 1H), 9.15 (s, 1H), 8.97 (s, 1H), 8.54 (d, J=5.6 Hz, 1H), 8.19 (d, J=5.8 Hz, 1H), 8.00 (d, J=8.1 Hz, 1H), 7.91 (d, J=5.8 Hz, 1H), 7.86-7.81 (m, 1H), 7.58-7.53 (m, 3H), 7.46-7.38 (m, 5H), 7.13 (s, 1H), 7.00 (d, J=8.8 Hz, 1H), 4.36 (t, J=6.5 Hz, 2H), 3.64-3.53 (m, 7H), 3.52-3.46 (m, 24H), 3.13 (brt, J=6.6 Hz, 2H), 2.59-2.55 (m, 2H), 2.45-2.35 (m, 6H). Theoretical number of H: 61, found: 61.

### Preparation of Intermediate 21

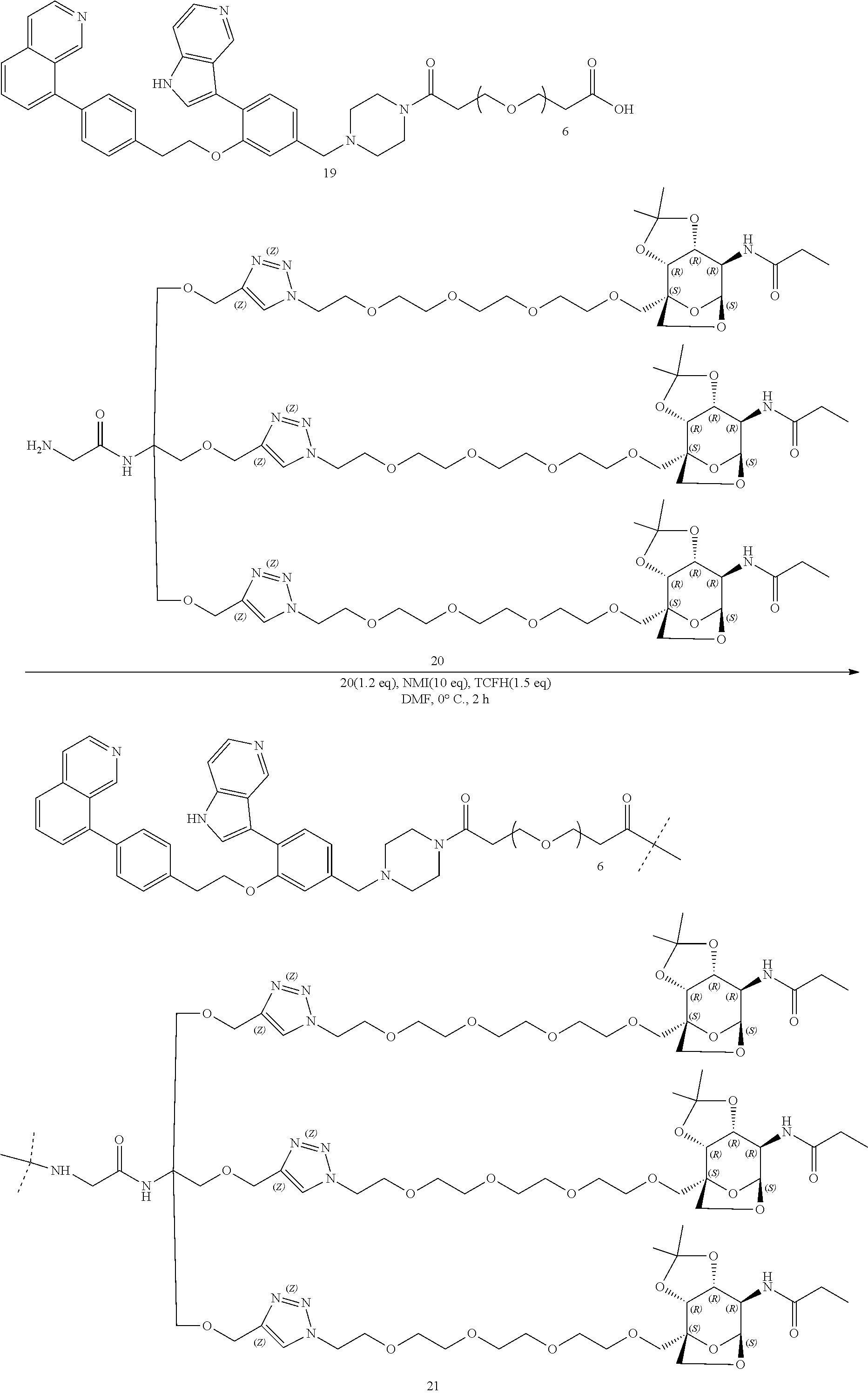

To a solution of Intermediate 19 (100 mg, 65.26 μmol, 59% purity, 1.00 equivalent) in DMF (1 mL), 1-methylimidazole (NMI) (53.58 mg, 652.60 μmol, 52.02 uL, 10.00 equivalent) was added, and the reaction mixture was cooled to 0° C. Then Intermediate 20 (137.67 mg, 78.31 μmol, 1.20 equivalent) and tetramethylchloroformamidinium hexafluorophosphate (TCFH) (27.47 mg, 97.89 μmol, 1.50 equivalent) in DMF (1.30 mL) was added to the above reaction mixture at 0° C. The reaction mixture was stirred at 0° C. for 2 hours. LCMS showed Intermediate 19 was consumed and the desired mass of the product was obtained. The reaction was used to the next step without further purification. LCMS: RT=2.150 min, MS cal.: 2642.3, found: [M/2+H]+=1323.0.

### Preparation of Intermediate Compound 102

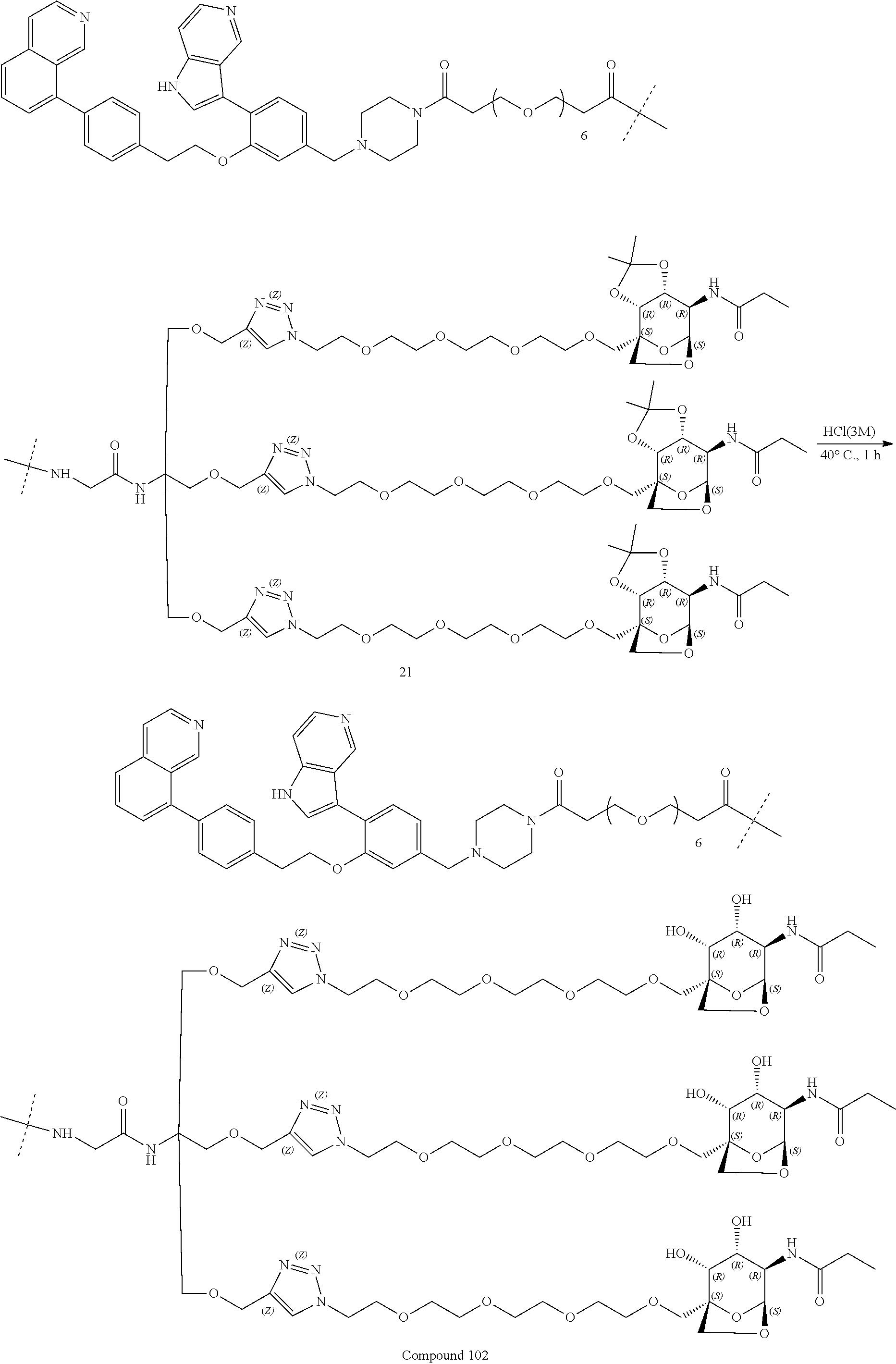

To a solution of Intermediate 21 (130 mg 49.17 μmol, 1.00 equivalent), HCl (3 M, 2.10 mL 98.18 equivalent) was added at 40° C. and was stirred for 1 hour. LCMS showed the reaction was complete and the desired mass of the product was obtained. The reaction mixture was concentrated under reduced pressure. The residue was purified by prep-HPLC (Column: Phenomenex C18 75*30 mm*3 μm; mobile phase: [water (NH₄HCO₃)-ACN]; B %: 15%-45%, 8 min.). Compound 102 (31.34 mg, 12.42 μmol, 19.33% yield) was obtained as a yellow solid. LCMS: RT=2.666 min, MS cal.: 2522.22, found: [M/3+H]+=842.1, [M/4+H]+=631.8. ¹H NMR (400 MHz, DMSO-d₆) δ=11.68-11.57 (m, 1H), 9.14 (s, 1H), 8.97 (s, 1H), 8.53 (d, J=5.6 Hz, 1H), 8.19 (d, J=5.8 Hz, 1H), 8.01 (s, 3H), 7.90 (d, J=5.8 Hz, 1H), 7.86-7.77 (m, 4H), 7.58-7.51 (m, 3H), 7.43-7.39 (m, 4H), 7.25 (s, 1H), 7.12 (s, 1H), 7.00 (d, J=7.8 Hz, 1H), 5.07 (d, J=1.3 Hz, 3H), 4.83 (d, J=5.4 Hz, 3H), 4.56 (br d, J=6.8 Hz, 3H), 4.53-4.44 (m, 12H), 4.35 (brt, J=6.6 Hz, 2H), 3.83-3.77 (m, 9H), 3.76-3.70 (m, 3H), 3.67-3.56 (m, 24H), 3.55-3.45 (m, 67H), 3.13 (brt, J=6.5 Hz, 2H), 2.58-2.53 (m, 3H), 2.43-2.35 (m, 6H), 2.15-2.08 (m, 6H), 0.97 (t, J=7.6 Hz, 9H). Theoretical number of H: 175, found: 175.

### Example 3. Procedure for Preparation of Compound 103

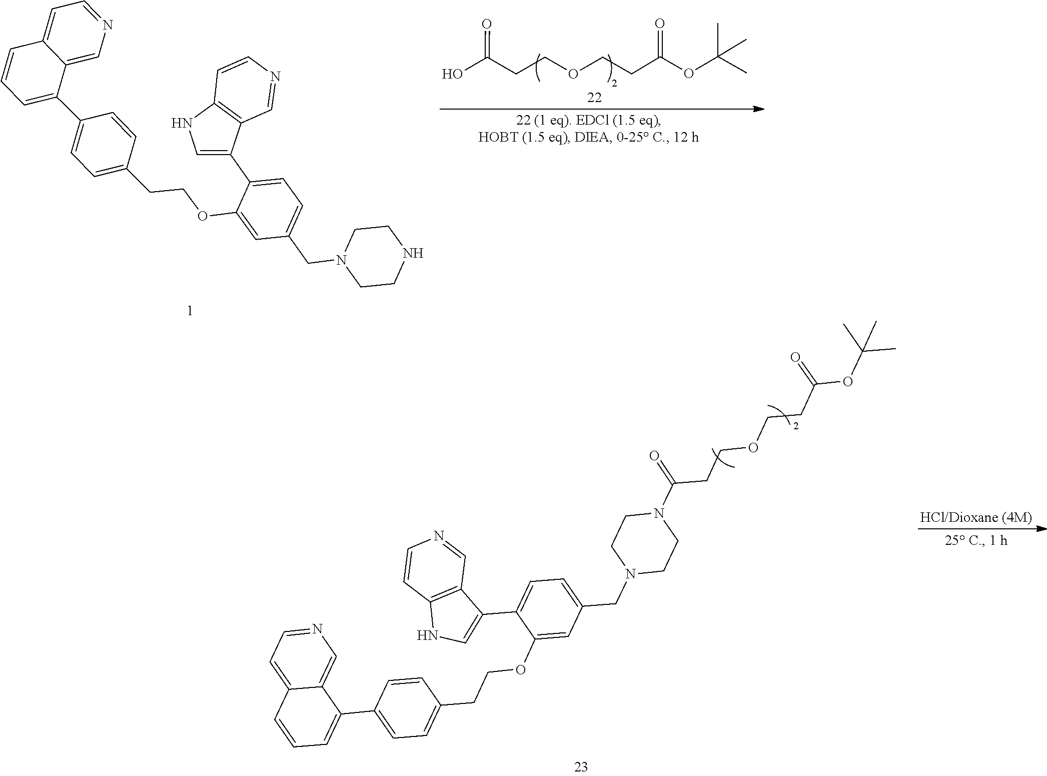

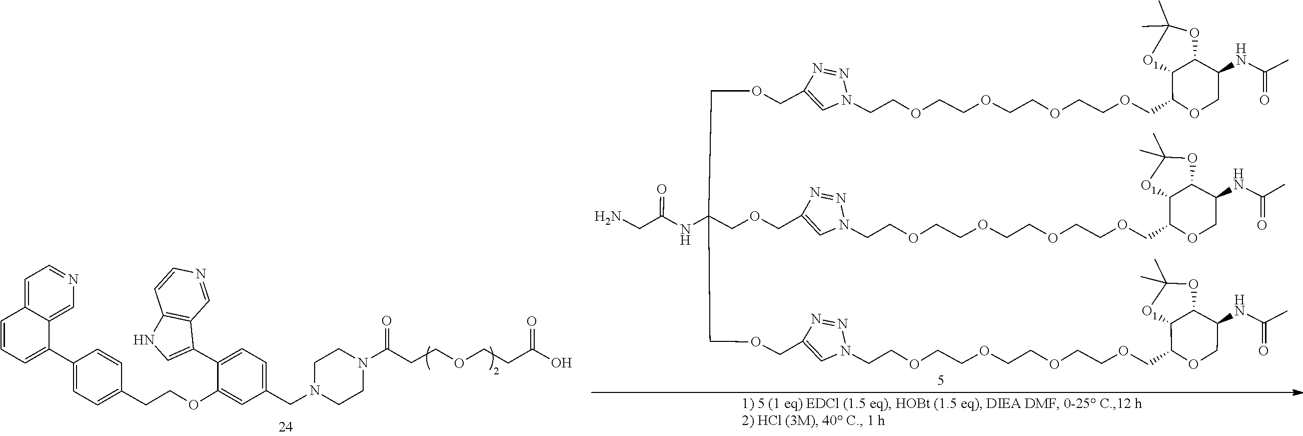

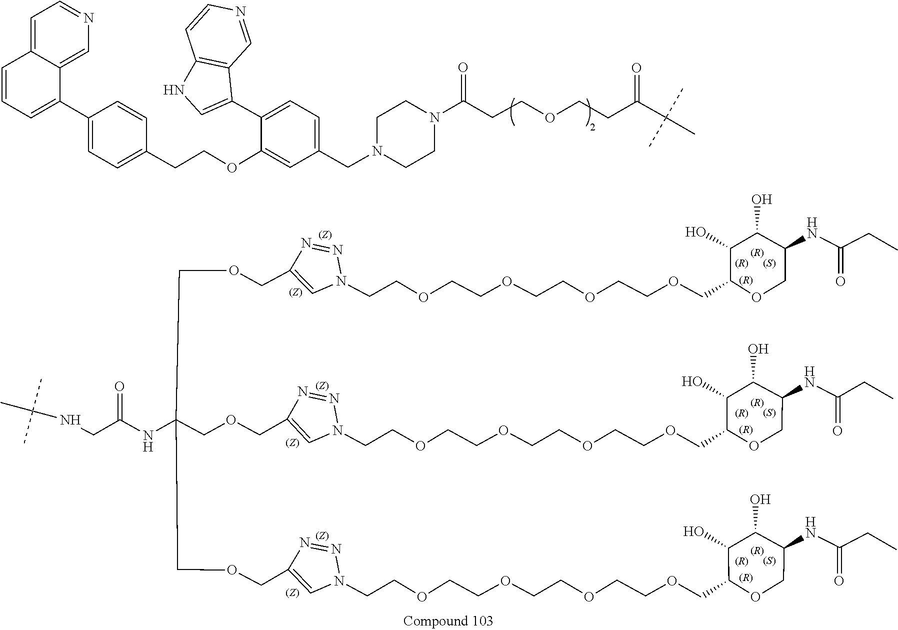

### Preparation of Intermediate 23

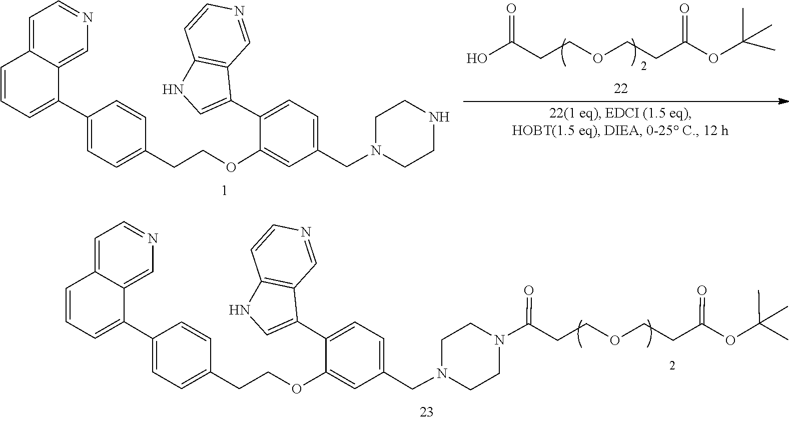

To a solution of Intermediate 22 (30.00 mg, 114.37 μmol, 1.00 equivalent) and the Intermediate 1 (61.72 mg, 114.37 μmol, 1 equivalent) in DMF (2 mL), EDCI (32.89 mg, 171.56 μmol, 1.50 equivalent) and HOBt (23.18 mg, 171.56 μmol, 1.50 equivalent) was added at 25° C. Then DIEA (44.35 mg, 343.12 μmol, 59.77 uL, 3.00 equivalent) was added dropwise to the above reaction mixture at 0° C. Then reaction mixture was stirred at 25° C. for 12 hours. LCMS showed the reactant was consumed and the desired mass of the product was detected. The reaction mixture was concentrated under reduced pressure to give a residue. The residue was purified by prep-HPLC (neutral condition) (column: Waters Xbridge Prep OBD C18 150*40 mm*10 μm; mobile phase: [water (NH₄HCO₃)-ACN]; B %: 40%-80%, 8 min) to afford Intermediate 23 (27 mg, 34.44 μmol, 30.11% yield) as white solid. LCMS: RT=3.208 min, MS cal.: 783.4, found: [M+H]⁺=784.5. ¹H NMR (400 MHz, DMSO-d₆) δ=11.61 (s, 1H), 9.14 (s, 1H), 8.97 (s, 1H), 8.53 (d, J=5.8 Hz, 1H), 8.19 (d, J=5.6 Hz, 1H), 7.99 (d, J=8.3 Hz, 1H), 7.91 (d, J=5.8 Hz, 1H), 7.86-7.80 (m, 1H), 7.58-7.51 (m, 3H), 7.45-7.37 (m, 5H), 7.12 (s, 1H), 7.00 (d, J=7.8 Hz, 1H), 4.35 (t, J=6.6 Hz, 2H), 3.62-3.52 (m, 6H), 3.46 (s, 8H), 3.13 (t, J=6.6 Hz, 2H), 2.54 (t, J=6.7 Hz, 2H), 2.40 (br t, J=6.3 Hz, 6H), 1.37 (s, 9H). Theoretical number of H: 53, found: 53.

### Preparation of Intermediate 24

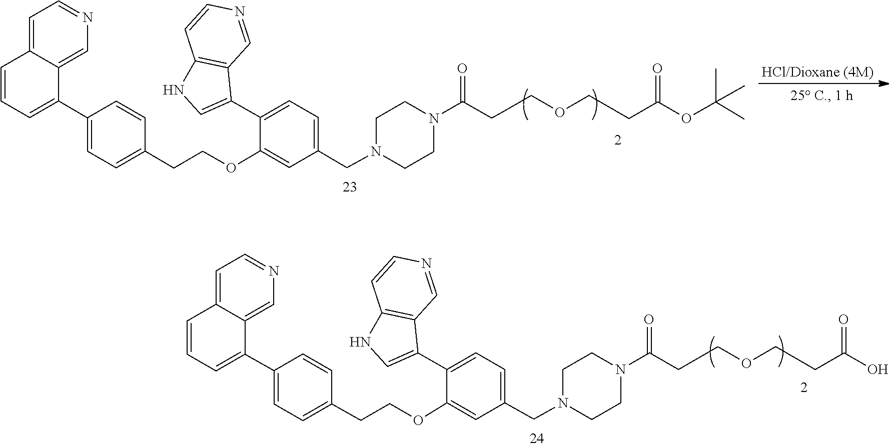

To a solution of Intermediate 23 (1.00 g, 1.28 mmol, 1.00 equivalent), HCl/dioxane (4 M, 5 mL, 15.68 equivalent) was added at 25° C. for 1 hour. LCMS showed the reaction was complete. The reaction mixture was concentrated under reduced pressure to give a residue. The residue was purified by prep-HPLC (column: Waters Xbridge BEH C18 250*50 mm*10 μm; mobile phase: [water (NH₄HCO₃)-ACN]; B %: 10%-50%, 10 min) to afford Intermediate 24 (29.7 mg, 40.76 μmol, 31.95% yield) as white solid. LCMS: RT=1.744 min, MS cal.: 727.3, found: [M/2+H]+=364.7. ¹H NMR (400 MHz, DMSO-d₆) δ=11.61 (br s, 1H), 9.14 (s, 1H), 8.97 (s, 1H), 8.53 (d, J=5.6 Hz, 1H), 8.19 (d, J=5.6 Hz, 1H), 7.99 (d, J=8.3 Hz, 1H), 7.91 (d, J=5.6 Hz, 1H), 7.86-7.81 (m, 1H), 7.58-7.51 (m, 3H), 7.45-7.37 (m, 5H), 7.12 (s, 1H), 7.00 (d, J=7.9 Hz, 1H), 4.36 (t, J=6.6 Hz, 2H), 3.63-3.56 (m, 5H), 3.53 (s, 2H), 3.47 (s, 9H), 3.13 (br t, J=6.5 Hz, 2H), 2.58-2.53 (m, 2H), 2.44-2.41 (m, 3H), 2.39-2.34 (m, 2H). Theoretical number of H: 45, found: 45.

### Preparation of Compound 103

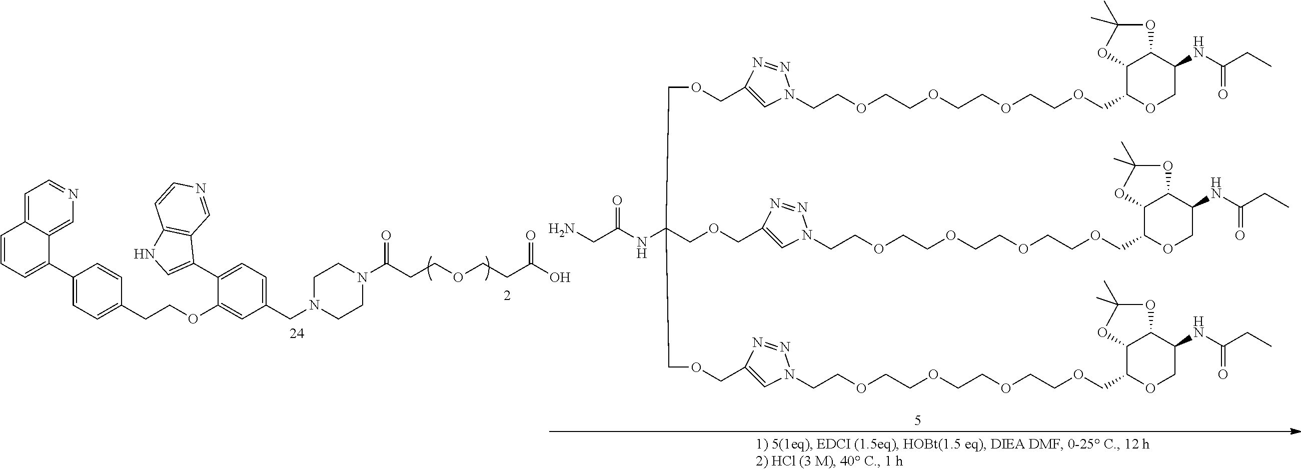

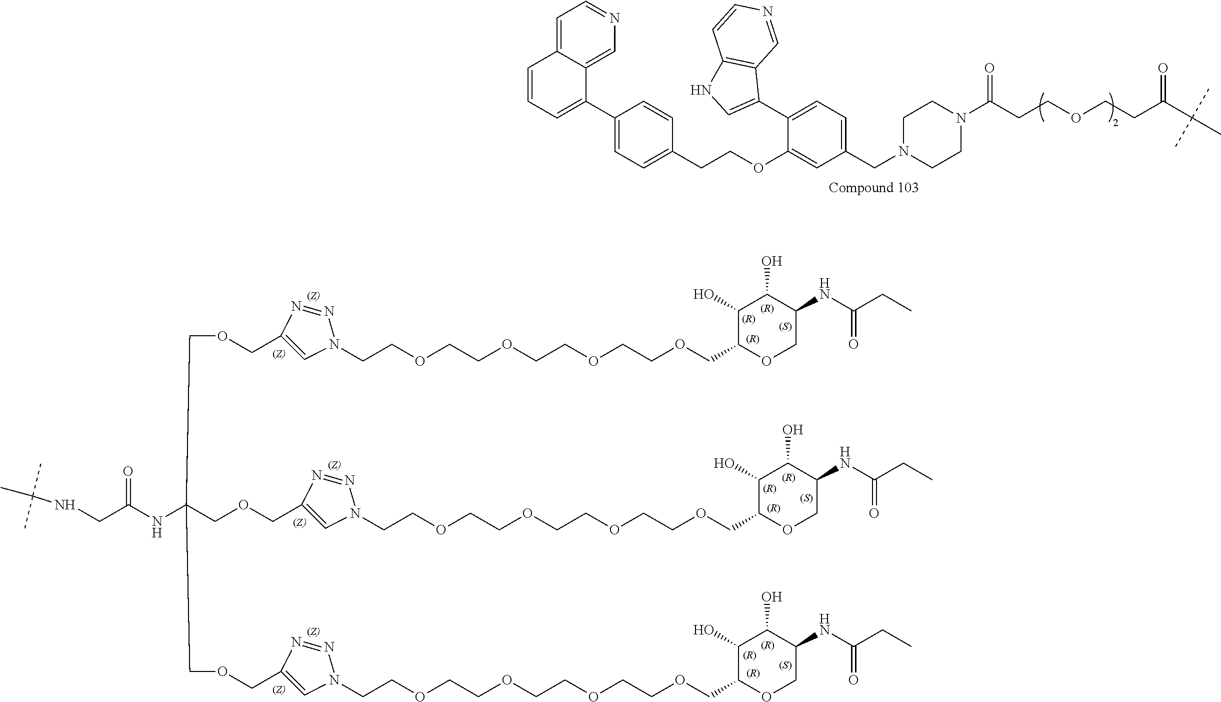

To a solution of Intermediate 24 (45.00 mg, 61.83 μmol, 1.00 equivalent) and Intermediate 5 (103.49 mg, 61.83 μmol, 1.00 equivalent) in DMF (1 mL), EDCI (17.78 mg, 92.74 μmol, 1.50 equivalent) and HOBt (12.53 mg, 92.74 μmol, 1.50 equivalent) was added at 25° C. Then DIEA (23.97 mg, 185.48 μmol, 32.31 uL, 3.00 equivalent) in DMF (0.50 mL) was added dropwise to the above reaction mixture at 0° C. Then reaction mixture was stirred at 25° C. for 12 hours. The reaction mixture was concentrated under reduced pressure to give a residue. The residue was used in the next step directly without purification. Then HCl (3 M, 3.00 mL, 79.44 equivalent) was added to the residue and the mixture was stirred at 40° C. for 1 hour. LCMS showed reactant was consumed and the desired mass of the product was detected. The reaction mixture was concentrated under reduced pressure to give a residue. The residue was purified by prep-HPLC (column: Phenomenex Luna C18 80*40 mm*3 μm; mobile phase: [water (HCl)-ACN]; B %: 1%-30%, 7 min) to afford Compound 103 (50.81 mg, 22.45 μmol, 76.44% yield) as white solid. LCMS: RT=1.865 min, MS cal.: 2263.5, found: [M/3+H]+=755.3. [M/4+H]+=566.8. ¹H NMR (400 MHz, MeOD-d₄) δ=9.42 (s, 1H), 9.24 (s, 1H), 8.63-8.60 (m, 1H), 8.58-8.55 (m, 1H), 8.40 (d, J=7.0 Hz, 1H), 8.36-8.27 (m, 2H), 8.03-8.00 (m, 5H), 7.95-7.92 (m, 1H), 7.72 (d, J=7.6 Hz, 1H), 7.57 (s, 1H), 7.48-7.43 (m, 2H), 7.40-7.35 (m, 2H), 7.33-7.29 (m, 1H), 4.58-4.49 (m, 17H), 4.17-4.08 (m, 3H), 3.90-3.83 (m, 13H), 3.82 (s, 2H), 3.77-3.70 (m, 11H), 3.63-3.52 (m, 53H), 3.52-3.49 (m, 4H), 3.21 (brt, J=6.5 Hz, 3H), 3.14-3.12 (m, 1H), 3.04 (t, J=11.0 Hz, 3H), 2.51 (t, J=6.1 Hz, 2H), 2.26-2.18 (m, 6H), 1.11 (t, J=7.6 Hz, 9H). Theoretical number of H: 159, found: 147, exchangeable H: 12.

### Example 4. Procedure for Preparation of Compound 104

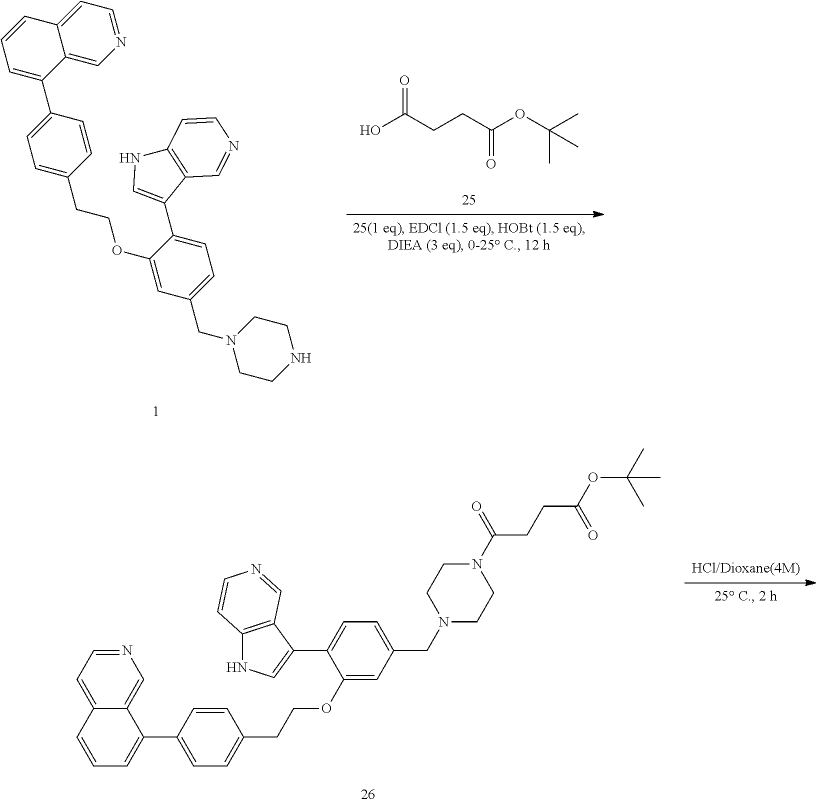 [SMILES: c1cc(-c2ccc(CCOc3cc(CN4CCNCC4)ccc3-c3c[nH]c4ccncc34)cc2)c2cnccc2c1 ; CC(C)(C)OC(=O)CCC(=O)O ; CC(C)(C)OC(=O)CCC(=O)N1CCN(Cc2ccc(-c3c[nH]c4ccncc34)c(OCCc3ccc(-c4cccc5ccncc45)cc3)c2)CC1]

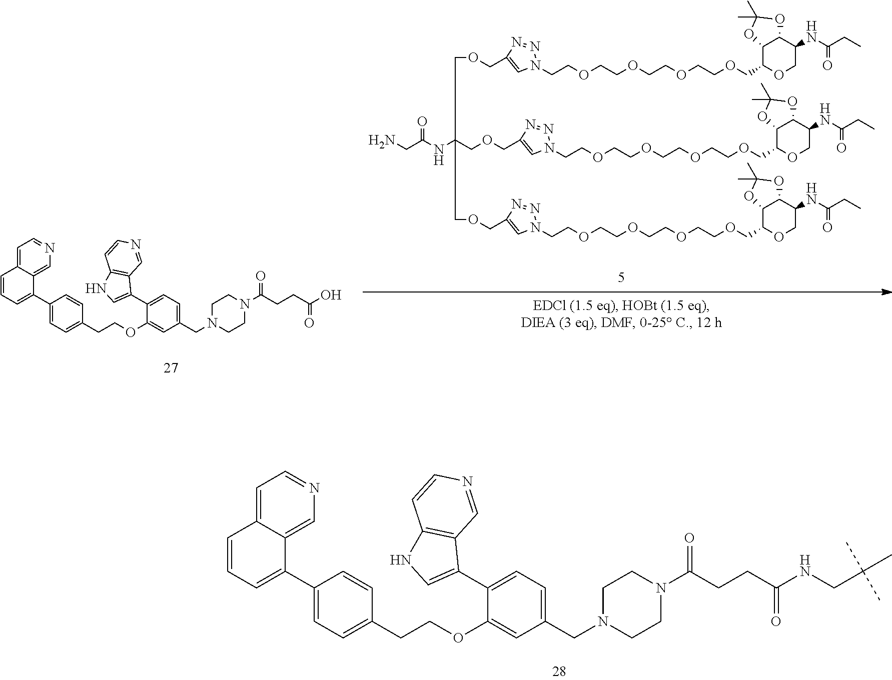

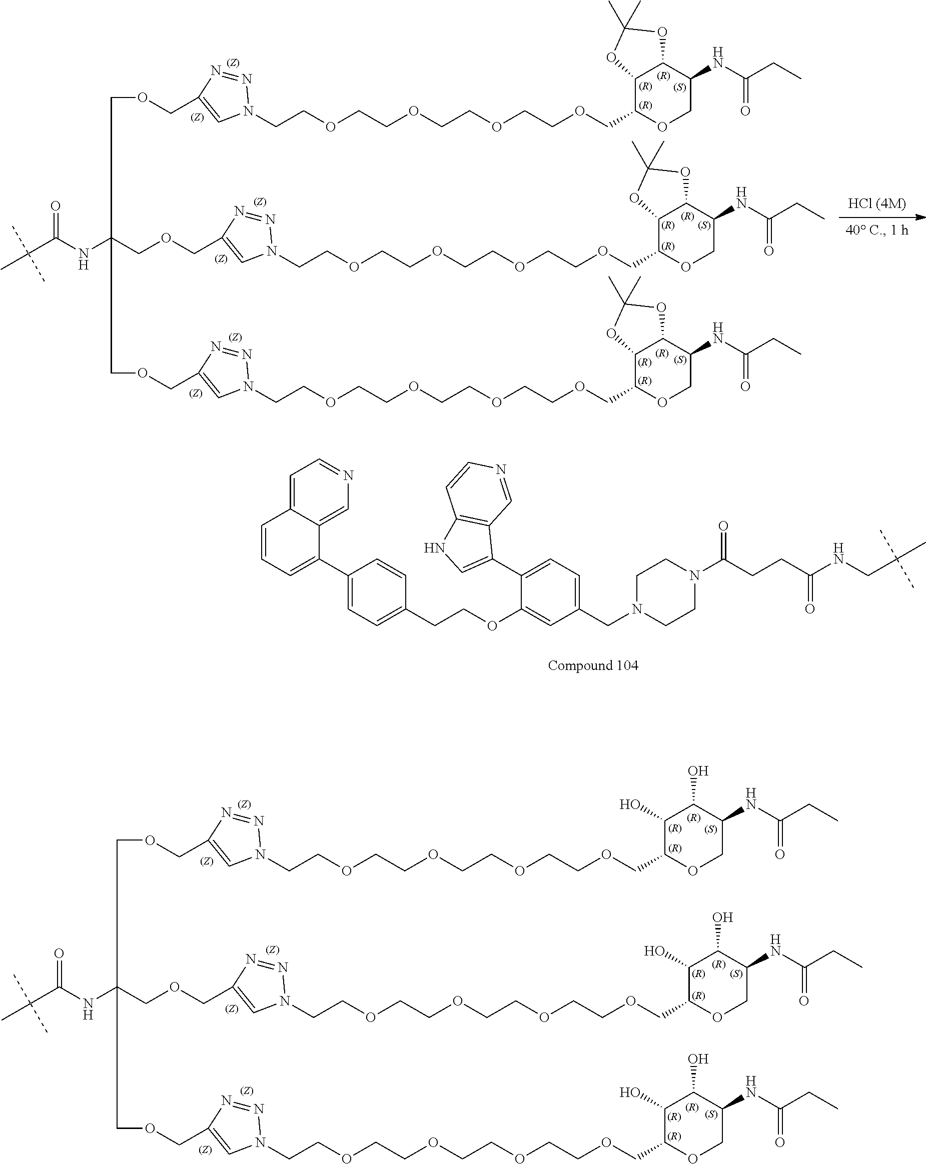

### Preparation of Intermediate 20

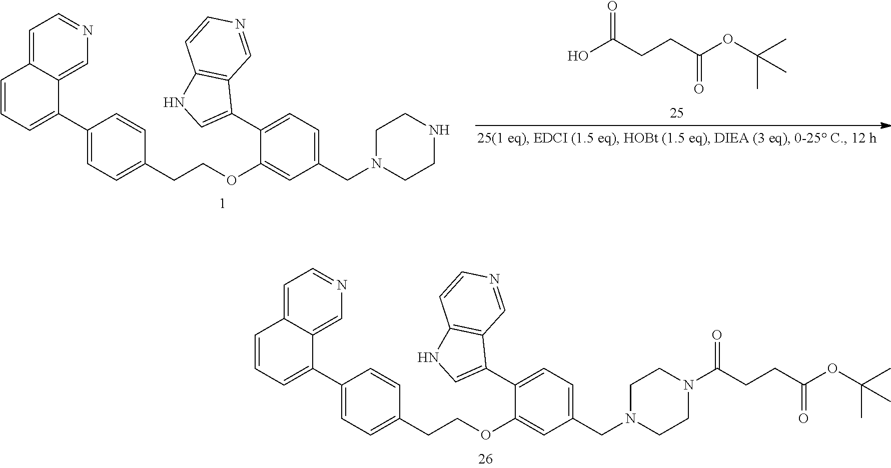 [SMILES: c1cc(-c2ccc(CCOc3cc(CN4CCNCC4)ccc3-c3c[nH]c4ccncc34)cc2)c2cnccc2c1 ; CC(C)(C)OC(=O)CCC(=O)O ; CC(C)(C)OC(=O)CCC(=O)N1CCN(Cc2ccc(-c3c[nH]c4ccncc34)c(OCCc3ccc(-c4cccc5ccncc45)cc3)c2)CC1]

To a solution of the Intermediate 1 (0.40 g, 741.20 μmol, 1.00 equivalent) and Intermediate 25 (129.11 mg, 741.20 μmol, 1.00 equivalent) in DMF (5 mL), EDCI (213.13 mg, 1.11 mmol, 1.50 equivalent) and HOBt (150.23 mg, 1.11 mmol, 1.50 equivalent) were added. Then DIEA (287.38 mg, 2.22 mmol, 387.31 uL, 3.00 equivalent) was added dropwise to the above reaction mixture at 0° C. Then reaction mixture was stirred at 25° C. for 12 hours. LCMS showed the reactant was consumed and the desired mass of the product was detected. The reaction mixture was concentrated under reduced pressure to give a residue. The residue was purified by prep-HPLC (column: Waters Xbridge BEH C18 100*30 mm*10 μm; mobile phase: [water (NH₄HCO₃)-ACN]; B %: 30%-75%, 8 min) to give Intermediate 26 (0.34 g, 488.61 μmol, 65.9% yield) as white solid. LCMS: RT=2.485 min, MS cal.: 695.8, [M+H]=696.4. ¹H NMR (400 MHz, DMSO-d₆) δ=11.60 (s, 1H), 9.14 (s, 1H), 8.97 (s, 1H), 8.53 (d, J=5.6 Hz, 1H), 8.19 (d, J=5.6 Hz, 1H), 7.99 (d, J=8.3 Hz, 1H), 7.91 (d, J=5.3 Hz, 1H), 7.86-7.80 (m, 1H), 7.59-7.51 (m, 3H), 7.44 (s, 1H), 7.43-7.37 (m, 5H), 7.13 (s, 1H), 7.02-6.98 (m, 1H), 4.36 (t, J=6.6 Hz, 2H), 3.54 (s, 2H), 3.49-3.44 (m, 4H), 3.13 (t, J=6.6 Hz, 2H), 2.43 (br s, 2H), 2.41-2.31 (m, 5H), 1.37 (s, 9H). Theoretical number of H: 45, found: 45.

### Preparation of Intermediate 27

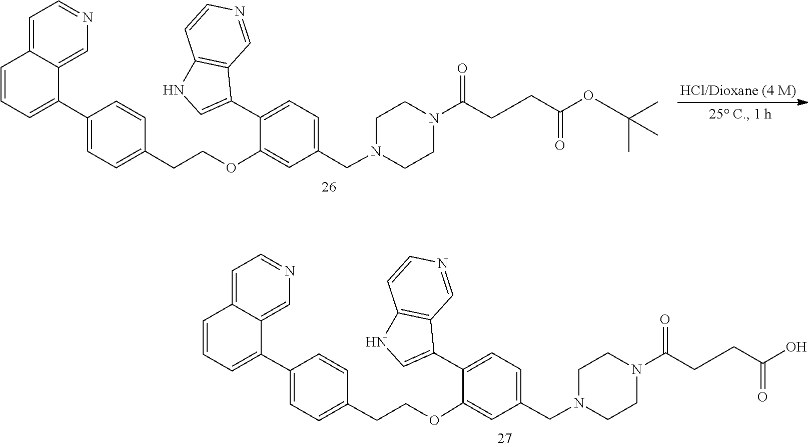 [SMILES: CC(C)(C)COC(=O)CCCC(=O)N1CCN(Cc2ccc(-c3c[nH]c4ccncc34)c(OCCc3ccc(-c4cccc5ccncc45)cc3)c2)CC1 ; O=C(O)CCCC(=O)N1CCN(Cc2ccc(-c3c[nH]c4ccncc34)c(OCCc3ccc(-c4cccc5ccncc45)cc3)c2)CC1]

To a solution of Intermediate 26 (0.34 g, 488.61 μmol, 1.00 equivalent), HCl/dioxane (4 M, 5 mL, 15.68 equivalent) was added at 25° C. for 2 hours. LCMS showed the reaction was complete. The reaction mixture was concentrated under reduced pressure to give a residue. The residue was purified by prep-HPLC (column: Waters Xbridge Prep OBD C18 150*40 mm*10 μm; mobile phase: [water (NH₄HCO₃)-ACN]; B %: 25%-55%, 8 min) to give Intermediate 27 (22.00 mg, 34.39 μmol, 7.0% yield) as white solid. LCMS: RT=2.485 min, MS cal.: 639.7, [M+H]+=640.4. ¹H NMR (400 MHz, DMSO-d₆) δ=11.62 (br s, 1H), 9.14 (s, 1H), 8.97 (s, 1H), 8.53 (d, J=5.6 Hz, 1H), 8.19 (d, J=5.6 Hz, 1H), 7.99 (d, J=8.1 Hz, 1H), 7.91 (d, J=5.6 Hz, 1H), 7.83 (t, J=7.7 Hz, 1H), 7.58-7.51 (m, 3H), 7.45-7.37 (m, 5H), 7.13 (s, 1H), 7.00 (d, J=7.8 Hz, 1H), 4.36 (brt, J=6.4 Hz, 2H), 3.54 (s, 2H), 3.47 (br s, 4H), 3.17 (br d, J=4.9 Hz, 1H), 3.13 (br t, J=6.3 Hz, 2H), 2.42 (br d, J=6.0 Hz, 5H), 2.38 (br d, J=8.5 Hz, 2H), 2.33 (br s, 1H). Theoretical number of H: 37, found: 37

### Preparation of Intermediate 28

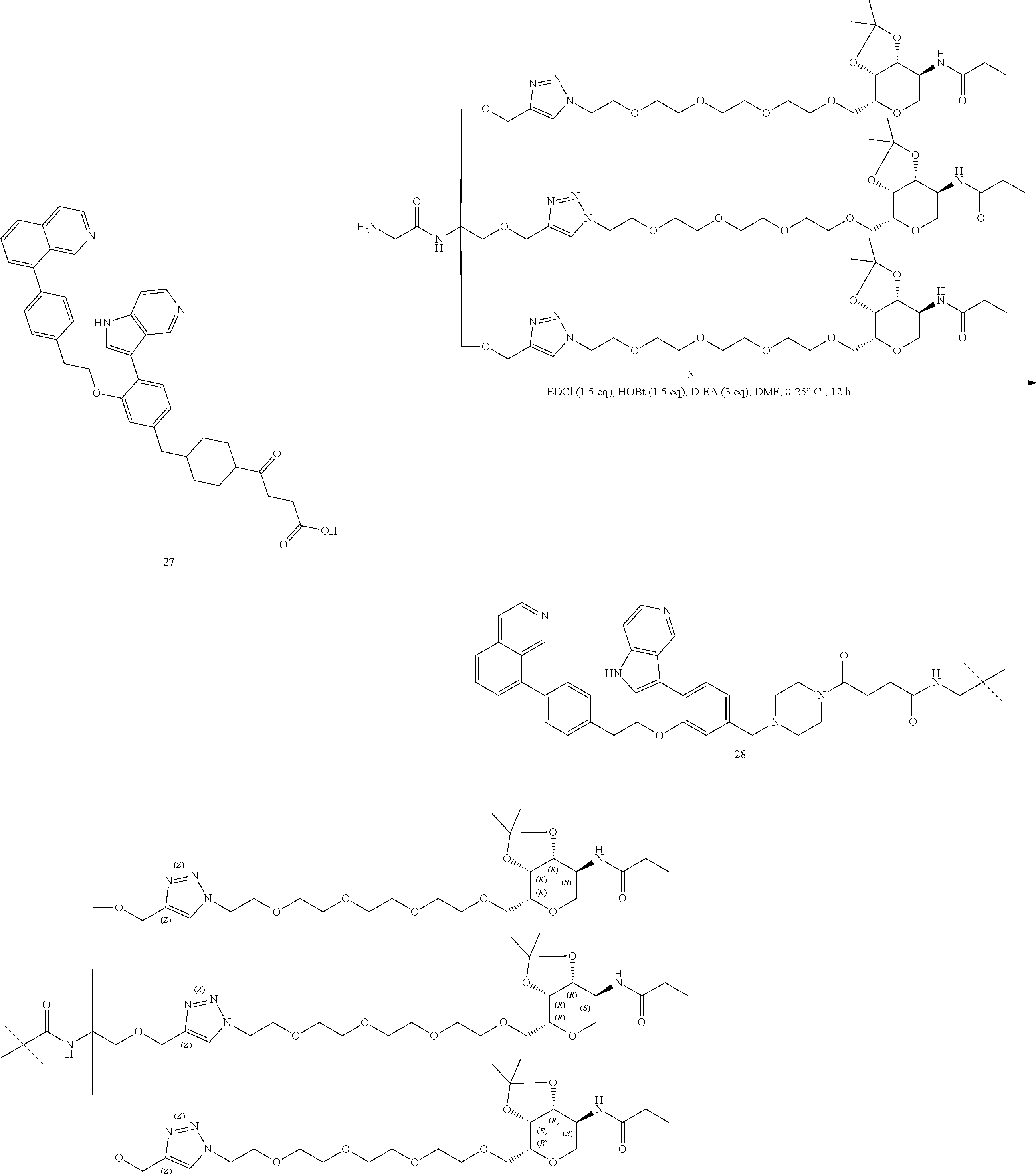

To a solution of Intermediate 27 (67.00 mg, 104.73 μmol, 1.00 equivalent) and Intermediate 5 (162.72 mg, 104.73 μmol, 1.00 equivalent) in DMF (1 mL) was added EDCI (30.12 mg, 157.09 μmol, 1.50 equivalent) and HOBt (21.23 mg, 157.09 μmol, 1.50 equivalent) at 25° C. Then DIEA (40.61 mg, 314.19 μmol, 54.72 uL, 3.00 equivalent) in DMF (0.5 mL) was added dropwise to the above reaction mixture at 0° C. Then the reaction mixture was stirred at 25° C. for 12 hours. LCMS showed the reaction was completed. The reaction was concentrated under reduced pressure to give crude product. The crude product was purified by prep-HPLC (column: Waters Xbridge BEH C18 100*30 mm*10 μm; mobile phase: [water (NH₄HCO₃)-ACN]; B %: 15%-45%, 8 min) to give Intermediate 28 (120.00 mg, 52.27 μmol, 49.9% yield) as yellow solid. LCMS: RT=1.251 min, MS cal.: 2295.62, found: [M/2+H]+=1148.5.

### Preparation of Compound 104

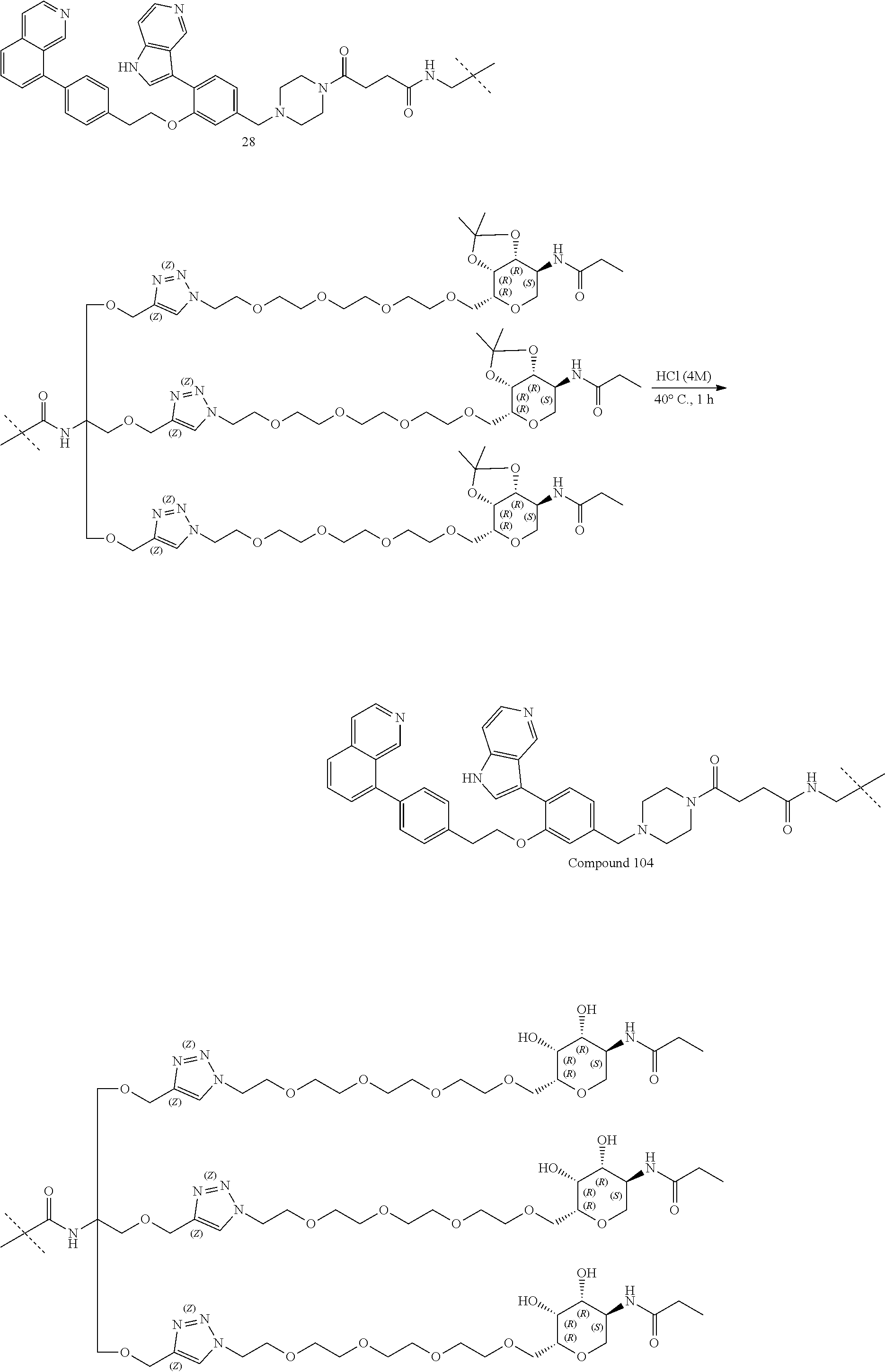

A mixture of Intermediate 28 (100.00 mg, 43.56 μmol, 1.00 equivalent) in HCl (4 M, 833.33 uL, 76.52 equivalent) was degassed and purged with N₂ for 3 times, and then the mixture was stirred at 40° C. for 1 hour under N₂ atmosphere. LCMS showed the reaction was completed. The reaction was concentrated under reduced pressure to give crude product. The crude product was purified by prep-HPLC (column: Waters Xbridge Prep OBD C18 150*40 mm*10 μm; mobile phase: [water (NH₄HCO₃)-ACN]; B %: 25%-65%, 8 min) to give Compound 104 (30.00 mg, 13.79 μmol, 31.7% yield) as white solid. LCMS: RT=2.487 min, MS cal.: 2175.47, found: [M/3+H]+=726.0. ¹H NMR (400 MHz, MeOD-d₄) δ=9.07 (s, 1H), 8.92 (s, 1H), 8.45 (d, J=5.8 Hz, 1H), 8.16 (d, J=5.8 Hz, 1H), 8.00 (s, 3H), 7.94 (d, J=8.3 Hz, 1H), 7.88 (d, J=5.5 Hz, 1H), 7.82 (dd, J=7.2, 8.2 Hz, 1H), 7.52 (d, J=7.8 Hz, 2H), 7.48 (s, 1H), 7.45 (dd, J=0.8, 5.9 Hz, 1H), 7.31-7.26 (m, 2H), 7.26-7.21 (m, 2H), 7.18 (s, 1H), 7.04 (d, J=7.6 Hz, 1H), 4.57-4.53 (m, 12H), 4.40 (t, J=6.3 Hz, 2H), 4.13 (dt, J=5.1, 10.7 Hz, 3H), 3.90 (d, J=5.3 Hz, 2H), 3.88-3.85 (m, 7H), 3.83 (d, J=3.0 Hz, 3H), 3.81 (s, 2H), 3.77 (s, 6H), 3.63-3.59 (m, 11H), 3.59-3.53 (m, 36H), 3.52-3.45 (m, 7H), 3.15-3.11 (m, 2H), 3.04 (t, J=10.9 Hz, 3H), 2.73-2.66 (m, 2H), 2.54-2.44 (m, 6H), 2.21 (dq, J=1.4, 7.6 Hz, 6H), 1.11 (t, J=7.6 Hz, 9H). Theoretical number of H: 151, found: 139, exchangeable H: 12.

### Example 5. Procedure for Preparation of Compound 105

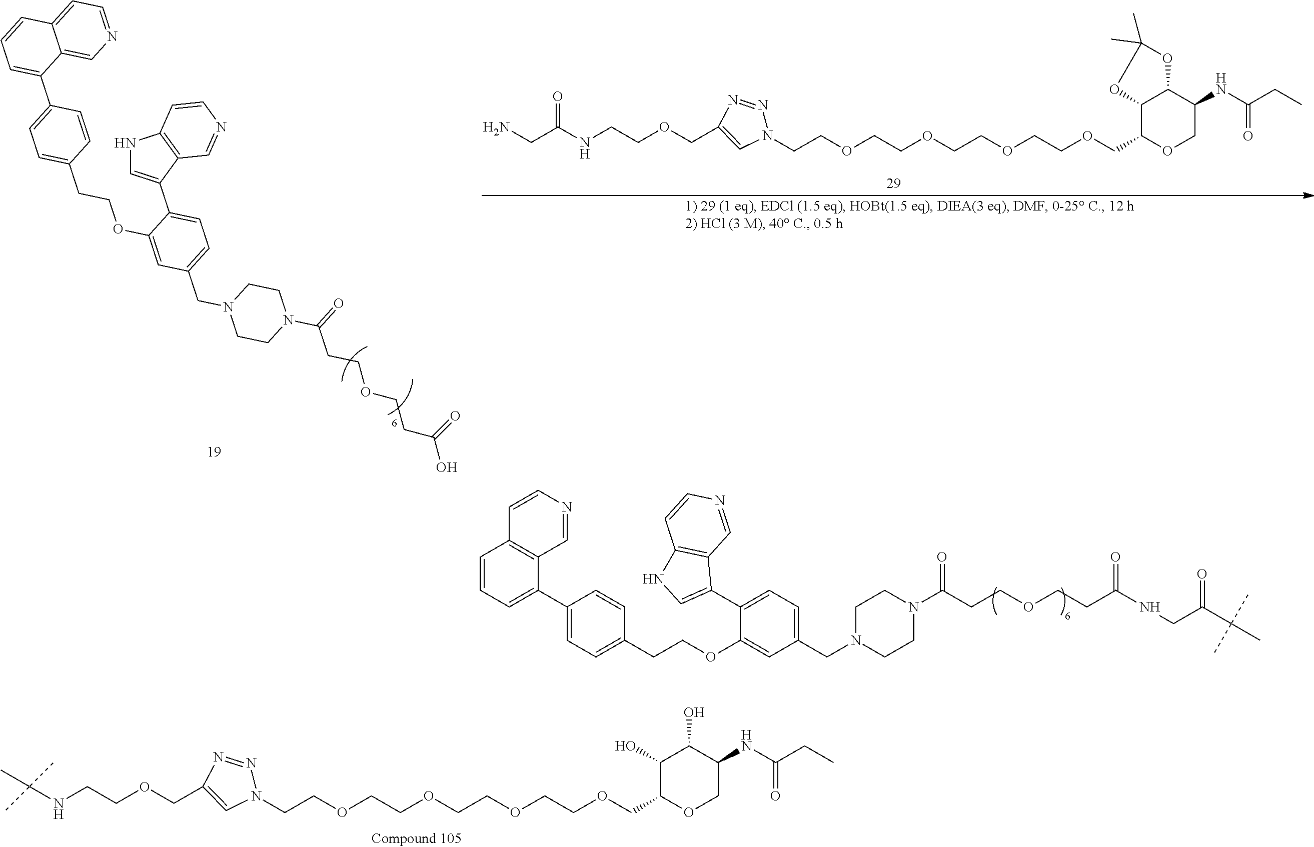

To a solution of Intermediate 19 (150 mg, 165.92 μmol, 1.00 equivalent) and Intermediate 29 (102.32 mg, 165.92 μmol, 1.00 equivalent) in DMF (3 mL), EDCI (47.71 mg, 248.88 μmol, 1.50 equivalent) and HOBt (33.63 mg, 248.88 μmol, 1.50 equivalent) was added at 25° C. Then DIEA (64.33 mg, 497.76 μmol, 86.70 uL, 3.00 equivalent) in DMF (0.50 mL) was added dropwise to the above reaction mixture at 0° C. Then the reaction mixture was stirred at 25° C. for 12 hours. The reaction mixture was concentrated under reduced pressure to give a residue. The residue was used in the next step directly without purification. HCl (3 M, 22.18 uL, 1 equivalent) was added to the residue at 40° C. and the solution was stirred at 40° C. for 30 minutes. LCMS showed the reactant was consumed and the desired mass of the product was detected. The reaction mixture was concentrated under reduced pressure to give a residue. The residue was purified by prep-HPLC (column: Waters Xbridge Prep OBD C18 150*40 mm*10 μm; mobile phase: [water (NH₄HCO₃)-ACN]; B %: 20%-70%, 8 min) to afford Compound 105 (30 mg, 20.51 μmol, 30.82% yield) as white solid. LCMS: RT=2.606 min, MS cal.: 1462.7, found: [M/2+H]+=732.0. ¹H NMR (400 MHz, MeOD-d₄) δ=9.06 (s, 1H), 8.92 (d, J=0.6 Hz, 1H), 8.45 (d, J=5.8 Hz, 1H), 8.16 (d, J=5.9 Hz, 1H), 8.02 (s, 1H), 7.95 (d, J=8.3 Hz, 1H), 7.89 (d, J=5.6 Hz, 1H), 7.85-7.80 (m, 1H), 7.54-7.51 (m, 2H), 7.49-7.45 (m, 2H), 7.31-7.27 (m, 2H), 7.25-7.21 (m, 2H), 7.19 (s, 1H), 7.06-7.03 (m, 1H), 4.59 (s, 4H), 4.40 (t, J=6.3 Hz, 2H), 4.16-4.08 (m, 1H), 3.91-3.86 (m, 3H), 3.85-3.82 (m, 3H), 3.76-3.70 (m, 5H), 3.64-3.56 (m, 41H), 3.52-3.46 (m, 3H), 3.41-3.37 (m, 2H), 3.13 (t, J=6.3 Hz, 2H), 3.04 (t, J=10.9 Hz, 1H), 2.66 (t, J=6.3 Hz, 2H), 2.54-2.47 (m, 5H), 2.25-2.18 (m, 2H), 1.11 (t, J=7.6 Hz, 3H). Theoretical number of H: 103, found: 97, exchangeable H:6.

### Example 6. Procedure for Preparation of Compound 106

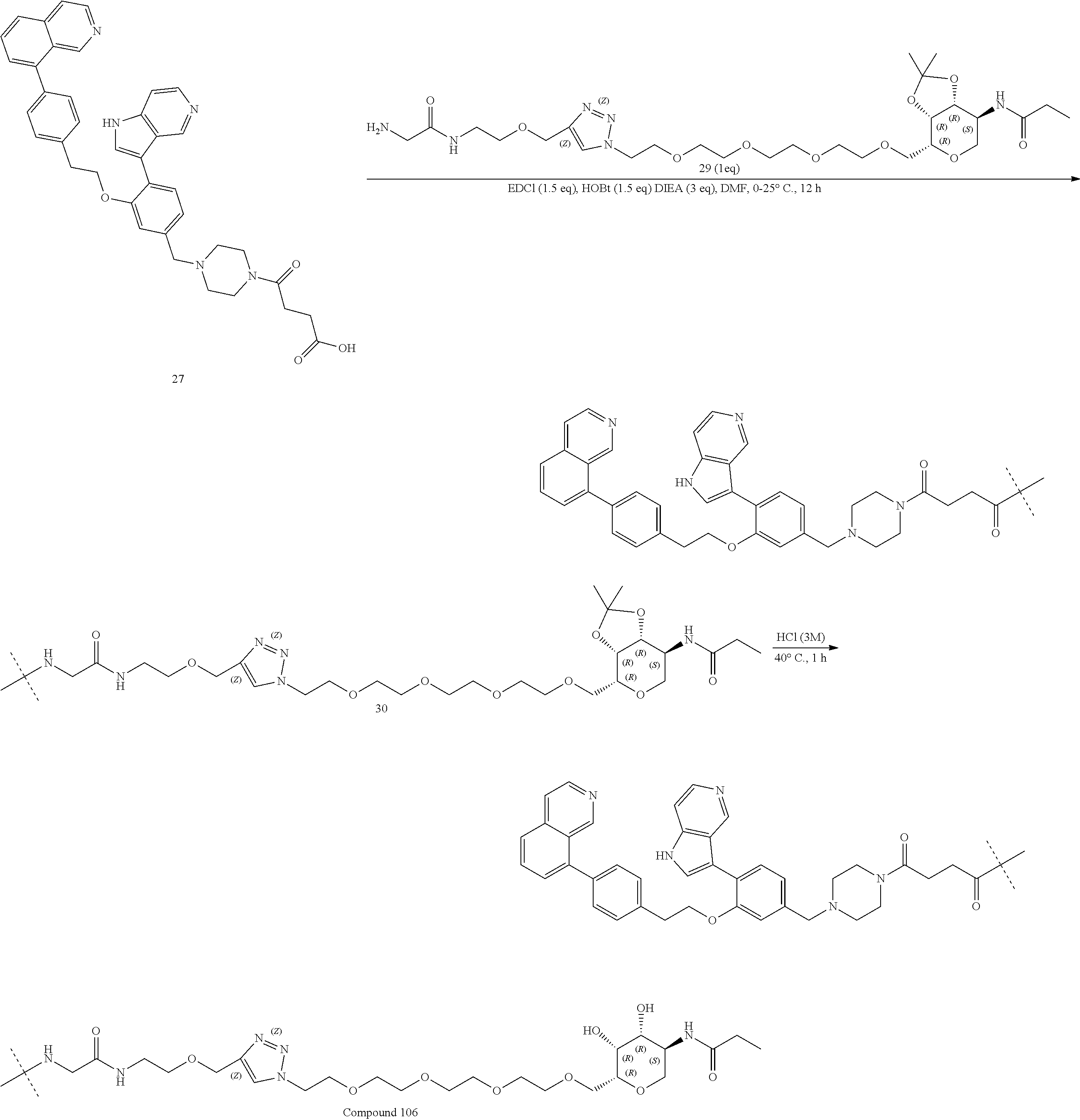

### Preparation of Intermediate 30

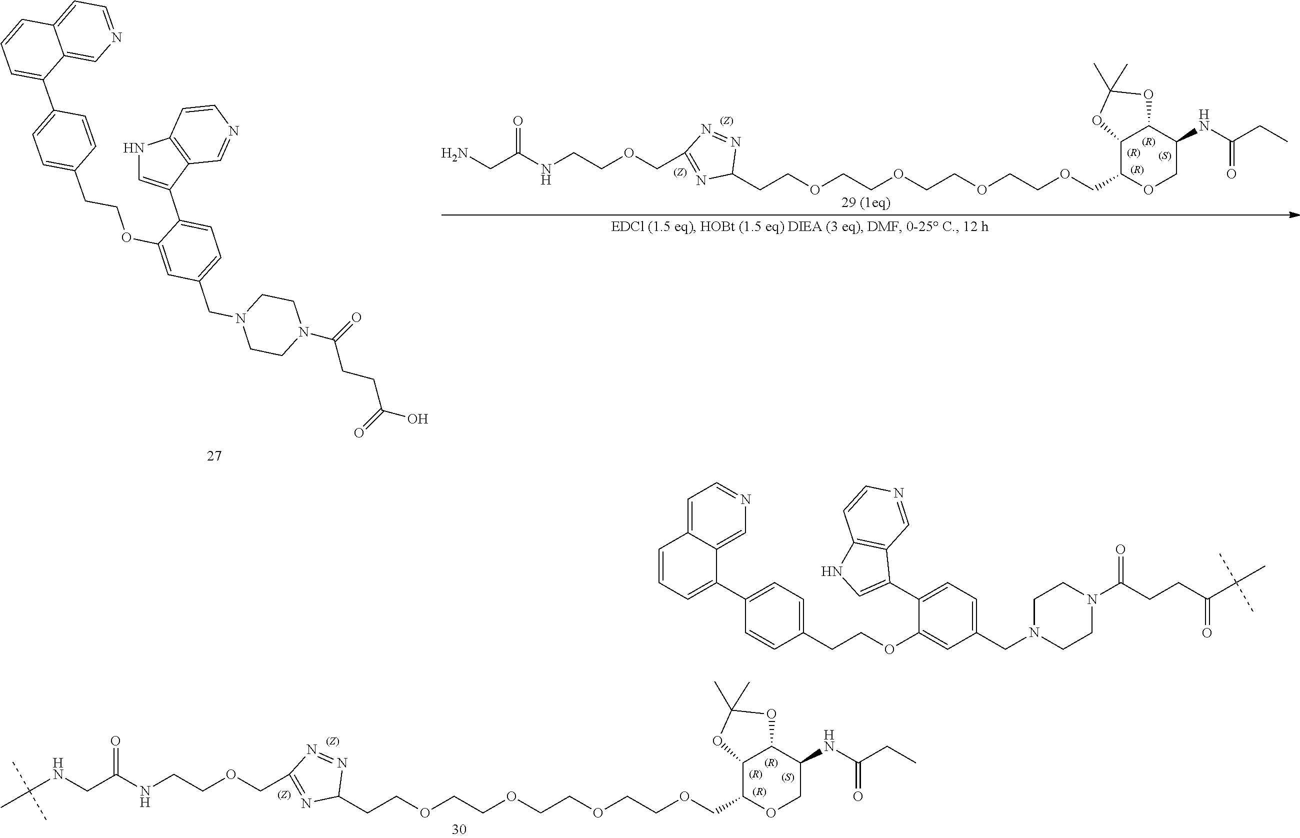

To a solution of Intermediate 27 (80.00 mg, 125.05 μmol, 1.00 equivalent) and Intermediate 29 (64.97 mg, 125.05 μmol, 1.00 equivalent) in DMF (1 mL), EDCI (35.96 mg, 187.58 μmol, 1.50 equivalent) and HOBt (25.35 mg, 187.58 μmol, 1.50 equivalent) was added at 25° C. Then DIEA (48.48 mg, 375.15 μmol, 65.34 uL, 3.00 equivalent) in DMF (0.5 mL) was added dropwise to the above reaction mixture at 0° C. Then the reaction mixture was stirred at 25° C. for 12 hours. LCMS showed the reaction was completed. The reaction was concentrated under reduced pressure to give crude product. The crude product was purified by prep-HPLC (column: Waters Xbridge BEH C18 100*30 mm*10 μm; mobile phase: [water (NH₄HCO₃)-ACN]; B %: 15%-45%, 8 min) to give Intermediate 30 (100.00 mg, 80.75 μmol, 64.6% yield) as yellow oil. LCMS: RT=1.170 min, MS cal.: 1238.43, found: [M/2+H]+=620.1.

### Preparation of Compound 106

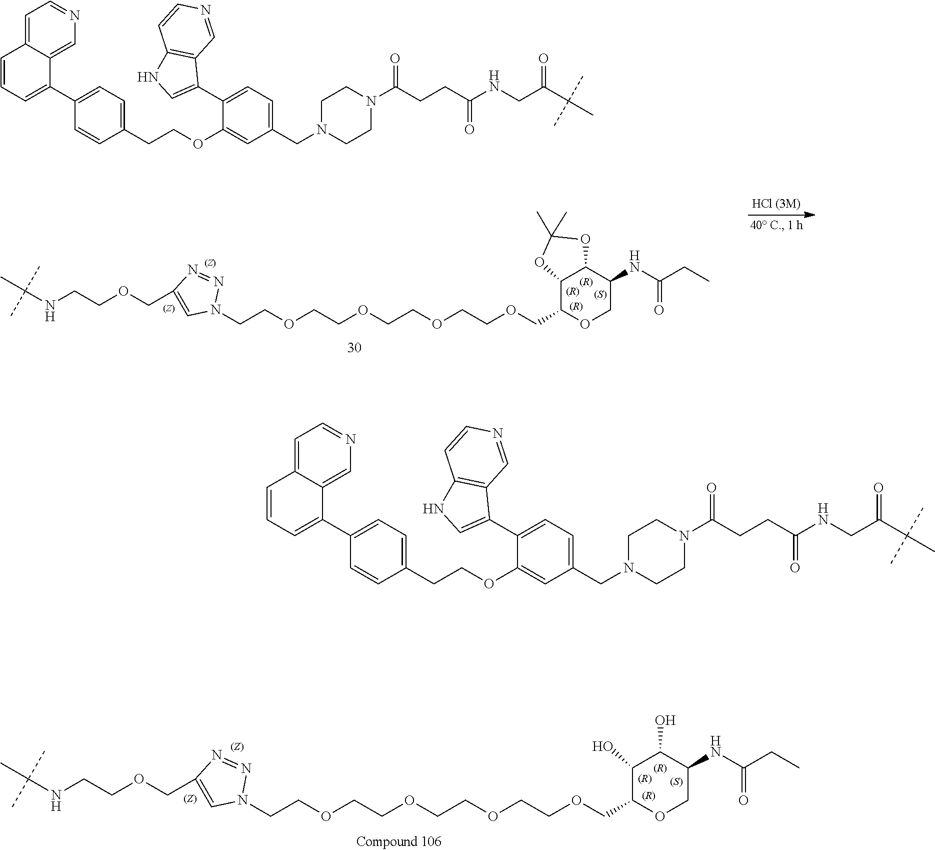

A mixture of Intermediate 30 (30.00 mg, 24.22 μmol, 1.00 equivalent) in HCl (3 M, 1.50 mL, 185.76 equivalent) was degassed and purged with N₂ for three times, and then the mixture was stirred at 40° C. for 1 hour under N₂ atmosphere. LCMS showed the reaction was completed. The reaction was concentrated under reduced pressure to give crude product. The crude product was purified by prep-HPLC (column: Waters Xbridge Prep OBD C18 150*40 mm*10 μm; mobile phase: [water (NH₄HCO₃)-ACN]; B %: 30%-70%, 8 min) to give Compound 106 (40.00 mg, 33.38 μmol, 68.9% yield) as white solid. LCMS: RT=1.613 min, MS cal.: 1198.4, found: [M/2+H]+=600.1. ¹HNMR (400 MHz, MeOD-d4) δ=9.05 (s, 1H), 8.91 (s, 1H), 8.44 (br d, J=5.8 Hz, 1H), 8.15 (br d, J=5.9 Hz, 1H), 8.00 (s, 1H), 7.92 (br d, J=8.1 Hz, 1H), 7.87 (br d, J=5.8 Hz, 1H), 7.83-7.77 (m, 1H), 7.52-7.48 (m, 2H), 7.47-7.42 (m, 2H), 7.28-7.25 (m, 2H), 7.23-7.19 (m, 2H), 7.17 (s, 1H), 7.02 (br d, J=7.6 Hz, 1H), 4.58 (s, 2H), 4.52 (br t, J=4.7 Hz, 2H), 4.37 (br t, J=6.1 Hz, 2H), 4.12 (dt, J=4.8, 10.4 Hz, 2H), 3.88 (br dd, J=5.1, 10.9 Hz, 2H), 3.83 (s, 5H), 3.63-3.56 (m, 14H), 3.54-3.46 (m, 7H), 3.39 (br t, J=5.6 Hz, 3H), 3.11 (br t, J=5.9 Hz, 2H), 3.03 (br t, J=10.9 Hz, 1H), 2.73 (br t, J=6.1 Hz, 2H), 2.50 (br d, J=6.3 Hz, 4H), 2.45 (br s, 2H), 2.21 (q, J=7.5 Hz, 2H), 1.10 (t, J=7.6 Hz, 3H). Theoretical number of H: 79, found: 73, exchangeable H: 6.

### Example 7. Procedure for Preparation of Compound 107

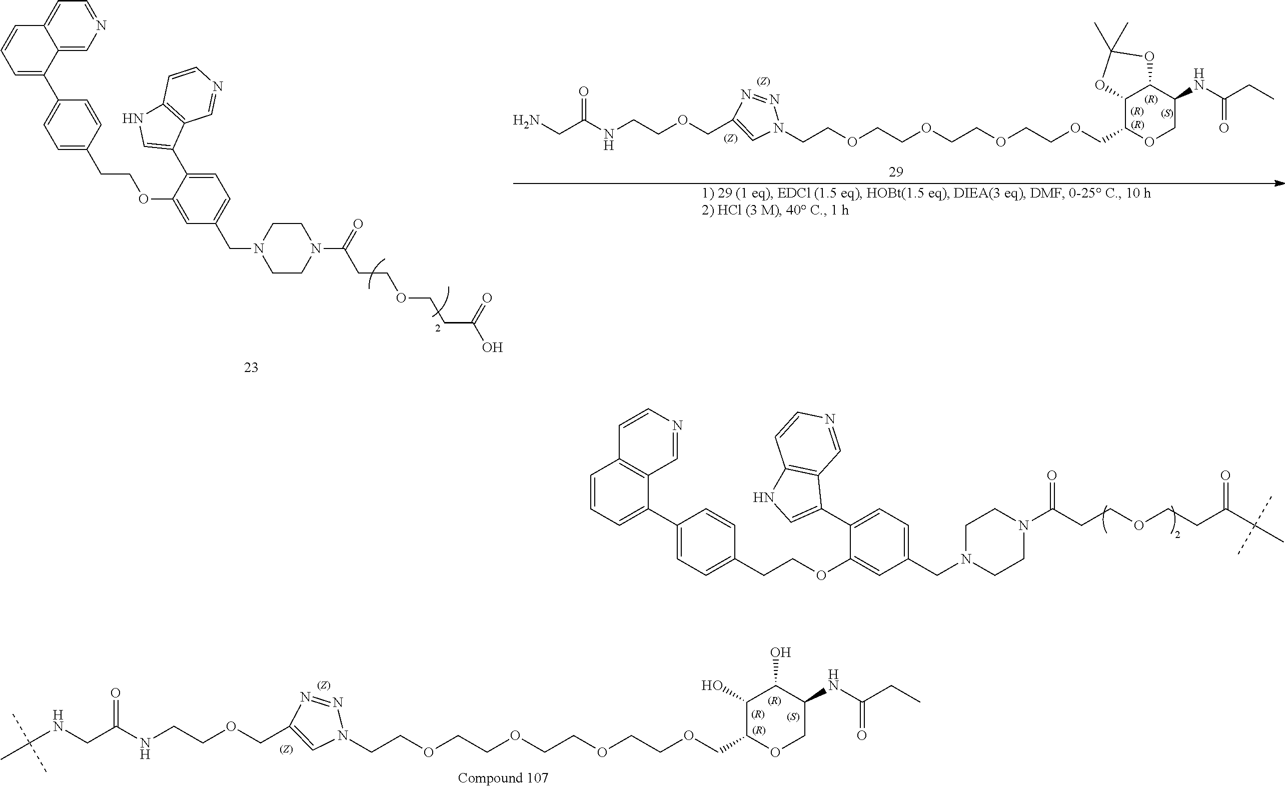

To a solution of Intermediate 23 (110 mg, 151.13 μmol, 1.00 equivalent) and Intermediate 29 (87.15 mg, 151.13 μmol, 1.00 equivalent) in DMF (3 mL), EDCI (43.46 mg, 226.70 μmol, 1.50 equivalent) and HOBt (30.63 mg, 226.70 μmol, 1.50 equivalent) was added at 25° C., then DIEA (58.60 mg, 453.39 μmol, 78.97 μL, 3.00 equivalent) in DMF (1 mL) was added dropwise at 0° C. The reaction mixture was stirred at 25° C. for 10 hours. The reaction mixture was concentrated under reduced pressure to give a residue. The residue was used in the next step directly without purification. HCl (3 M, 1 mL, 79.59 equivalent) was added to the residue and stirred at 40° C. for 1 hour. LCMS showed the reactant was consumed and the desired mass of the product was detected. The reaction was concentrated under reduced pressure to give a residue. The residue was purified by prep-HPLC (column: Waters Xbridge Prep OBD C18 150*40 mm*10 μm; mobile phase: [water (NH₄HCO₃)-ACN]; B %: 30%-70%, 8 min) to afford Compound 107 (30.00 mg, 23.32 μmol, 61.87% yield) as white solid. LCMS: RT=2.580 min, MS cal.: 1286.5, found: [M/2+H]+=644.0. ¹H NMR (400 MHz, MeOD-d₄) δ=9.05 (s, 1H), 8.91 (s, 1H), 8.44 (d, J=5.8 Hz, 1H), 8.15 (d, J=5.9 Hz, 1H), 8.00 (s, 1H), 7.92 (d, J=8.4 Hz, 1H), 7.86 (d, J=5.5 Hz, 1H), 7.82-7.77 (m, 1H), 7.53-7.48 (m, 2H), 7.47-7.42 (m, 2H), 7.28-7.24 (m, 2H), 7.23-7.19 (m, 2H), 7.17 (s, 1H), 7.02 (br d, J=7.6 Hz, 1H), 4.57 (s, 2H), 4.54 (t, J=5.1 Hz, 2H), 4.37 (brt, J=6.2 Hz, 2H), 4.17-4.09 (m, 1H), 3.92-3.83 (m, 6H), 3.72 (t, J=6.1 Hz, 5H), 3.63-3.54 (m, 26H), 3.51-3.49 (m, 1H), 3.48 (s, 1H), 3.40-3.36 (m, 2H), 3.11 (brt, J=6.2 Hz, 2H), 3.04 (t, J=11.0 Hz, 1H), 2.63 (t, J=6.3 Hz, 2H), 2.50 (br t, J=6.1 Hz, 5H), 2.25-2.17 (m, 2H), 1.11 (t, J=7.6 Hz, 3H). Theoretical number of H: 87, found: 81, exchangeable H:6.

### Example 8. Procedure for Preparation of Intermediate 25

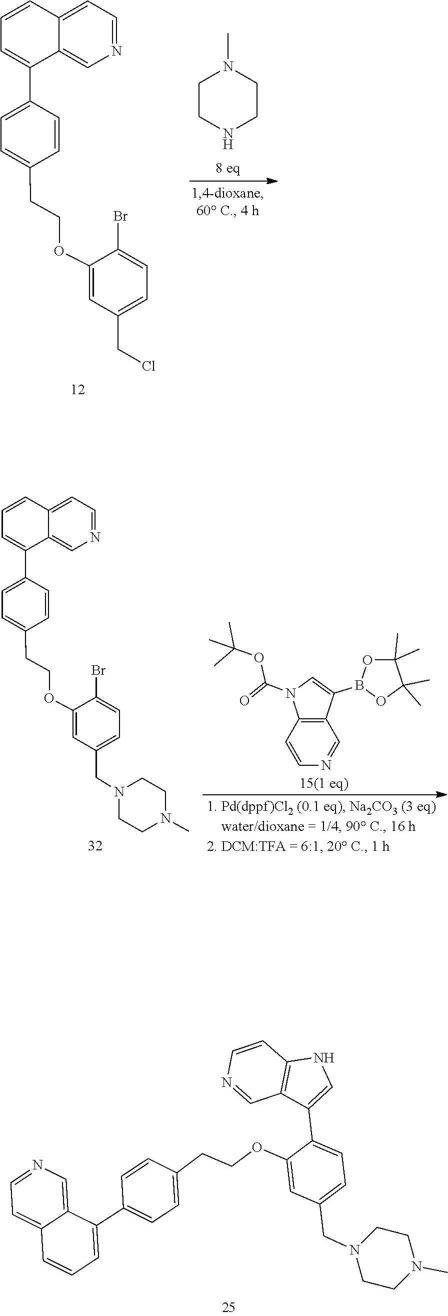

### Preparation of Intermediate 32

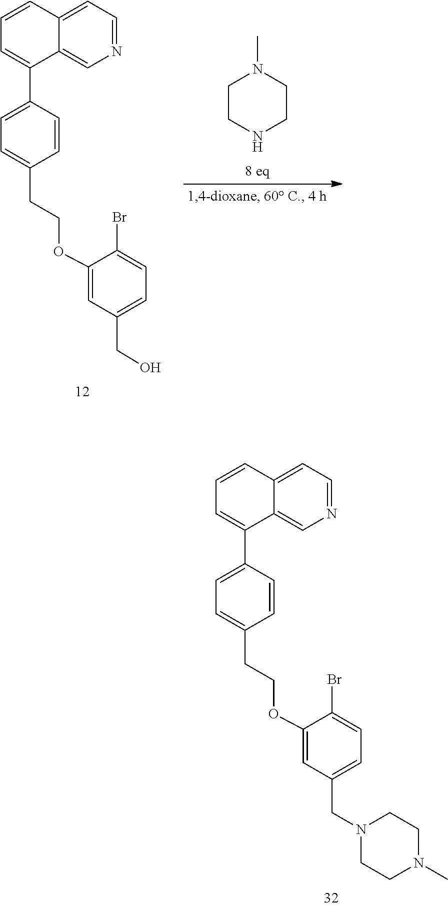

To a solution of Intermediate 12 (500 mg, 1.10 mmol, 1 equivalent) in 1, 4-dioxane (15 mL), 1-methylpiperazine (884.88 mg, 8.83 mmol, 979.94 uL, 8.00 equivalent) was added. The mixture was stirred at 60° C. for 4 hours. LCMS showed the reactant was consumed and the desired mass of product was detected. The reaction mixture was diluted with H₂O (30 mL) and extracted with chloroform/isopropyl alcohol (3:1, 30 mL*3). The combined organic layers dried over Na₂SO₄, filtered, and concentrated under reduced pressure to give a residue. The residue was purified by column chromatography (SiO₂, DCM:methanol=30/1 to 10/1) to give Intermediate 32 (515.00 mg, 997.15 μmol, 90.30% yield), obtained as a white solid. LCMS: RT=0.977 min, MS cal.: 516.6, found: [M+H]⁺=517.3. ¹H NMR (400 MHz, CHLOROFORM-d) δ ppm 9.33 (s, 1H), 8.55 (d, J=5.63 Hz, 1H), 7.82 (d, J=8.25 Hz, 1H), 7.65-7.77 (m, 2H), 7.39-7.60 (m, 6H), 6.94 (d, J=1.25 Hz, 1H), 6.79 (dd, J=8.07, 1.69 Hz, 1H), 4.32 (t, J=6.75 Hz, 2H), 3.40-3.52 (m, 2H), 3.27 (t, J=6.75 Hz, 2H), 2.21-2.71 (m, 11H). Theoretical number of H: 30, found: 30.

### Preparation of Intermediate 25

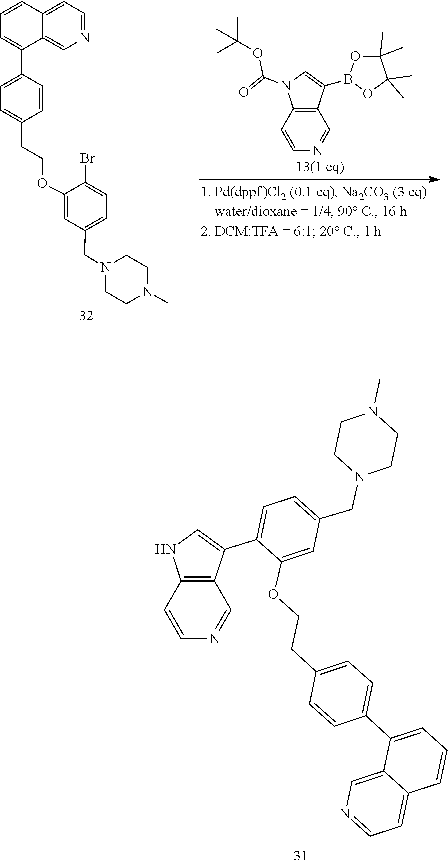 [SMILES: CN1CCN(Cc2ccc(Br)c(OCCc3ccc(-c4cccc5ccncc45)cc3)c2)CC1 ; CC(C)(C)OC(=O)n1cc(B2OC(C)(C)C(C)(C)O2)c2cnccc21 ; CN1CCN(Cc2ccc(-c3c[nH]c4ccncc34)c(OCCc3ccc(-c4cccc5ccncc45)cc3)c2)CC1]

To a solution of Intermediate 32 (100.00 mg, 193.62 μmol, 1.00 equivalent) in H₂O (0.20 mL) and dioxane (0.8 mL), Na₂CO₃ (61.57 mg, 580.87 μmol, 3 equivalent) and Intermediate 15 (66.65 mg, 193.62 μmol, 1.00 equivalent), Pd(dppf)Cl₂ (14.17 mg, 19.36 μmol, 0.10 equivalent) was added. The mixture was stirred at 90° C. for 16 hours. TLC showed formation of one new spot with lower polarity. The reaction mixture was concentrated under reduced pressure to remove dioxane. The residue was diluted with water (2 mL) and extracted with DCM (2 mL*3). The combined organic layers were washed with brine, filtered, and concentrated under reduced pressure to give a residue. The crude product was stirred in DCM:TFA=6:1 at 20° C. for 1 hour. LCMS showed the desired mass of compound was obtained. The reaction mixture was purified by reversed-phase HPLC (column: Phenomenex Luna 80*30 mm*3 μm; mobile phase: [water (HCl)-ACN]; B %: 10%-25%, 8 min) to give Intermediate 25 (57 mg, 87.18 μmol, 45.03% yield) as a yellow solid. LCMS: RT=1.247 min, MS cal.: 553.0, found: [M+H]⁺=554.2. ¹H NMR (400 MHz, DMSO-d₆) δ=13.16 (s, 1H), 9.26 (s, 1H), 9.20 (s, 1H), 8.67 (d, J=6.1 Hz, 1H), 8.44 (d, J=6.8 Hz, 1H), 8.39 (br d, J=5.4 Hz, 1H), 8.25 (d, J=8.4 Hz, 1H), 8.13 (brt, J=7.8 Hz, 1H), 8.08 (d, J=2.3 Hz, 1H), 8.01 (d, J=6.6 Hz, 1H), 7.79 (d, J=7.0 Hz, 1H), 7.68 (br d, J=7.8 Hz, 2H), 7.52-7.43 (m, 2H), 7.42-7.35 (m, 2H), 7.31 (br d, J=7.5 Hz, 1H), 4.47 (br t, J=6.7 Hz, 2H), 4.35 (br d, J=3.0 Hz, 2H), 3.61 (br s, 2H), 3.55-3.39 (m, 4H), 3.38-3.22 (m, 2H), 3.18 (brt, J=6.7 Hz, 2H), 2.82 (s, 3H). Theoretical number of H: 35, found: 35.

### Procedure for Preparation of Intermediate 5

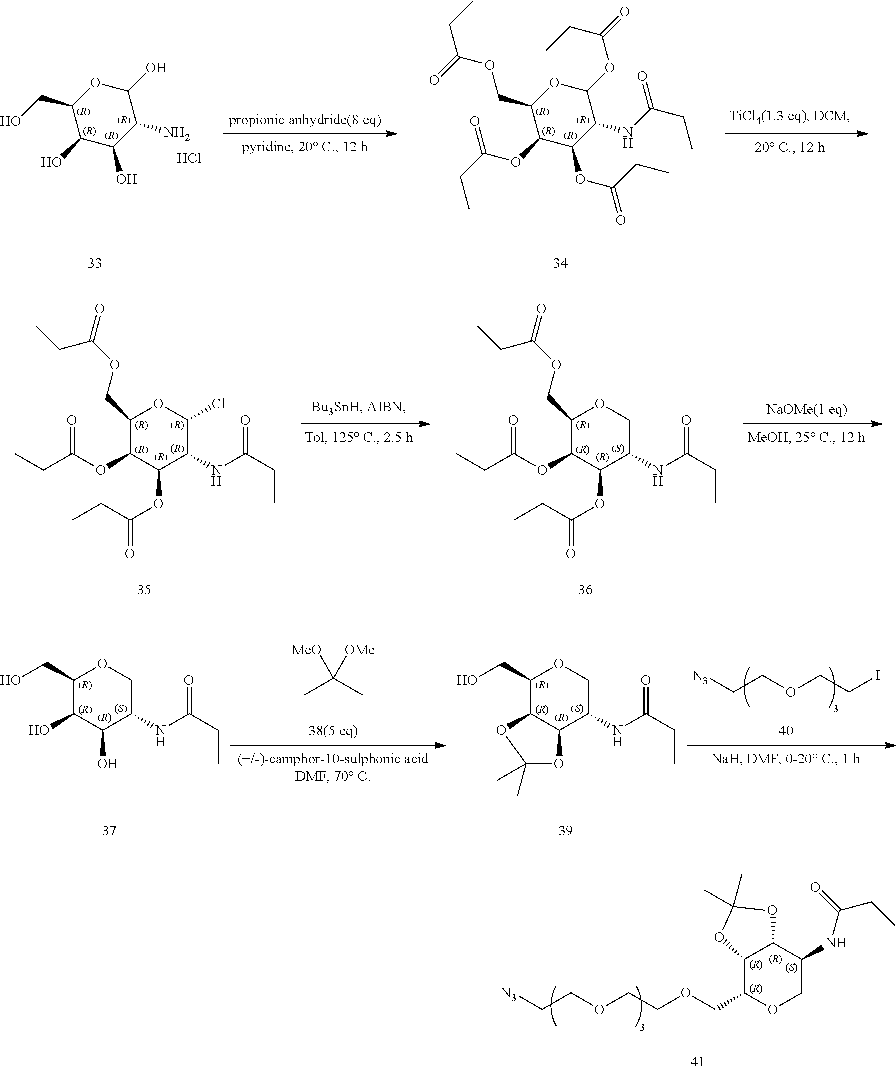

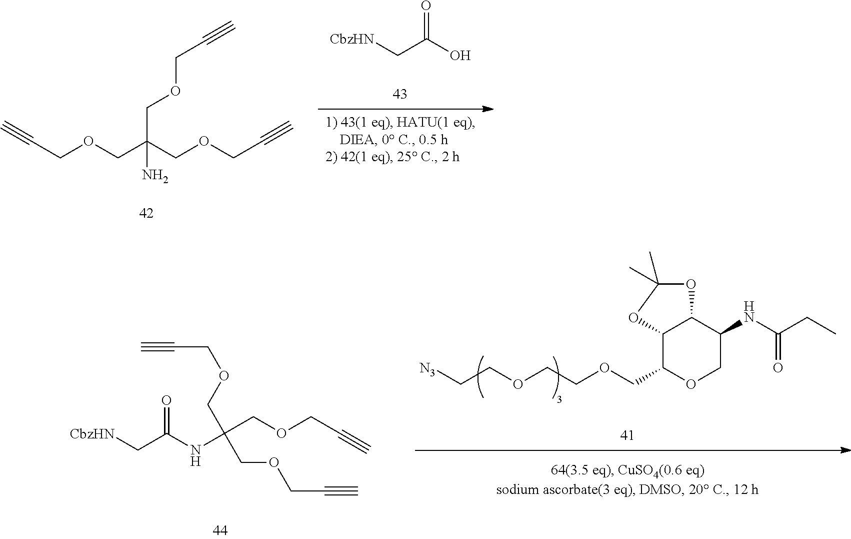

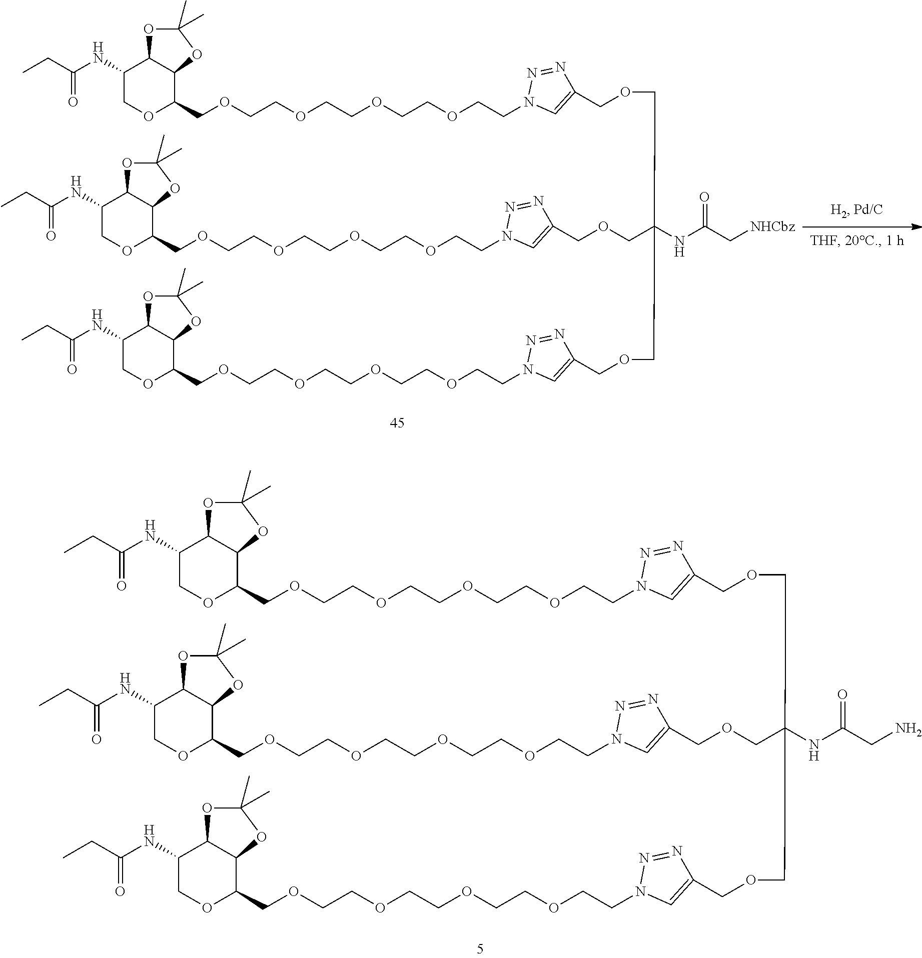 [SMILES: CCC(=O)N[C@H]1CO[C@H](COCCOCCOCCOCCn2cc(COCC(COCc3cn(CCOCCOCCOCCOC[C@H]4OC[C@H](NC(=O)CC)[C@H]5OC(C)(C)O[C@H]54)nn3)(COCc3cn(CCOCCOCCOCCOC[C@H]4OC[C@H](NC(=O)CC)[C@H]5OC(C)(C)O[C@H]54)nn3)NC(=O)CNC(=O)OCc3ccccc3)nn2)[C@@H]2OC(C)(C)O[C@@H]21 ; CCC(=O)N[C@H]1CO[C@H](COCCOCCOCCOCCn2cc(COCC(COCc3cn(CCOCCOCCOCCOC[C@H]4OC[C@H](NC(=O)CC)[C@H]5OC(C)(C)O[C@H]54)nn3)(COCc3cn(CCOCCOCCOCCOC[C@H]4OC[C@H](NC(=O)CC)[C@H]5OC(C)(C)O[C@H]54)nn3)NC(=O)CN)nn2)[C@@H]2OC(C)(C)O[C@@H]21]

### Preparation of Intermediate 34

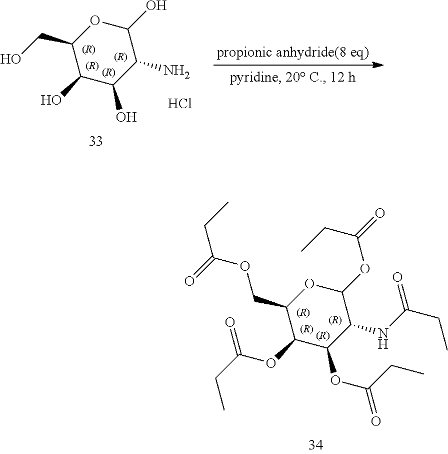

To a solution of Intermediate 33 (10 g, 46.38 mmol, 1 equivalent, HCl) in pyridine (200 mL), propanoyl propanoate (48.28 g, 371.00 mmol, 47.81 mL, 8 equivalent) was added. The mixture was stirred at 20° C. for 12 hours. LCMS showed the desired compound was detected and the reactant was consumed. The mixture was diluted with HCl (100 mL) and extracted with EA (100 mL*3). The combined organic layers were dried over Na₂SO₄, filtered, and concentrated under reduced pressure to give a residue. The residue was purified by column chromatography (SiO₂, petroleum ether/ethyl acetate=100/1 to 1/1) to give Intermediate 34 (10 g, 21.76 mmol, 46.93% yield) as a white oil. LCMS: RT=0.812 min, MS cal.: 459.5, found: [M+H]⁺=386.3. ¹H NMR (400 MHz, CDCl₃-d) 6=1.05-1.20 (m, 15H), 2.11 (q, J=7.58 Hz, 2H), 2.23-2.49 (m, 8H), 4.01-4.21 (m, 3H), 5.11 (dd, J=11.25, 3.30 Hz, 1H), 5.29 (s, 1H), 5.36-5.48 (m, 2H), 5.72 (d, J=8.80 Hz, 1H). Theoretical number of H: 33, found: 33.

### Preparation of Intermediate 35

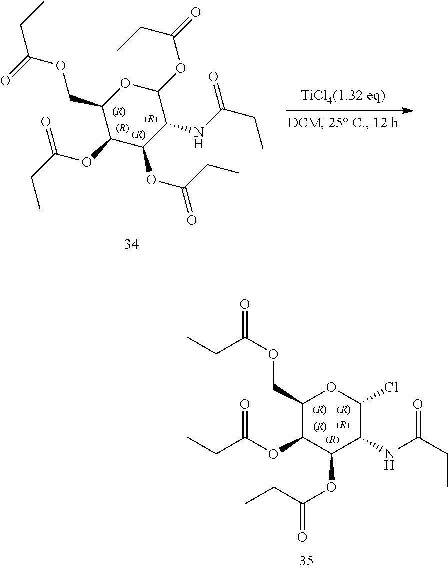

To a solution of Intermediate 34 (10 g, 21.76 mmol, 1 equivalent) in DCM (150 mL), TiCl₄ (5.45 g, 28.73 mmol, 1.32 equivalent) was added. The mixture was stirred at 20° C. for 12 hours in a dark environment. TLC showed the reactant was consumed and one main spot was formed. The reaction mixture was concentrated under reduced pressure to remove solvent. The residue was purified by column chromatography (SiO₂, petroleum ether/ethyl acetate=5/1 to 0/1) to give Intermediate 35 (9.1 g, crude) as yellow oil.

### Preparation of Intermediate 36

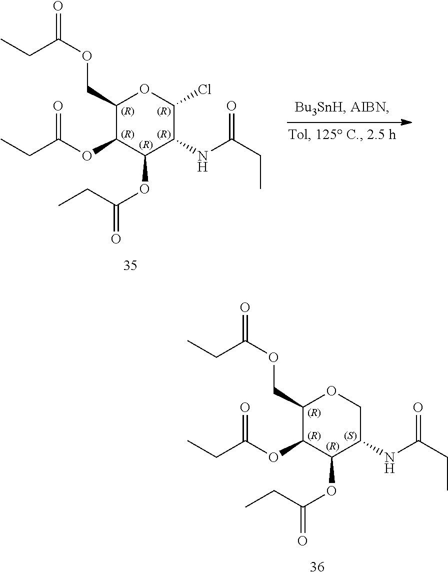

A solution of Intermediate 35 (9.1 g, 21.33 mmol, 1 equivalent) in Tol. (100 mL) was stirred at 20° C. for 30 minutes, then azobisisobutyronitrile (AIBN) (1.05 g, 6.40 mmol, 0.3 equivalent) and Bu₃SnH (12.42 g, 42.67 mmol, 11.29 mL, 2 equivalent) were added at 20° C. The mixture was stirred at 125° C. for 2 hours. LCMS showed the desired mass of the product was detected and the reactant was consumed. The reaction mixture was added to aqueous solution of KF (200 mL) and extracted with EtOAc (200 mL*3). The combined organic layers were washed with brine (150 mL*3), dried over Na₂SO₄, filtered, and concentrated under reduced pressure to give a residue. The residue was purified by column chromatography (SiO₂, petroleum ether/ethyl acetate=10/1 to 1/1) to give Intermediate 36 as a white solid. LCMS: RT=1.805 min, MS cal.: 387.4, found: [M+H]⁺=388.3. ¹H NMR (400 MHz, MeOD-d₄) δ=5.42 (d, J=2.8 Hz, 1H), 4.99 (dd, J=3.3, 11.1 Hz, 1H), 4.34 (dt, J=5.3, 11.1 Hz, 1H), 4.13-4.05 (m, 2H), 4.01-3.90 (m, 2H), 3.28 (s, 1H), 2.45 (q, J=7.6 Hz, 2H), 2.36-2.27 (m, 2H), 2.27-2.11 (m, 4H), 1.16 (t, J=7.6 Hz, 3H), 1.12-1.04 (m, 9H). Theoretical number of H: 29, found: 28. Exchangeable H: 1.

### Preparation of Intermediate 37

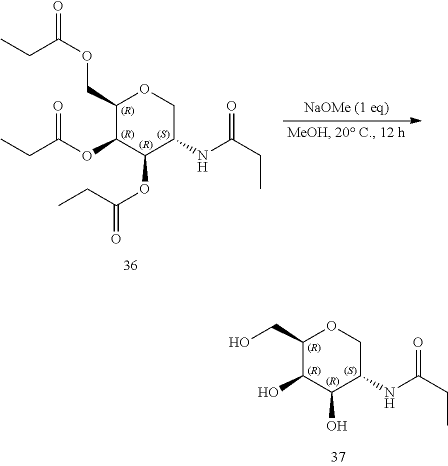

To a solution of Intermediate 36 (30 g, 77.43 mmol, 1 equivalent) in methanol (300 mL) was added NaOMe (4.18 g, 77.43 mmol, 1 equivalent). The mixture was stirred at 20° C. for 12 hours. LCMS showed the desired mass of the product was detected and the reactant was consumed. The reaction mixture was filtered and concentrated under reduced pressure to give a residue. The residue was purified by column chromatography (SiO₂, DCM/methanol=10/1 to 5/1) to give Intermediate 37 (9 g, 41.05 mmol, 53.02% yield) as a white solid. LCMS: RT=0.176 min, MS cal.: 219.2, found: [M+H]⁺=220.1. H NMR (400 MHz, MeOD-d₄) δ=4.13 (dt, J=5.3, 10.7 Hz, 1H), 3.92 (dd, J=5.3, 11.0 Hz, 1H), 3.98-3.89 (m, 1H), 3.77-3.61 (m, 2H), 3.49 (dd, J=3.2, 10.6 Hz, 1H), 3.41-3.36 (m, 1H), 3.06 (t, J=11.0 Hz, 1H), 2.21 (dq, J=1.5, 7.6 Hz, 2H), 1.11 (t, J=7.7 Hz, 3H). Theoretical number of H: 17, found: 13. Exchangeable H: 4.

### Preparation of Intermediate 39

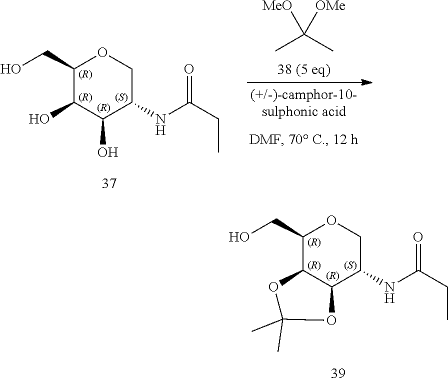

To a solution of Intermediate 37 (1 g, 4.56 mmol, 1 equivalent) in DMF (10 mL) was added Intermediate 38 (2.38 g, 22.81 mmol, 2.79 mL, 5 equivalent) and [(1S,4R)-7,7-dimethyl-2-oxo-norbornan-1-yl]methanesulfonic acid hydrate (570.88 mg, 2.28 mmol, 0.5 equivalent). The mixture was stirred at 70° C. for 12 hours. LCMS showed the desired mass of the product was detected and the reactant was consumed. The reaction mixture was diluted with NaHCO₃ (3 mL) and extracted with EtOAc (5 mL*3). The combined organic layers were washed with brine (5 mL*3), dried over Na₂SO₄, filtered, and concentrated under reduced pressure to give a residue. The residue was purified by column chromatography (SiO₂, DCM/methanol=10/1 to 5/1) to give Intermediate 39 (0.43 g, 1.66 mmol, 36.36% yield) as a white solid. LCMS: RT=0.789 min, MS cal.: 259.3, found: [M+H]⁺=260.1. ¹H NMR (400 MHz, MeOD-d₄) δ=4.20 (br d, J=4.5 Hz, 1H), 4.09-3.92 (m, 2H), 3.90-3.62 (m, 4H), 3.11-2.98 (m, 1H), 2.31-2.16 (m, 2H), 1.49 (s, 3H), 1.32 (s, 3H), 1.12 (t, J=7.6 Hz, 3H). Theoretical number of H: 21, found: 19. Exchangeable H: 2.

### Preparation of Intermediate 41

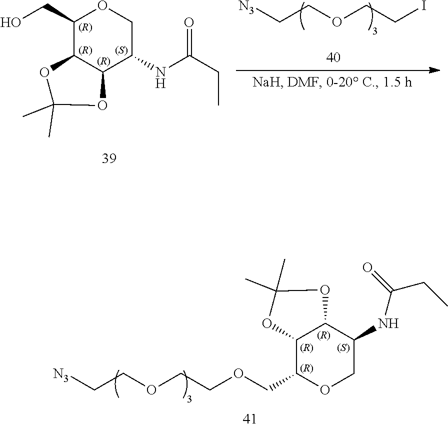

To a solution of Intermediate 39 (4 g, 15.43 mmol, 1 equivalent) in DMF (40 mL), NaH (6.17 g, 154.26 mmol, 60% purity, 10 equivalent) was added at 0° C. The mixture was stirred at 0° C. for 30 minutes, then Intermediate 40 (10.15 g, 30.85 mmol, 2 equivalent) was added at 0° C. The mixture was stirred at 20° C. for 1 hour. LCMS showed the desired mass of the product was detected and the reactant was consumed. The reaction mixture was poured into ice H₂O (60 ml) slowly, and extracted with DCM (60 mL*3). The combined organic layers were washed with brine (60 mL*3), dried over Na₂SO₄, filtered, and concentrated under reduced pressure to give a residue. The residue was purified by column chromatography (SiO₂, DCM:methanol=20/1 to 10/1) to give Intermediate 41 (3.4 g, 7.38 mmol, 47.86% yield) as yellow oil. LCMS: RT=1.590 min, MS cal.: 460.5, found: [M+H]⁺=461.3. ¹H NMR (400 MHz, MeOD-d₄) δ=4.21 (dd, J=2.1, 4.8 Hz, 1H), 4.11-3.97 (m, 2H), 3.90-3.82 (m, 1H), 3.81-3.75 (m, 1H), 3.73-3.59 (m, 17H), 3.41-3.35 (m, 2H), 3.05 (t, J=11.1 Hz, 1H), 2.28-2.15 (m, 2H), 1.49 (s, 2H), 1.32 (s, 3H), 1.12 (t, J=7.6 Hz, 3H) Theoretical number of H: 36, found: 35. Exchangeable H: 1.

### Preparation of Intermediate 44

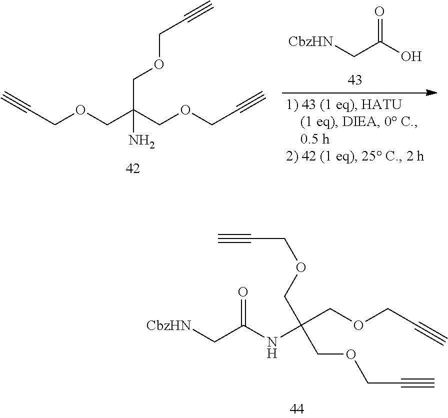 [SMILES: C#CCOCC(N)(COCC#C)COCC#C ; O=C(O)CNC(=O)OCc1ccccc1 ; C#CCOCC(COCC#C)(COCC#C)NC(=O)CNC(=O)OCc1ccccc1]

To a solution of Intermediate 43 (1.56 g, 7.44 mmol, 1 equivalent) in DMF (10 mL), hexafluorophosphate azabenzotriazole tetramethyl uronium (HATU) (2.83 g, 7.44 mmol, 1 equivalent) and DIEA (4.81 g, 37.19 mmol, 6.48 mL, 5 equivalent) were added at 0° C. The mixture was stirred at 0° C. for 30 minutes, and then Intermediate 42 (1.75 g, 7.44 mmol, 1 equivalent) was added. The mixture was stirred at 20° C. for another 2 hours. LCMS showed Intermediate 43 was consumed. Several new peaks were shown on LCMS and the desired compound was detected. The reaction mixture was diluted with water (20 mL) and extracted with EtOAc (20 mL*3). The combined organic layers were washed with brine (20 mL*3), dried over Na₂SO₄, filtered, and concentrated under reduced pressure to give a residue. The residue was purified by column chromatography (SiO₂, petroleum ether/ethyl acetate=10/1 to 5/1) to give Intermediate 44 (1.02 g, 2.39 mmol, 32.16% yield) as a white solid. LCMS: RT=2.188 min, MS cal.: 426.2, found: [M+H]⁺=427.3. ¹H NMR (400 MHz, MeOD-d₄) δ=7.40-7.29 (m, 5H), 6.08 (s, 1H), 5.40 (br s, 1H), 5.13 (s, 2H), 4.13 (d, J=1.7 Hz, 6H), 3.84 (s, 8H), 2.44 (t, J=2.3 Hz, 3H). Theoretical number of H: 26, found: 26.

### Preparation of Intermediate 45

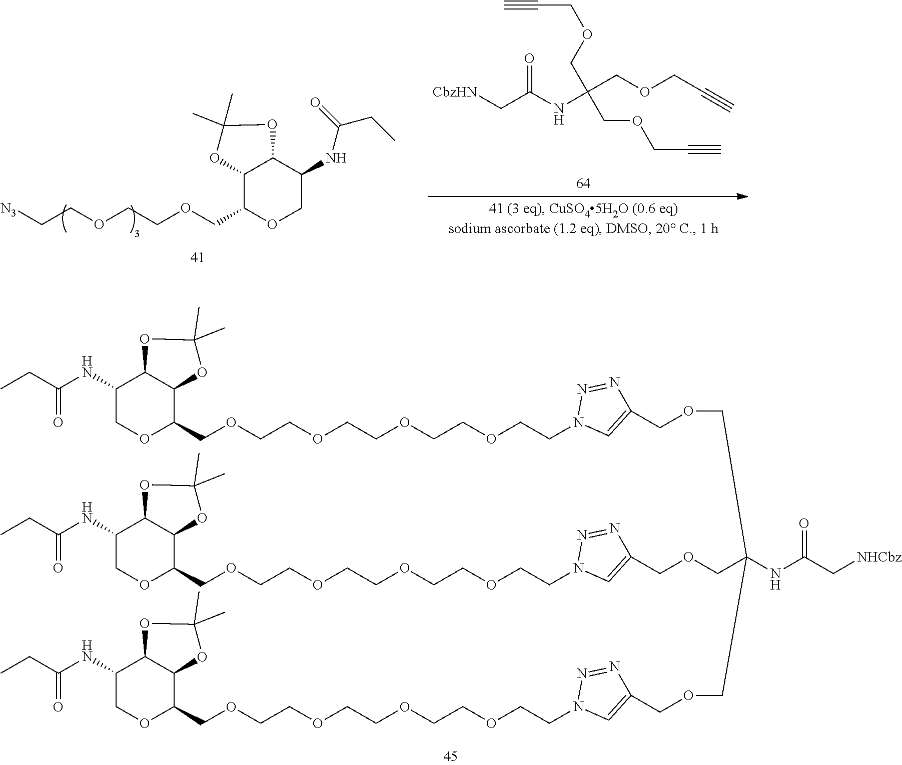

To a solution of Intermediate 41 (1.9 g, 4.13 mmol, 3 equivalent) and Intermediate 44 (586.49 mg, 1.38 mmol, 1 equivalent) in DMSO (5 mL), CuSO₄·5H₂O (206.03 mg, 825.15 μmol, 0.6 equivalent) and sodium; (2R)-2-[(1S)-1,2-dihydroxyethyl]-4-hydroxy-5-oxo-2H-furan-3-olate (326.94 mg, 1.65 mmol, 1.2 equivalent) were added. The mixture was stirred at 20° C. for 1 hour. LCMS showed the desired mass of the product was detected and the reactant was consumed. The reaction mixture was filtered and concentrated under reduced pressure to give a residue. The residue was purified by prep-HPLC (column: Xtimate C18 10 u 250 mm*80 mm; mobile phase: [water (NH₄HCO₃)-ACN]; B %: 10%-45%, 33 min) to give Intermediate 45 (1.4 g, 774.33 μmol, 56.30% yield) as yellow oil. LCMS: RT=1.971 min, MS cal.: 1808.0, found: [M/2+H]⁺=905.0.

### Preparation of Intermediate 5

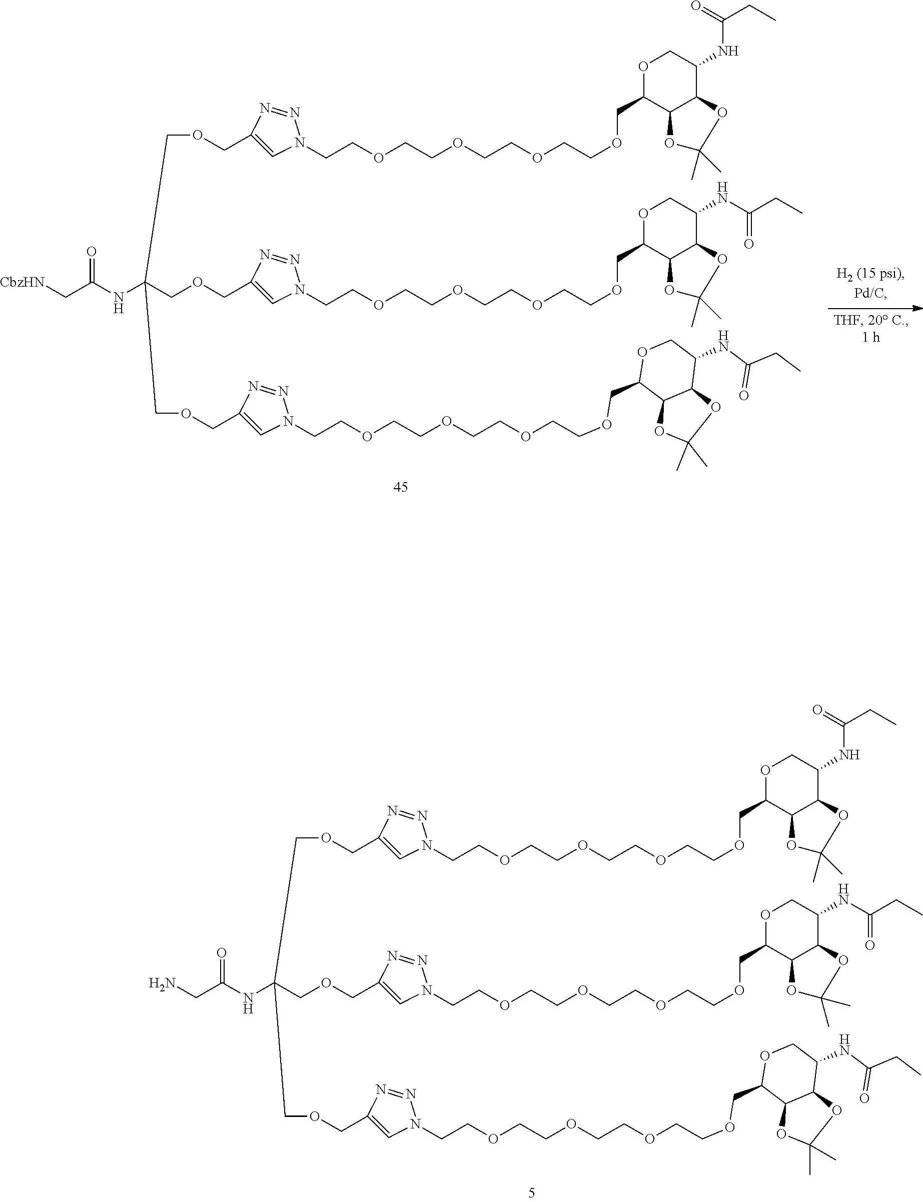 [SMILES: CCC(=O)N[C@H]1CO[C@H](COCCOCCOCCOCCn2cc(COCC(COCc3cn(CCOCCOCCOCCOC[C@H]4OC[C@H](NC(=O)CC)[C@H]5OC(C)(C)O[C@H]54)nn3)(COCc3cn(CCOCCOCCOCCOC[C@H]4OC[C@H](NC(=O)CC)[C@H]5OC(C)(C)O[C@H]54)nn3)NC(=O)CNC(=O)OCc3ccccc3)nn2)[C@@H]2OC(C)(C)O[C@@H]21 ; CCC(=O)N[C@H]1CO[C@H](COCCOCCOCCOCCn2cc(COCC(COCc3cn(CCOCCOCCOCCOC[C@H]4OC[C@H](NC(=O)CC)[C@H]5OC(C)(C)O[C@H]54)nn3)(COCc3cn(CCOCCOCCOCCOC[C@H]4OC[C@H](NC(=O)CC)[C@H]5OC(C)(C)O[C@H]54)nn3)NC(=O)CN)nn2)[C@@H]2OC(C)(C)O[C@@H]21]

To a solution of Intermediate 45 (1.2 g, 663.71 μmol, 1 equivalent) in THF (50 mL), palladium on carbon (Pd/C) (300 mg, 663.71 μmol, 10% purity, 1.00 equivalent) was added. The mixture was stirred at 20° C. for 1 hour. LCMS showed reactant was consumed completely and one main peak with the desired mass of the product was detected. The reaction mixture was filtered and concentrated under reduced pressure to give Intermediate 5 (1.03 g, 615.33 μmol, 92.71% yield) as a yellow oil. LCMS: RT=1.693 min, MS cal.: 1672.9, found: [M/2+H]+=837.9. ¹H NMR (400 MHz, MeOD-d₄) δ=8.00 (s, 3H), 4.65-4.53 (m, 13H), 4.18 (dd, J=2.2, 4.8 Hz, 3H), 4.10-3.95 (m, 5H), 3.89 (t, J=5.0 Hz, 6H), 3.86-3.81 (m, 3H), 3.80-3.74 (m, 9H), 3.70-3.65 (m, 6H), 3.65-3.55 (m, 38H), 3.03 (t, J=11.1 Hz, 3H), 2.28-2.16 (m, 6H), 1.48 (s, 9H), 1.30 (s, 9H), 1.11 (t, J=7.7 Hz, 9H) Theoretical number of H: 128, found: 122. Exchangeable H: 6.

### Procedure for Preparation of Intermediate 20

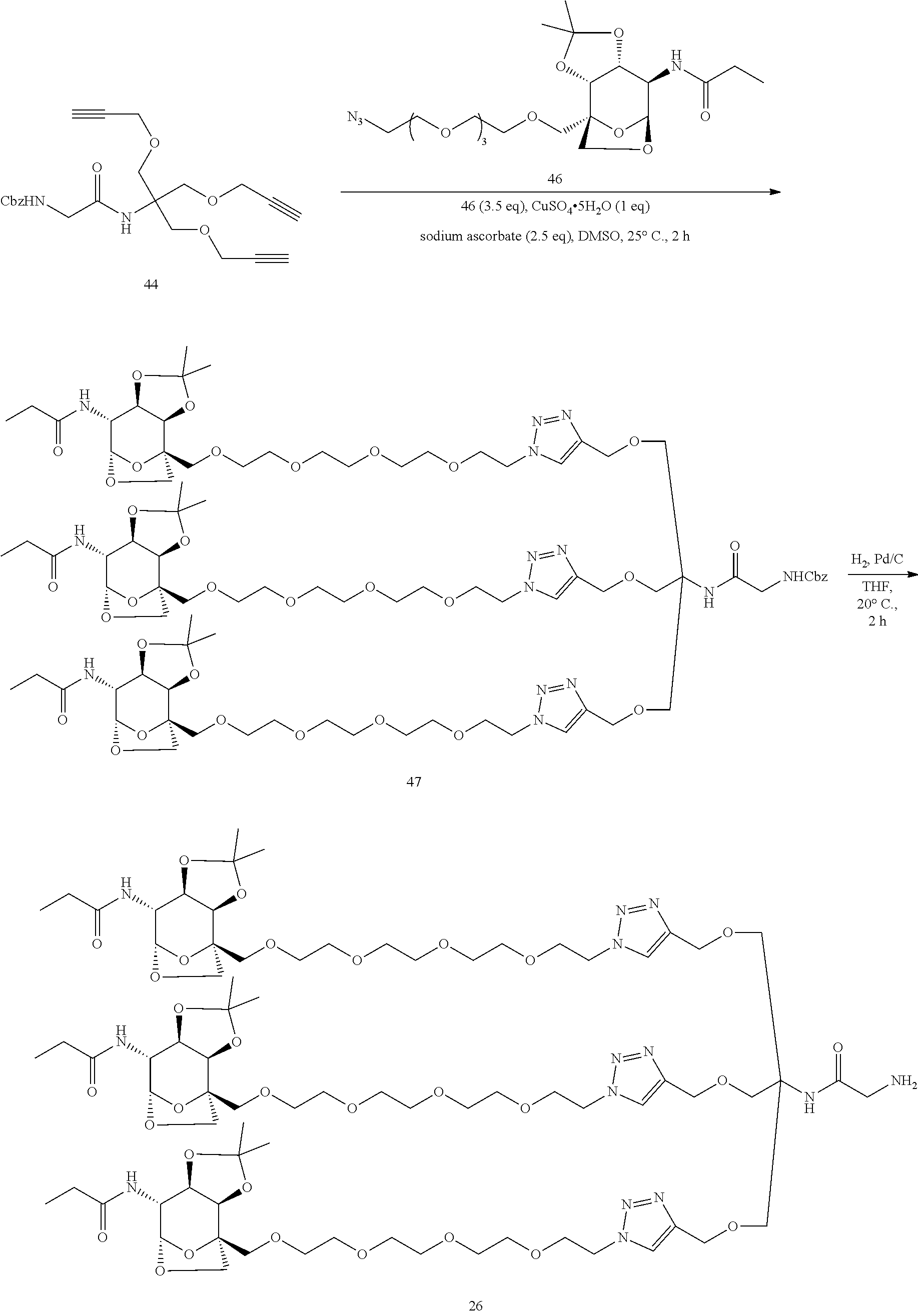

### Preparation of Intermediate 47

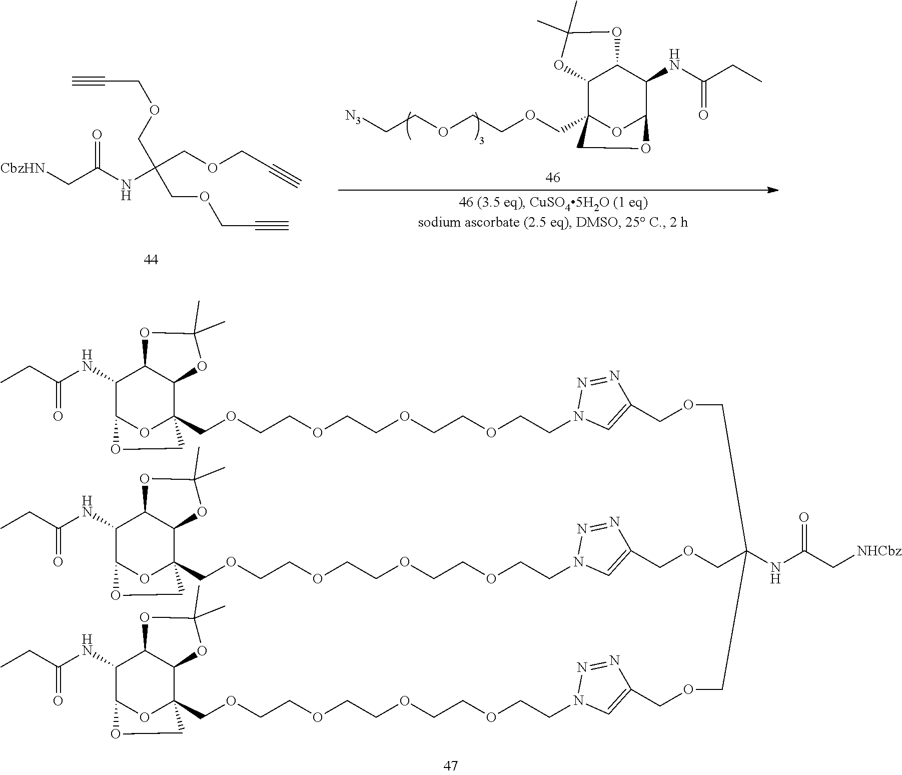

To a solution of Intermediate 44 (552 mg, 1.28 mmol, 99% purity, 1 equivalent) and Intermediate 46 (1.88 g, 3.84 mmol, 3 equivalent) in DMSO (8 mL), sodium ascorbate (634.65 mg, 3.20 mmol, 2.5 equivalent) and CuSO₄·5H₂O (319.95 mg, 1.28 mmol, 1 equivalent) was added. The mixture was stirred at 25° C. for 2 hours. LCMS showed the desired mass of the product was detected and the reactant was consumed. The reaction mixture was diluted with water (20 mL) and extracted with DCM (10 mL*3). The combined organic layers were washed with brine (10 mL*2), dried over Na₂SO₄, filtered, and concentrated under reduced pressure to give a residue. The residue was purified by pre-HPLC (column: Waters Xbridge Prep OBD C18 150*40 mm*10 μm; mobile phase: [water (NH4HCO3)-ACN]; B %: 30%-60%, 8 min) to give Intermediate 47 (1.25 g, 660.66 μmol, 51.56% yield) as yellow oil. LCMS: RT=2.065 min, MS cal.: 1891.9, [M/2+H]+=947.2. ¹H NMR (ET44948-208-P1A) (400 MHz, DMSO-d6) δ ppm=8.06-7.99 (m, 6H), 7.40-7.27 (m, 6H), 5.15 (d, J=1.9 Hz, 3H), 5.03 (s, 2H), 4.55-4.43 (m, 12H), 4.24 (d, J=5.9 Hz, 3H), 4.17-4.11 (m, 3H), 3.84-3.78 (m, 12H), 3.76-3.69 (m, 6H), 3.67-3.62 (m, 9H), 3.62-3.55 (m, 7H), 3.54-3.43 (m, 32H), 2.13 (q, J=7.6 Hz, 6H), 1.39 (s, 9H), 1.30-1.22 (m, 9H), 0.99 (t, J=7.6 Hz, 9H). Theoretical number of H: 134, found: 134.

### Preparation of Intermediate 20

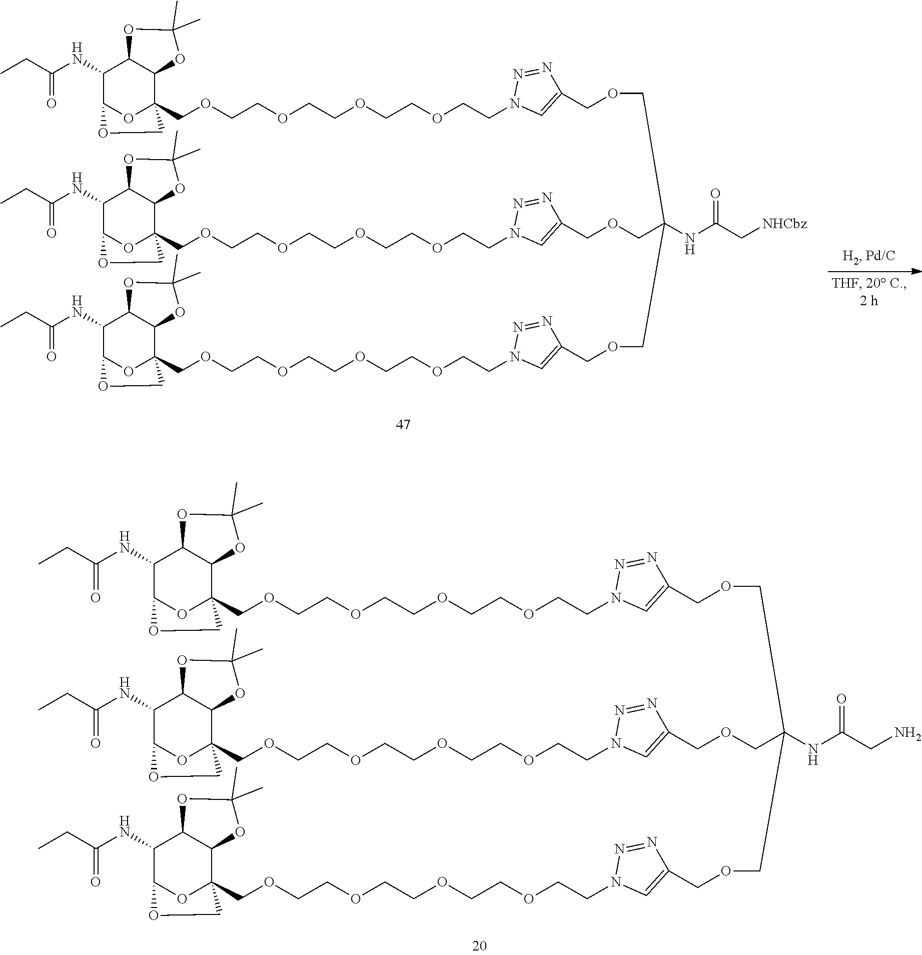

To a solution of Intermediate 47 (400 mg, 211.41 μmol, 1 equivalent) in THF (1 mL), Pd/C (5 mg, 21.14 μmol, 1000 purity, 0.1 equivalent) was added. The mixture was stirred at 20° C. for 2 hours under H₂ (15 Psi). LCMS showed the desired mass of the product was detected and the reactant was consumed. The reaction mixture was filtered and concentrated under reduced pressure to give Intermediate 20 (330.3 mg, 187.89 μmol, 88.88% yield) as yellow oil. LCMS RT=1.810 min, MS cal.: 1757.9, [M/2+H]+=880.1. ¹H NMR (400 MHz, DMSO-d6) δ ppm=8.05-7.99 (m, 6H), 5.16 (d, J=2.0 Hz, 3H), 4.55-4.45 (m, 13H), 4.25 (d, J=5.9 Hz, 3H), 4.15-4.10 (m, 3H), 3.84-3.56 (m, 35H), 3.53-3.46 (m, 32H), 2.13 (q, J=7.5 Hz, 6H), 1.40 (s, 9H), 1.27 (s, 9H), 0.99 (t, J=7.6 Hz, 9H). Theoretical number of H: 128, found: 128.

### Procedure for Preparation of Intermediate 29

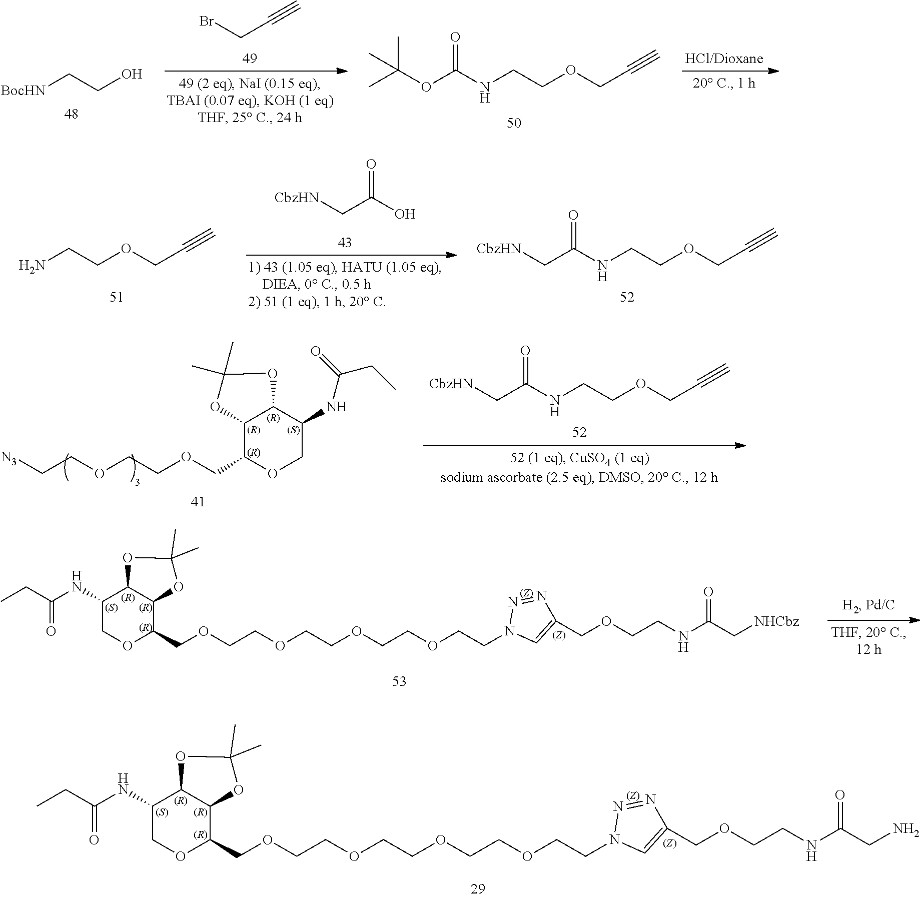

### Preparation of Intermediate 50

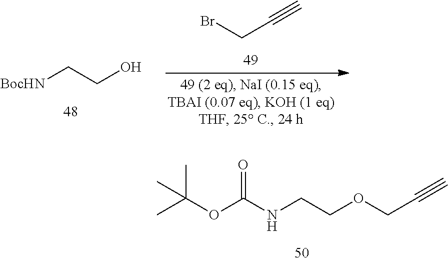

To a solution of Intermediate 48 (25 g, 155.09 mmol, 24.04 mL, 1 equivalent) in THF (250 mL), Intermediate 49 (36.90 g, 310.18 mmol, 26.74 mL, 2 equivalent), NaI (3.49 g, 23.26 mmol, 0.15 equivalent), tetra-n-butylammonium iodide (TBAI) (4.01 g, 10.86 mmol, 0.07 equivalent), and KOH (8.70 g, 155.09 mmol, 1 equivalent) were added. The mixture was stirred at 25° C. for 24 hours. TLC (petroleum ether (PE):ethyl acetate (EA)=2:1, R_{f}=0.8) indicated Intermediate 48 was consumed completely and one new spot formed. The reaction was clean with TLC. The reaction mixture was diluted with H₂O (200 mL) and extracted with 1500 mL ethyl acetate (EA) (500 mL*3). The combined organic layers were washed with Brine 600 mL (200 mL*3), dried over Na₂SO₄, filtered, and concentrated under reduced pressure to give a residue. The residue was purified by column chromatography (SiO₂, petroleum ether/ethyl acetate=100/1 to 1/1) to give Intermediate 50 (17.5 g, 82.05 mmol, 52.91% yield) as yellow oil. ¹H NMR (400 MHz, METHANOL-d₄) δ=4.16 (d, J=2.5 Hz, 2H), 3.55 (t, J=5.6 Hz, 2H), 3.23 (t, J=5.6 Hz, 2H), 2.83 (t, J=2.4 Hz, 1H), 1.44 (s, 9H). Theoretical number of H: 17, found: 17. Exchangeable H: 1.

### Preparation of Intermediate 51

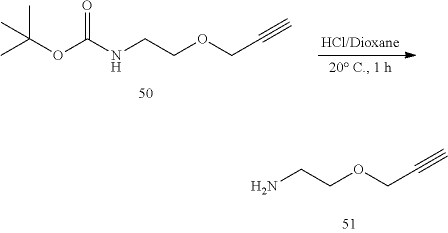

A mixture of Intermediate 50 (34 g, 159.42 mmol, 1 equivalent) and HCl/dioxane (100 mL) was stirred at 20° C. for 1 hour. TLC (PE:EA=2:1, R_{f}=0.2) indicated Intermediate 50 was consumed completely and one new spot formed. The reaction mixture was concentrated under reduced pressure to give Intermediate 51 (21 g, 140.36 mmol, 88.04% yield, HCl) as a white solid. ¹H NMR (400 MHz, METHANOL-d₄) δ=4.26 (d, J=2.5 Hz, 2H), 3.78-3.74 (m, 2H), 3.17-3.14 (m, 2H), 2.94 (t, J=2.4 Hz, 1H). Theoretical number of H: 9, found: 7. Exchangeable H: 2.

### Preparation of Intermediate 52

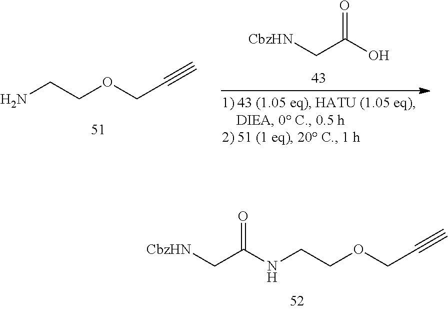

To a solution of Intermediate 43 (32.40 g, 154.88 mmol, 1.05 equivalent) in DMF (150 mL), HATU (58.89 g, 154.88 mmol, 1.05 equivalent) and DIEA (57.19 g, 442.50 mmol, 77.08 mL, 3 equivalent) were added. The mixture was stirred at 0° C. for 30 minutes, then Intermediate 51 was added (20 g, 147.50 mmol, 1 equivalent, HCl) at 0° C. The mixture was stirred at 20° C. for another 1 hour. LC-MS showed it was finished and one main peak with the desired mass of the product was detected. The reaction mixture was dried by N₂ and purified by reverse-phase HPLC (0.10% NH₃·H₂O) to give Intermediate 52 (22 g, 75.78 mmol, 51.38% yield) as a white solid. LCMS: RT=1.630 min, MS cal.: 290.13, found: [M+H]⁺=291.2. ¹H NMR (400 MHz, CHLOROFORM-d) δ=7.40-7.30 (m, 5H), 6.46-6.27 (m, 1H), 5.60-5.40 (m, 1H), 5.13 (s, 2H), 4.14 (br d, J=1.5 Hz, 2H), 3.88 (br d, J=5.6 Hz, 2H), 3.63-3.58 (m, 2H), 3.53-3.48 (m, 2H), 2.45 (t, J=2.3 Hz, 1H). Theoretical number of H: 18, found: 18.

### Preparation of Intermediate 53

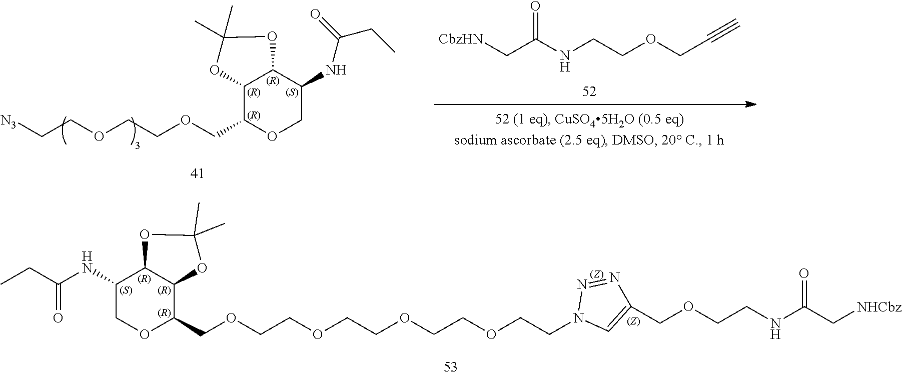

To a solution of Intermediate 41 (779.99 mg, 1.69 mmol, 1 equivalent), Intermediate 52 (491.71 mg, 1.69 mmol, 1 equivalent) in DMSO (10 mL), sodium; (2R)-2-[(1S)-1,2-dihydroxyethyl]-4-hydroxy-5-oxo-2H-furan-3-olate (369.09 mg, 1.86 mmol, 1.1 equivalent) and CuSO₄·5H₂O (253.74 mg, 1.02 mmol, 0.6 equivalent) were added. The mixture was stirred at 20° C. for 1 hour. LCMS showed Reactant 1 was consumed and the desired compound was detected. The reaction mixture was filtered and concentrated under reduced pressure to give a residue. The residue was purified by prep-HPLC (column: Waters Xbridge BEH C18 250*50 mm*10 μm; mobile phase: [water (NH₄HCO₃)-ACN]; B %: 20%-45%, 10 min) to give Intermediate 53 (1 g, 1.33 mmol, 78.64% yield) as a white solid. LCMS: RT=1.724 min, MS cal.: 751.3, found: [M+H]⁺=751.4.

### Preparation of Intermediate 29

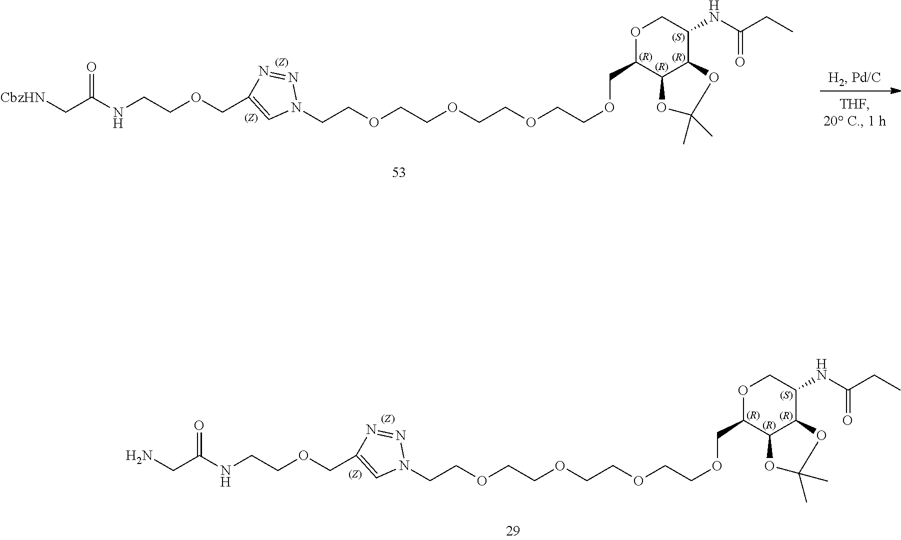

To a solution of Intermediate 53 (1 g, 1.33 mmol, 1 equivalent) in THF (20 mL), Pd/C (500 mg, 663.71 μmol, 10% purity, 1.00 equivalent) was added. The mixture was stirred at 20° C. for 1 hour. LCMS showed Intermediate 53 was consumed completely and one main peak with the desired mass of the product was detected. The reaction mixture was filtered and concentrated under reduced pressure to give Intermediate 29 (780 mg, 1.26 mmol, 94.96% yield) as yellow oil. LCMS: RT=1.310 min, MS cal.: 616.3, found: [M+H]⁺=617.4. ¹H NMR (400 MHz, MeOD-d₄) δ=8.04 (s, 1H), 4.64-4.56 (m, 4H), 4.19 (dd, J=2.1, 5.0 Hz, 1H), 4.07-3.94 (m, 2H), 3.92-3.87 (m, 2H), 3.86-3.77 (m, 2H), 3.70-3.67 (m, 2H), 3.66-3.57 (m, 14H), 3.44-3.39 (m, 2H), 3.26 (s, 2H), 3.04 (t, J=11.1 Hz, 1H), 2.22 (dq, J=1.4, 7.6 Hz, 2H), 1.49 (s, 3H), 1.31 (s, 3H), 1.12 (t, J=7.6 Hz, 3H). Theoretical number of H: 48, found: 44. Exchangeable H: 4.

### Example 9. Evaluation of Biological Activity

The following assays are useful for determining whether the bifunctional compounds of the disclosure bind to TNF-α or inhibit TNF-α activity.

### Example 9A. Tnf-α Functional Assay Protocol

Human HEK TNF-αcells (hHEK-TNF-α cells; InvivoGen Catalog #hkb-tnfdmyd; San Diego, CA, USA) are human embryonic kidney HEK293 cells that are stably transfected with the reporter gene, secreted alkaline phosphatase (SEAP). The hHEK-TNF-α cells allow for the detection of bioactive human and murine TNF-α via the activation of the NF-kB-inducible promoter and subsequent production of SEAP. Bioactivity of commercial human TNF-α protein (ActiveMax® Human TNF-α Protein, Tag Free, Low Endotoxin; available by ACROBiosystems Catalog #TNA-H4211; Newark, DE, USA) was determined in this reporter system. The EC₅₀ and EC₅₀ of the cytokine was determined to be 0.399 pM and 1.034 pM, respectively. The EC₈₀ of 1.034 pM was used to evaluate the functional inhibition by monofunctional and bifunctional TNF-α-specific binder molecules.

hHEK-TNF-α cells were harvested with a cell scrapper and seeded at 50,000 cells/well in a clear, 96-well TC-treated flat bottom plate (Corning™ Catalog #3585; Corning, NY, USA). Test molecules were dissolved in 100% DMSO and serially diluted by either 3-fold serial dilution or 4-fold serial dilution. Test molecules were further diluted 1:100 into an intermediate aqueous phase with Assay Media (DMEM 4.5 g/L glucose (Thermo Fisher Catalog #11995123; Waltham, MA, USA)-10% HI FBS (Thermo Fisher Catalog #10438026; Waltham, MA, USA)-1% Penicillin-Streptomycin (Thermo Fisher Catalog #151401222; Waltham, MA, USA)), and 10× concentration solution was then transferred to assay wells, so that the final assay conditions contained test molecules in 0.1% DMSO and in most cases at a 10 M high concentration. TNF-α protein was added at the previously determined ECso. Plates containing cells, test molecules, and cytokine were incubated overnight for 18-24 hours at 37° C., 5% CO₂.

The level of SEAP in the supernatant of each well was quantified using the Quanti Blue™ Solution (InvivoGen Catalog #rep-qba2; San Diego, CA, USA), a reagent with a colorimetric change in the presence of SEAP. To perform the Quanti-Blue assay: 20 L of the sample supernatant after the overnight 18-24 hour incubation was added to 180 L of Quanti Blue™ Reagent and incubated for 45 minutes at 37° C. The absorbance at 620 nm and 650 nm was obtained using the Synergy H1 Microplate Reader (BioTek Catalog #17010853; Winooski, VT, USA). Inhibition of TNF-α signaling was calculated utilizing GraphPad Prism (San Diego, CA, USA).

The effects of test molecules on the cellular viability of reporter cells were determined using CellTiterGlo® Luminescent Cell Viability Assay (Promega Catalog #G7572; Madison, WI, USA). Assay plates were brought to room temperature, and the supernatant removed so that only 100 μL of media (DMEM 4.5 g/L glucose-10% HI Fetal Bovine Syndrome-1% Penicillin-Streptomycin) containing adherent reporter cells remained per well. An equal volume of CellTiterGlo® reagent was added to each well and incubated at room temperature for 15 minutes. Luminescence signal, which reflects cell viability, was detected with a luminometer. Percent viability was calculated utilizing GraphPad Prism (San Diego, CA, USA) from the luminescence signal.

### Example 9B. Production and Purification of TNF A and the Y87S Isoform

A plasmid that encodes and produces a fusion protein comprised of glutathione-S transferase (GST) and TNF-α was synthesized by GenScript and transformed into Escherichia coli DH10B™ and later BL21(NiCo) from New England BioLabs© (NEB; Ipswish, MA, USA). The amino acid sequence of the fusion protein contains a HRV3c protease cut site between the GST sequence and the TNF-α sequence. The sequence is shown and discussed in greater detail below. Selection of transformants was accomplished by growing the cells in the presence of ampicillin (100 μg/ml).

Production was initiated by transferring 2.5 ml of overnight culture to 50 ml of Luria Broth (LB) in 250 ml flask, supplemented with 100 μg ampicillin and 0.2 mM isopropyl β-D-1-thiogalactopyranoside (IPTG). The flasks were incubated for 20 hours at 30° C., shaking at 140 rotations per minute (rpm). The cells were harvested via centrifugation (7197×g for 5 minutes) and the cell pellet was frozen at −80° C. The bacterial cells per lysed with B-PER™ Bacterial Protein Extraction Reagent, with enzymes (ThermoFisher; Waltham, MA, USA) following the manufacturer's instructions. Cellular debris was removed via centrifugation at 21,300×g for 3 minutes. The clarified supernatant was applied to two 1 ml Pierce™ Glutathione Agarose purification columns (ThermoFisher; Waltham, MA, USA) linked in series, washed with 5 column volumes of buffer (20 mM HEPES, pH 7.9, 150 mM NaCl), and eluted with column buffer containing 15 mM reduced glutathione. The eluted fractions were monitored for the presence of protein of the correct size, pooled, and concentrated 5-10 fold using a centrifugal filter with a cut off weight of 10 kd. One μl of HRV3c protease was added for each milliliter of eluant and incubated at 4° C. overnight. If cutting, as judged by sodium dodecyl-sulfate polyacrylamide gel electrophoresis (SDS-PAGE) analysis, was not sufficiently complete, another aliquot of protease was added and incubated further. After cutting was complete, salts from the elution buffer were removed by dialysis. The GST tag and TNF-α were separated via ion exchange chromatography, using a 20 mM HEPES pH 8.0 buffer system ramping from 0 to 1 M NaCl over 12 columns volumes with 5 ml of Q Sepharose (HiScreen Q HP, Cytiva™, Marlborough, MA, USA). The purity of the collected fractions was assessed via SDS-PAGE. Fractions containing only TNF-α were pooled, concentrated, and rinsed with 25 mM HEPES buffered saline (pH 7.4, 150 mM NaCl). Aliquots were stored at −80° C. without the need for a cryopreservant. While the two step purification method described above proved to be sufficient to produce protein that was >90% pure, protocols for purification by size-exclusion chromatography (SEC) or with a secondary pass over GST resin were also developed, but not routinely used.

The Y87S mutant was created via site directed mutagenesis, which was confirmed by Sanger sequencing. The “87” designation (Zhang et al., Site-directed mutational analysis of human tumor necrosis factor-alpha receptor binding site and structure-functional relationship. J Biol Chem. 1992 Nov. 25; 267(33):24069-75) does not hold here as the numbering scheme differs from the wild type. The mutated tyrosine residue (319, before removal of GST tag) is bold and underlined. Production and purification were done as described above.

Amino Acid Sequence of the Glutathione-S Transferase-TNF-α
Fusion Protein
(SEQ. ID. NO.: 1)
MSPILGYWKIKGLVQPTRLLLEYLEEKYEEHLYERDEGDKWRNKKFELGL 50
EFPNLPYYIDGDVKLTQSMAIIRYIADKHNMLGGCPKERAEISMLEGAVL 100
DIRYGVSRIAYSKDFETLKVDFLSKLPEMLKMFEDRLCHKTYLNGDHVTH 150
PDFMLYDALDVVLYMDPMCLDAFPKLVCFKKRIEAIPQIDKYLKSSKYIA 200
WPLQGWQATFGGGDHPPKSDLEVLFQGPLGSGVRSSSRTPSDKPVAHVVA 250
NPQAEGQLQWLNRRANALLANGVELRDNQLVVPSEGLYLIYSQVLFKGQG 300
CPSTHVLLTHTISRIAVSYQTKVNLLSAIKSPCQRETPEGAEAKPWYEPI 350
YLGGVFQLEKGDRLSAEINRPDYLDFAESGQVYFGIIA

The amino acids comprising the GST affinity tag are italicized, the HRV3c cut site is show in bold, and the sequence that remains after proteolysis and purification is underlined.

### Example 9C. TNF-α Thermal Melt Assay

Amino acids 77-233 of Human TNF-α protein with an N-terminal GST tag were produced in E. coli. Protein was purified using glutathione agarose purification columns (Pierce™, Waltham, MA, USA). Following purification, the GST tag was removed using HRV3c protease (Pierce™, Waltham, MA, USA) and the GST tag and the TNF-α were separated via ion exchange chromatography using a 20 mM HEPES pH 8.0 buffer system ramping from 0 to 1 M NaCl over 12 column volumes with 5 ml of Q Sepharose (HiScreen Q HP, Cytiva™, Marlborough, MA, USA). The final protein includes the amino acids GPLGSG at the N-terminus of the protein.

In order to determine the effect on protein melting, compounds were mixed with 125 μg TNF-α protein in Hepes Buffered Salt (20 mM Hepes pH 7.4, 150 mM NaCl) and 20×SYPRO™ Orange (Life Technologies, Catalog #S6651; Carlsbad, CA, USA) in a 10 μl reaction. Protein was heated from 20° C. to 95° C. at one degree per minute in a Roche Opus384 qPCR machine. Melting temperatures were calculated using Bio-Rad CFX Maestro software and Kd was calculated as in Matulis et al. (Biochemistry. 2005. 44(13):5258-5266).

### Example 9D. GalNAc-PAA Human ASGPR1 Competition Binding Assay

Protocol. Recombinant human ASGPR protein (R&D Systems Catalog #4394-AS; Minneapolis, MN, USA) was reconstituted to 200 μg/ml in Assay Buffer (20 mM HEPES (TEKnova Catalog #H1030; Hollister, CA, USA), 150 mM NaCl (Promega Catalog #V4221; Madison, WI, USA), 1 mM CaCl₂ (Alfa Aesar Catalog #J63122; Tewksbury, MA, USA)). Nunc-Immuno™ MicroWell™ 96 well solid plates (Sigma Catalog #M9410; Burlington, MA, USA) were coated with 300 ng of ASGPR protein per well by diluting the protein to 3 μg/ml in Assay Buffer and adding 100 μl to each well. A few wells were left without ASGPR protein for controls in which 100 μl of Assay Buffer were added. The plate was incubated overnight at 4° C. After overnight incubation, coating solution was discarded and the wells were blocked with 150 μl/well of Blocking Buffer (1% Bovine Serum Albumin (BSA) in Assay Buffer; Sigma Catalog #A3059-100G; Burlington, MA, USA) for 2 hours at 4° C. Frozen stock of GalNAc-PAA-biotin (GlycoNZ Catalog #GNZ-0031-BP, 1 mg/ml in Assay Buffer; Auckland, NZ) was thawed and diluted 1:1000 in Assay Buffer to create a 1000 ng/ml 2× working solution. Competitor compounds were reconstituted to 100 mM in DMSO. If compounds were insoluble at this concentration, they were further diluted in DMSO. Compounds were diluted 1 to 12.5 (10 μl DMSO stock diluted to 125 μl) in Assay Buffer, then 5 fold serially diluted. The DMSO concentration in the 2× working solution of the first well is 8%, for a final starting DMSO concentration of 4%. In a separate deep-well plate (NEST Catalog #503501; Woodbridge, NJ, USA), equal amounts of 1000 ng/ml GalNAc-PAA and serially diluted compounds were combined, for a final concentration of 500 ng/ml GalNAc-PAA and 4 mM compound (top concentration), assuming that the compound concentration of the DMSO stock solution was 100 mM. Plates with Blocking Buffer were removed from refrigerator and blocking solution was discarded. The wells were washed with 200 μl of Assay Buffer three times. After the last wash, plates were tapped dry on paper towels to remove excess liquid.

Next, 100 μl of GalNAc-PAA-biotin premixed with serially diluted compounds was added into each well. For control, several wells containing 500 ng/ml GalNAc-PAA-biotin were included. Plates were incubated for 2 hours at room temperature on an orbital shaker at 100 rpm. After incubation, compound solutions were discarded and each well was washed with 200 μl Assay Buffer three times. Streptavidin-HRP (R&D Systems catalogue #DY998; Minneapolis, MN, USA) was diluted 1:200 in Assay Buffer. 100 μl of the diluted Streptavidin-HRP was added to each well and incubated at room temperature for 45 minutes, protected from light. Streptavidin-HRP was then discarded and each well was washed with 200 μl of Assay Buffer three times. 3,3′,5,5′-Tetramethylbenzidine (TMB) Substrate color reagents A and B (R&D Systems catalog #DY999; Minneapolis, MN, USA) were mixed at a ratio of 1:1 and 100 μl was added to each well. After a 5 minute incubation, 50 μl of Stop Solution (R&D Systems catalog #DY994; Minneapolis, MN, USA) was added to each well. Plates were shaken for 10 seconds using the orbital shaker in the plate reader and read at 450 nm. Data were plotted in GraphPad Prism (San Diego, CA, USA) and normalized as follows: 0% was non-specific GalNAc-PAA binding to wells without ASGPR protein and maximal 100% is highest number in each data series. Normalized data were analyzed using a nonlinear-fit of normalized data with a four-parameter sigmoidal dose-response variable slope model.

### Example 9E. Octet-Binding to TNF-α

Protocol. The binding to IL-17A was measured by Bio-Layer Interferometry (BLI) using the Sartorius Octet 96e platform (Goettingen, Germany). The binding assay was performed using Streptavidin (SA) Dip and Read Biosensors (Sartorius Catalog #18-5019; Goettingen, Germany) in OptiPlate-96F, Black, 96-well, Pinch bar design, plates (Perkin Elmer Catalog #6005270; Waltham, MA, USA) with the following steps: (1) 60 seconds of baseline; (2) 600 seconds of loading with 50 nM TNF-α (Biotinylated Human TNF-α Protein, His, Avitag (active trimer); ACROBiosystems Catalog #TNA-H82E3; Newark, DE, USA); (3) 60 seconds of baseline; (4) 120 seconds of quenching with 50 μg/ml of Biocytin (Thermo Fisher Catalog #B1592; Waltham, MA, USA); (5) 60 seconds of baseline; (6) 180 seconds of association with a dilution series of ligand consisting of seven concentrations. The eighth tip in the column was run in Assay Buffer (Phosphate Buffered Saline (PBS; Gibco Catalog #10010; Pottstown, PA, USA)+0.05% Tween 20 (v/v) (Fisher BioReagents Catalog BP337-500; Pittsburgh, PA, USA)+0.1% BSA (w/v) (Rockland Catalog #BSA-50; Pottstown, PA, USA)) to serve as a reference sensor, the signal of which was subtracted from the sample sensors. (7) 180 seconds of dissociation in Assay Buffer.

Results. Data analysis was done using the Octet Data Analysis HT software (Sartorius, Goettingen, Germany). Processed data was globally fit to a 1:1 binding model for determination of the equilibrium dissociation constant (K_{D}).

### Example 9F. Octet-Binding to ASGPR Protein

Protocol. The binding to native human ASGPR protein was measured by Bio-Layer Interferometry (BLI) using the Sartorius Octet 96e platform (Goettingen, Germany). The binding assay was performed using the Ni-NTA (NTA) Dip and Read Biosensors (Sartorius Catalog #18-5101; Goettingen, Germany) in OptiPlate-96F, Black, 96-well, Pinch bar design, plates (Perkin Elmer Catalog #6005270; Waltham, MA, USA) with the following steps: (1) 60 seconds of baseline in Assay Buffer (PBS (Gibco Catalog #10010; Pottstown, PA, USA)+0.05% Tween 20 (v/v) (Fisher BioReagents Catalog BP337-500; Pittsburgh, PA, USA)+0.1% BSA (w/v) (Rockland Catalog #BSA-50; Pottstown, PA, USA) +1 mM CaCl₂; Sigma Catalog #21115; Burlington, MA, USA); (2) 600 seconds of loading with 100 nM ASGPR protein; (3) 60 seconds of baseline; (4) 180 seconds of association with a dilution series of ligand consisting of seven concentrations. The eighth tip in the column was run in Assay Buffer to serve as a reference sensor, the signal of which was subtracted from the sample sensors. (5) 180 seconds of dissociation in Assay Buffer.

Results. Data analysis was done using the Octet Data Analysis HT software (Sartorius, Goettingen, Germany). Processed data was globally fit to a 1:1 binding model for determination of the equilibrium dissociation constant (K_{D}).

TABLE 1

| | | TNF-α | | | PAA |
|---|---|---|---|---|---|
| | Function | TM | | | ASGPR |
| Compound | IC₅₀ | Kd | Octet Kd (nM) | | Ki |
| No. | (μM) | (μM) | ASGPR | TNF-α | (μM) |
| Intermediate 25 | 0.24 | <0.001 | N.A. | N.A. | N.A. |
| Intermediate 24 | 0.247 | 0.22 | N.A. | N.A. | N.A. |
| Intermediate 23 | 0.337 | 0.031 | N.A. | N.A. | N.A. |
| Intermediate 3 | 1 | 0.12 | N.A. | N.A. | N.A. |
| Intermediate 4 | 0.23 | 0.069 | N.A. | N.A. | N.A. |
| Intermediate 18 | 0.81 | N.A. | N.A. | N.A. | N.A. |
| Intermediate 19 | 0.37 | N.A. | N.A. | N.A. | N.A. |
| 101 | 1.04 | 0.2 | 4.83 | N.B. | 0.10 |
| 102 | 1.45 | 0.11 | 1.5 | N.B. | 0.04 |
| 103 | >10,000 | 1 | 2.13 | N.B. | 0.2 |
| 104 | 1 | 1 | 1.0 | N.B. | 0.2 |
| 105 | 0.334 | 0.065 | weak | N.B. | 0.6 |
| | | | binding | | |
| 106 | 0.356 | 0.05 | >1000 | N.B. | 1.1 |
| 107 | 0.319 | 0.055 | 963 | N.B. | 0.8 |

N.A.—Not Applicable.
N.B.—No Binding.

What is claimed is:

1. A bifunctional Tumor Necrosis Factor-alpha (TNF-α) binding compound the formula: 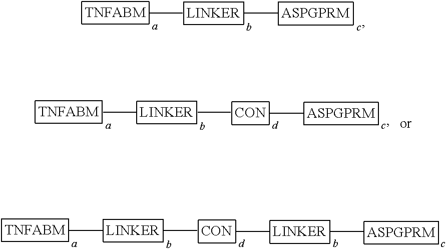
wherein,
[TNFABM] is a Tumor Necrosis Factor-Alpha (TNF-α) Binding Moiety that is capable of binding to TNF-α;
[ASPGPRM] is an Asialoglycoprotein Receptor Binding Moiety that binds to an asialoglycoprotein receptor (ASGPR) on an outer surface of a cell (e.g., a hepatocyte or other degrading cell, such as a cell in a subject);
[LINKER] is an optional chemical moiety that is covalently attached to one or more [TNFABM] groups and either one or more [CON] groups or one or more [ASPGPRM] groups;
[CON] is an optional chemical connecting moiety that is covalently attached to one or more [ASPGPRM] groups and one or more [LINKER] groups;
a is an integer from 1 to 15 (e.g. 1, 2, 3, 4, 5, 6, 7, 8, 9, 10, 11, 12, 13, 14, or 15);
b is an integer, independently selected at each occurrence from 0 to 15 (e.g. 0, 1, 2, 3, 4, 5, 6, 7, 8, 9, 10, 11, 12, 13, 14, or 15), preferably 0 or 1;
c is an integer from 1 to 15 (e.g. 1, 2, 3, 4, 5, 6, 7, 8, 9, 10, 11, 12, 13, 14, or 15); and
d is an integer from 0 to 15 (e.g. 0, 1, 2, 3, 4, 5, 6, 7, 8, 9, 10, 11, 12, 13, 14, or 15, preferably 0, 1, 2, or 3); or a pharmaceutically acceptable salt thereof.

2. The bifunctional compound, or a pharmaceutically acceptable salt thereof, of claim 1,
wherein at least one of
a is 1, 2 or 3;
b is 1, 2 or 3;
c is 1, 2 or 3; or
d is 1, 2 or 3.

3. The bifunctional compound, or a pharmaceutically acceptable salt thereof, of claim 1 or 2, wherein each [ASPGPRM] is independently selected from: 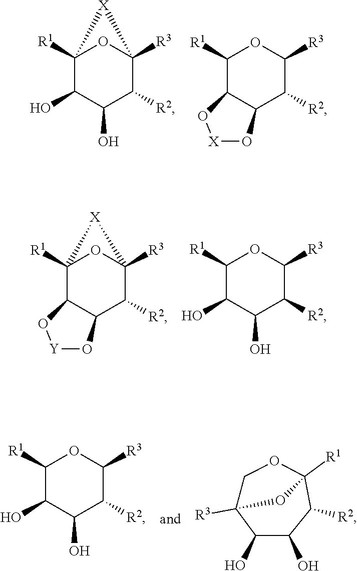
wherein:
X is 1, 2, 3, or 4 atoms in length, comprising one or more groups independently selected from O, S, N(R^{N1}), and C(R^{C1})(R^{C1}), wherein
when X is 1 atom in length, X is O, S, N(R^{N1}), or C(R^{C1})(R^{C1});
when X is 2 atoms in length, no more than 1 atom of X is O, S, or N(R^{N1});
when X is 3 or 4 atoms in length, no more than 2 (e.g., 0, 1, or 2) atoms of X are O, S, or N(R^{N1});
Y is 1 or 2 atoms in length, comprising one or two independently chosen O and C(R^{C1})(R^{C1}) groups;
each R^{N1} is independently H or C1-C₃ alkyl optionally substituted with 1, 2, or 3 independently selected halogen atoms; and
each R^{C1} is independently hydrogen, halogen, or C₁-C₃ alkyl optionally substituted with 1, 2, or 3 independently selected halogen atoms;
the point of attachment of the [ASGPRBM] to the [LINKER] or [CON] is through R¹ or R³,
R¹ and R³ are each independently selected from (i), (ii), (iii), (iv), and (v):
(i) hydrogen, which is replaced by a covalent bond when the R¹ or R³ is the point of attachment to the [LINKER] or [CON], or hydroxyl in which the hydrogen is replaced by a covalent bond when the R¹ or R³ is the point of attachment to the [LINKER] or [CON];
(ii) straight or branched C₁-C₈alkyl, optionally containing 1 or 2 double or triple bonds, optionally including 1 or 2 groups in the alkyl chain independently selected from O, S, NH, and N(C₁-C₄alkyl), and optionally substituted with one or more substituents independently selected from hydroxyl (—OH), halogen, cyano (—CN), oxo (═O), amino (—NH₂), imino (═NH), and —COOH, and is covalently bound to [LINKER] or [CON] via a terminal N, O, or C atom when the R¹ or R³ is the point of attachment to the [LINKER] or [CON];
(iii) a (5- or 6-membered heterocycle)(CH₂)_{K}— that is optionally substituted with one or more (e.g., 1, 2, 3, 4, 5, 6, or more) R^{A}, wherein
R^{A} is one or more C₁-C₄alkyl groups optionally substituted with halogen or hydroxyl;
R^{A}, when bound to a nitrogen, is no more than one substituent chosen from (phenyl)(CH₂)_{K}—, (C₃-C₇cycloalkyl)(CH₂)_{K}—, 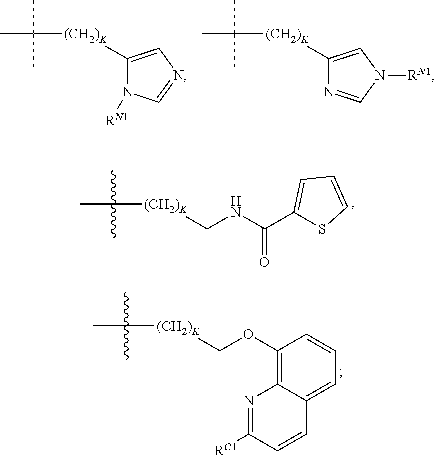
and
R^{A}, when bound to carbon atom, is selected from any of previously listed R^{A} groups and is also selected from halogen, hydroxyl, C₁-C₄alkoxy optionally substituted with 1-3 (e.g. 1, 2, or 3) independently selected halogen atoms or 1-2 (e.g. 1 or 2) hydroxyl groups, and mono- and di(C₁-C₄alkyl)amino,
wherein any (5- or 6-membered heterocycle)(CH₂)_{K}— group within the (iv) definition is covalently bound to [LINKER] or [CON] via a terminal N, O, or C atom when the R¹ or R³ is the point of attachment to the [LINKER] or [CON]; or
(iv) a (phenyl)(CH₂)_{K}— that is:
optionally substituted with 1 or more substituents independently chosen from halogen, hydroxyl, amino (—NH₂), —CN, and a straight or branched C₁-C₈alkyl that optionally contains 1 or 2 double or triple bonds, optionally includes 1 or 2 groups in the alkyl chain independently selected from O, S, NH, and N(C₁-C₄alkyl), wherein the C₁-C₄ alkyl is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently chosen from hydroxyl, halogen, cyano, oxo, imino, and —COOH,
wherein any (phenyl)(CH₂)_{K}— group within the (v) definition is covalently bound to [LINKER] or [CON] via a terminal N, O, or C atom when the R¹ or R³ is the point of attachment to the [LINKER] or [CON]; and
(v) a C₃-C₈cyclolalkyl-(CH₂)k- optionally substituted with R^{A} and is covalently bound to [LINKER] or [CON] via a ring C atom, or a terminal N, O, or C atom when the R¹ or R³ is the point of attachment to the [LINKER] or [CON],
wherein each K is independently 0, 1, 2, 3, or 4, preferably 0 or 1;
R₂ is chosen from (i), (ii), (iii), (iv), (v), (vi), and (vii) below:
(i) hydroxyl, halogen, amino, N₃, or —COOH;
(ii) straight or branched C₁-C₈alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently chosen from hydrogen, hydroxyl, halogen, oxo, and —COOH;
(iii) 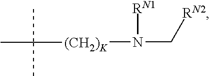
where R^{N1} and K are the same as above;
R^{N2} is H, —C₀-C₄alkylNR^{N3}R^{N1}, or straight or branched C₁-C₈alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C1-C₈alkyl is optionally substituted with one or more substituents independently selected from hydroxyl, halogen, cyano, oxo, amino, imino (═NH), and —COOH; and
R^{N3} is H, 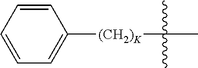
group, or a C₁-C₃ alkyl group optionally substituted with 1-3 independently selected halogen atoms or 1-2 hydroxyl groups, or
(iv) 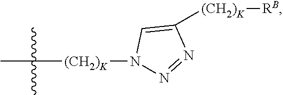
wherein
R^{B} is H, CN, straight or branched C₁-C₈alkyl that optionally contains 1 or 2 double or triple bonds, optionally includes 1 or 2 groups in the alkyl chain selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently selected from hydroxyl, halogen, cyano, oxo, amino, imino (═NH), and —COOH; or
R^{B} is a C₃-C₁₀ aryl or a three- to ten-membered heteroaryl group containing up to 5 heteroaryl atoms, each of said aryl or heteroaryl groups is optionally substituted with one or more (preferably 1) halogen, hydroxyl, CN, NR^{N1}R^{N2}, straight or branched C₁-C₈alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently selected from hydroxyl, halogen, cyano, oxo, and —COOH; or
R^{B} is 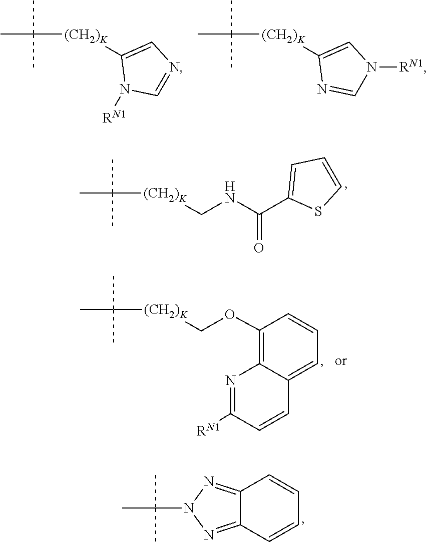
each of which is optionally substituted with up to three (e.g., 1, 2, or 3) substituents independently selected from halogen, hydroxyl, cyano, C₁-C₃alkyl, C₁-C₃alkoxy, C₁-C₃haloalkyl, and C₁-C₃haloalkoxy;
(v) 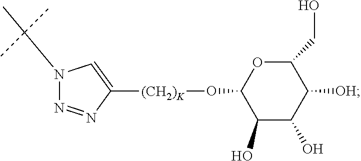
(vi) a carbocyclic or a heterocyclic group that is directly bound to the [ASGPRBM] group or bound to the [ASGPRBM] group via a O, S, NH, NHC(═O), C(═O)NH, or C₁-C₄alkylene that optionally has 1 double or triple bond and optionally has one —CH₂— group replaced by a 0, S, or NH, wherein each of which carbocyclic or heterocyclic group is unsubstituted or substituted with one or more substituents independently selected from halogen, hydroxyl, amino, azido, cyano, —COOH, oxo, imino, and a straight, branched, or cyclic C₁-C₈alkyl that optionally contains 1 or 2 double or triple bonds, optionally includes 1 or 2 groups in the alkyl chain selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is unsubstituted or substituted with one or more substituents independently selected from hydroxyl, halogen, SF₅, cyano, oxo, amino, azido, imino (═NH), and —COOH, and substituted with 0 or 1 substituents selected from an aryl and a heterocyclic substituent, which aryl or heterocyclic substituent is optionally substituted with one or more R^{C1} substituents;
(vii) —NR^{N4}—S(O)—R^{D}, —NR^{N4}—C(S)—R^{D}, —NR^{N4}—S(O)(NR^{N4})R^{N4}, —N═S(O)(R^{D})₂, —NR^{N4}C(O)NR^{N4}S(O)₂R^{D}, —NR^{N4}—S(O)₂—R^{D}, —S(═O)(═NR^{N4})R^{D}, or —NR^{N4}C(NR^{N4})R^{N4}, wherein each R^{D} is independently selected from
(a) hydrogen,
(b) heterocycle-(CH₂)_{K} or carbocycle-(CH₂)_{K}, each of which is optionally substituted with one or more substituents independently selected from halogen, hydroxyl, cyano, amino, C1-C₄alkyl, C₁-C₄alkoxy, C₁-C₂haloalkyl, and C₁-C₂haloalkoxy,
(c) -ORE, —NR^{N4}R^{N4}, and
(d) straight or branched C₁-C₆alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₆alkyl is optionally substituted with one or more substituents independently chosen from hydroxyl, halogen, cyano, oxo, amino, imino (═NH), and —COOH;
each R^{N4} and R^{E} is independently selected from
(a) hydrogen,
(b) heterocycloalkyl-(CH₂)_{K}, heteroaryl-(CH₂)_{K}, aryl-(CH₂)_{K}, C₃-C₆cycloalkyl-(CH₂)_{K}, each of which is optionally substituted with one or more substituents independently selected from halogen, hydroxyl, cyano, amino, C₁-C₄alkyl, C₁-C₄alkoxy, C₁-C₂haloalkyl, and C₁-C₂haloalkoxy, and
(c) straight or branched C₁-C₆alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₆alkyl is optionally substituted with one or more substituents independently chosen from hydroxyl, halogen, cyano, oxo, amino, imino (═NH), and —COOH or
R² and R³ are joined to form a 5- to 6-membered saturated or partially unsaturated cyclic group optionally containing 1, 2, or 3 heteroatoms independently selected from N, O, and S, and optionally substituted with 1 or more substituents independently selected from oxo, halogen, hydroxyl, cyano, amino, and straight or branched C1-C₆alkyl that optionally contains 1 or 2 double or triple bonds, optionally includes 1 or 2 groups in the alkyl chain selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₆alkyl is optionally substituted with one or more substituents independently selected from hydroxyl, halogen, cyano, oxo, and —COOH.

4. The bifunctional compound, or a pharmaceutically acceptable salt thereof, of claim 3, wherein each [ASPGPRM] is independently selected from: 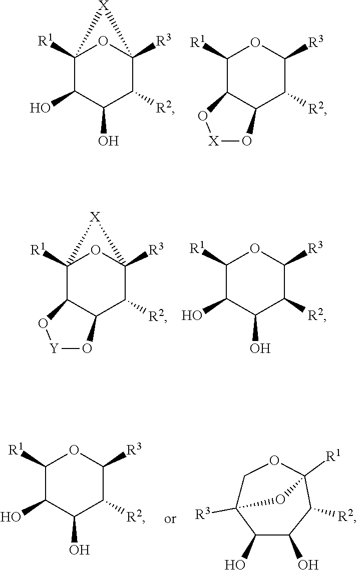
Where
X and Y are independently selected from —CH₂—, —CH₂CH₂—, —CH₂O—, and —OCH₂—;
R¹ and R³ are independently:
(i) hydrogen, which is replaced by a covalent bond when the R¹ or R³ is the point of attachment to the [LINKER] or [CON], or hydroxyl in which the hydrogen is replaced by a covalent bond when the R¹ or R³ is the point of attachment to the [LINKER] or [CON];
(ii) straight or branched C₁-C₈alkyl, optionally containing 1 or 2 double or triple bonds, optionally including 1 or 2 groups in the alkyl chain independently selected from O, S, NH, and N(C1-C₄alkyl), and optionally substituted with one or more substituents independently selected from hydroxyl (—OH), halogen, cyano (—CN), oxo (═O), amino (—NH₂), imino (═NH), and —COOH, and is covalently bound to [LINKER] or [CON] via a terminal N, O, or C atom when the R¹ or R³ is the point of attachment to the [LINKER] or [CON];
R² is
(i) hydroxyl, halogen, amino, N₃, or —COOH; or
(ii) straight or branched C₁-C₈alkyl that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently chosen from hydrogen, hydroxyl, halogen, oxo, and —COOH.

5. The bifunctional compound, or a pharmaceutically acceptable salt thereof, of claim 3, wherein at least one of, or each of, the [ASPGPRM] is:

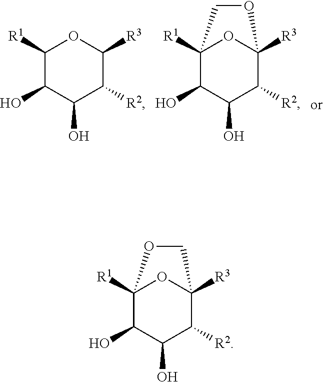

6. The bifunctional compound, or a pharmaceutically acceptable salt thereof, of claim 3 or 5, wherein R¹ and R³, in at least one of or each of the [ASPGPRM], are independently chosen from definitions (i), (ii), and (iii).

7. The bifunctional compound, or a pharmaceutically acceptable salt thereof, of any one of claims 3-5, wherein R¹, in at least one of or each of the [ASPGPRM], is [L]-OCH₂—, [L]-CH₂CH₂—, or [L]-NHCH₂—, where [L] represent the point of attachment to the [LINKER] or [CON], and the dash (“-”) is the point of attachment of the R¹ substituent to the group it substitutes.

8. The bifunctional compound, or a pharmaceutically acceptable salt thereof, of any one of claim 3 or 5-7, wherein at least one of, or each of, the [ASGPRBM] is 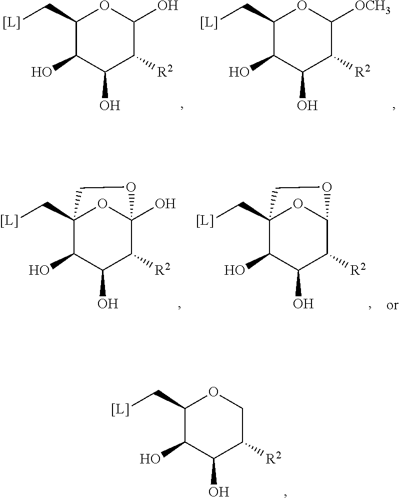
wherein [L] denotes the point of attachment to the [LINKER] or [CON].

9. The bifunctional compound, or a pharmaceutically acceptable salt thereof, of any one of claim 3 or 5-8, wherein R², in at least one of or each of the [ASPGPRM], is a carbocyclic or heterocyclic group that is directly bound to the [ASGPRBM] group or bound to the [ASGPRBM] group via a 0, S, NH, NHC(═O), C(═O)NH, or C₁-C₄alkylene group, the C₁-C₄alkylene group optionally having 1 double or triple bond and optionally having one —CH₂— group replaced by a O, S, or NH, where the carbocyclic or heterocyclic group is phenyl, pyridine, pyridazine, pyrimidine, pyrazine, triazine, furan, pyrrole, thiadiazole, thiatriazole, pyrazole, triazole, isoxazole, oxazole, oxadiazole, imidazole, thiazole, cyclopropane, cyclobutane, cyclopentane, cyclohexane, oxetane, piperazine, piperidine, morpholine, azasilinane, diazaspiro[3.3]heptane, benzodiazole, benzimidazole, benzothiazole, pyrrolotriazine, pyrrolopyridine, pyrrolopyrimidine, each of which is unsubstituted or substituted with one or more substituents independently selected from halogen, hydroxyl, amino, azido, cyano, —COOH, oxo, imino, and straight, branched, or cyclic C₁-C₈alkyl, wherein the C₁-C₈alkyl optionally contains 1 or 2 double or triple bonds, optionally includes 1 or 2 groups in the alkyl chain independently selected from O, S, NH, and N(C₁-C₄alkyl) which N(C₁-C₄alkyl) is optionally substituted with 1-3 (e.g., 1, 2, or 3) independently selected halogen atoms or 1-2 (e.g., 1 or 2) hydroxyl groups, and the C1-C₈alkyl is unsubstituted or substituted with one or more substituents independently chosen from hydrogen, hydroxyl, halogen, SF₅, cyano, oxo, amino, azido, imino (═NH), and —COOH and is substituted with 0 or 1 aryl or heterocyclic substituent which 0 or 1 aryl or heterocyclic substituent is optionally substituted with one or more R^{C1} substituents.

10. The bifunctional compound, or a pharmaceutically acceptable salt thereof, of any one of claim 3 or 5-9, wherein
R², in at least one of or each of the [ASPGPRM], is a carbocyclic or heterocyclic group independently selected from 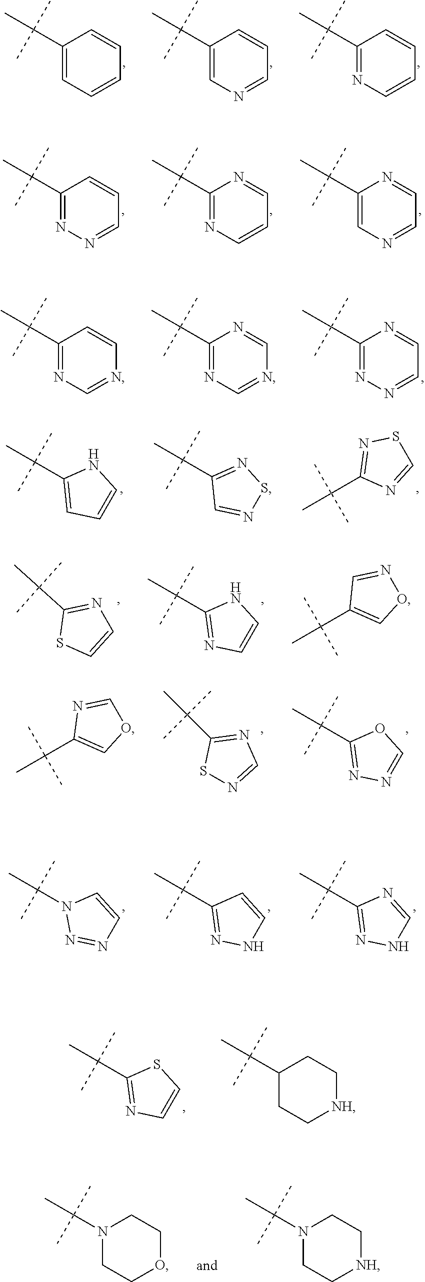
each of which is either directly bound to the [ASGPRBM] group or bound to the [ASGRPBM] group via a O, S, NH, or C₁-C₄alkylene group, the C₁-C₄alkylene group optionally having 1 double bond and optionally having one —CH₂— group replaced by a O, S, or NH, and the carbocyclic or heterocyclic group is unsubstituted or substituted with one or more substituents independently chosen from halogen, hydroxyl, amino, azido, cyano, —COOH, oxo, imino, and straight, branched, or cyclic C₁-C₈alkyl, that optionally contains 1 or 2 double or triple bonds, optionally includes 1 or 2 groups in the alkyl chain independently selected from O, S, NH, and N(C₁-C₄alkyl) which N(C₁-C₄alkyl) is optionally substituted with 1-3 (e.g., 1, 2, or 3) independently selected halogen atoms or 1-2 hydroxyl groups, and the C₁-C₈alkyl is unsubstituted or substituted with one or more substituents independently chosen from hydroxyl, halogen, SF₅, cyano, oxo, amino, azido, imino (═NH), and —COOH and is substituted with 0 or 1 aryl or heterocyclic substituent which 0 or 1 aryl or heteroaryl substituent is a phenyl, piperazine, piperidine, or morpholine substituent, optionally substituted with one or more R^{C1} substituents.

11. The bifunctional compound, or a pharmaceutically acceptable salt thereof, of any one of claims 3-7, wherein R², in at least one of or each of the [ASPGPRM], is

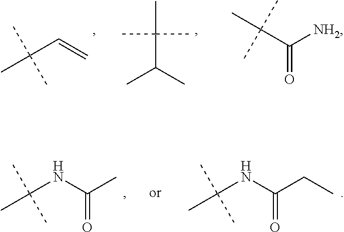

12. The bifunctional compound, or a pharmaceutically acceptable salt thereof, of any one of claim 3 or 5-7, wherein R², in at least one of or each of the [ASPGPRM], is 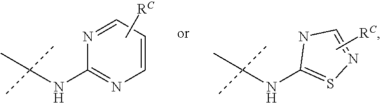
wherein R^{C} is 0, 1, or more substituents independently selected from halogen, hydroxyl cyano, amino, C₁-C₄alkyl, C₁-C₄alkoxy, C₁-C₂haloalkyl, and C₁-C₂haloalkoxy.

13. The bifunctional compound, or a pharmaceutically acceptable salt thereof, of claim 3, wherein R² and R³, in at least one of or each of the [ASPGPRM], are joined to form a ring such that the [ASGPRBM] has a structure of formula 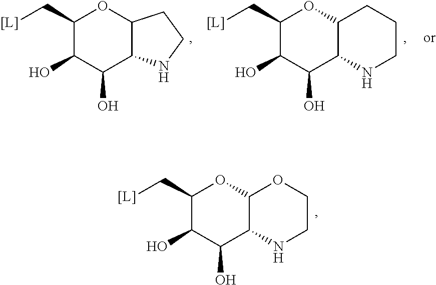
wherein [L] denotes the point of attachment to the [LINKER] or [CON], and the [ASGPRBM] is optionally substituted with 1 or more substituents (e.g., 1, 2, 3, 4, 5, or more) independently selected from oxo, halogen, hydroxyl, cyano, amino, and straight or branched C₁-C₆alkyl, wherein the alkyl group
(i) optionally contains 1 or 2 double or triple bonds,
(ii) optionally includes 1 or 2 groups in the alkyl chain independently selected from O, S, NH, and N(C₁-C₄alkyl) wherein the N(C₁-C₄alkyl) group is optionally substituted with 1-3 (e.g., 1, 2, or 3) halogen atoms, 1-2 (e.g. 1 or 2) hydroxyl groups, or a combination thereof, and
(iii) optionally substituted with one or more substituents (e.g., 1, 2, 3, 4, 5, or more) independently chosen from hydroxyl, halogen, cyano, oxo, and —COOH.

14. The bifunctional compound, or a pharmaceutically acceptable salt thereof, of any one of claims 3-7, wherein at least one of, or each of, the [ASGPRBM] is 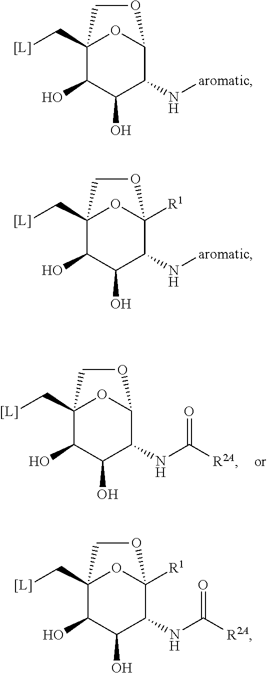
wherein
[L] denotes the point of attachment to the [LINKER] or [CON];
each aromatic is independently a 5-membered to 10-membered aromatic ring (e.g., a 6-membered to 10-membered aryl or a 5-membered to 10-membered heteroaryl) that optionally has one or more (e.g., 1, 2, 3, 4, or 5) independently selected heteroatoms (e.g., O, N, or S) and is optionally substituted with one or more substituents independently selected from halogen, hydroxyl, amino, CN, straight, branched, and cyclic C₁-C₈alkyl (e.g. a C₁-C₆alkyl) that optionally contains 1 or 2 double or triple bonds, optionally includes 1 or 2 groups in the alkyl chain independently selected from O, S, NH, and N(C₁-C₄alkyl), which N(C₁-C₄alkyl) is optionally substituted with 1-3 (e.g. 1, 2, or 3) halogen atoms or 1-2 (e.g. 1 or 2) hydroxyl groups, and the C₁-C₈alkyl is unsubstituted or substituted with one or more substituents independently chosen from hydroxyl, halogen, SF₅, cyano, oxo, amino, imino (═NH), and —COOH and is substituted with 0 or 1 aryl or heterocyclic substituent which 0 or 1 aryl or heterocyclic substituent is optionally substituted with one or more R^{C1} substituents; and
each R^{2A} is independently a C₁-C₄alkyl that is optionally substituted with 1 to 5 (e.g., 1, 2, 3, 4, or 5) independently selected halogen atoms.

15. The bifunctional compound, or a pharmaceutically acceptable salt thereof, of claim 3, wherein at least one of, or each of, the [ASGPRBM] is 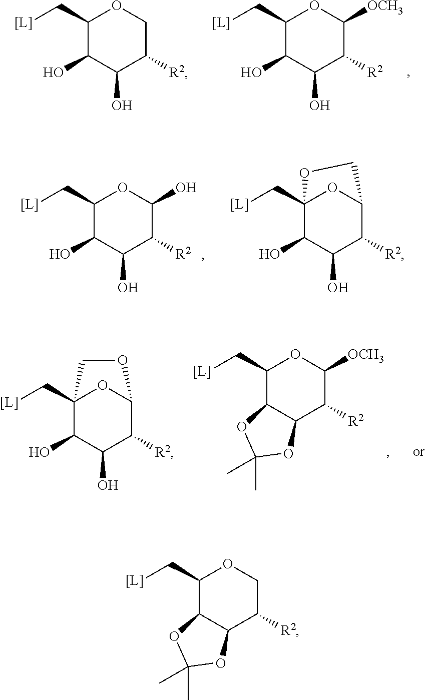
wherein:
[L] denotes the point of attachment to the [LINKER] or [CON]; and
Each R² is independently selected from —CF₃, —CH₃, —N₃, —OCH₃, —COOH, —C(═O)CH₃, —C(═O)OCH₃, —CH₂N₃, —CH₂OH, —(CH₂)₂OH, —(CH₂)₃OH, —C(═O)CF₃, —C(═O)NHCH₃, —(CH₂)₃OH, —(CH₂)C(═O)NH₂, —CH₂NHC(═O)NH₂, —(CH₂)₂C(═O)CH₃, —NHC(═O)CH₃, —NHC(═O)CH₂CH₃, —NHC(═O)CH₂F, —NHC(═O)CHF₂, —NHC(═O)CF₃, —NHC(═O)cyclopropyl, —NHC(═O)cyclobutyl, —NHC(═O)cyclopentyl, —NHC(═O)cyclohexyl, —NHSO₂CH₃, —NHSO₂CH₃, —NHSO₂CF₃, —NHC(═O)NHSO₂CH₃,

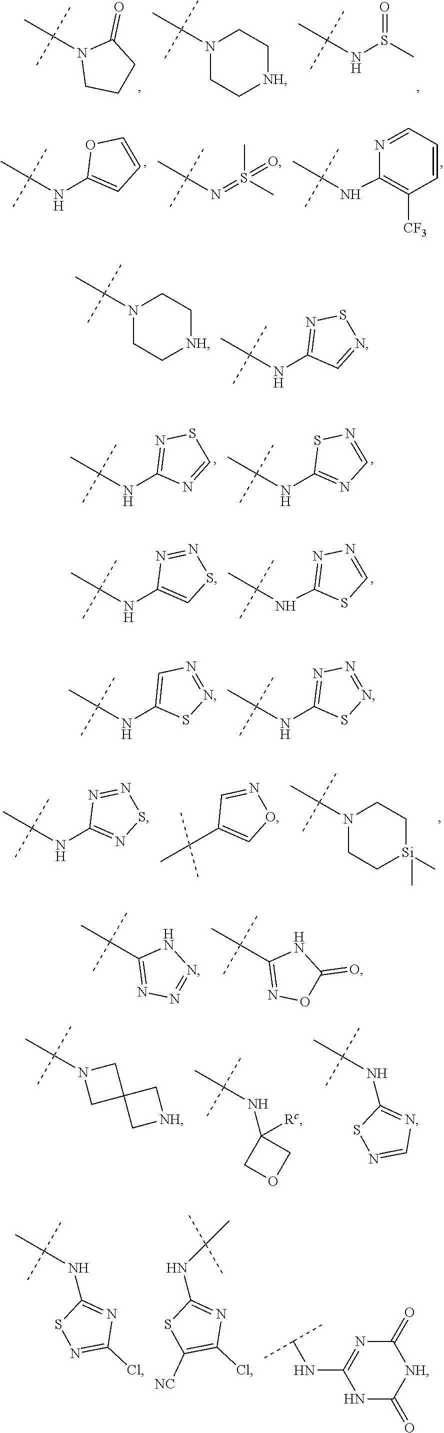

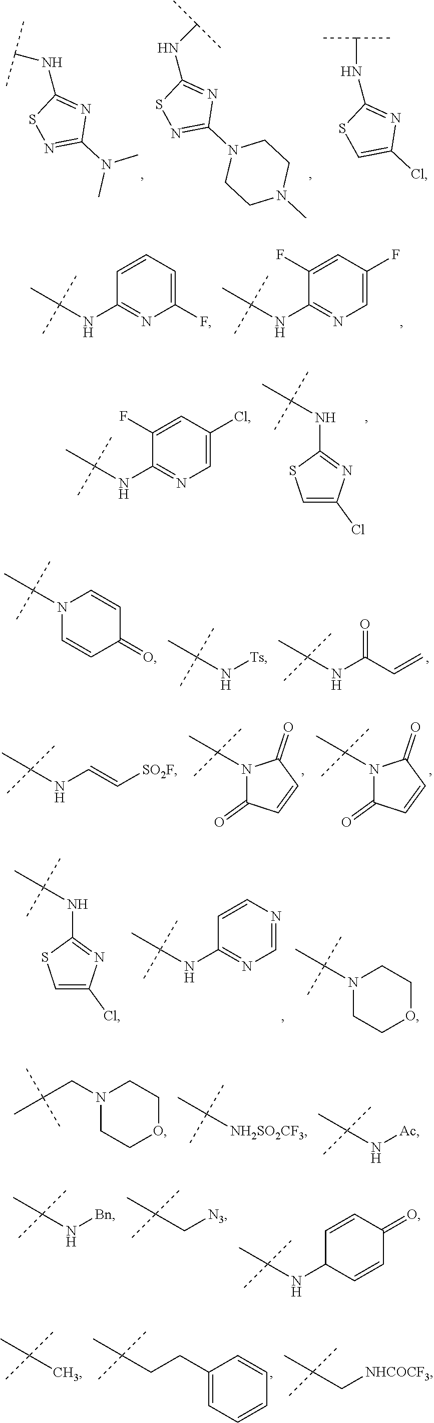

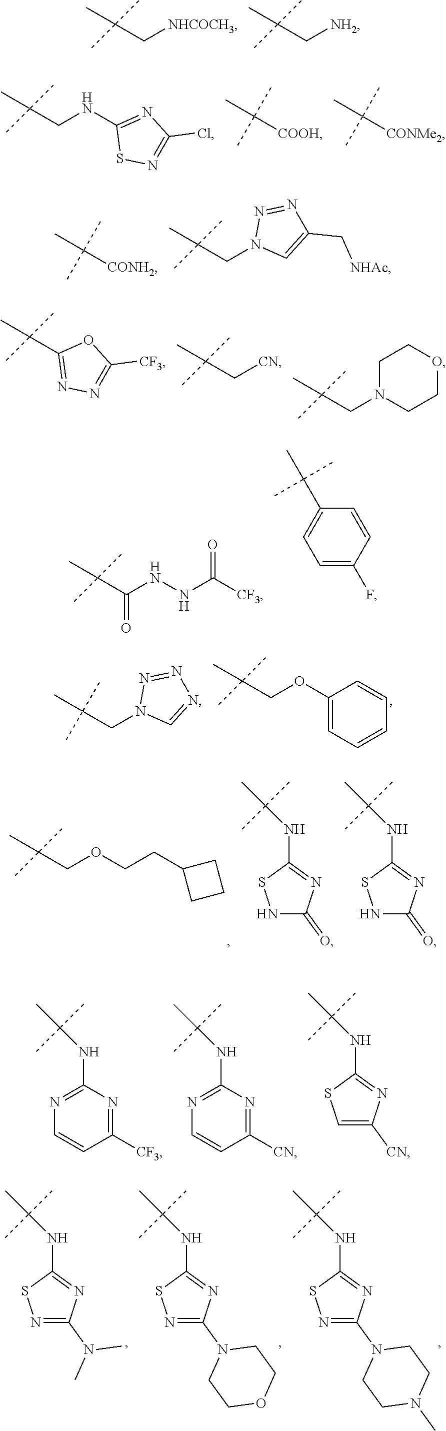

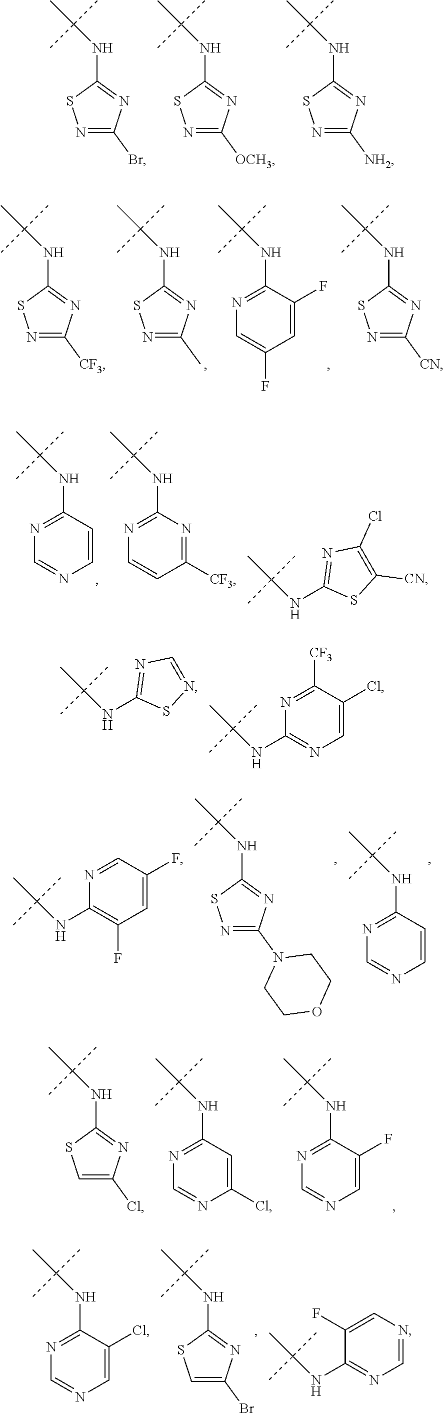

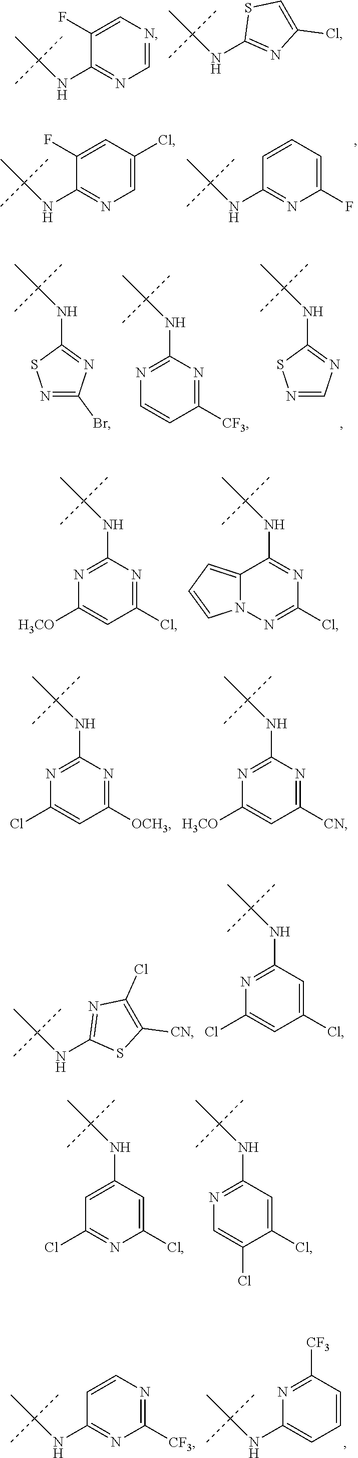

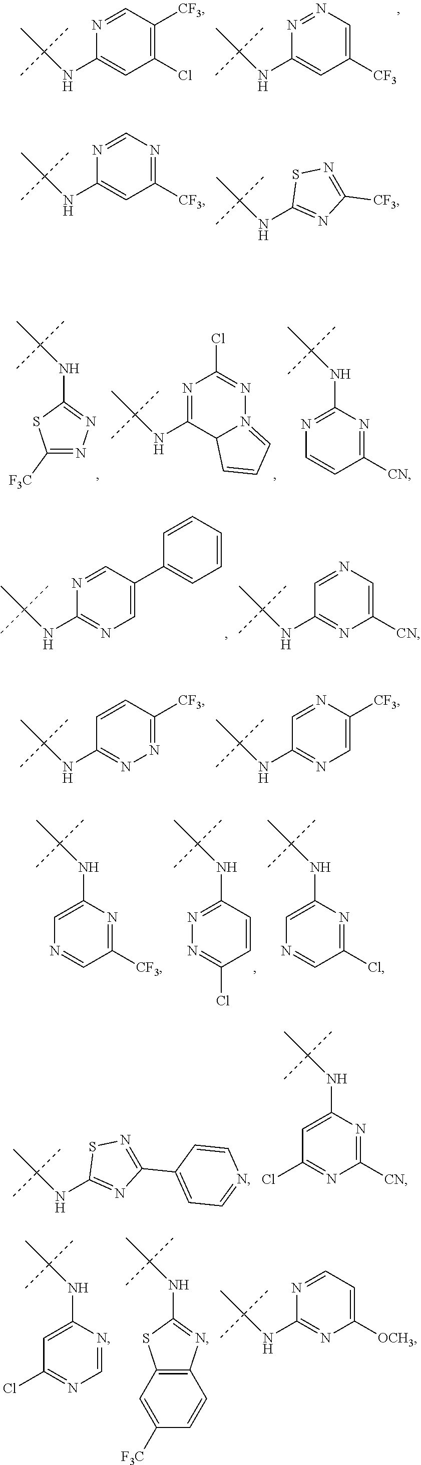

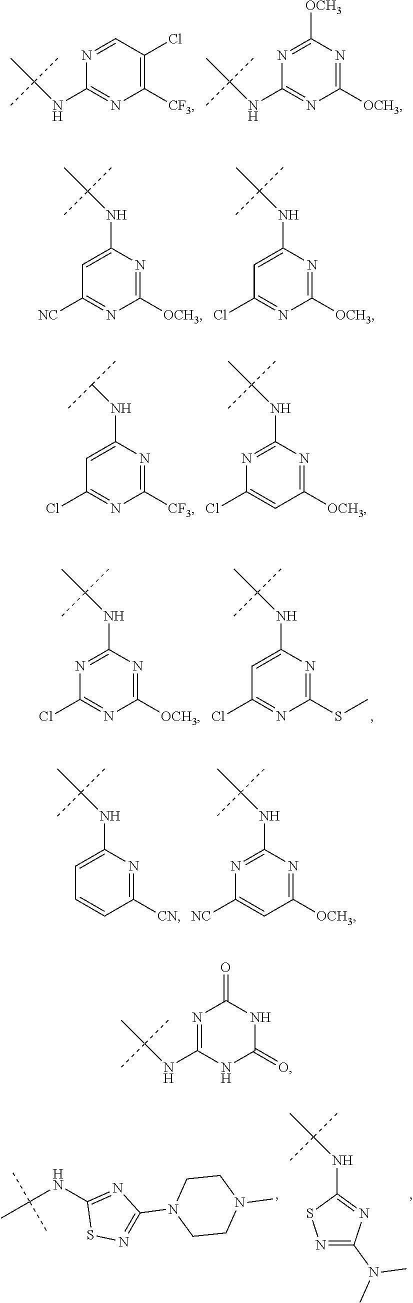

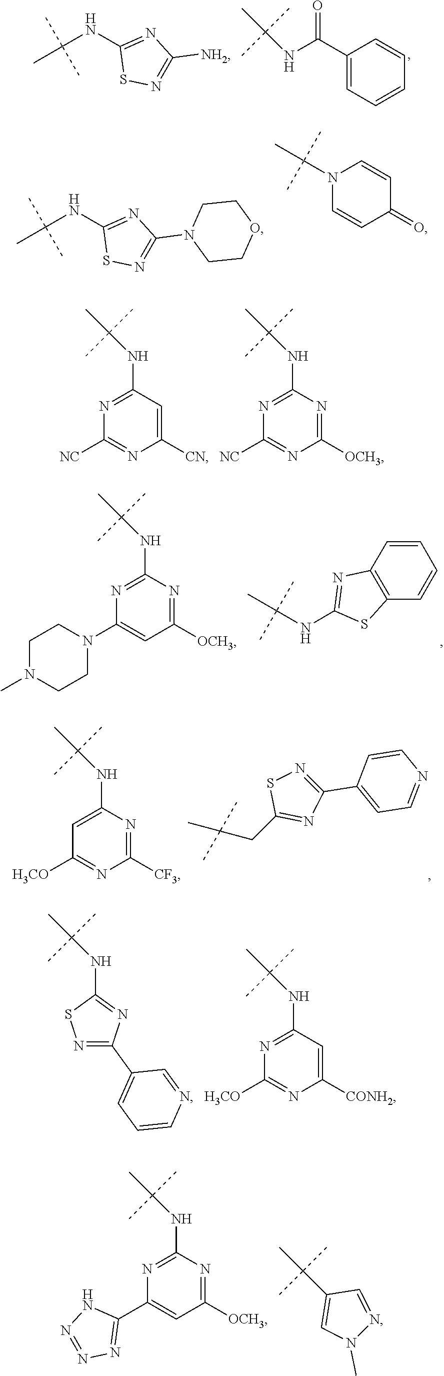

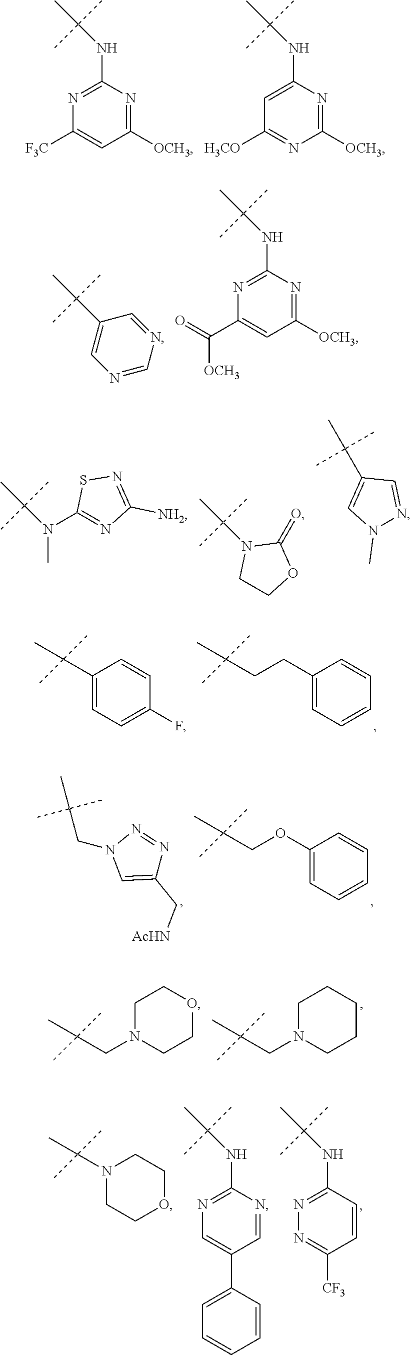

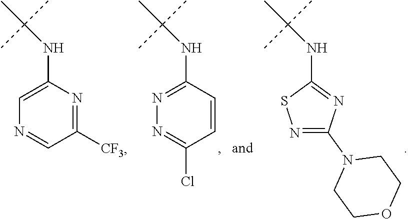

16. The bifunctional compound, or a pharmaceutically acceptable salt thereof, of claim 3, wherein at least one of, or each of, the [ASGPRBM] is 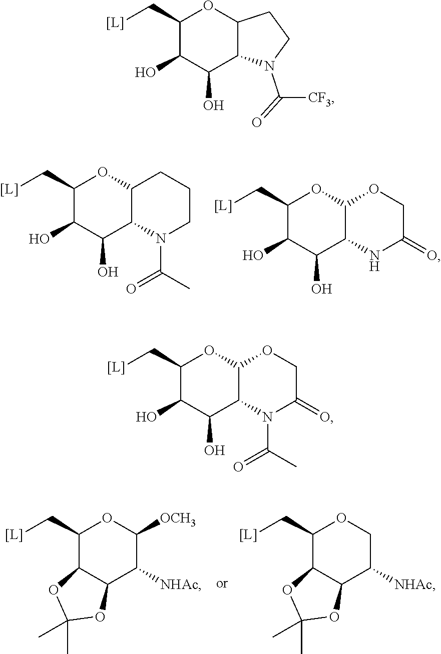
wherein [L] denotes the point of attachment to the [LINKER] or [CON].

17. The bifunctional compound, or a pharmaceutically acceptable salt thereof, of any one of claims 3, 5, or 6, wherein at least one of, or each of, the [ASGPRBM] is 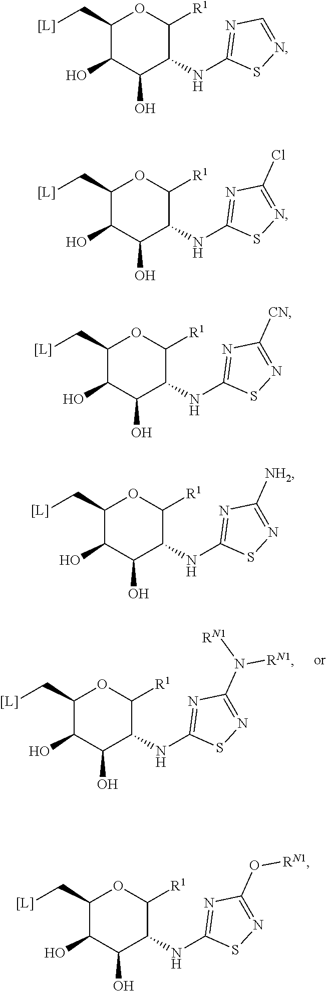
wherein [L] denotes the point of attachment to the [LINKER] or [CON].

18. The bifunctional compound, or a pharmaceutically acceptable salt thereof, of any one of claim 3 or 5-7, wherein R², in at least one of or each of the [ASPGPRM], is

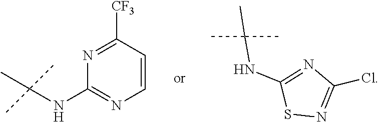

19. The bifunctional compound, or a pharmaceutically acceptable salt thereof, of claim 3 or 4, wherein R¹ or R³, in at least one of or each of the [ASPGPRM], is
(iii) a (5- or 6-membered heterocycle)(CH₂)_{K}— that is optionally substituted with one or more (e.g., 1, 2, 3, 4, 5, 6, or more) R^{A}, wherein
R^{A} is one or more C1-C₄alkyl groups optionally substituted with halogen or hydroxyl;
R^{A}, when bound to a nitrogen, is no more than one substituent chosen from (phenyl)(CH₂)_{K}—, (C₃-C₇cycloalkyl)(CH₂)_{K}—, 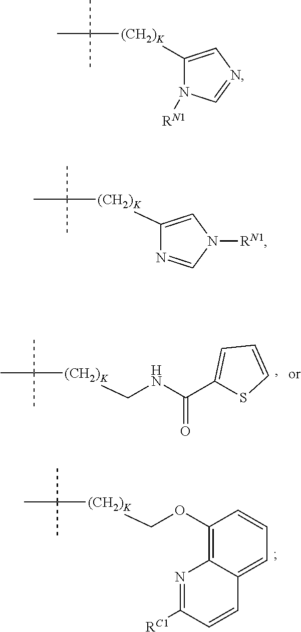
and
R^{A}, when bound to carbon atom, is selected from any of previously listed groups and is also selected from halogen, hydroxyl, C₁-C₄alkoxy optionally substituted with 1-3 (e.g. 1, 2, or 3) independently selected halogen atoms or 1-2 (e.g. 1 or 2) hydroxyl groups, and mono- and di(C₁-C₄alkyl)amino, wherein any (5- or 6-membered heterocycle)(CH₂)_{K}— group within the (iii) definition is covalently bound to [LINKER] or [CON] via a terminal N, O, or C atom when the R¹ or R³ is the point of attachment to the [LINKER] or [CON]; or
(iv) a (phenyl)(CH₂)_{K}— that is: optionally substituted with 1 or more substituents independently chosen from halogen, hydroxyl, amino (—NH₂), —CN, and a straight or branched C₁-C₈alkyl that optionally contains 1 or 2 double or triple bonds, optionally includes 1 or 2 groups in the alkyl chain independently selected from O, S, NH, and N(C₁-C₄alkyl), wherein the N(C₁-C₄ alkyl) is optionally substituted with 1-3 (e.g. 1, 2, or 3) independently selected halogen atoms or 1-2 (e.g. 1 or 2) hydroxyl groups, and the C₁-C₈alkyl is optionally substituted with one or more substituents independently chosen from hydroxyl, halogen, cyano, oxo, imino, and —COOH,
wherein any (phenyl)(CH₂)_{K}— group within the (iv) definition is covalently bound to [LINKER] or [CON] via a terminal N, O, or C atom when the R¹ or R³ is the point of attachment to the [LINKER] or [CON].

20. The bifunctional compound, or a pharmaceutically acceptable salt thereof, of claim 3 or 4, wherein R¹ or R, in at least one of or each of the [ASPGPRM], is 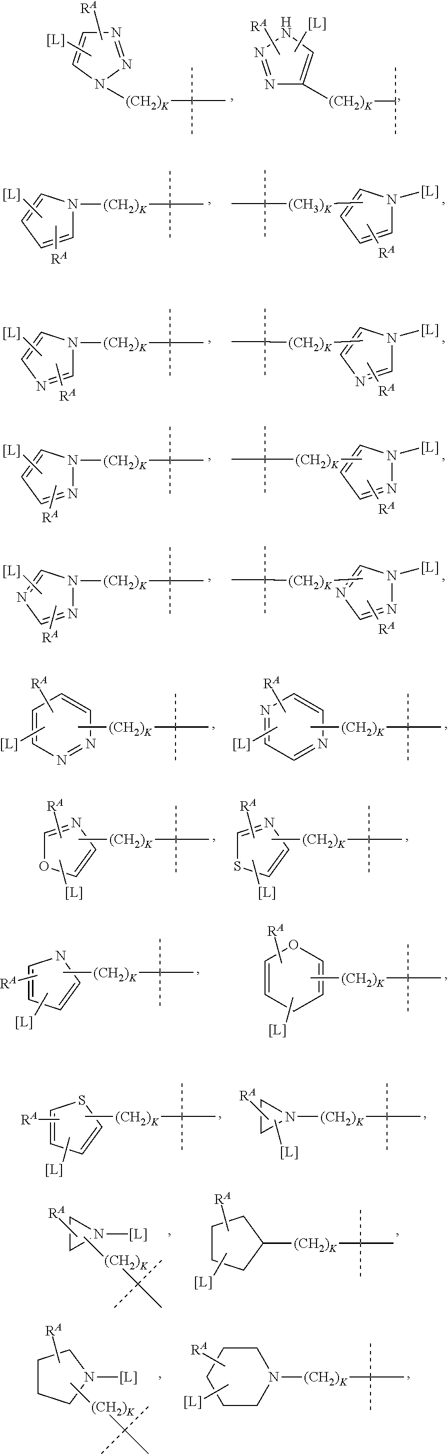 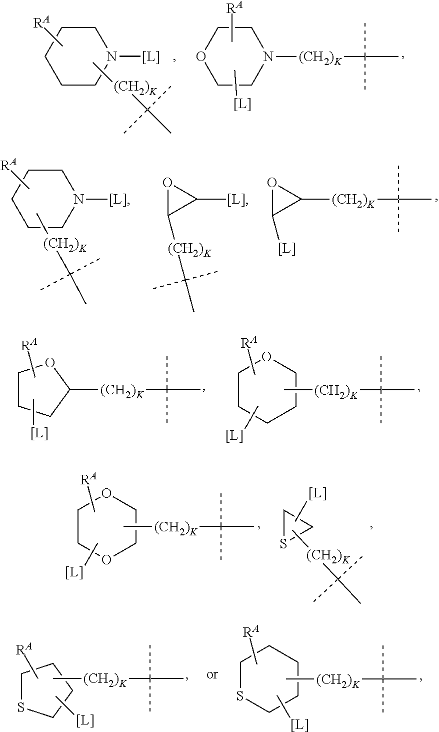
wherein [L] denotes the point of attachment to the [LINKER] or [CON].

21. The bifunctional compound, or a pharmaceutically acceptable salt thereof, of claim 3, wherein at least one of, or each of, the [ASGPRBM] is independently selected from 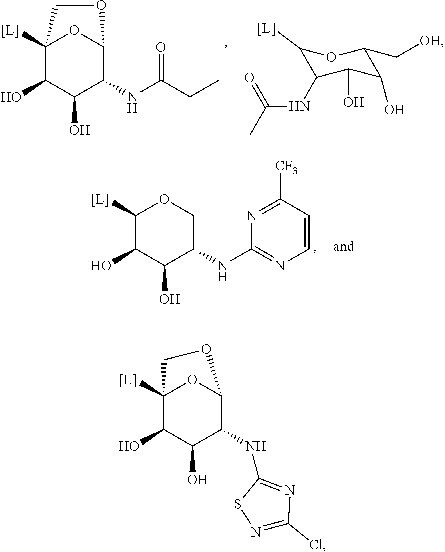
wherein [L] denotes the point of attachment to the [LINKER] or [CON].

22. The bifunctional compound, or a pharmaceutically acceptable salt thereof, of any one of claims 1-21, wherein the [LINKER] is selected from: 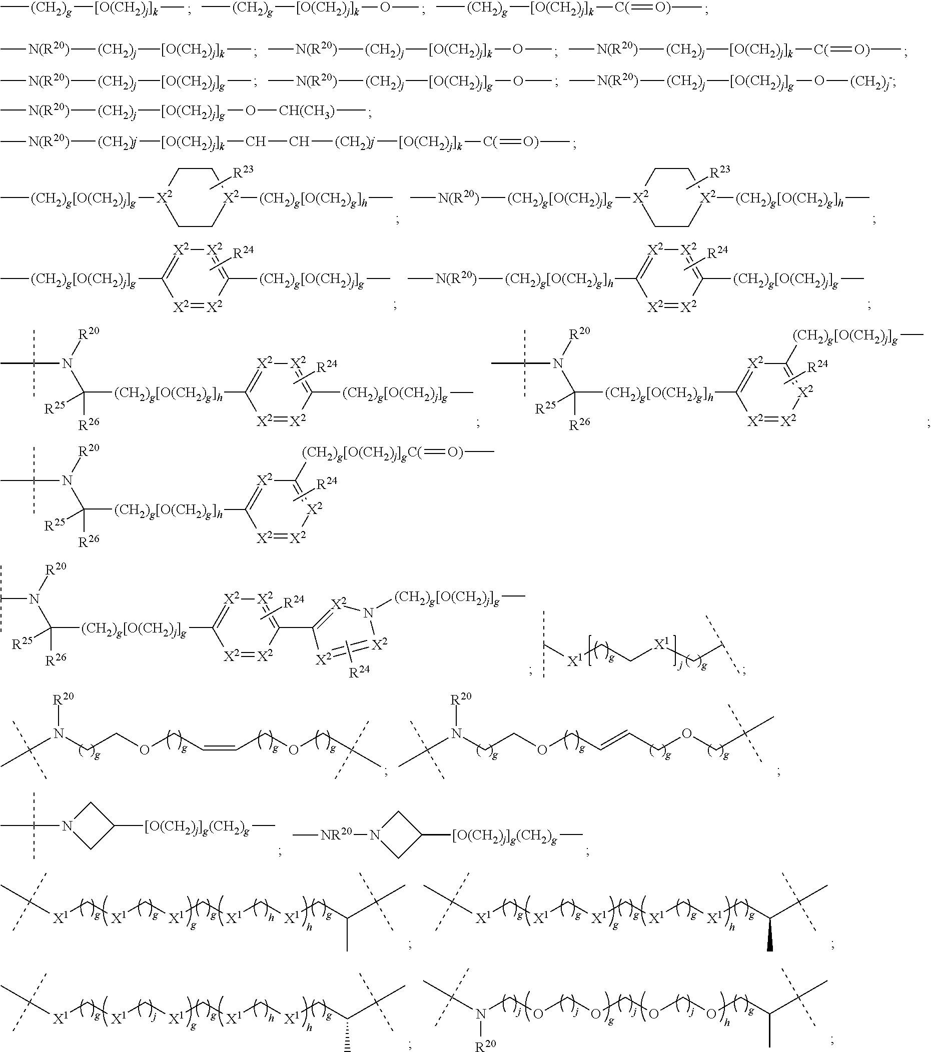 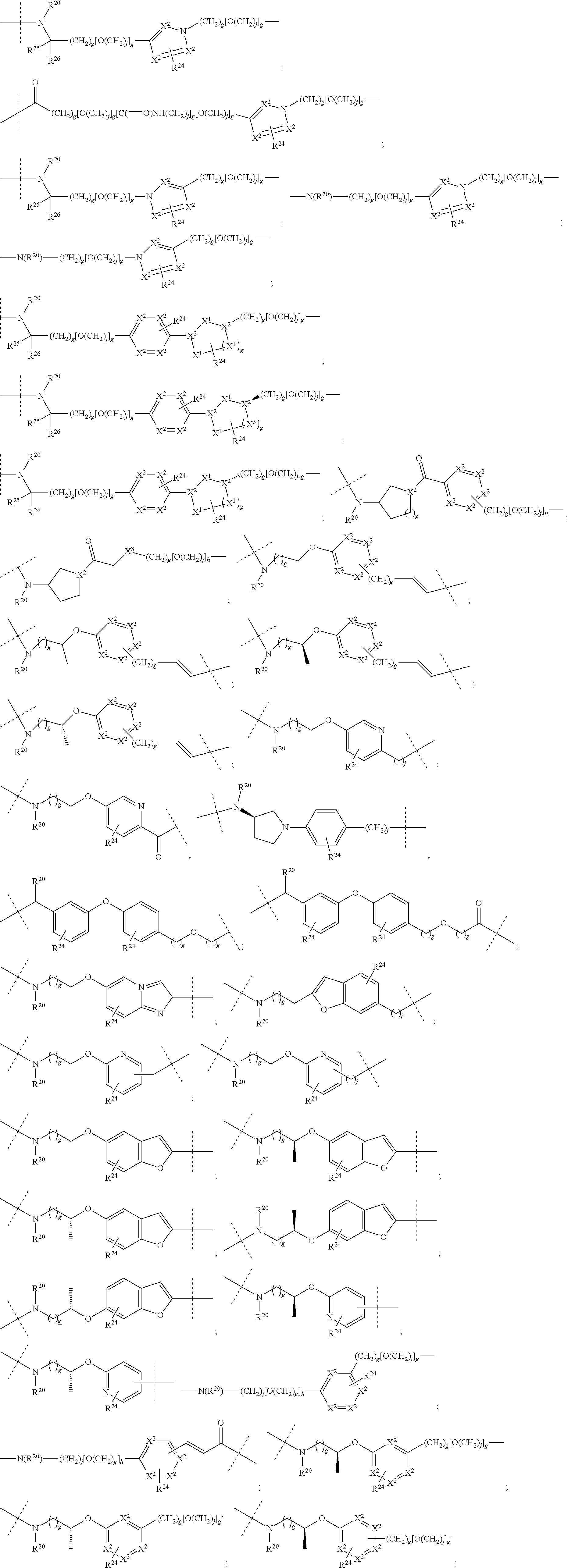 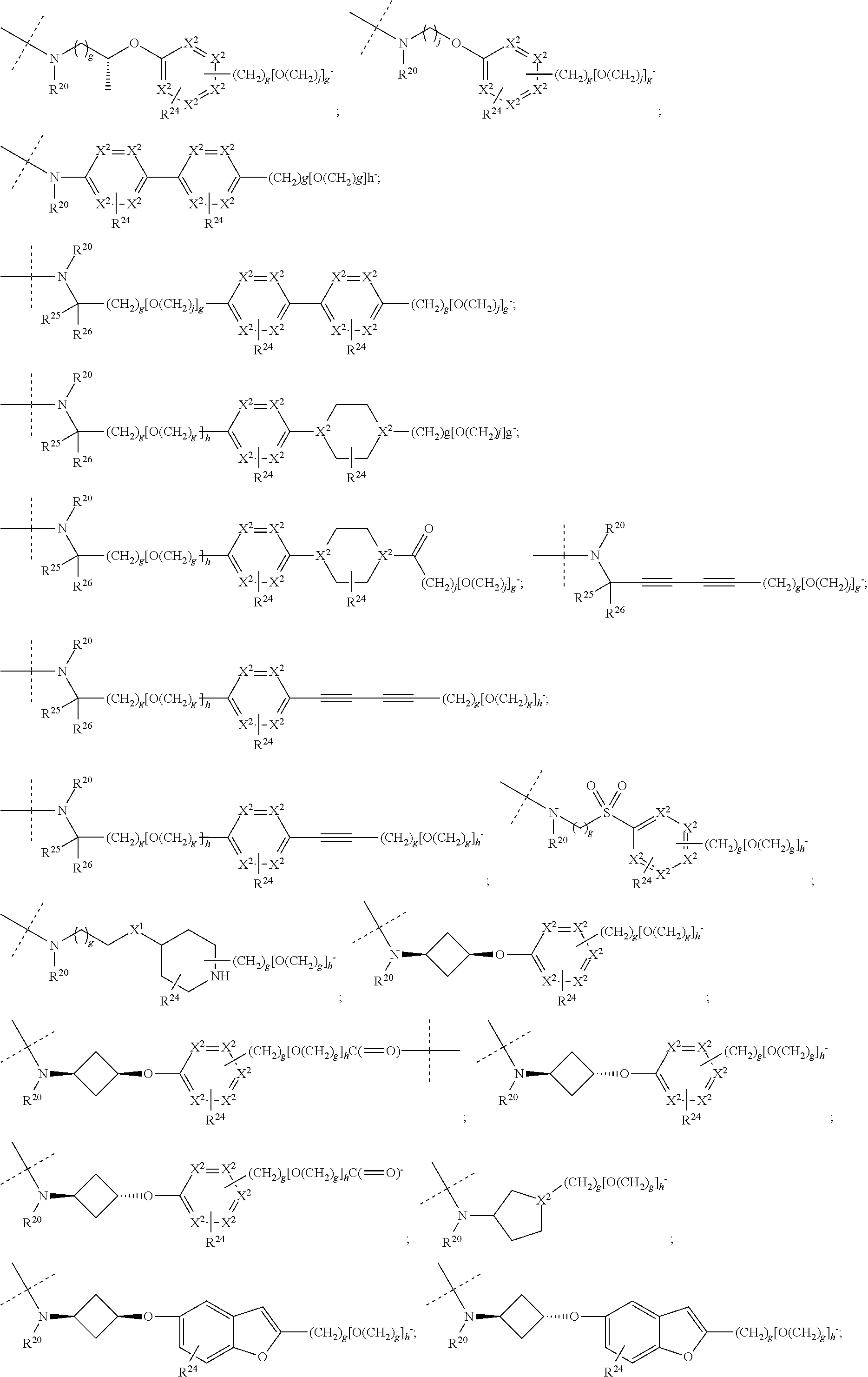 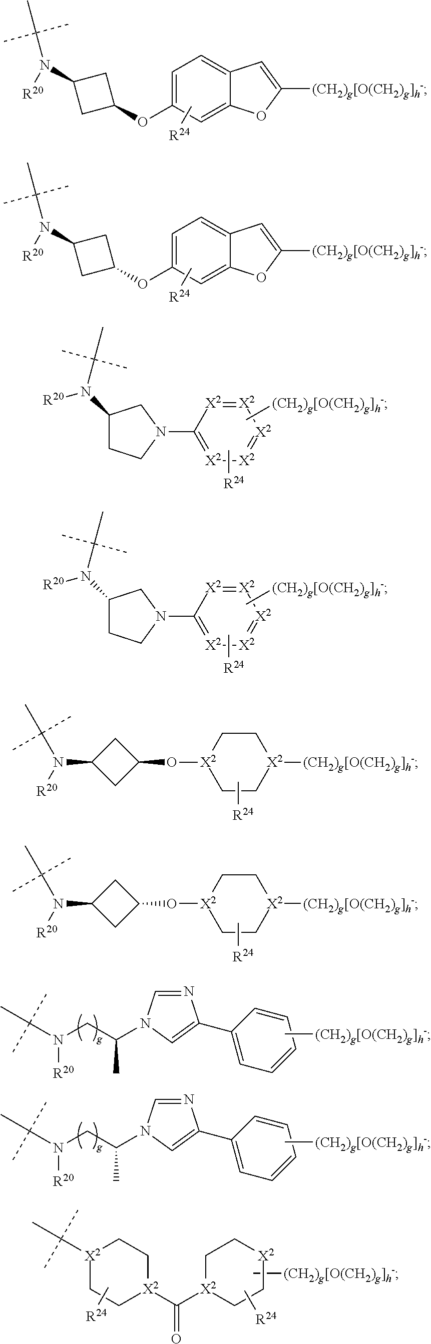 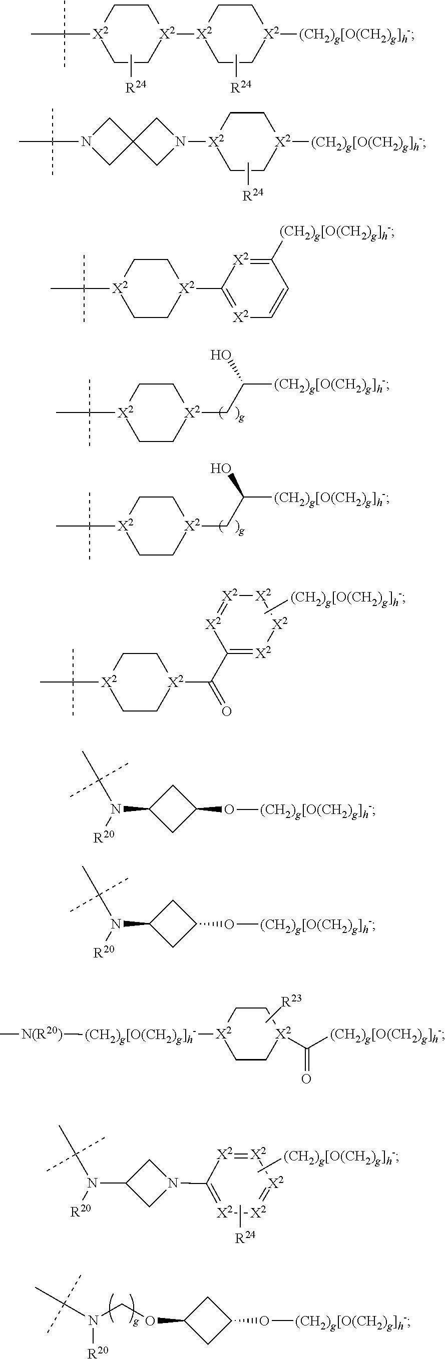 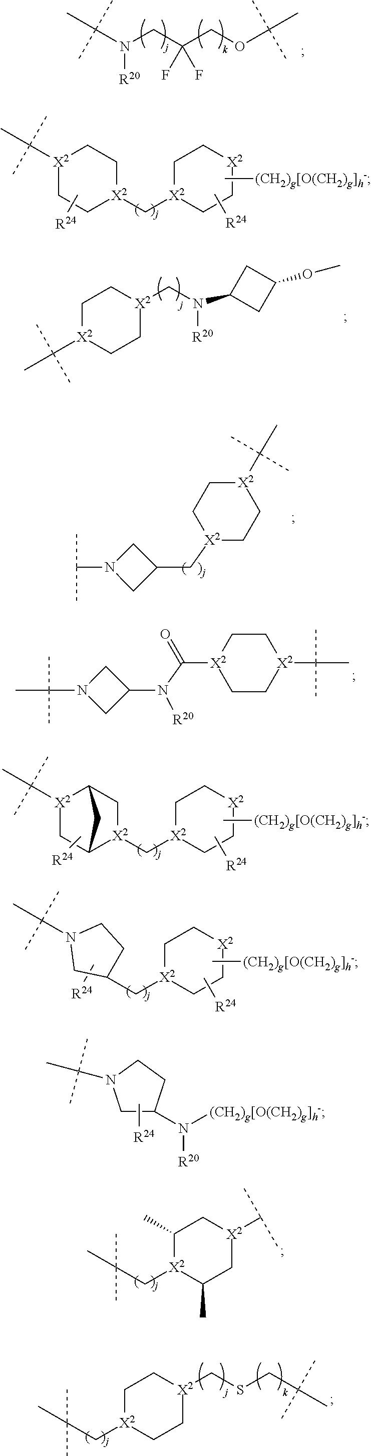 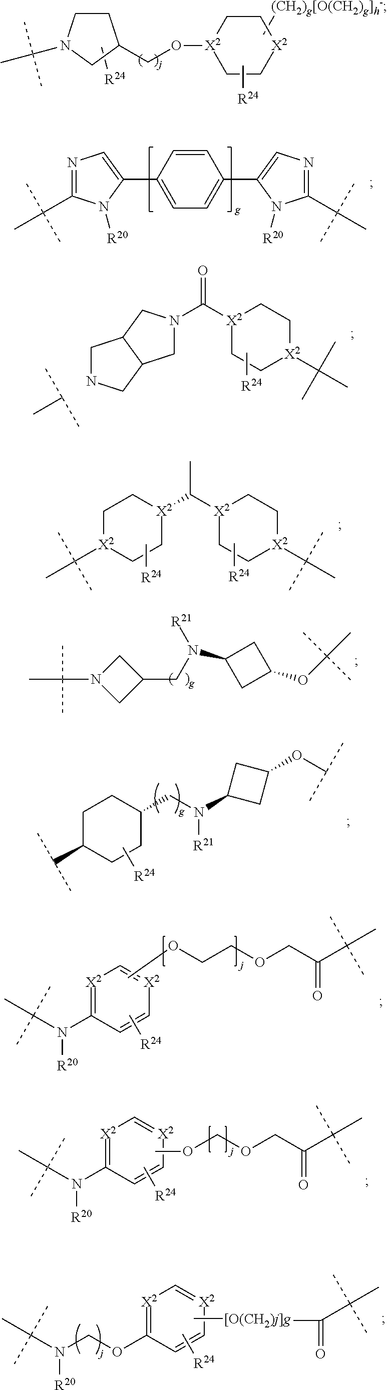 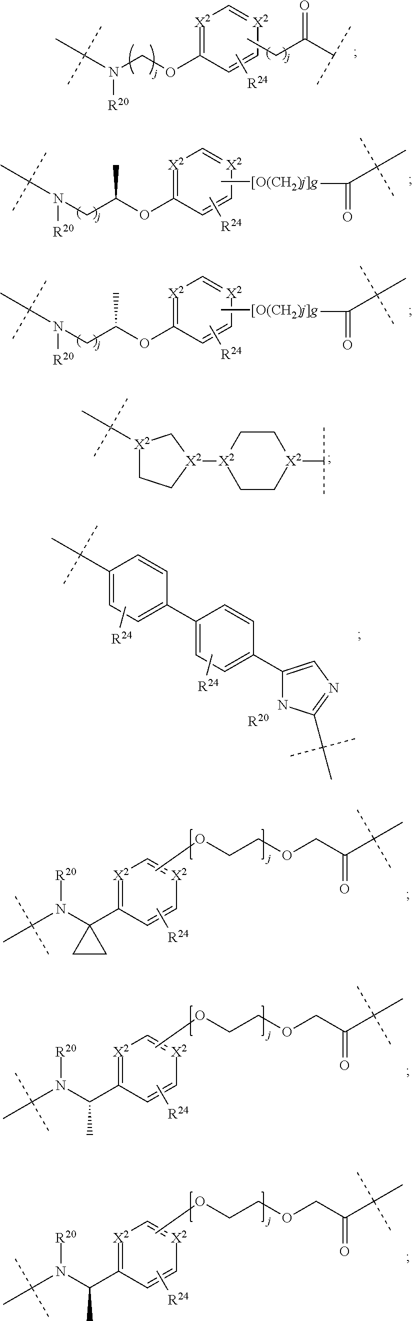 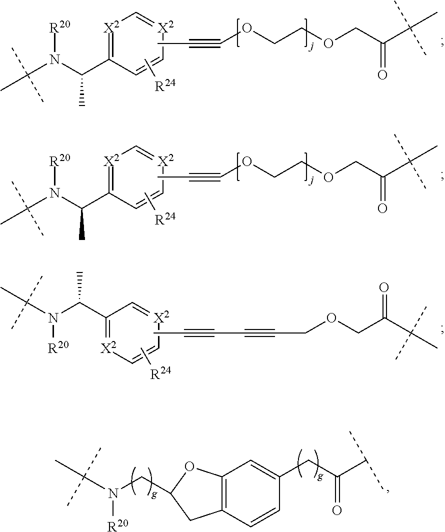
wherein
each R²⁰ is independently selected from hydrogen, C₁-C₄alkyl, and (C₃-C₆alkyl)C₀-C₂alkyl;
each R²¹ is independently selected from hydroxyl, halogen, C1-C₄alkyl, and C₁-C₄alkoxy;
R²³ is 0 to 4 substituents independently selected from halogen, hydroxyl, amino, C₁-C₂alkyl, and C₁-C₂alkoxy;
R²⁴ is 0 to 4 substituents independently selected from halogen, hydroxyl, amino, cyano, C₁-C₄alkyl, C₁-C₄alkoxy, C₁-C₂haloalkyl, and C₁-C₂haloalkoxy;
each R²⁵ and R²⁶ is independently selected from hydrogen and methyl, or R²⁵ and R²⁶ are joined to form a spiro attached C₃-C₅cycloalkyl group, or R²⁵ and R²⁶ can be taken together along with the carbon atom to which they are bound form a 3- to 8-membered monocyclic or bicyclic cycloalkyl ring;
each X¹ is independently selected from CH₂, NR²⁰, O, SO, SO₂, and S;
each X² is independently selected from CH and N;
each X³ is independently selected from CH₂, NR²⁰, O, SO, SO₂, and S;
each g is independently selected from 0, 1, 2, 3, 4, 5, 6, 7, or 8;
each h is independently selected from 0, 1, 2, 3, 4, 5, 6, 7, or 8;
each j is independently selected from 1, 2, 3, 4, 5, 6, 7, or 8; and
each k is independently selected from 1, 2, 3, 4, 5, 6, 7, or 8.

23. The bifunctional compound, or pharmaceutically acceptable salt thereof, of claim 22 wherein the [LINKER] is

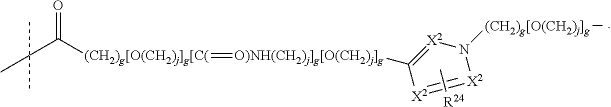

24. The bifunctional compound, or a pharmaceutically acceptable salt thereof, of any one of claims 1-21, wherein the [LINKER] is

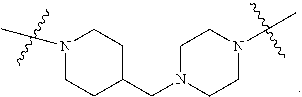 [SMILES: *N1CCC(CN2CCN(*)CC2)CC1]

25. The bifunctional compound, or a pharmaceutically acceptable salt thereof, of any one of claims 1-21, wherein the [LINKER] is a polyalkyleneglycol (e.g. polyethyleneglycol) containing linker having from 1 to 12 alkylene (e.g. ethylene) glycol residues.

26. The bifunctional compound, or a pharmaceutically acceptable salt thereof, of any one of claims 1-21, wherein the [LINKER] is a polypropylene glycol or polypropylene-co-polyethylene glycol linker containing any combination of the groups, 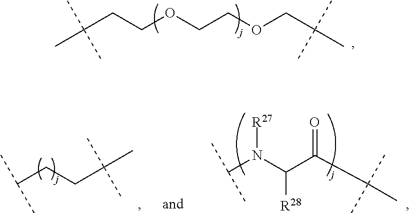
and optionally containing 1 group 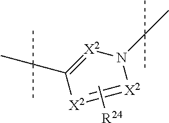
wherein
X² is independently chosen from N and CH;
R²⁴ is 0 to 4 substituents independently selected from halogen, hydroxyl, amino, cyano, C₁-C₄alkyl, C₁-C₄alkoxy, C₁-C₂haloalkyl, and C₁-C₂haloalkoxy;
R²⁷ is H, C₁-C₃ alkyl, C₁-C₃ alkanol, or forms a cyclic ring with R³ to form a pyrrolidine or hydroxypyrroline group;
R²⁸ is H or a side chain derived from a D- or L amino acid selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline, hydroxyproline, serine (methanol), threonine (ethanol, 1-hydroxyethane), tryptophan (methyleneindole), tyrosine (methylene phenol), or valine (isopropyl); and
j is 0, 1, 2, 3, 4, 5, 6, 7, 8, 9, 10, 11, 12, 13, 14, or 15.

27. The bifunctional compound, or a pharmaceutically acceptable salt thereof, of any one of claims 1-21, wherein the [LINKER] comprises: 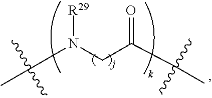
wherein
each R²⁹ is H or a C₁-C₃alkyl that is optionally substituted with one or two hydroxyl groups;
each j is independently 1, 2, 3, 4, 5, 6, 7, 8, 9, 10, 11, 12, 13, 14, or 15; and
k is an integer from 1 to 100.

28. The bifunctional compound, or a pharmaceutically acceptable salt thereof, of any one of claims 1-21, wherein the [LINKER] comprises: 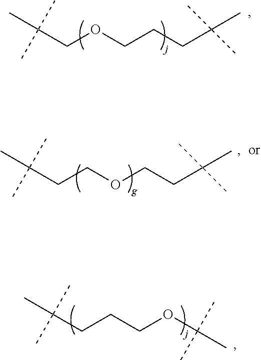
wherein
j is an integer from 1 to 25, 1 to 15, 1 to 12, 2 to 11, 2 to 10, 2 to 8, 2 to 6, 2 to 5, 2 to 4 or 2 to 3 (e.g. 1, 2, 3, 4, 5, 6, 7, or 8); and g is 0, 1, 2, 3, 4, 5, 6, 7, or 8.

29. The bifunctional compound, or a pharmaceutically acceptable salt thereof, of any one of claims 1-21, wherein the [LINKER] is:

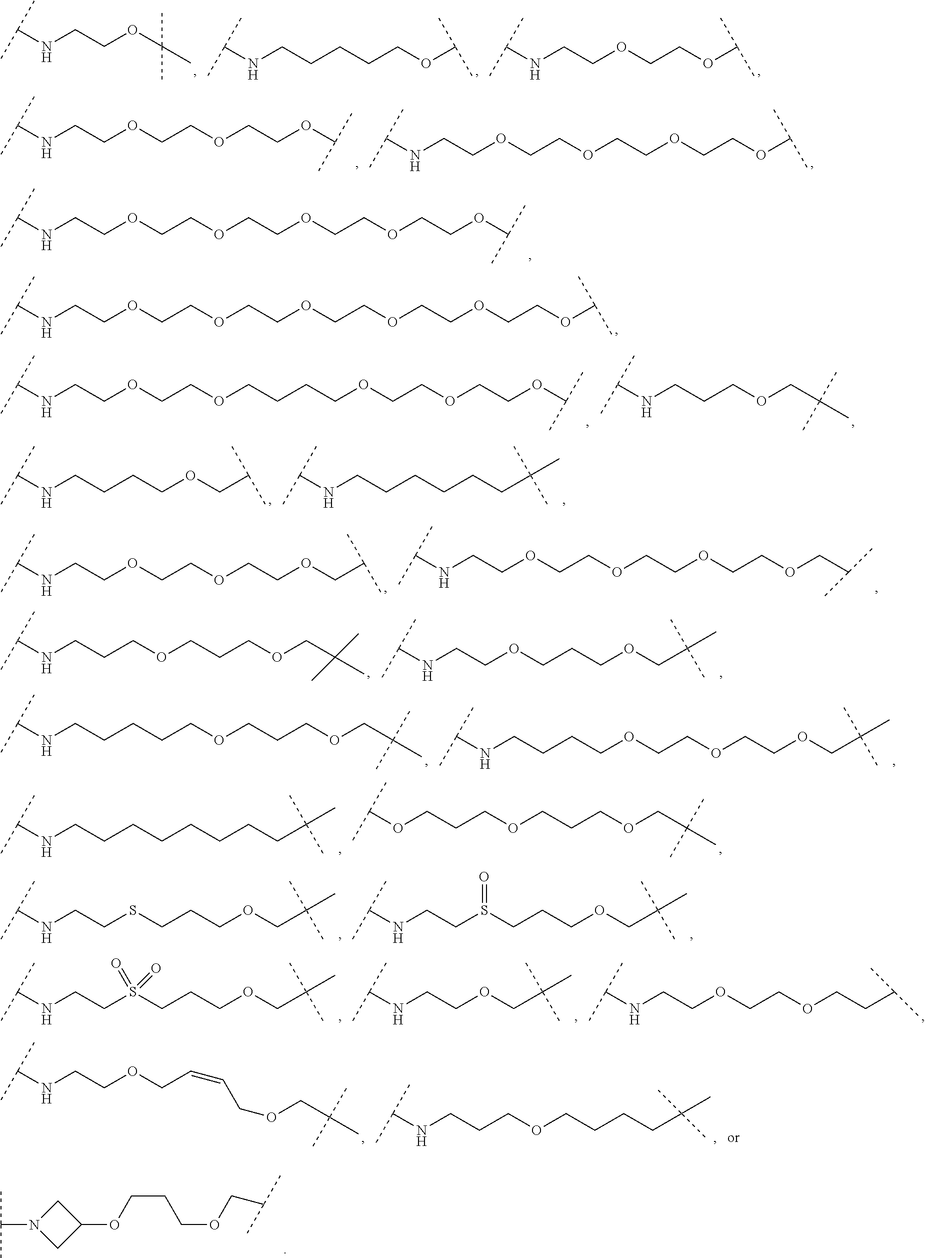

30. The bifunctional compound, or a pharmaceutically acceptable salt thereof, of any one of claims 1-21, where the [LINKER] is

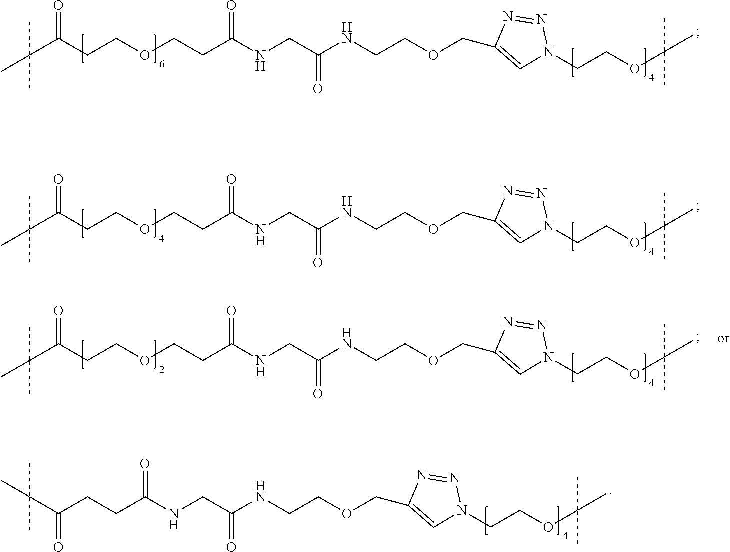

31. The bifunctional compound, or a pharmaceutically acceptable salt thereof, of any one of claims 1-30, wherein [CON] is a bond.

32. The bifunctional compound, or a pharmaceutically acceptable salt thereof, of any one of claims 1-21, wherein the [LINKER] is a C₁-C₅₀ alkylene group (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkylene), wherein:
the compound comprises one to fourteen —C₁-C₂₅ alkylene-[TNFABM] covalently bound to the [LINKER];
the compound comprises one to fourteen —C₁-C₂₅ alkylene-[ASPGPRM] covalently bound to the [LINKER];
each C₁-C₂₅alkylene group of the [LINKER] optionally contains 0 or 1 or more double or triple bonds, each CH₂ group of the [LINKER] is optionally replaced with a group independently selected from O, S, SO, SO₂, NH, CH═CH, C≡C, 5-7 membered heterocycle, and 3-8 membered carbocycle, and is optionally bound to a [CON]; and
the [LINKER] is optionally substituted with one or more (e.g., 1, 2, 3, 4, 5, 6, 7, 8, 9, 10 or more) groups independently selected from halogen, hydroxyl, cyano, oxo (═O), amino, imino (═NH), —COOH, C₁-C₄alkyl, C₁-C₄haloalkyl, and C₁-C₄alkoxy.

33. The bifunctional compound, or a pharmaceutically acceptable salt thereof, of claim 32, wherein the compound comprises a[LINKER]—[CON] group in either orientation, which [LINKER]—[CON] group includes at least one [CON] comprising: 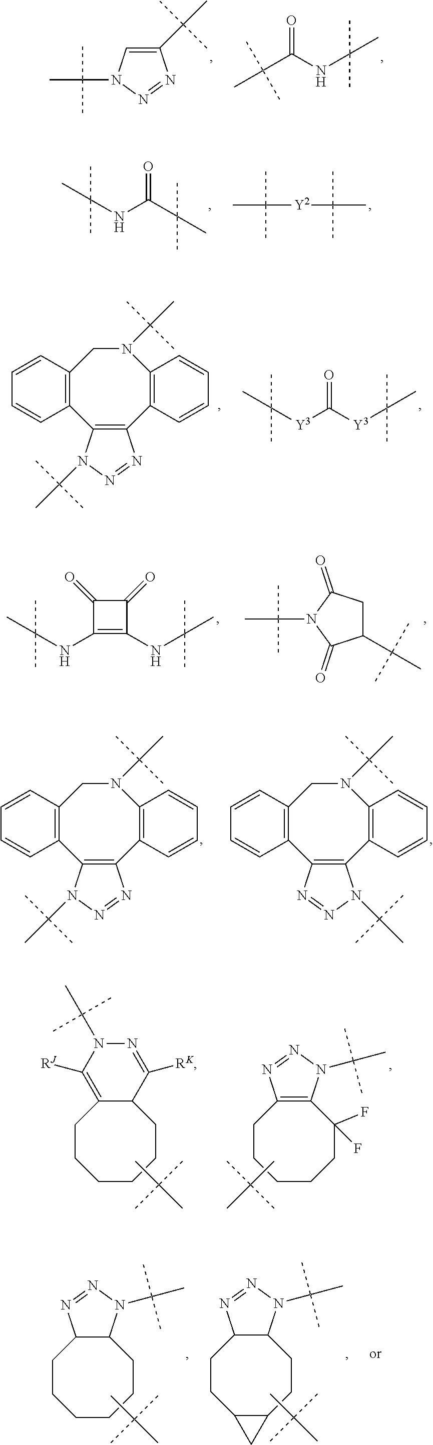 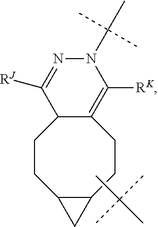
wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure: 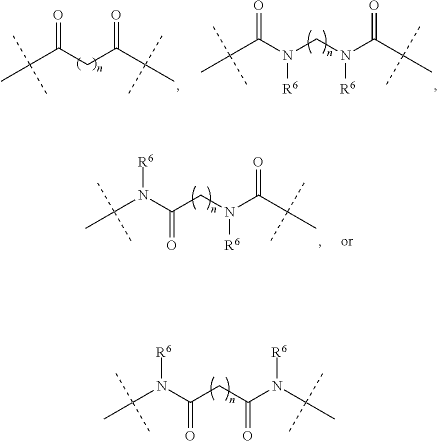
(e.g., one end is for attachment to [TNFABM], [LINKER], or [ASGPRBM]), wherein
each R⁶ is independently H or a C₁-C₃ alkyl group (preferably H);
n is an integer from 1 to 10 (n is preferably 1, 2, 3, 4, 5, or 6).

34. The bifunctional compound, or a pharmaceutically acceptable salt thereof, of any one of claims 1-33, wherein
(a) the compound comprises a [CON]-[LINKER] group in either orientation of the formula [C1]—[PAG], wherein
each [PAG] is a polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein the [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) 0 atoms of the [PAG] is optionally replaced with NH or NC₁-C₃alkyl; and
each [C1] is independently selected from: 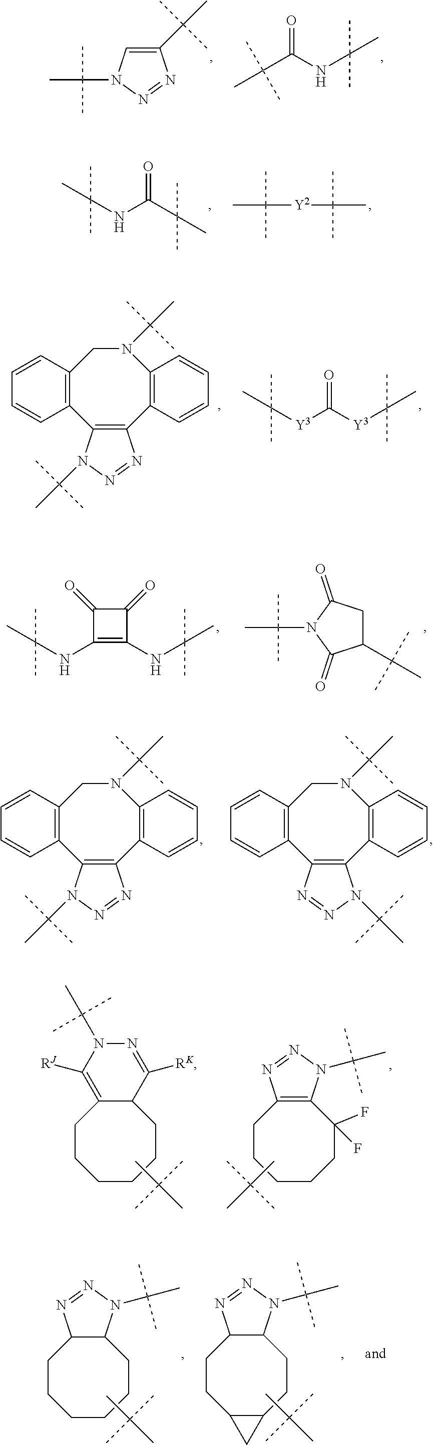 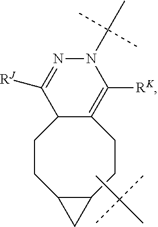
wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure: 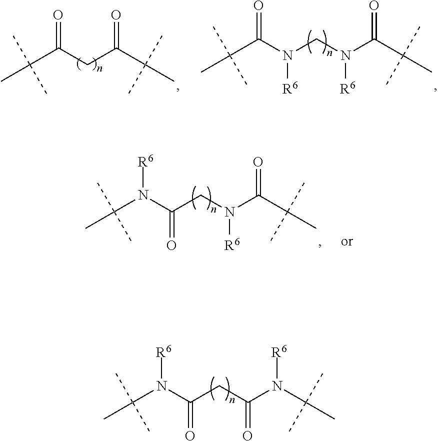
(e.g., one end is for attachment to the [TNFABM], [PAG], [LINKER], or [ASGPRBM]), wherein each R⁶ are independently H or a C₁-C₃ alkyl group (preferably H); and n is an integer from 1 to 10, or 1 to 8, or 1 to 6 (e.g. 1, 2, 3, 4, 5, or 6);
(b) the [CON]-[LINKER] group has the formula —CH₂(CH₂O—CH₂—[C1]—[PAG])₁, —CH₁(CH₂O—CH₂—[C1]—[PAG])₂, or —C(CH₂O—CH₂—[C1]—[PAG])₃, wherein
each [PAG] is an independently chosen polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein the [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) 0 atoms of the [PAG] is optionally replaced with NH or NC₁-C₃alkyl; and
each [C1] is independently selected from: 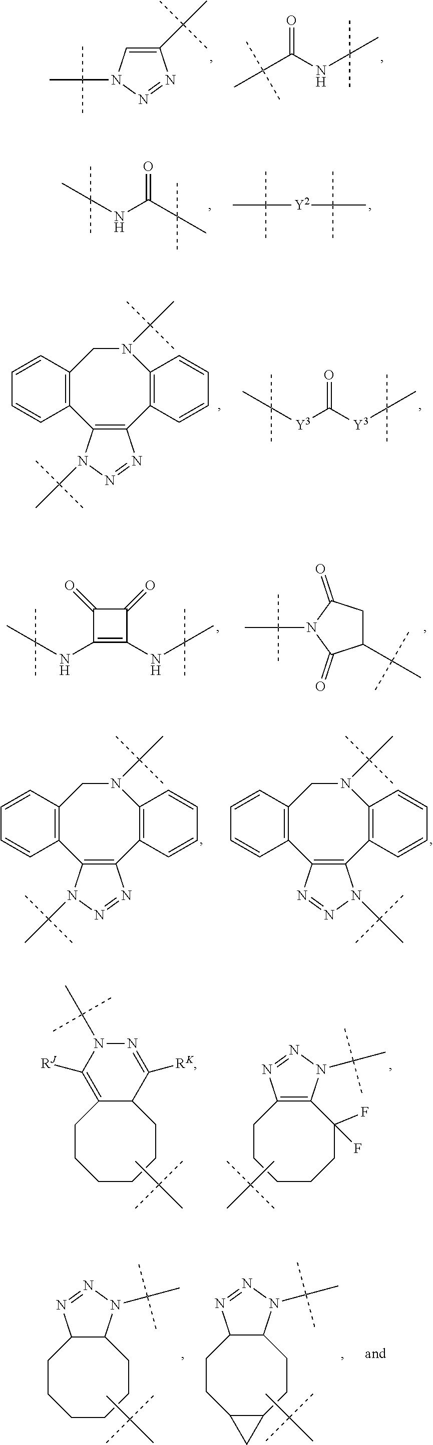 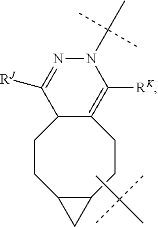
wherein
(c) the [CON] is: 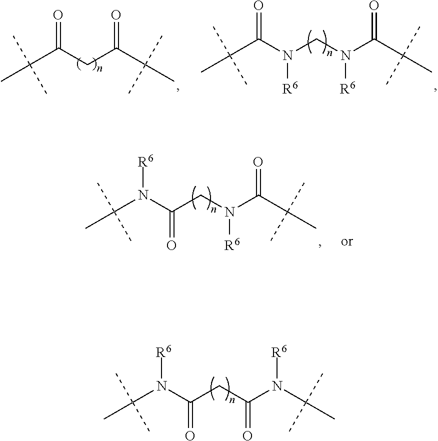
wherein
(d) the [CON] has formula: 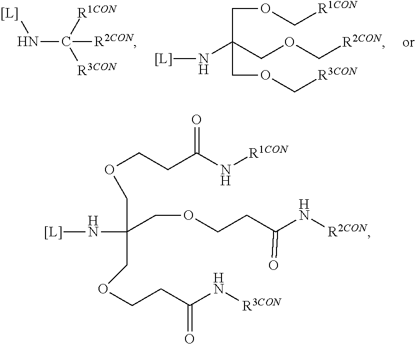
wherein
[L] represent the point of attachment to a first [LINKER]; and
R^{1CON}, R^{2CON}, and R^{3CON} are each independently H, a bond, or a chemical moiety, wherein the bond and the chemical moiety are covalently attached to an [ASPGPRM] or a second, third, or fourth independently chosen [LINKER] and at least one of R^{1CON}, R^{2CON}, and R^{3CON} least one of R^{1CON}, R²CON and R³CON is not H.

35. The bifunctional compound, or a pharmaceutically acceptable salt thereof, of claim 34, wherein
(a) one or more (e.g., 1, 2, or 3) of R^{1CON}, R^{2CON}, and R^{3CON} includes a group selected from: 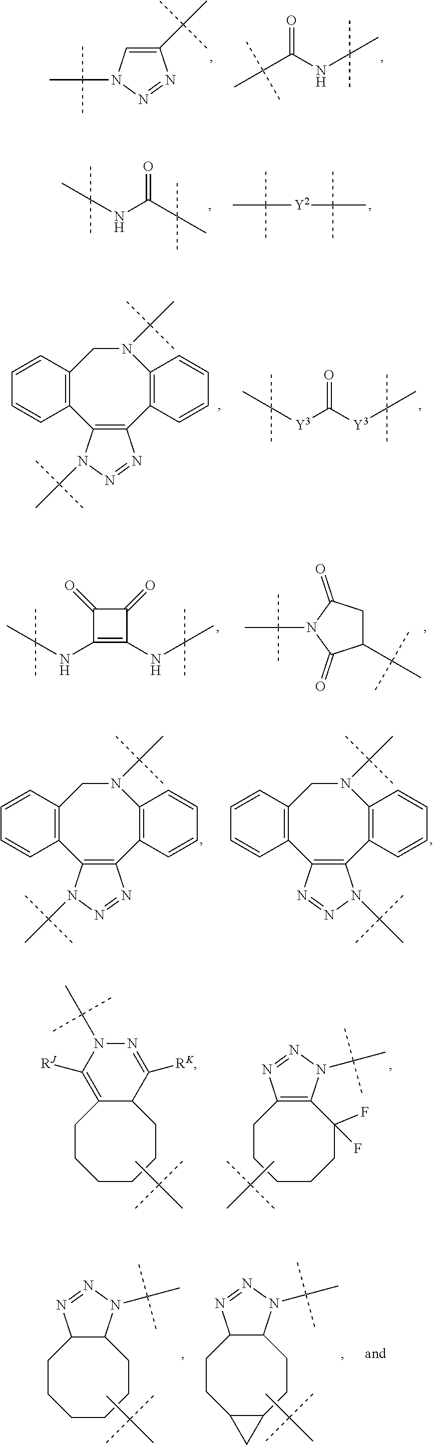 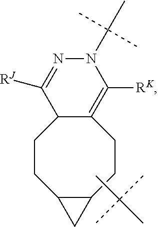
wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure: 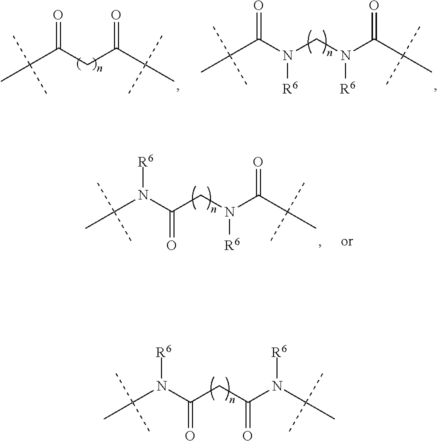
(e.g., one end is for attachment to [TNFABM], [LINKER], or [ASGPRBM]), wherein each R⁶ is independently H or C₁-C₃ alkyl (preferably H); and n is independently an integer from n to 10, or 1 to 8, 8 or 1 to 7 (e.g. 1, 2, 3, 4, 5, or 6).

36. The bifunctional compound or salt thereof, of claim 34, wherein
[CON] is 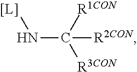
wherein
(a) at least one of R^{1CON}, R^{2CON}, and R^{3CON} is not H, and each of R^{1CON}, R^{2CON}, and R^{3CON} is independently selected from: H, bond, 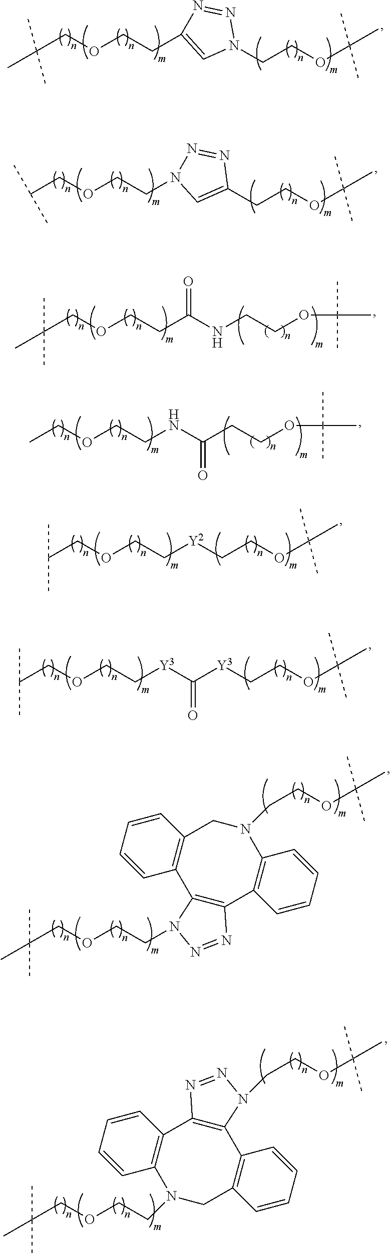 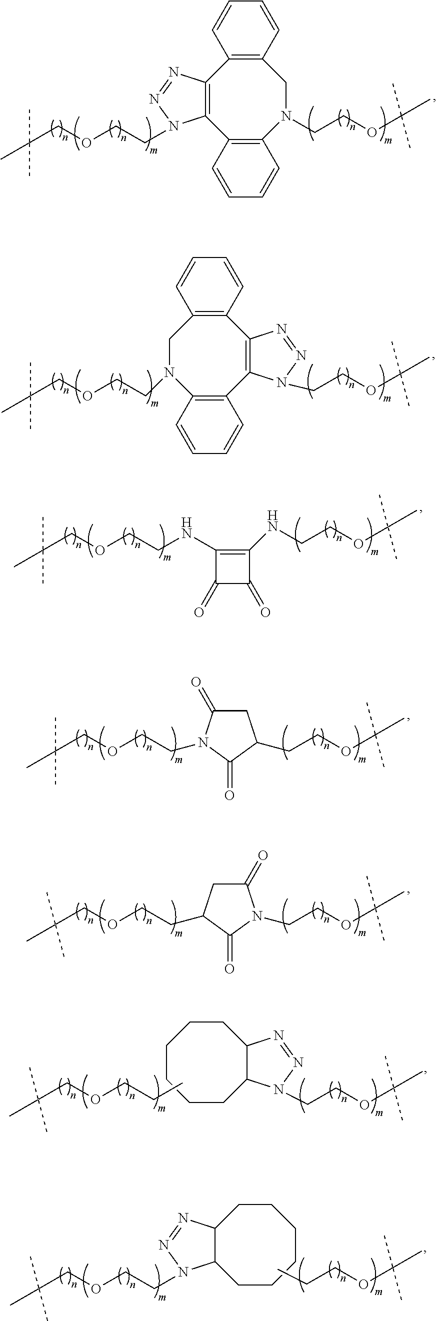 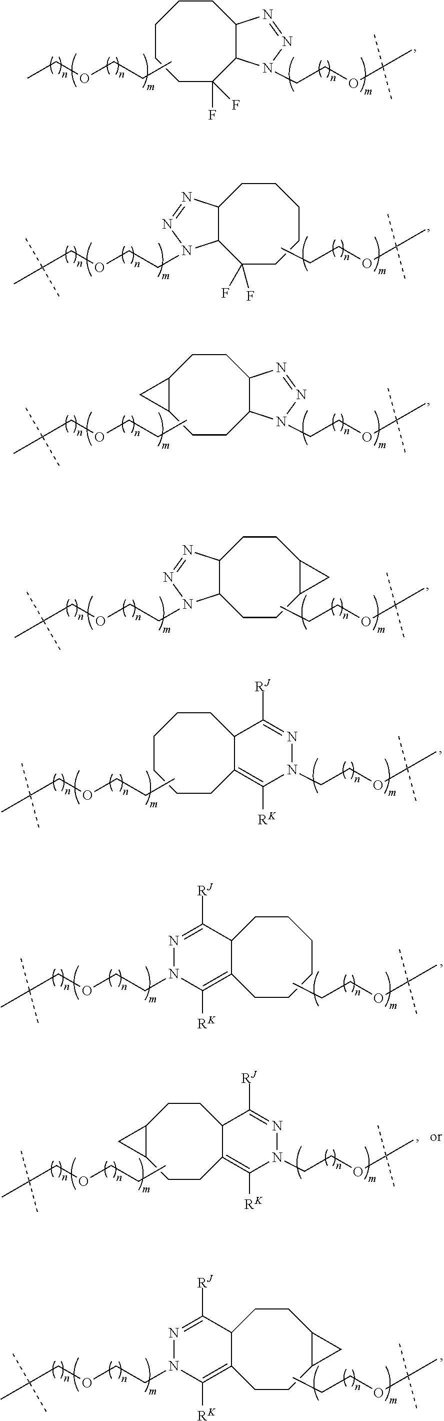
wherein
each n is independently 1, 2, 3, 4, 5, 6, 7, 8, 9, or 10;
each m is independently 0, 1, 2, 3, 4, 5, 6, 7, 8, 9, or 10;
each Y² is independently CH₂, O, S, NR⁷, C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure: 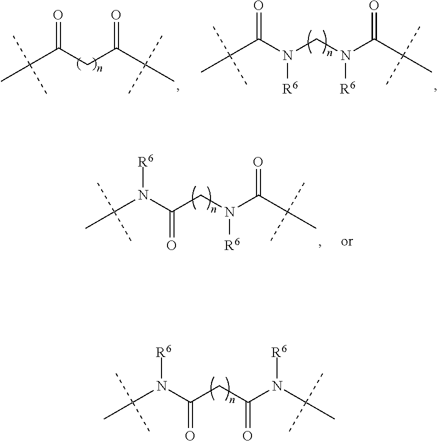
(e.g., one end is for attachment to [TNFABM], [LINKER], or [ASGPRBM]), wherein each R⁶ is independently H or a C₁-C₃ alkyl group (preferably H) n is an integer from 1 to 10 (is preferably and integer from 0 to 8, or 0 to 7, or 0, 1, 2, 3, 4, 5, or 6); or
(b) R^{1CON}, R^{2CON}, and R^{3CON} are each independently H, —(CH₂)ᵣ—, —(CH₂)ₛC(O)_{XA}(NR⁸)_{XA}—(CH₂)ₛ—, —(CH₂)ₛ(NR⁴)_{XA}C(O)_{XA}—(CH₂)ₛ—, and —(CH₂)ₛO—(CH₂)ᵣ—C(O)NR⁸—, with the proviso that R^{1CON}, R^{2CON}, and R^{3CON} are not simultaneously H, wherein
each r is independently 1, 2, 3, or 4 (preferably 1 or 2);
each s is independently 0, 1, 2, 3, or 4 (preferably 0, 1 or 2);
each XA is independently 0 or 1; and
each R⁸ is independently H, C₁-C₃ alkyl, C₁-C₃ alkanol, or —C(O)(C₁-C₃), with the proviso that s and
XA in a moiety are not all simultaneously 0.

37. The bifunctional compound, or a pharmaceutically acceptable salt thereof, of any one of claims 1-21, wherein
(a) the [LINKER]—[CON] has the formula: [PEG]-[C1]—[PEG],
wherein each [PEG] is independently a polyethylene glycol group containing from 1, 2, 3, 4, 5, 6, 7, 8, 9, 10, 11, or 12 ethylene glycol residues, and [C1] is a triazole group 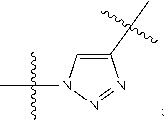 [SMILES: *c1cn(*)nn1]
(b) the [LINKER]—[CON] has the formula: [PAG]-[C1]—[PAG], wherein
each [PAG] is independently a polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein each [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) O atoms of each [PAG] is optionally replaced with NH or NC₁-C₃alkyl; and
[C1] is selected from: 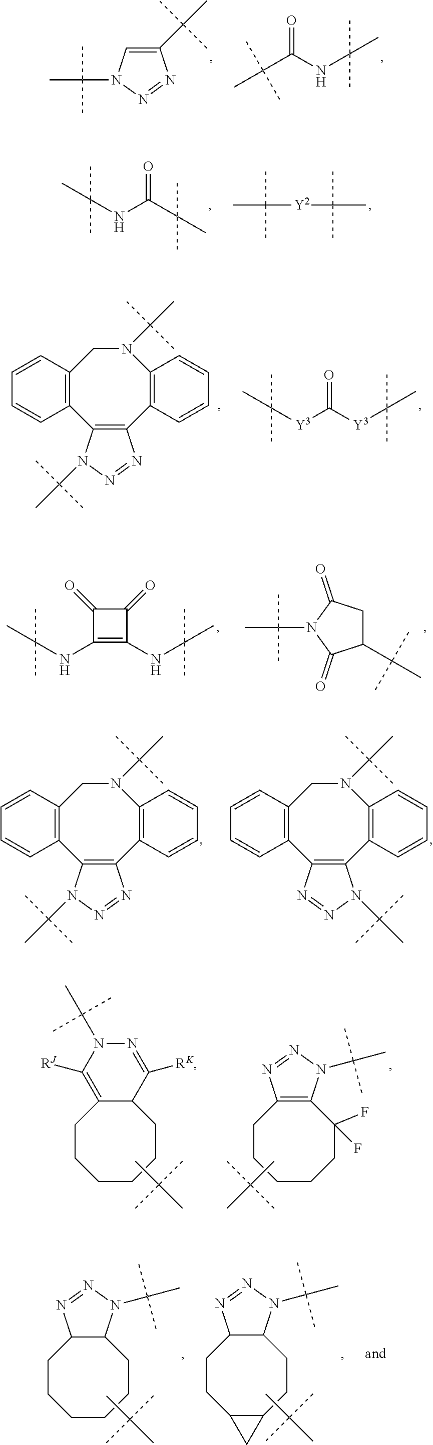 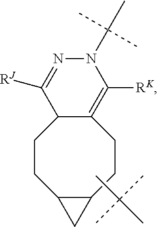
wherein
each Y² is independently CH₂, O, S, NR⁷ (e.g. NC₁-C₃alkyl)), C(O), S(O), S(O)₂, —S(O)₂^{o}, —OS(O)₂, or OS(O)₂^{o};
each Y³ is independently O, S, or NR⁷;
each R⁷ is independently H, C₁-C₃ alkyl, C₁-C₃ alkanoyl, or —C(0)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure: 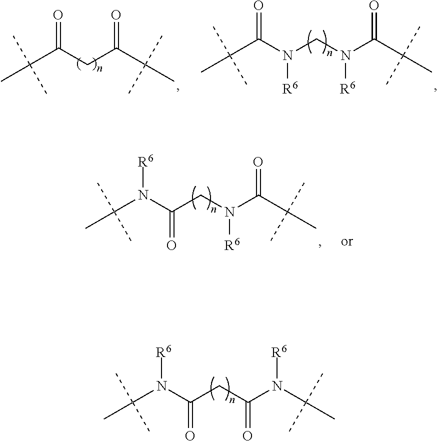
(e.g., one end is for attachment to [TNFABM], [LINKER], or [ASGPRBM]), wherein each R⁶ is independently H or a C₁-C₃ alkyl group (preferably H); and z is independently an integer from 0 to 8 or 1 to 7 (e.g. 1, 2, 3, 4, 5, or 6); or
(c) the [LINKER]—[CON] has the formula [PAG]-CH₂(CH₂O—CH₂—[C1]—[PAG])₁, [PAG]-CH₁(CH₂O—CH₂—[C1]—[PAG])₂, or [PAG]-C(CH₂O—CH₂—[C1]—[PAG])₃, wherein each [PAG] is a polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein the [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) 0 atoms of the [PAG] is optionally replaced with NH or NC₁-C₃alkyl; and
each [C1] is independently selected from: 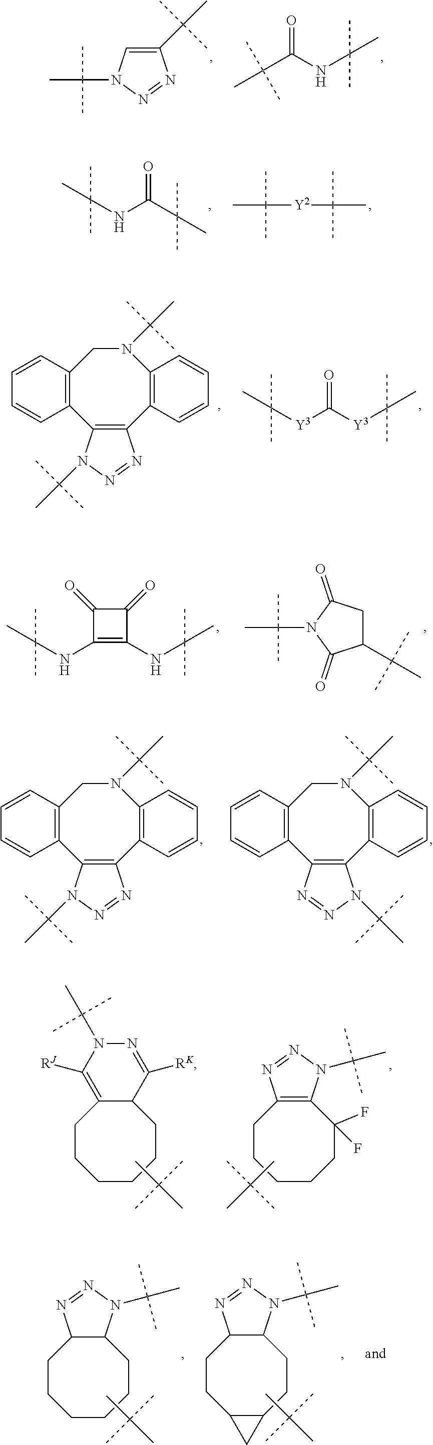 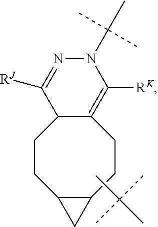
wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure: 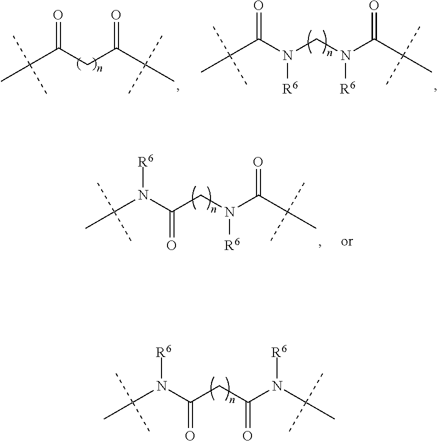
(e.g., one end is for attachment to [TNFABM], [LINKER], or [ASGPRBM])

38. The bifunctional compound, or salt form thereof, of any one of claims 1-30, or a salt thereof wherein the bifunctional compound contains at least one [CON]-[LINKER]₃-[ASPGPRM]₃ group selected from (a) and (b) or at least one [CON]-[LINKER]-[ASGPRM] group (c):
(a) 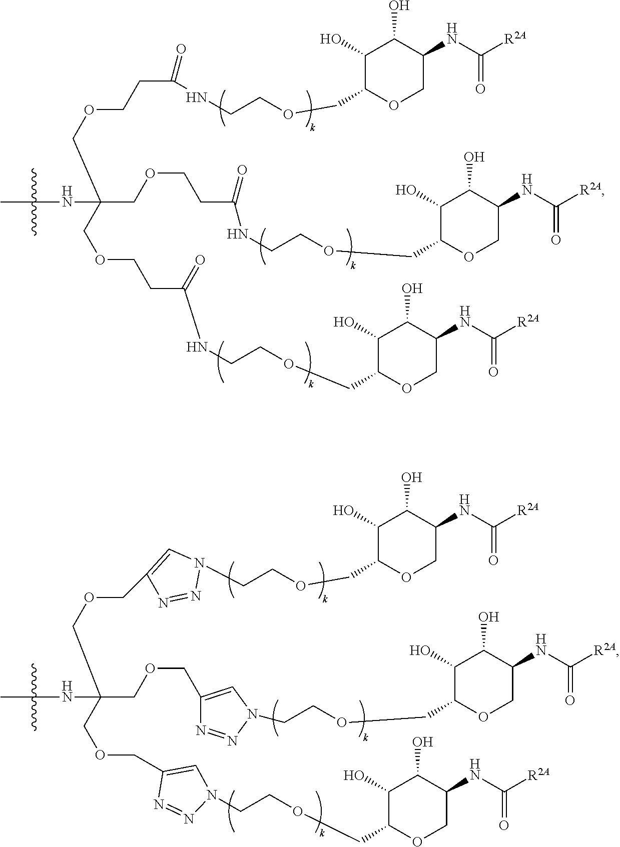 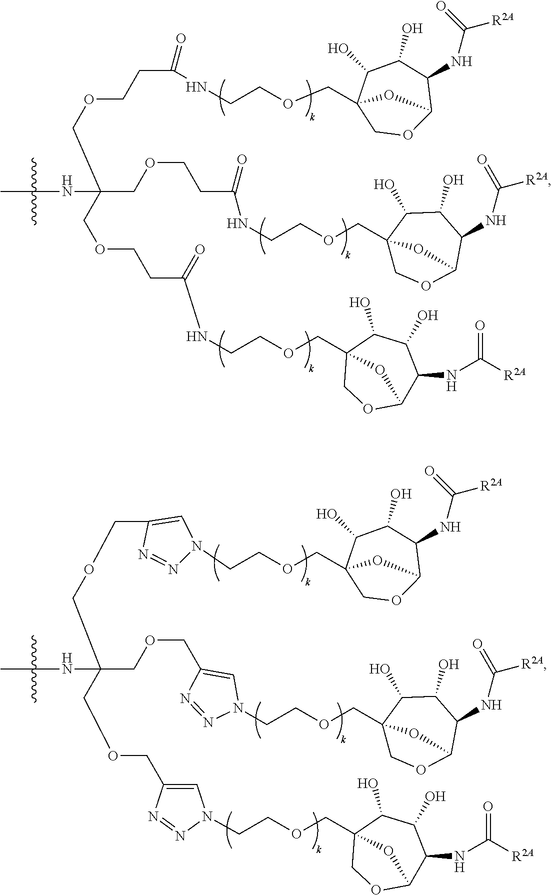 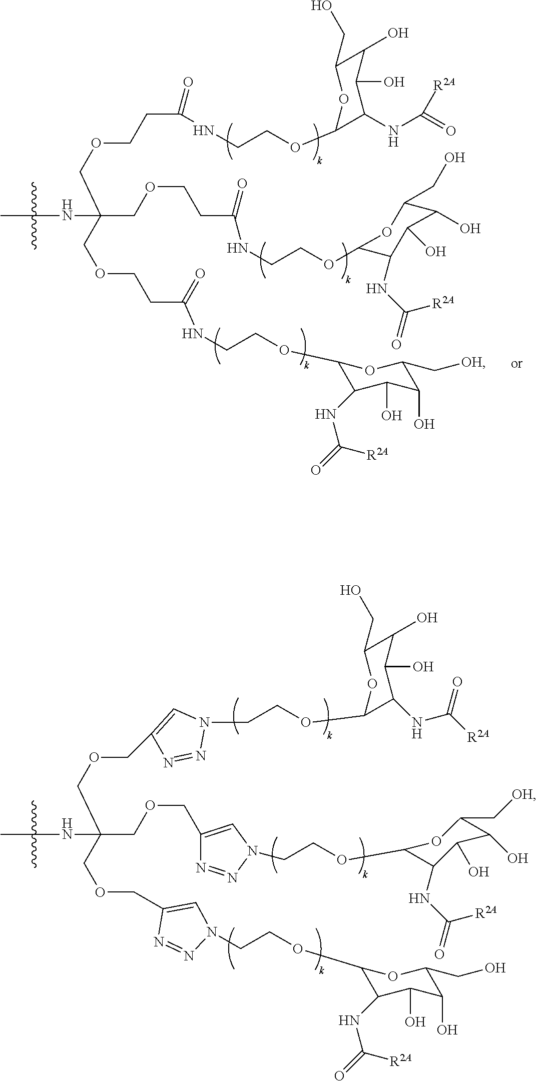
wherein each k is independently 0, 1, 2, 3, 4, 5, 6, 7, 8, 9, or 10 (preferably, 0, 1, 2, 3, 4, or 5), and each R^{2A} is independently a C₁-C₄alkyl (e.g., C₁-C₂alkyl) that is optionally substituted with 1 to 5 (e.g., 1, 2, 3, 4, or 5) independently selected halogen atoms; or
(b) 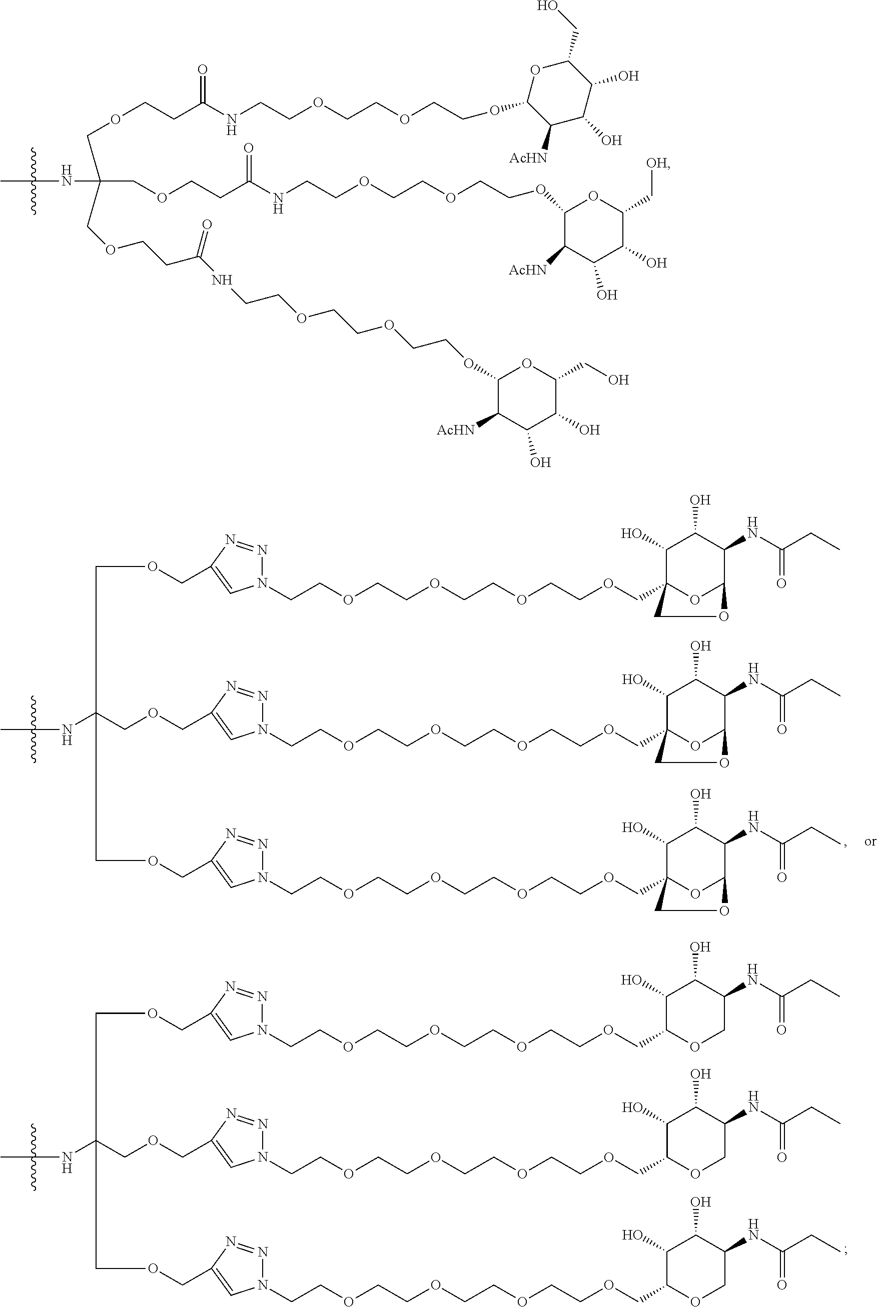
or
(c)

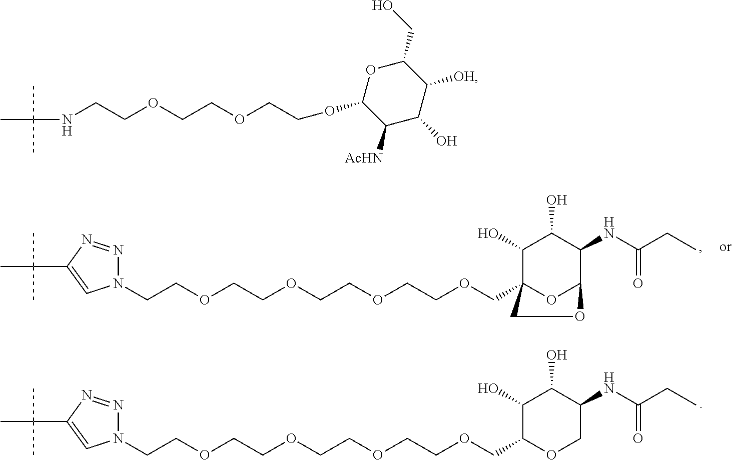

39. A bifunctional Tumor Necrosis Factor Alpha (TNF-α) binding compound of the formula: 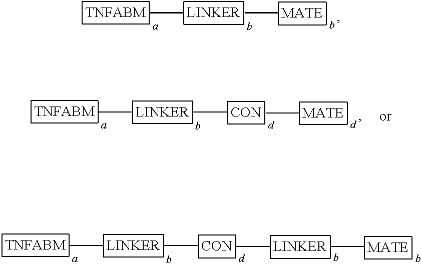
wherein,
[TNFABM] is an Tumor Necrosis Factor Alpha (TNF-α) Binding Moiety that is capable of binding to TNF-α
[MATE] is Fc binding reagent comprising an Fc binding moiety [ABM] and a reactive group [RG] capable of covalently and specifically binding to a Lys in the Fc domain of an TNF-α antibody and releasing the [ABM];
[LINKER] is an optional chemical moiety that is covalently attached to one or more [TNFABM] groups and either one or more [CON] groups or a [MATE] groups;
[CON] is an optional chemical connecting moiety that is covalently attached to one or more [MATE] groups and one or more [LINKER] groups;
a is an integer from 1 to 15 (e.g. 1, 2, 3, 4, 5, 6, 7, 8, 9, 10, 11, 12, 13, 14, or 15);
b is an integer, independently selected at each occurrence from 0 to 15 (e.g. 0, 1, 2, 3, 4, 5, 6, 7, 8, 9, 10, 11, 12, 13, 14, or 15), preferably 0 or 1;
and
d is an integer from 0 to 15 (e.g. 0, 1, 2, 3, 4, 5, 6, 7, 8, 9, 10, 11, 12, 13, 14, or 15, preferably 0, 1, 2, or 3); or a pharmaceutically acceptable salt thereof.

40. The bifunctional compound of claim 53, or a pharmaceutically acceptable salt thereof, wherein the [RG] covalently and specifically binds to a Lys 246 or Lys 248 of an IgG1 heavy chain of the Fc domain, K251 or K253 of an IgG2 heavy chain, or K239 or K241 of an IgG4 heavy chain, of an TNF-α antibody.

41. The bifunctional compound of claim 39 or 40, or a pharmaceutically acceptable salt thereof, wherein the [RG] covalently and specifically binds to a Lys 246 or Lys 248 of an IgG1 heavy chain of the Fc domain.

42. The bifunctional compound, or a pharmaceutically acceptable salt thereof of any of claims 39-41, wherein at least one of
a is 1, 2 or 3;
b is 1, 2 or 3; or
d is 1, 2 or 3.

43. The bifunctional compound of claim 39 or 40, or a pharmaceutically acceptable salt thereof, wherein the [MATE] is

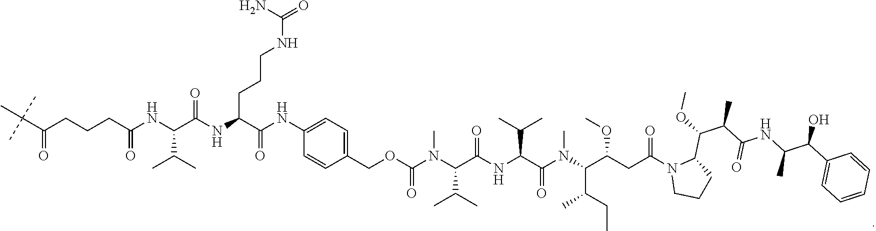

44. The bifunctional compound of claim 39 or 40, or a pharmaceutically acceptable salt thereof, wherein the [RG] is one or more of:

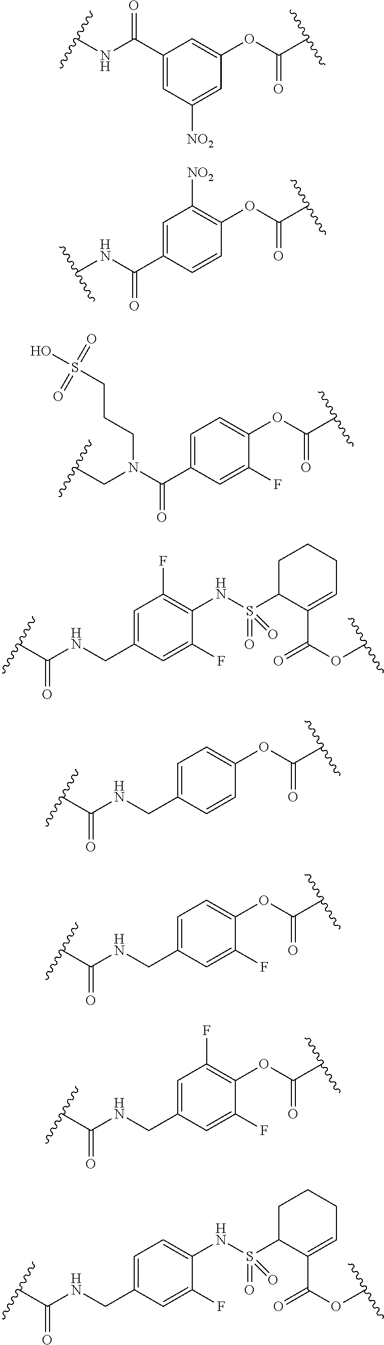

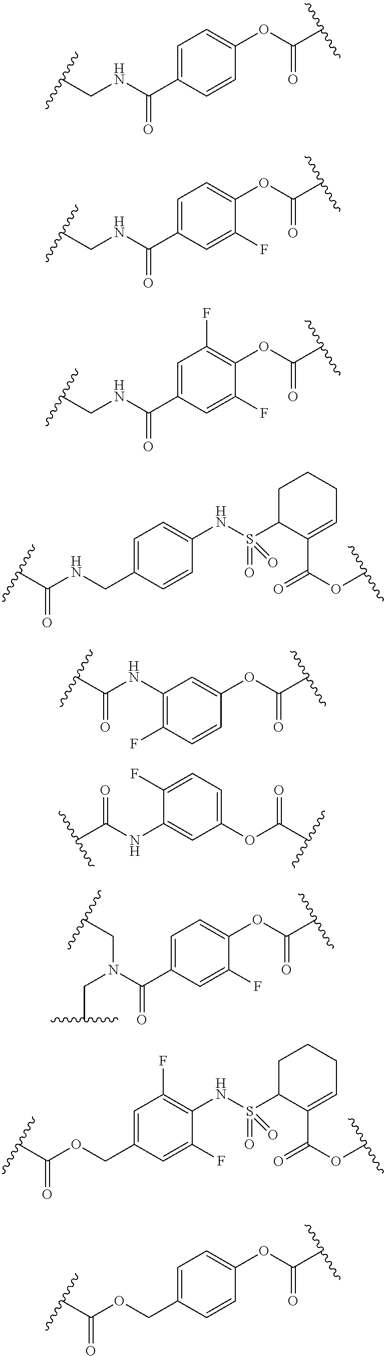

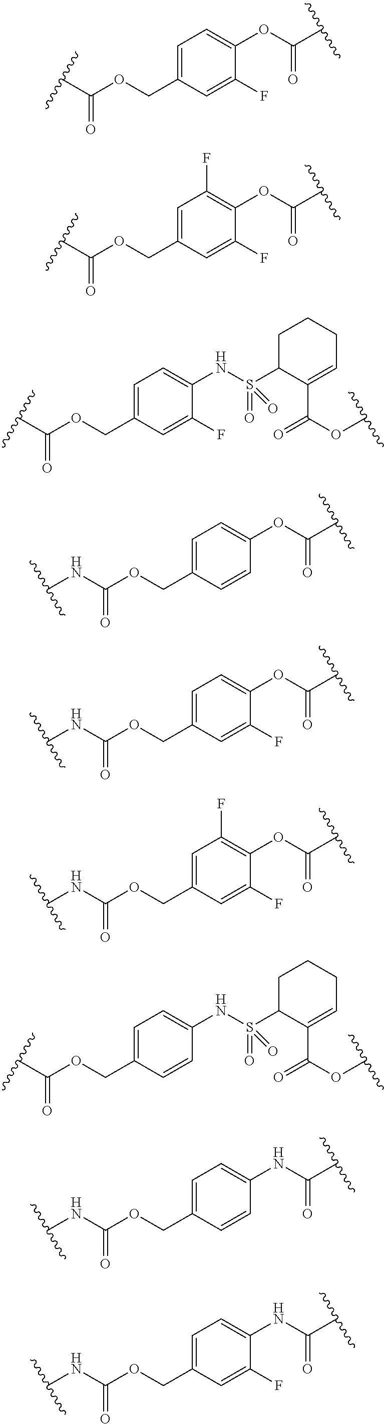

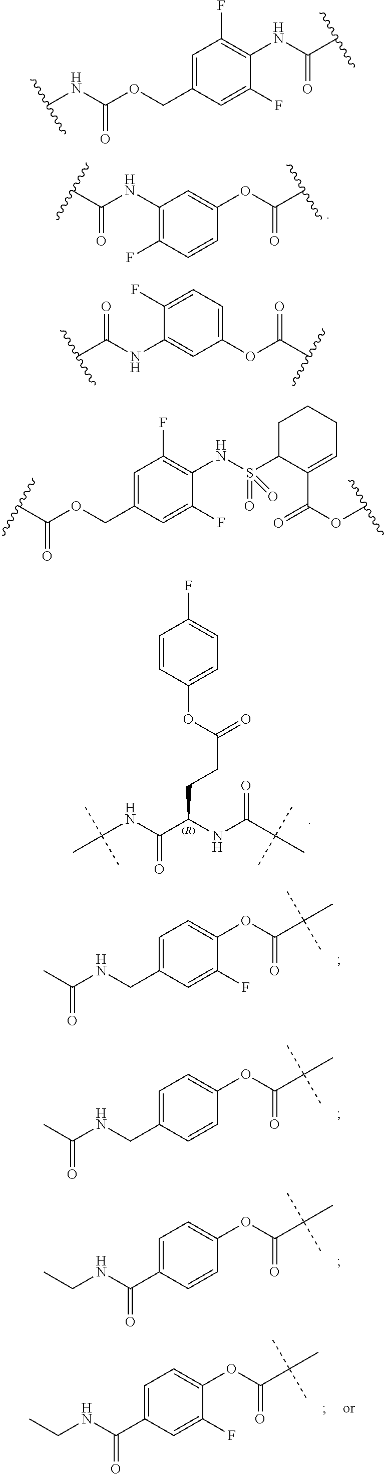

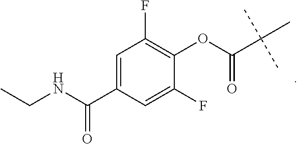

45. The bifunctional compound of claim 39 or 40, or a pharmaceutically acceptable salt thereof, wherein the [ABM] is

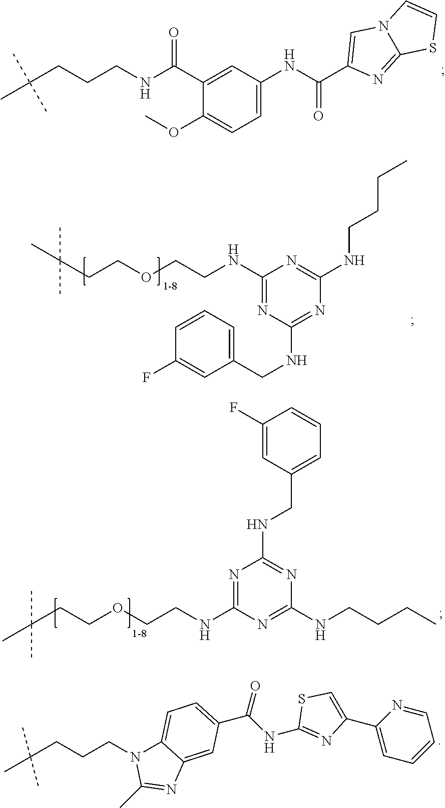

46. The bifunctional compound of claim 39 or 40, or a pharmaceutically acceptable salt thereof, wherein the [MATE] is

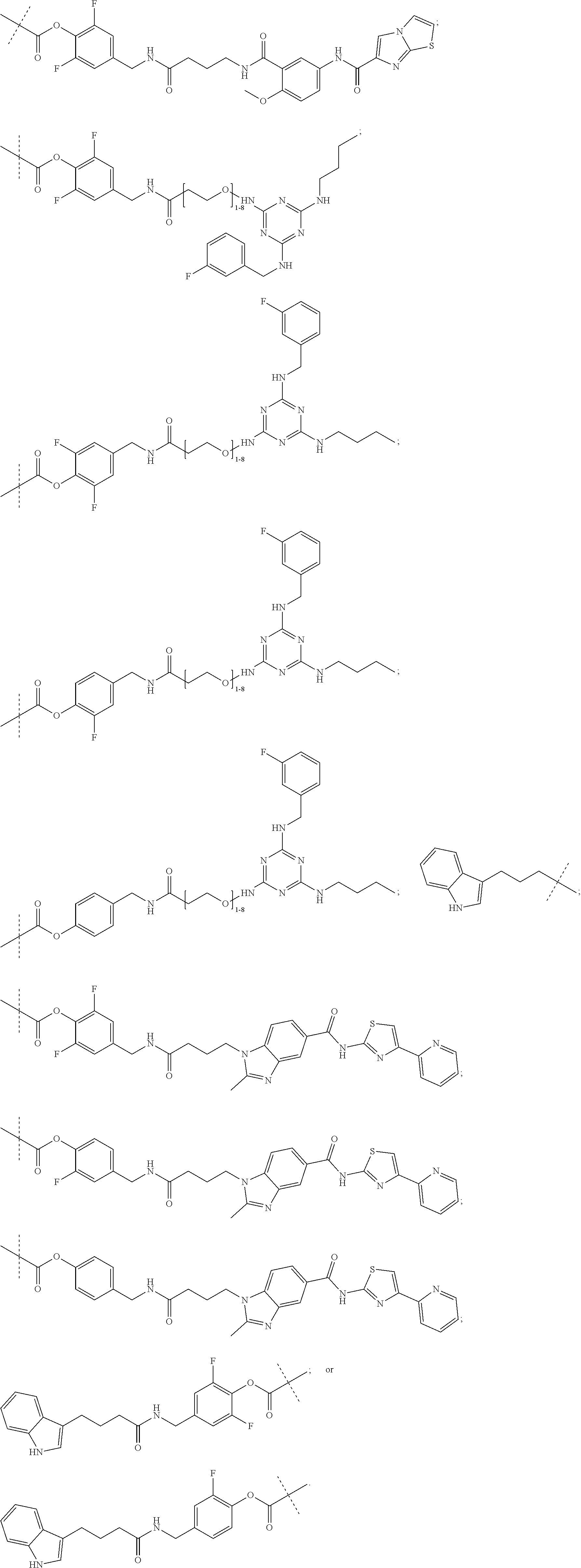

47. The bifunctional compound, or a pharmaceutically acceptable salt thereof, of any one of claims 1-46, where the [TNFABM] is

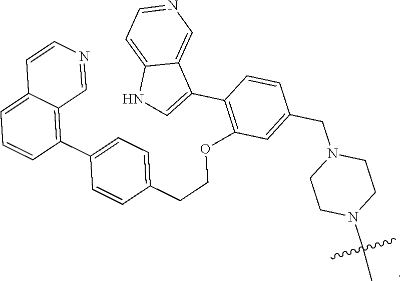 [SMILES: *N1CCN(Cc2ccc(-c3c[nH]c4ccncc34)c(OCCc3ccc(-c4cccc5ccncc45)cc3)c2)CC1]

48. The bifunctional compound, or a pharmaceutically acceptable salt thereof, of any one of claims 1-46, where the [TNFABM] is

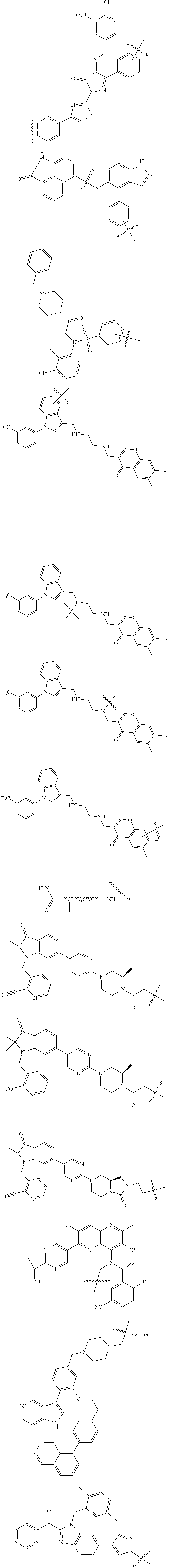

49. The bifunctional compound, or a pharmaceutically acceptable salt thereof, of any one of claims 39-48, wherein the [LINKER] is selected from: 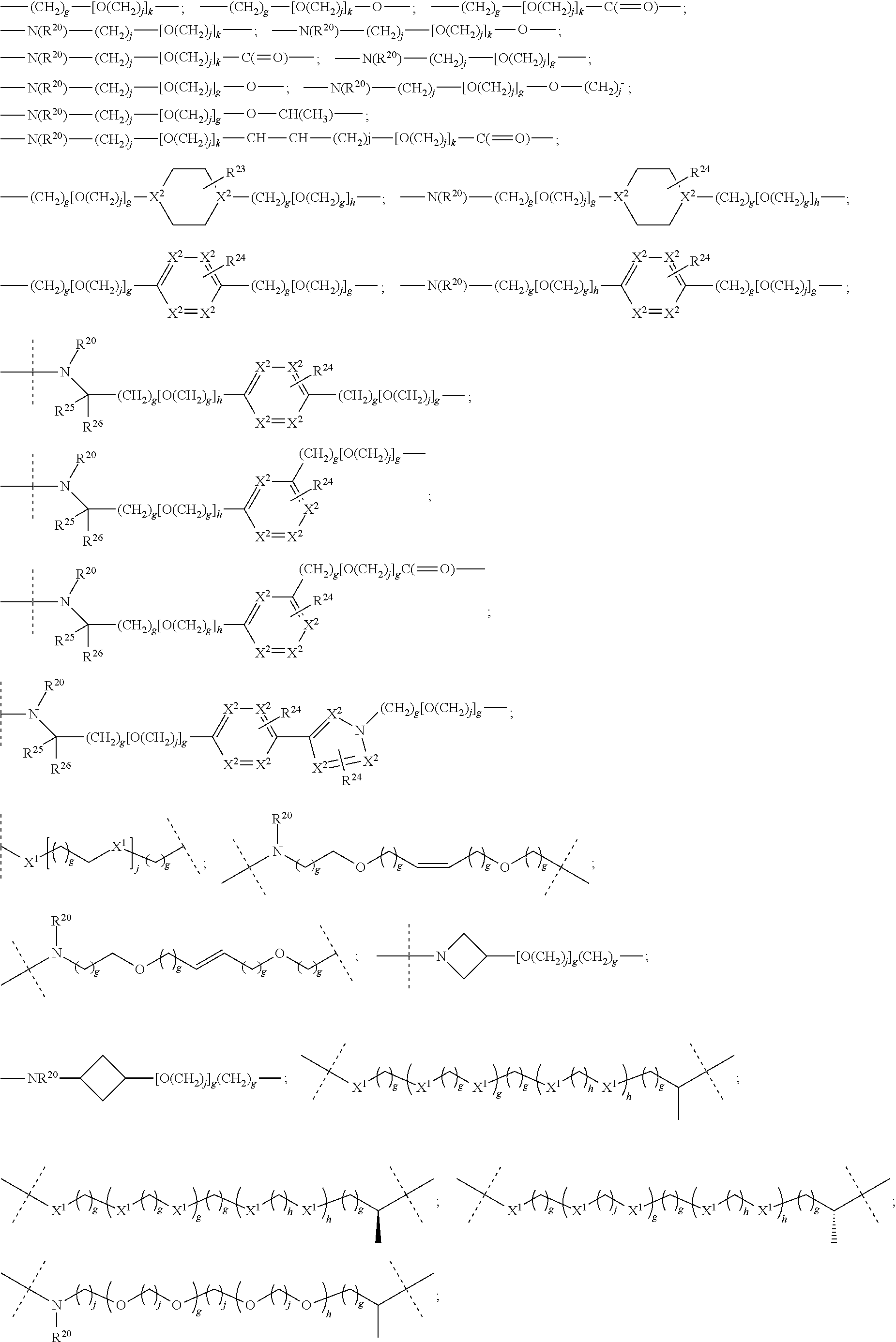 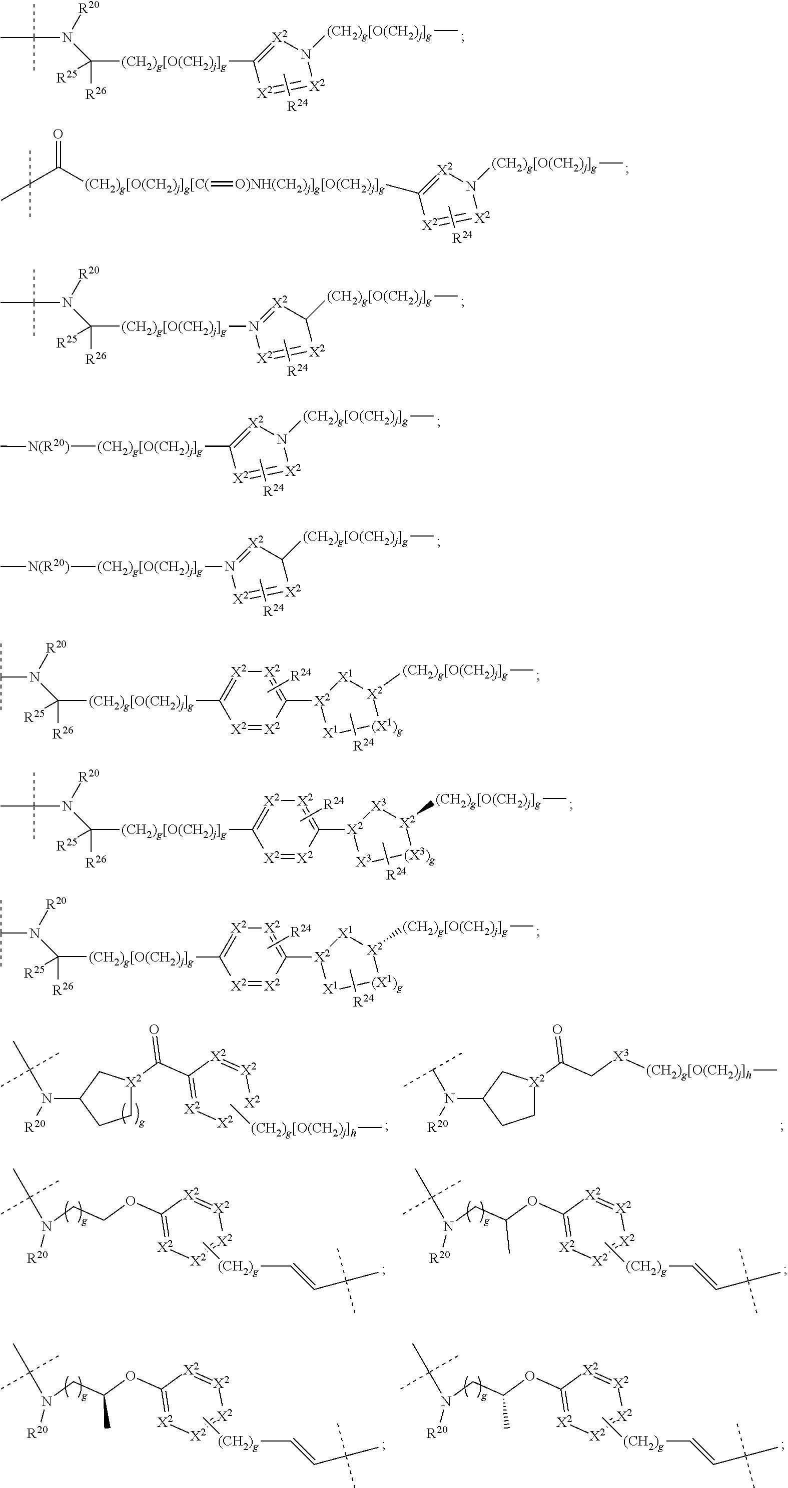 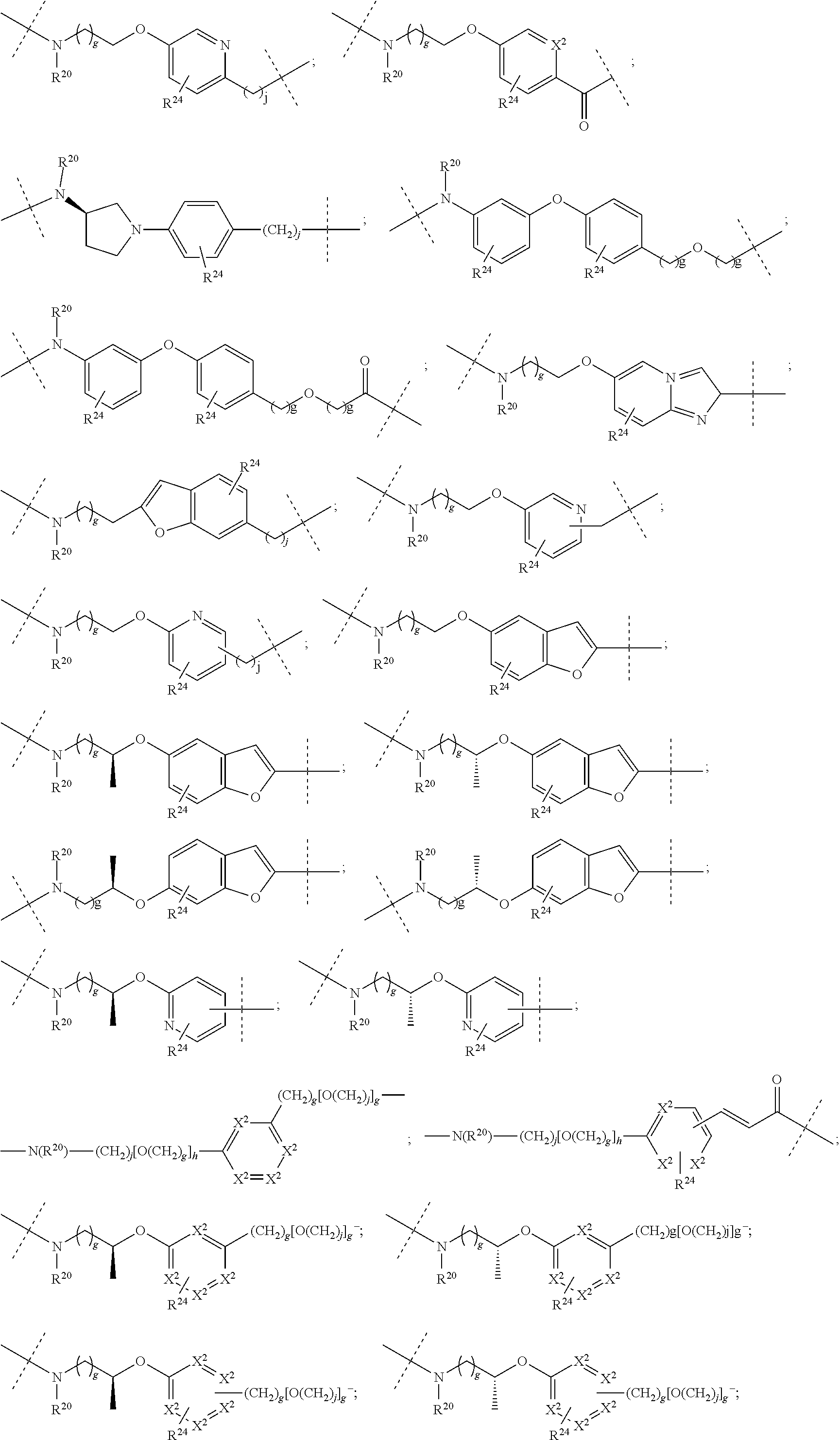 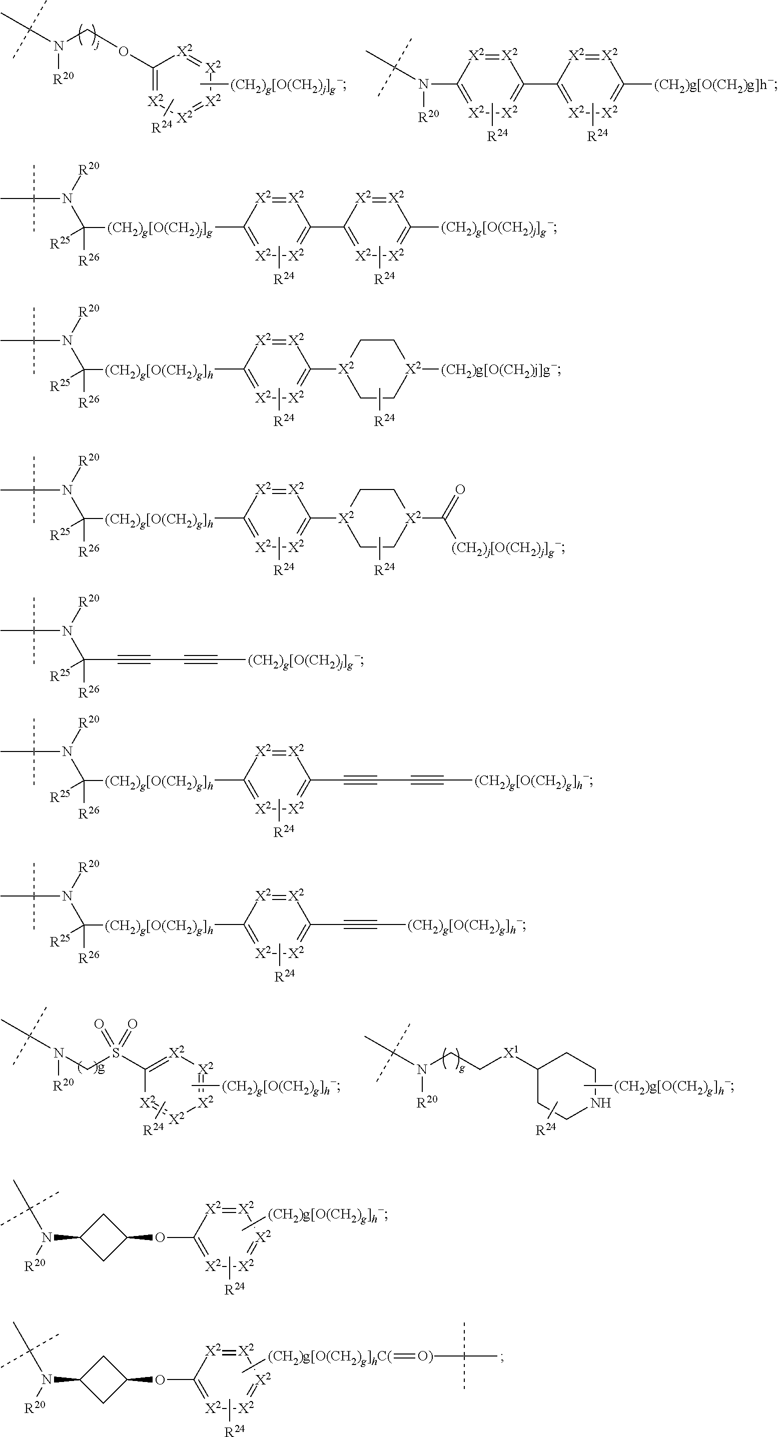 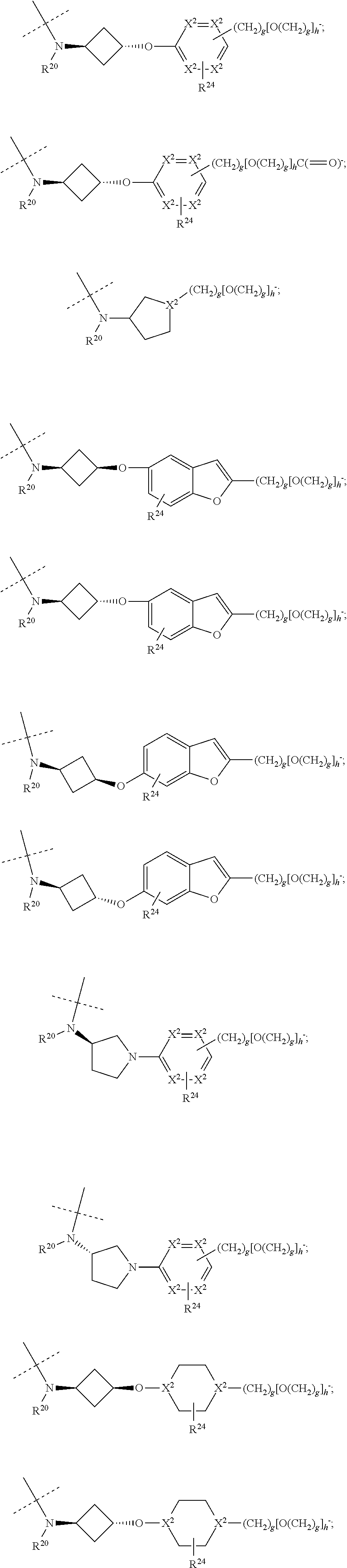 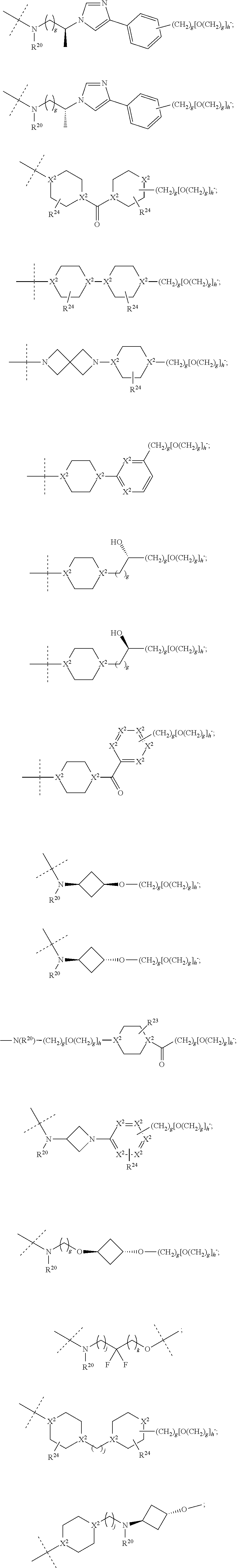 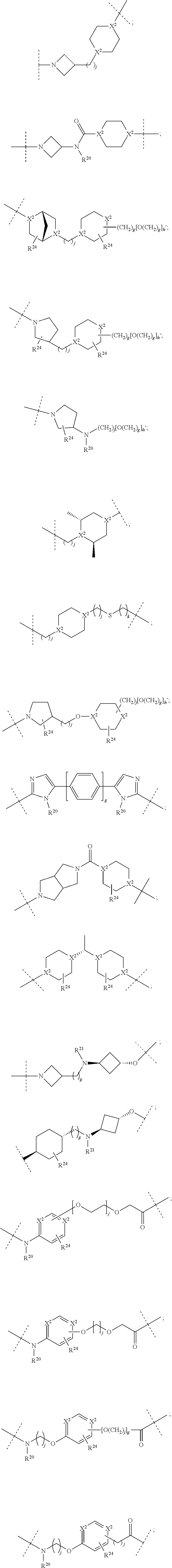 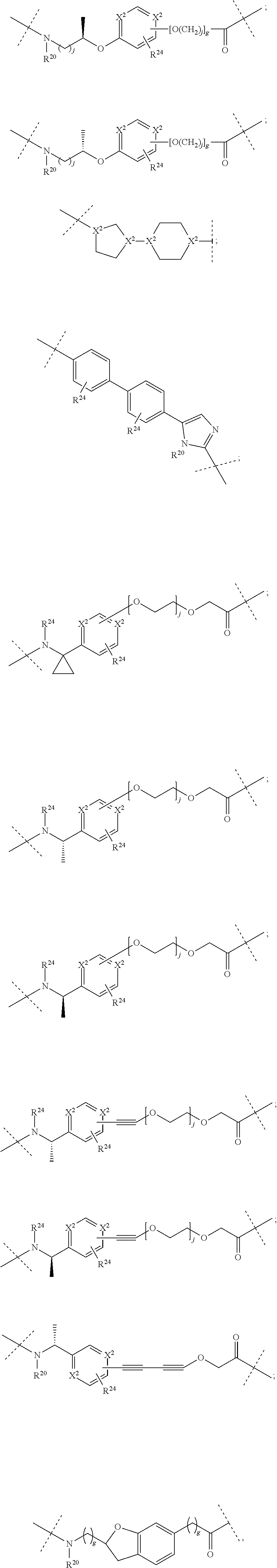
wherein
each R²⁰ is independently selected from hydrogen, C₁-C₄alkyl, and (C₃-C₆alkyl)C₀-C₂alkyl;
each R²¹ is independently selected from hydroxyl, halogen, C₁-C₄alkyl, and C₁-C₄alkoxy;
R²³ is 0 to 4 substituents independently selected from halogen, hydroxyl, amino, C₁-C₂alkyl, and C₁-C₂alkoxy;
R²⁴ is 0 to 4 substituents independently selected from halogen, hydroxyl, amino, cyano, C₁-C₄alkyl, C₁-C₄alkoxy, C₁-C₂haloalkyl, and C₁-C₂haloalkoxy;
each R²⁵ and R²⁶ is independently selected from hydrogen and methyl, or R²⁵ and R²⁶ are joined to form a spiro attached C₃-C₅cycloalkyl group, or R²⁵ and R²⁶ can be taken together along with the carbon atom to which they are bound form a 3- to 8-membered monocyclic or bicyclic cycloalkyl ring;
each X¹ is independently selected from CH₂, NR²⁰, O, SO, SO₂, and S;
each X² is independently selected from CH and N;
each X³ is independently selected from CH₂, NR²⁰, O, SO, SO₂, and S;
each g is independently selected from 0, 1, 2, 3, 4, 5, 6, 7, or 8;
each h is independently selected from 0, 1, 2, 3, 4, 5, 6, 7, or 8;
each j is independently selected from 1, 2, 3, 4, 5, 6, 7, or 8; and
each k is independently selected from 1, 2, 3, 4, 5, 6, 7, or 8.

50. The bifunctional compound, or pharmaceutically acceptable salt thereof, of claim 49 wherein the [LINKER] is

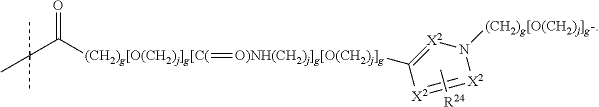

51. The bifunctional compound, or a pharmaceutically acceptable salt thereof, of any one of claims 39-48, wherein the [LINKER] is

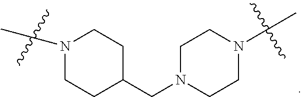 [SMILES: *N1CCC(CN2CCN(*)CC2)CC1]

52. The bifunctional compound, or a pharmaceutically acceptable salt thereof, of any one of claims 39-48, wherein the [LINKER] is a polyalkyleneglycol (e.g. polyethyleneglycol) containing linker having from 1 to 12 alkylene (e.g. ethylene) glycol residues.

53. The bifunctional compound, or a pharmaceutically acceptable salt thereof, of any one of claims 39-48, wherein the [LINKER] is a polypropylene glycol or polypropylene-co-polyethylene glycol linker containing any combination of the groups, and 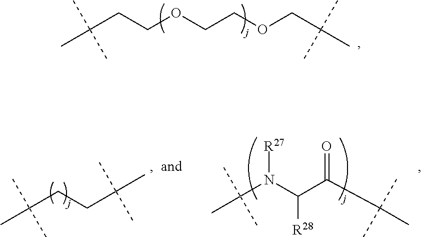
and optionally containing 1 group 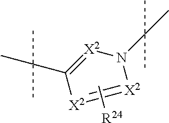
wherein
X² is independently chosen from N and CH;
R²⁴ is 0 to 4 substituents independently selected from halogen, hydroxyl, amino, cyano, C₁-C₄alkyl, C₁-C₄alkoxy, C₁-C₂haloalkyl, and C₁-C₂haloalkoxy;
R²⁷ is H, C₁-C₃ alkyl, C₁-C₃ alkanol, or forms a cyclic ring with R³ to form a pyrrolidine or hydroxypyrroline group;
R²⁸ is H or a side chain derived from a D- or L amino acid selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline, hydroxyproline, serine (methanol), threonine (ethanol, 1-hydroxyethane), tryptophan (methyleneindole), tyrosine (methylene phenol), or valine (isopropyl); and
j is 0, 1, 2, 3, 4, 5, 6, 7, 8, 9, 10, 11, 12, 13, 14, or 15.

54. The bifunctional compound, or a pharmaceutically acceptable salt thereof, of any one of claims 39-48, wherein the [LINKER] comprises: 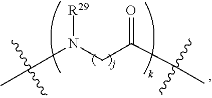
wherein
each R²⁹ is H or a C₁-C₃alkyl that is optionally substituted with one or two hydroxyl groups;
each j is independently 1, 2, 3, 4, 5, 6, 7, 8, 9, 10, 11, 12, 13, 14, or 15; and
k is an integer from 1 to 100.

55. The bifunctional compound, or a pharmaceutically acceptable salt thereof, of any one of claims 39-48, wherein the [LINKER] comprises: 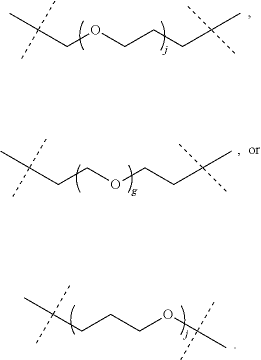
wherein
j is an integer from 1 to 25, 1 to 15, 1 to 12, 2 to 11, 2 to 10, 2 to 8, 2 to 6, 2 to 5, 2 to 4 or 2 to 3 (e.g. 1, 2, 3, 4, 5, 6, 7, or 8); and g is 0, 1, 2, 3, 4, 5, 6, 7, or 8.

56. The bifunctional compound, or a pharmaceutically acceptable salt thereof, of any one of claims 39-48, wherein the [LINKER] is:

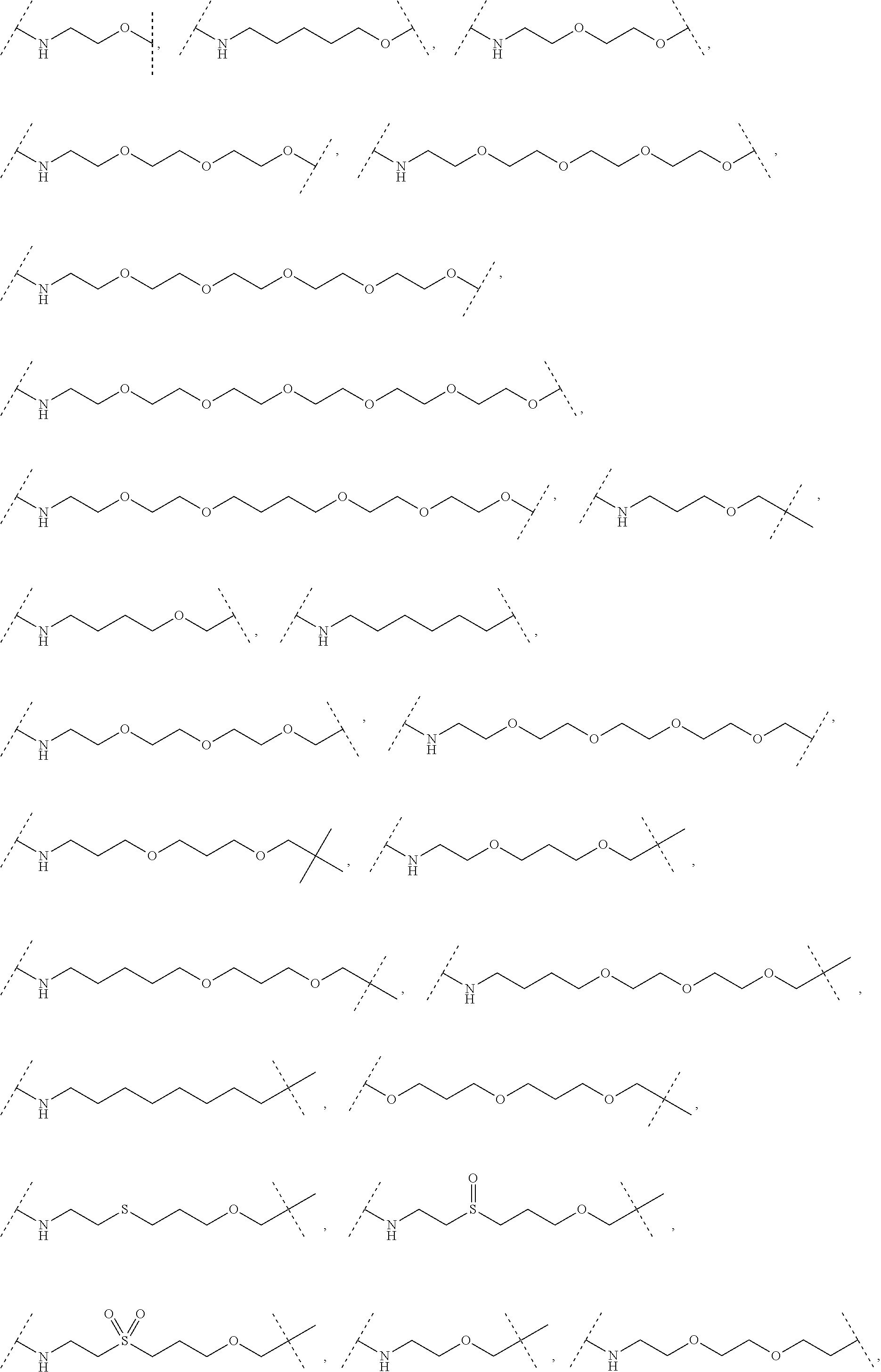

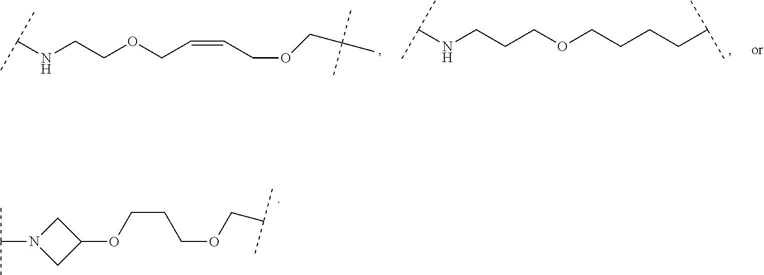

57. The bifunctional compound, or a pharmaceutically acceptable salt thereof, of any one of claims 39-48, where the [LINKER] is

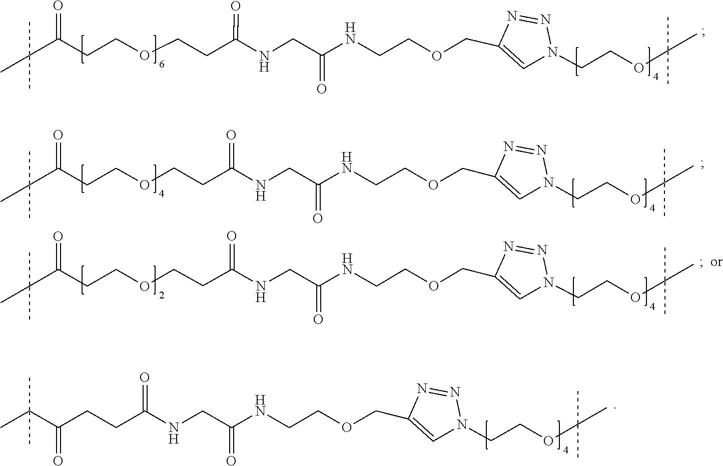

58. The bifunctional compound, or a pharmaceutically acceptable salt thereof, of any one of claims 39-57, wherein [CON] is a bond.

59. The bifunctional compound, or a pharmaceutically acceptable salt thereof, of any one of claims 39-57, wherein the [LINKER] is a C₁-C₅₀ alkylene group (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkylene), wherein:
the compound comprises one to fourteen —C₁-C₂₅ alkylene-[TNFABM] covalently bound to the [LINKER];
the compound comprises one to fourteen —C₁-C₂₅ alkylene-[MATE] covalently bound to the [LINKER];
each C1-C₂₅alkylene group of the [LINKER] optionally contains 0 or 1 or more double or triple bonds, each CH₂ group of the [LINKER] is optionally replaced with a group independently selected from O, S, SO, SO₂, NH, CH═CH, C≡C, 5-7 membered heterocycle, and 3-8 membered carbocycle, and is optionally bound to a [CON]; and
the [LINKER] is optionally substituted with one or more (e.g., 1, 2, 3, 4, 5, 6, 7, 8, 9, 10 or more) groups independently selected from halogen, hydroxyl, cyano, oxo (═O), amino, imino (═NH), —COOH, C₁-C₄alkyl, C₁-C₄haloalkyl, and C₁-C₄alkoxy.

60. The bifunctional compound, or a pharmaceutically acceptable salt thereof, of claim 59, wherein the compound comprises a[LINKER]—[CON] group in either orientation, which [LINKER]—[CON] group includes at least one [CON] comprising: 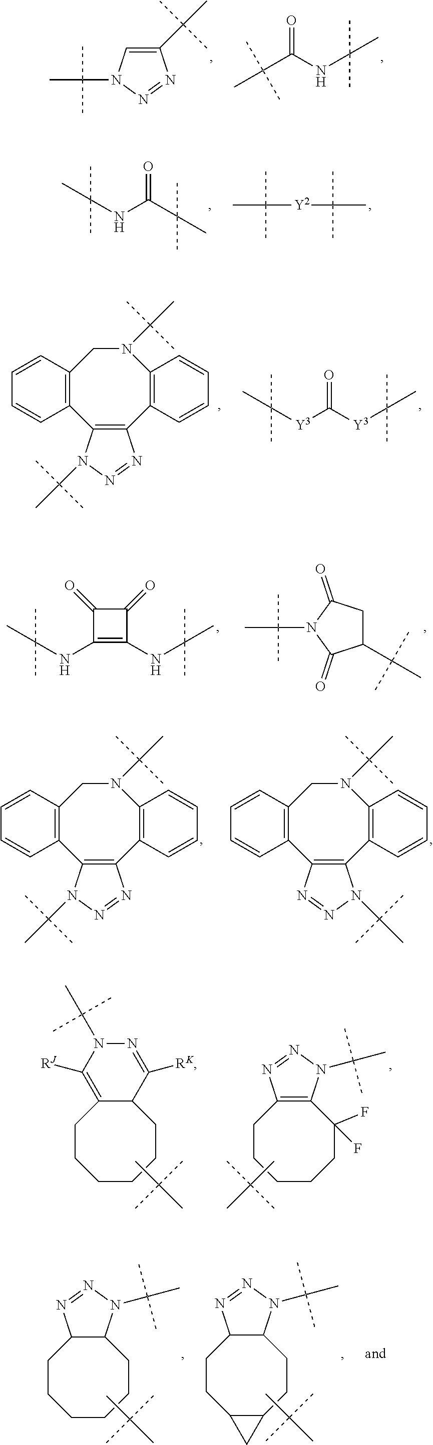 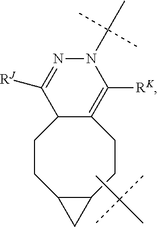
wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [MATE]), or a dioxo or diamide group according to the structure: 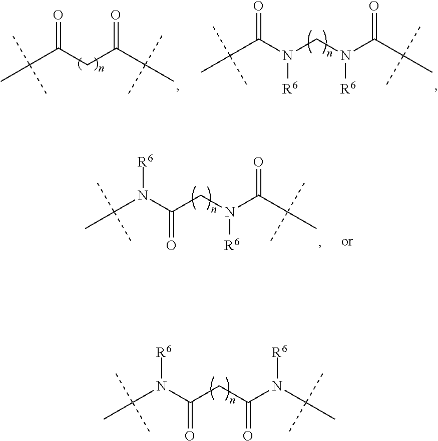
(e.g., one end is for attachment to [TNFABM], [LINKER], or [MATE]), wherein
each R⁶ is independently H or a C₁-C₃ alkyl group (preferably H);
n is an integer from 1 to 10 (n is preferably 1, 2, 3, 4, 5, or 6).

61. The bifunctional compound, or a pharmaceutically acceptable salt thereof, of any one of claims 39-57, wherein
(a) the compound comprises a [CON]-[LINKER] group in either orientation of the formula [C1]—[PAG], wherein
each [PAG] is a polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein the [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) 0 atoms of the [PAG] is optionally replaced with NH or NC₁-C₃alkyl; and
each [C1] is independently selected from: 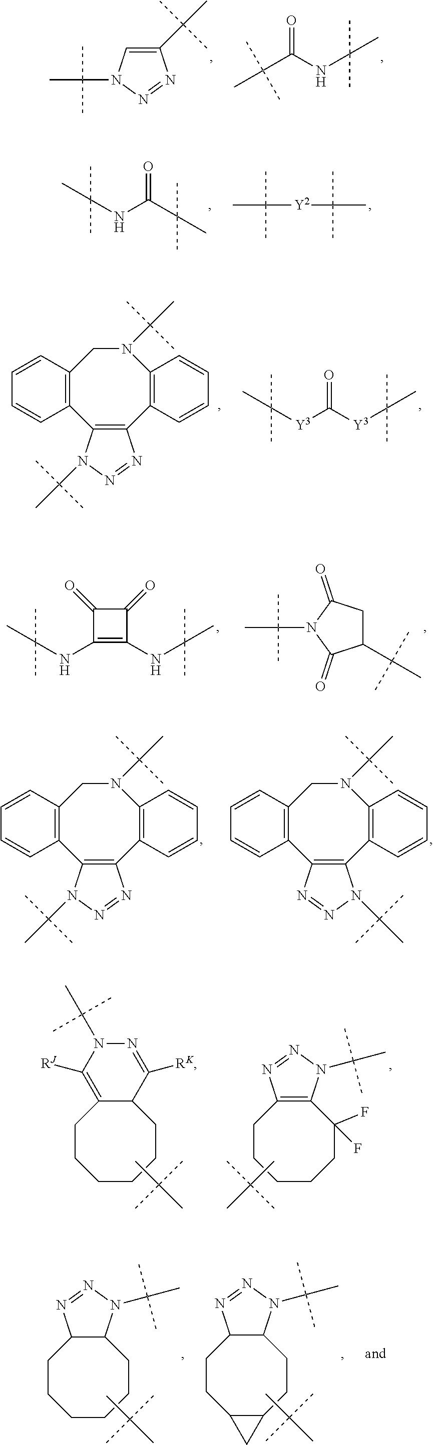 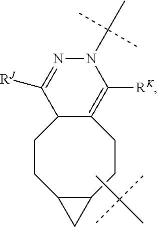
wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [MATE]), or a dioxo or diamide group according to the structure: 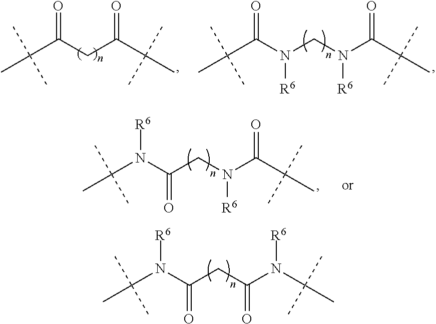
(e.g., one end is for attachment to the [TNFABM], [PAG], [LINKER], or [MATE]), wherein each R⁶ are independently H or a C₁-C₃ alkyl group (preferably H); and n is an integer from 1 to 10, or 1 to 8, or 1 to 6 (e.g. 1, 2, 3, 4, 5, or 6);
(b) the [CON]-[LINKER] group has the formula —CH₂(CH₂O—CH₂—[C1]—[PAG])₁, —CH₁(CH₂O—CH₂—[C1]—[PAG])₂, or —C(CH₂O—CH₂—[C1]—[PAG])₃, wherein
each [PAG] is an independently chosen polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein the [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) 0 atoms of the [PAG] is optionally replaced with NH or NC₁-C₃alkyl; and
each [C1] is independently selected from: 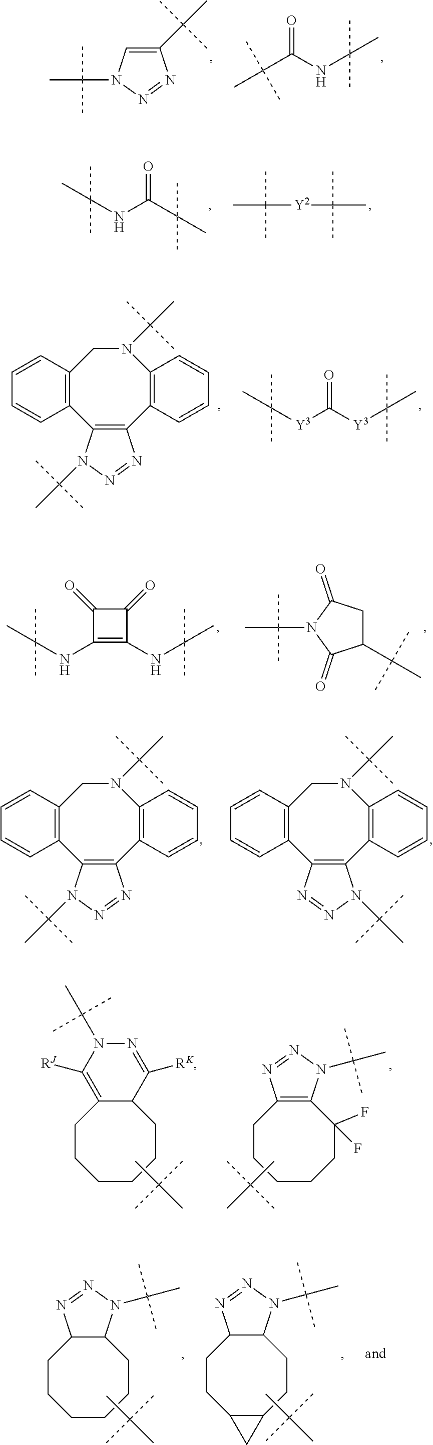 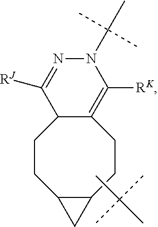
wherein
(c) the [CON] is: 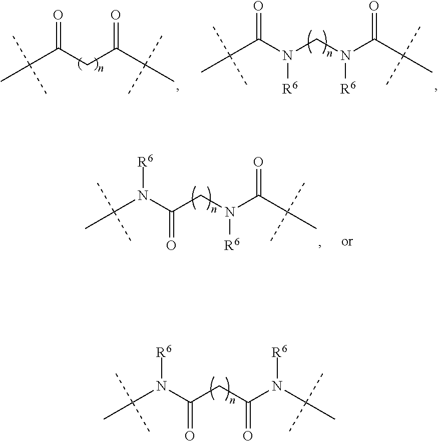
wherein
(d) the [CON] has formula: 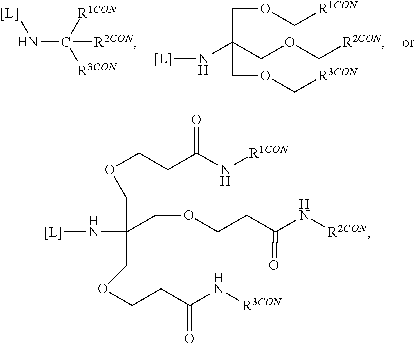
wherein
[L] represent the point of attachment to a first [LINKER]; and
R^{1CON}, R^{2CON}, and R^{3CON} are each independently H, a bond, or a chemical moiety, wherein the bond and the chemical moiety are covalently attached to an [MATE] or a second, third, or fourth independently chosen [LINKER] and at least one of R^{1CON}, R^{2CON}, and R^{3CON} least one of R^{1CON}, R^{2CON}, and R³CON is not H.

62. The bifunctional compound, or a pharmaceutically acceptable salt thereof, of claim 61, wherein
(a) one or more (e.g., 1, 2, or 3) of R^{1CON}, R^{2CON}, and R^{3CON} includes a group selected from: 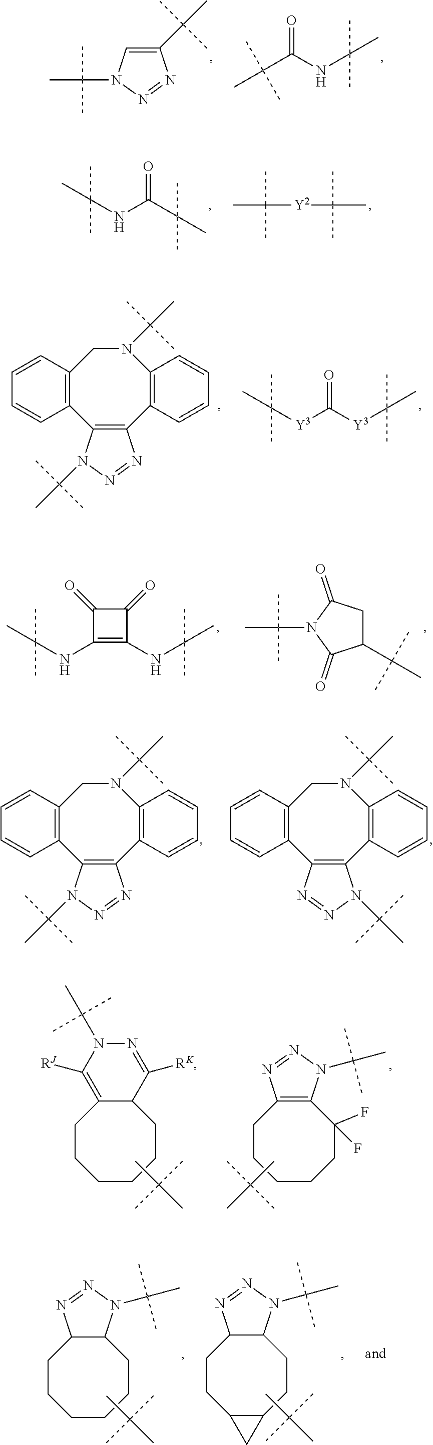 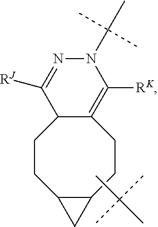
wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure: 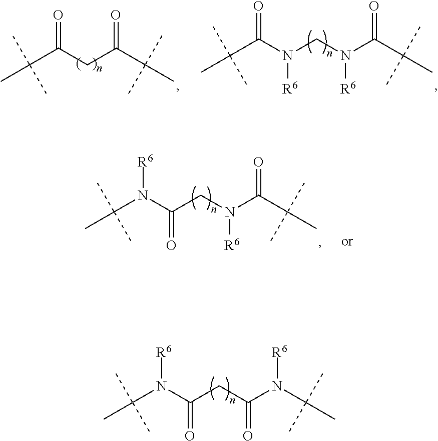
(e.g., one end is for attachment to [TNFABM], [LINKER], or [ASGPRBM]), wherein each R⁶ is independently H or C₁-C₃ alkyl (preferably H); and n is independently an integer from n to 10, or 1 to 8, 8 or 1 to 7 (e.g. 1, 2, 3, 4, 5, or 6).

63. The bifunctional compound or salt thereof, of claim 61, wherein
[CON] is 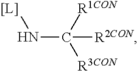
wherein
(a) at least one of R^{1CON}, R^{2CON}, and R^{3CON} is not H, and each of R^{1CON}, R^{2CON}, and R^{3CON} is independently selected from: H, bond, 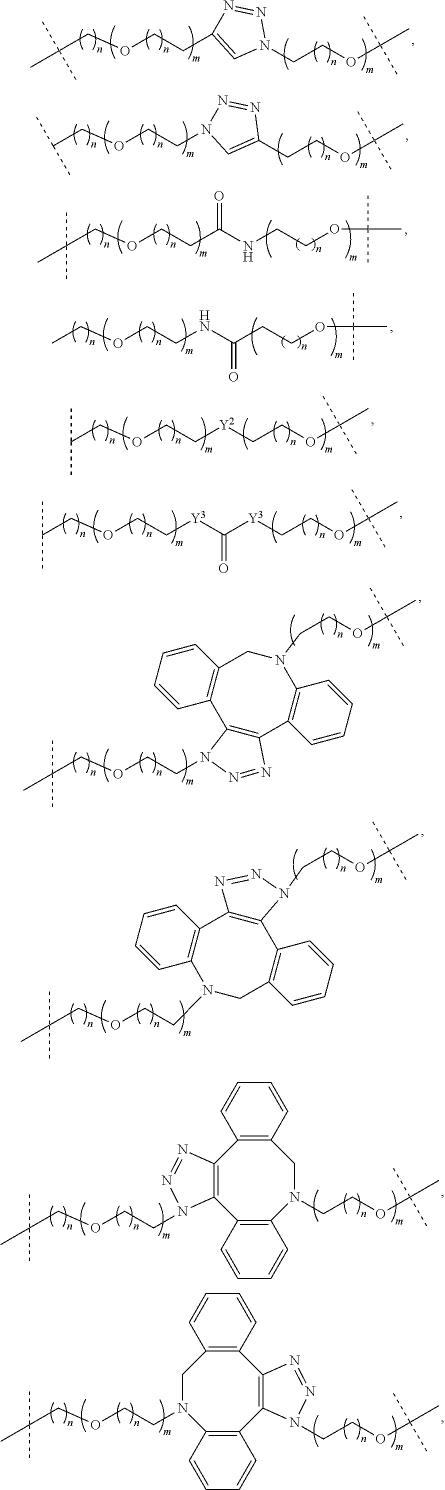 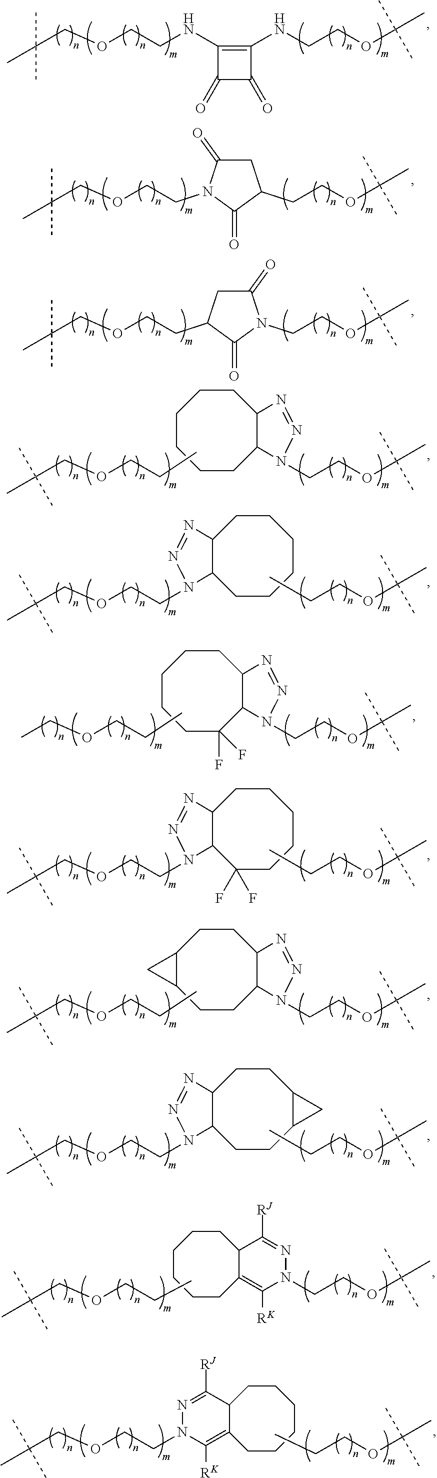 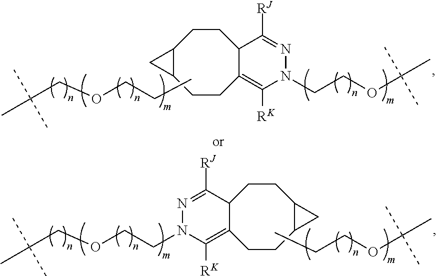
wherein
each n is independently 1, 2, 3, 4, 5, 6, 7, 8, 9, or 10;
each m is independently 0, 1, 2, 3, 4, 5, 6, 7, 8, 9, or 10;
each Y² is independently CH₂, O, S, NR⁷, C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [MATE]), or a dioxo or diamide group according to the structure: 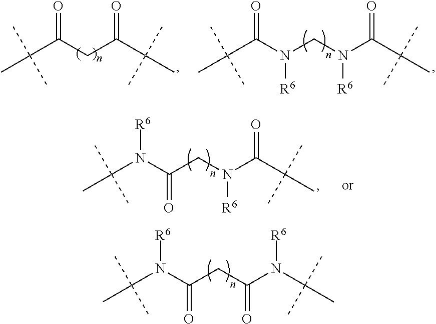
(e.g., one end is for attachment to [TNFABM], [LINKER], or [MATE]), wherein each R⁶ is independently H or a C₁-C₃ alkyl group (preferably H) n is an integer from 1 to 10 (is preferably and integer from 0 to 8, or 0 to 7, or 0, 1, 2, 3, 4, 5, or 6); or
(b) R^{1CON}, R^{2CON}, and R^{3CON} are each independently H, —(CH₂)ᵣ—, —(CH₂)ₛC(O)_{XA}(NR⁸)_{XA}—(CH₂)ₛ—, —(CH₂)ₛ(NR₄)_{XA}C(O)_{XA}—(CH₂)ₛ—, and —(CH₂)ₛO—(CH₂)ᵣ—C(O)NR⁸—, with the proviso that R^{1CON}, R^{2CON}, and R^{3CON} are not simultaneously H, wherein
each r is independently 1, 2, 3, or 4 (preferably 1 or 2);
each s is independently 0, 1, 2, 3, or 4 (preferably 0, 1 or 2);
each XA is independently 0 or 1; and
each R⁸ is independently H, C₁-C₃ alkyl, C₁-C₃ alkanol, or —C(O)(C₁-C₃), with the proviso that s and XA in a moiety are not all simultaneously 0.

64. The bifunctional compound, or a pharmaceutically acceptable salt thereof, of any one of claims 39-48, wherein
(a) the [LINKER]—[CON] has the formula: [PEG]-[C1]—[PEG],
wherein each [PEG] is independently a polyethylene glycol group containing from 1, 2, 3, 4, 5, 6, 7, 8, 9, 10, 11, or 12 ethylene glycol residues, and [C1] is a triazole group 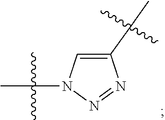 [SMILES: *c1cn(*)nn1]
(b) the [LINKER]—[CON] has the formula: [PAG]-[C1]—[PAG], wherein each [PAG] is independently a polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein each [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) O atoms of each [PAG] is optionally replaced with NH or NC₁-C₃alkyl; and
[C1] is selected from: 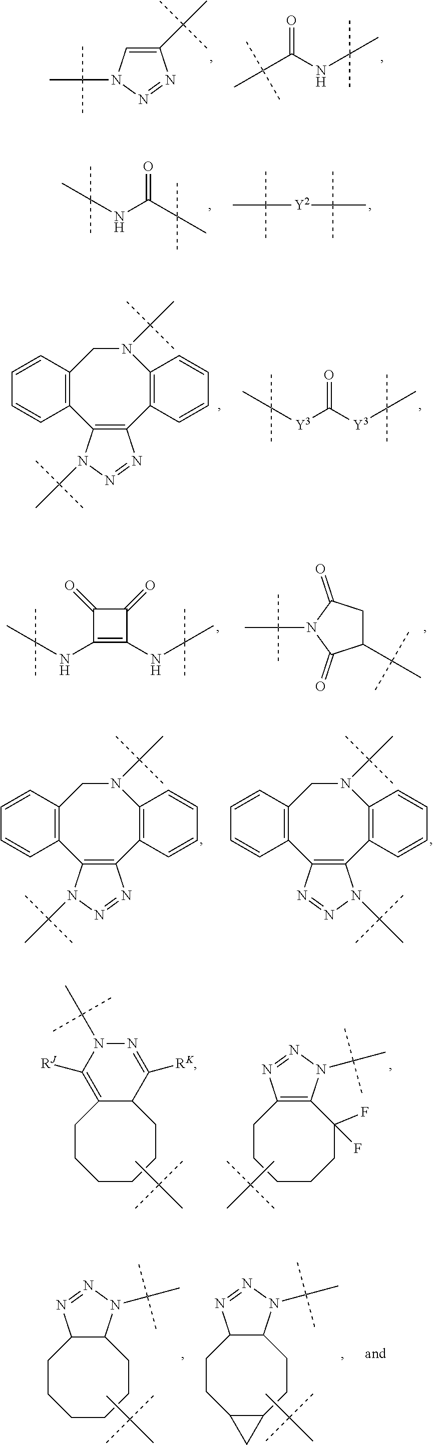 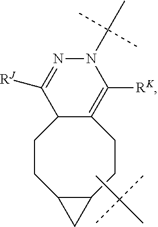
wherein
each Y² is independently CH₂, O, S, NR⁷ (e.g. NC₁-C₃alkyl)), C(O), S(O), S(O)₂, —S(O)₂^{o}, —OS(O)₂, or OS(O)₂^{o};
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [MATE]), or a dioxo or diamide group according to the structure: 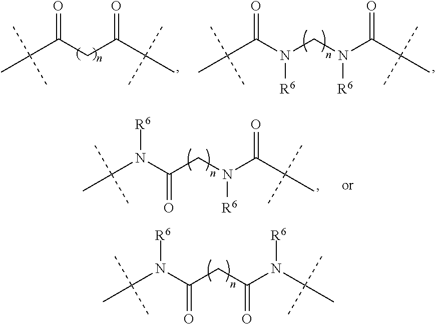
(e.g., one end is for attachment to [TNFABM], [LINKER], or [MATE]), wherein each R⁶ is independently H or a C₁-C₃ alkyl group (preferably H); and z is independently an integer from 0 to 8 or 1 to 7 (e.g. 1, 2, 3, 4, 5, or 6); or
(c) the [LINKER]—[CON] has the formula [PAG]-CH₂(CH₂O—CH₂—[C1]—[PAG])₁, [PAG]-CH₁(CH₂O—CH₂—[C1]—[PAG])₂, or [PAG]-C(CH₂O—CH₂—[C1]—[PAG])₃, wherein each [PAG] is a polyalkylene glycol (e.g., polyethylene glycol) group containing from 1, 2, 3, 4, 5, 6, 7, 8, 9, 10, 11, or 12 alkylene glycol (e.g., ethylene glycol) residues, wherein the [PAG] is optionally substituted with one or more (e.g. 1, 2, 3, 4, or 5) groups independently selected from hydroxyl, halogen, cyano, oxo, amino, C₁-C₃alkyl, and —COOH, and one or more (e.g., 1, 2, 3, 4, or 5) 0 atoms of the [PAG] is optionally replaced with NH or NC₁-C₃alkyl; and
each [C1] is independently selected from: 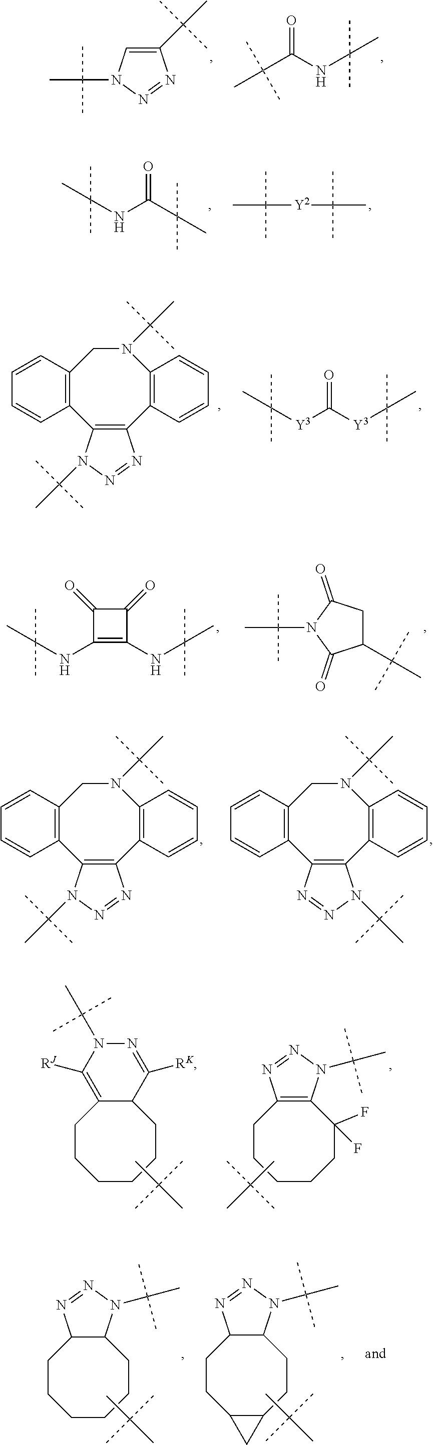 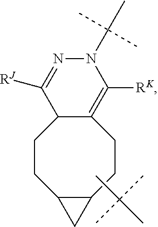
wherein
each Y² is independently CH₂, O, S, NR⁷ (e.g. NC₁-C₃alkyl)), C(O), S(O), S(O)₂, —S(O)₂O, —OS(O)₂, or OS(O)₂O;
each Y³ is independently O, S, or NR⁷;
each R⁷ is independently H, C₁-C₃ alkyl, C₁-C₃ alkanoyl, or —C(O)(C₁-C₃alkyl) group; and
R^{J} and R^{K} are each independently H, methyl, a bond (e.g., for attachment to [TNFABM], [LINKER], or [ASGPRBM]), or a dioxo or diamide group according to the structure: 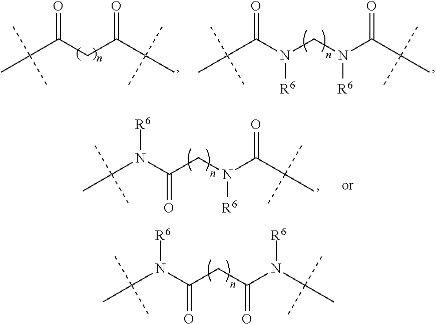
(e.g., one end is for attachment to [TNFABM], [LINKER], or [MATE])

65. The bifunctional compound, or a pharmaceutically acceptable salt thereof, of claim 1, wherein the bifunctional compound is selected from: 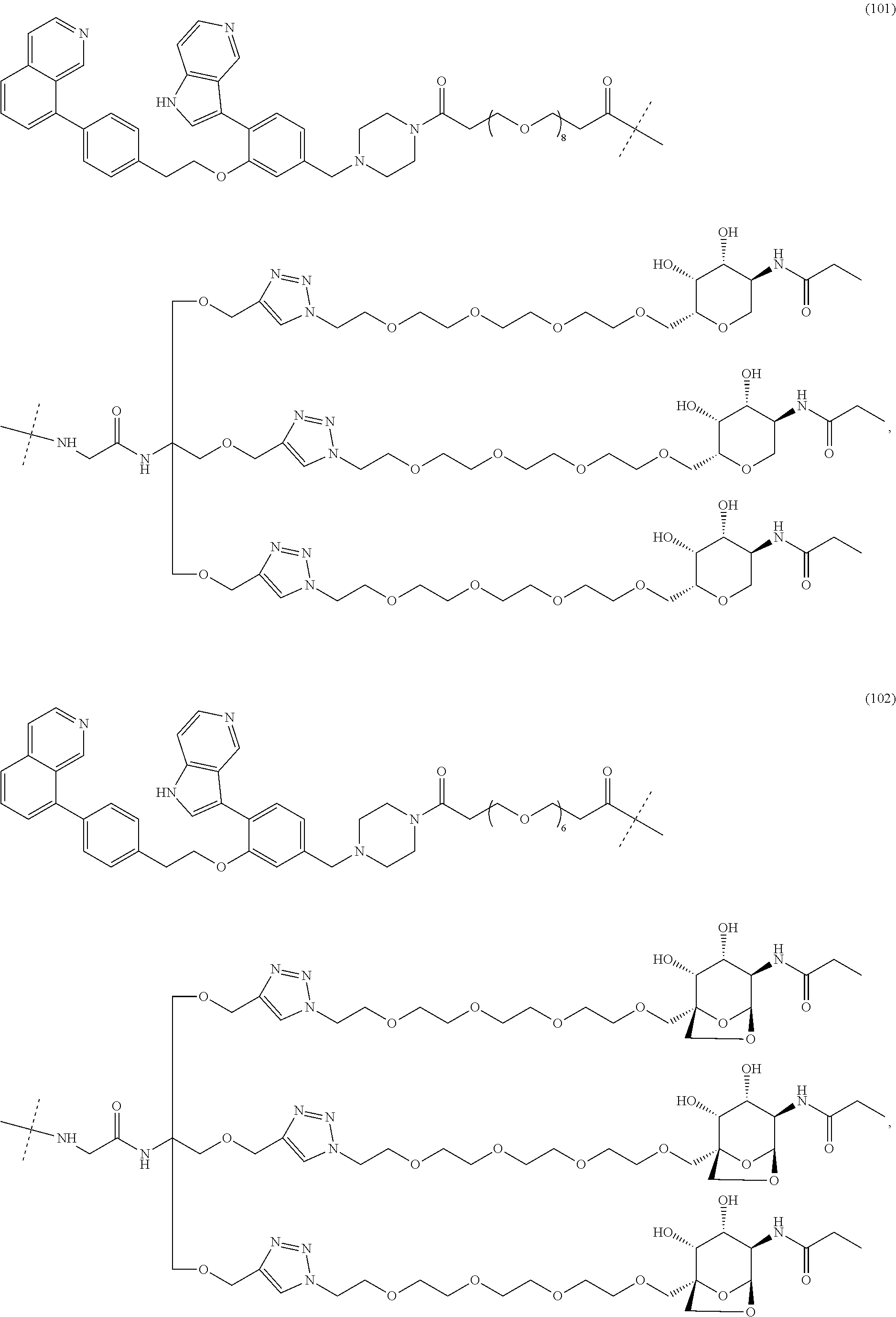 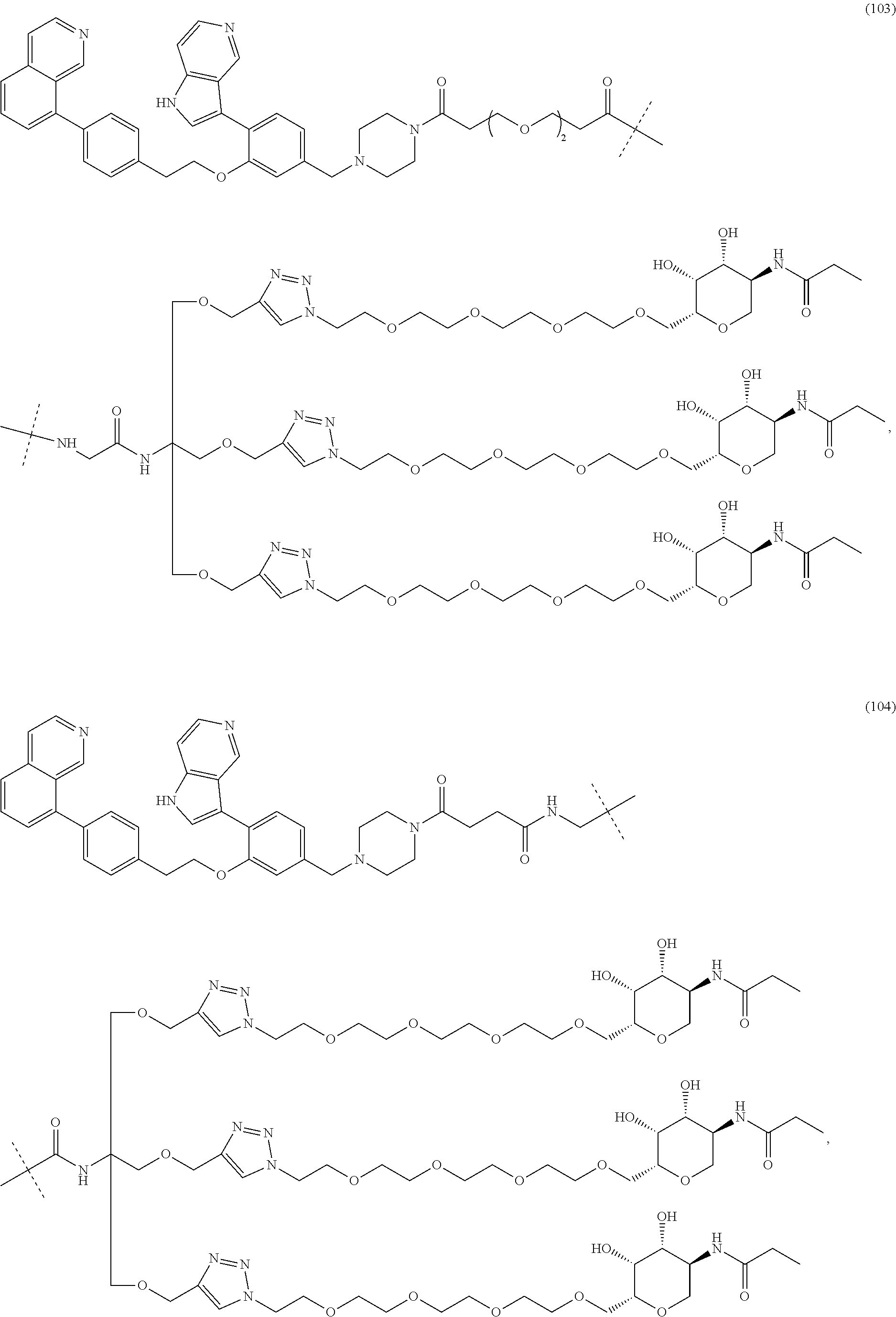 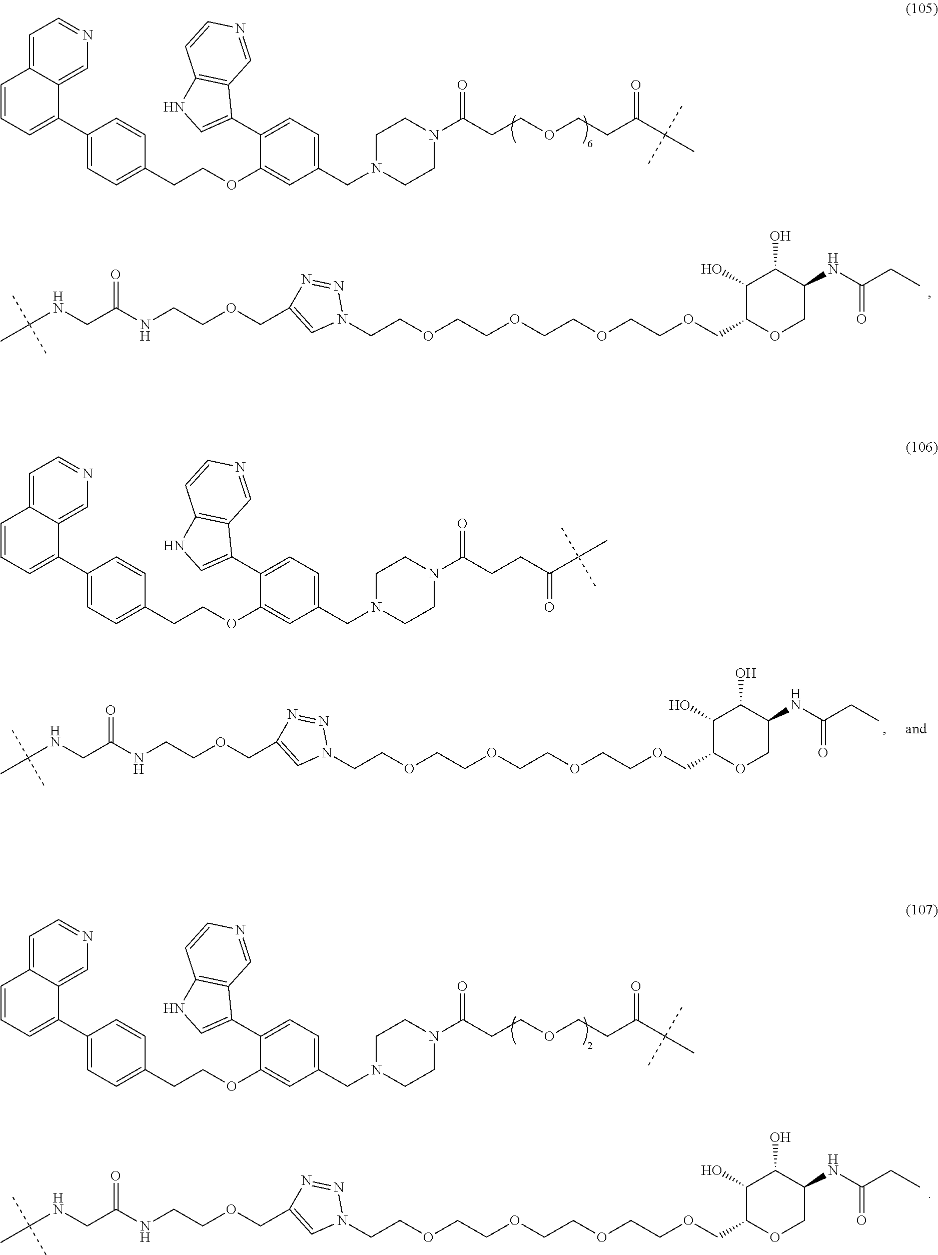
or a pharmaceutically acceptable salt thereof.

66. A pharmaceutical composition comprising the bifunctional compound of any one of claims 1-41, or a pharmaceutically acceptable salt thereof, and a pharmaceutically acceptable carrier or excipient.

67. A method of treating a disease or disorder responsive to TNF-α inhibition in a patient having a disease or disorder responsive to TNF-α inhibition, the method comprising administering a therapeutically effective amount of a compound of any one of claims 1-65, a pharmaceutically acceptable salt thereof, or the pharmaceutical composition of claim 36, to the patient.

68. The method of claim 67, wherein the disease or disorder is Alzheimer's disease, cancer, atherosclerosis, depression (such as, major depressive disorder), sepsis, diabetes, obesity, or autoimmune or inflammatory disease or disorder.

69. The method of claim 68, wherein the disease or disorder is a cancer selected from breast cancer, pancreatic cancer, ovarian cancer, endometrial cancer, oral cancer, gastric cancer, liver cancer, prostate cancer, bladder cancer, colorectal cancer, multiple myeloma, lymphoma, and leukemia.

70. The method of claim 67, wherein the disease or disorder in an autoimmune or inflammatory disease or disorder is selected from rheumatoid arthritis, psoriatic arthritis, inflammatory bowel disease (IBD), Crohn's disease, ulcerative colitis, psoriasis, and noninfectious or autoimmune uveitis.

71. The method of any one of claims 67-70-46, wherein the bifunctional compound is effective at ameliorating one or more symptoms of, or treating, the disease or disorder.

72. The method of any one of claims 67-71, wherein the method effectuates a decrease in the level of circulating TNF-α.

73. The method of claim 72, wherein the decrease in TNF-α is effected by internalizing the circulating TNF-α into a cell (e.g. hepatocyte) through an ASGPR.

74. The method of claim 73, wherein the internalized TNF-α is degraded.


SEQUENCE LISTING (1 sequence)

SEQ ID NO: 1 - protein, 388 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIA